(19) United States
(12) Patent Application Publication
(10) Pub. No.: US 2012/0094964 A1
(43) Pub. Date: Apr. 19, 2012

(54) SULFAMOYL BENZOIC ACID DERIVATIVES AS TRPM8 ANTAGONISTS

(76) Inventors: Tadashi Inoue, Aichi (JP); Masashi Ohmi, Aichi (JP); Kiyoshi Kawamura, Aichi (JP); Kazuo Ando, Aichi (JP); Yuji Shishido, Aichi (JP)

(21) Appl. No.: 13/318,212

(22) PCT Filed: May 6, 2010
(86) PCT No.: PCT/JP2010/003121

§ 371 Date: Dec. 15, 2011

Related U.S. Application Data
(60) Provisional application No. 61/213,049, filed on May 1, 2009.
(60) Provisional application No. 61/213,357, filed on Jun. 2, 2009.
(60) Provisional application No. 61/272,759, filed on Oct. 30, 2009.

(51) Int. Cl. A61K 31/635; A61K 31/18; A61K 31/495; A61K 31/4453; A61K 31/5377; A61K 31/444; A61K 31/4439; C07D 213/76; C07C 311/21; C07D 295/192; C07D 413/12; C07D 401/12; A61P 29/00; A61P 13/00; A61P 19/08; A61P 25/00; A61P 25/06; A61P 9/10; A61P 1/00; A61P 25/22; A61P 25/24; A61P 11/06; A61P 11/00; A61P 9/12; A61P 13/10; A61P 35/00; A61P 13/02; A61K 31/44

(52) U.S. Cl. 514/156; 514/352; 514/604; 514/255.01; 514/330; 514/237.2; 514/332; 514/343; 546/312; 564/92; 544/391; 546/226; 544/131; 546/264; 546/278.4

(57) ABSTRACT

The present invention relates to sulfamoyl benzoic acid derivatives of formula (I) or a pharmaceutically acceptable salt thereof, processes for their preparation, pharmaceutical compositions containing them and their use in the treatment of various disorders which are mediated via the TRPM8 receptor.

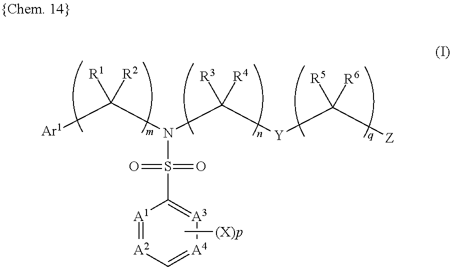

### TECHNICAL FIELD

This invention relates to sulfamoyl benzoic acid derivatives that act as modulators of the TRPM8 receptor. The present invention also relates to processes for the preparation of novel sulfamoyl benzoic acid derivatives and to their use in the treatment of a wide range of diseases, syndromes, and disorders, in particular for the treatment of inflammatory, pain and urological diseases or disorders.

### BACKGROUND ART

Transient receptor potential (TRP) channels are one of the largest groups of ion channels, and they are divided into 6 sub-families (TRPV, TRPM, TRPA, TRPC, TRPP and TRPML). TRP channels are cation-selecive channels that are activated by a variety of physical (e.g., temperature, osmolarity, mechanical) and chemical stimuli. TRPM8 is a member of TRP channel family. The receptor was cloned in 2002 (McKemy, D. D., et al., Nature 416, 52-58, 2002; Peier, A. D., Cell 108, 705-715, 2002) and it was found to be sensitive to cold temperature and menthol, and therefore named as cold menthol receptor-1 (CMR-1). TRPM8 can sense temperature changes in the range of both innocuous cold (15-28° C.) and noxious cold (<15° C.) as well as by chemical agents such as menthol and icilin.

TRPM8 is located on primary nociceptive neurons including A-delta and C-fibers and is also modulated by inflammation-mediated second messenger signals (Abe, J., et al Neurosci Lett 2006, 397(1-2), 140-144; Premkumar, L. S., et al, J. Neurosci, 2005, 25(49), 11322-11329). The localization of TRPM8 on both A-delta and C-fibers may provide a basis for abnormal cold sensitivity in pathologic conditions wherein these neurons are altered, resulting in pain, often of a burning nature (Kobayashi, K., et al, J Comp Neurol, 2005, 493(4), 596-606; Roza, C, et al. Pain, 2006, 120(1-2), 24-35; and Xing, H., et al, J Neurophysiol, 2006, 95(2), 1221-30). Gauchan et al. reported that the expression of TRPM8 in the primary afferents was increased in oxaliplatin-induced cold allodynia model in mice (Gauchan, P., et al. Neurosci Lett, 2009, 458, 93-95). Oxaliplatin, a third-generation platinum-based chemotherapy drug, induces serious sensory neurotoxicity in patients, which is aggravated by exposure to cold.

Cold intolerance and paradoxical burning sensations induced by chemical or thermal cooling closely parallel symptoms seen in a wide range of clinical disorders and thus provide a strong rationale for the development of TRPM8 modulators as novel antihyperalgesic or antiallodynic agents. TRPM8 is also known to be expressed in the brain, lung, bladder, gastrointestinal tract, blood vessels, prostate and immune cells, thereby providing the possibility for therapeutic modulation in a wide range of maladies.

International patent application WO 2006/040136 A1 from Bayer Healthcare AG purportedly describes substituted 4-benzyloxy-phenylmethylamide derivatives as cold menthol receptor-1 (CMR-1) antagonists for the treatment of urological disorders. International patent application WO 2006/040103 A1 from Bayer Healthcare AG purportedly describes methods and pharmaceutical compositions for treatment and/or prophylaxis of respiratory diseases or disorders. An international patent application, WO 2009/012430, describes sulfonamides for the treatment of diseases associated with the cold menthol receptor (CMR), also known as TRPM8.

### SUMMARY OF INVENTION

### Technical Problem

There is a need in the art for TRPM8 antagonists that can be used to treat a disease, syndrome, or condition in a mammal in which the disease, syndrome, or condition is affected by the modulation of TRPM8 receptors, such as chronic pain, neuropathic pain including cold allodynia and diabetic neuropathy, postoperative pain, osteoarthritis, rheumatoid arthritic pain, cancer pain, neuralgia, neuropathies, algesia, nerve injury, migraine, cluster and tension headaches, ischaemia, irritable bowel syndrome, neurodegeneration, fibromyalgia, stroke, itch, psychiatric disorders including anxiety and depression and inflammatory disorders such as asthma and chronic obstructive pulmonary, or airways, disease i.e., COPD, pulmonary hypertension, anxiety, including other stress-related disorders, urological diseases or disorders such as detrusor overactivity or overactive bladder, urinary incontinence, neurogenic detrusor overactivity or detrusor hyperflexia, idiopathic detrusor overactivity or detrusor instability, benign prostatic hyperplasia, and lower urinary tract symptoms, and combinations thereof.

TRPM8 antagonists should be well absorbed from the GI tract, be metabolically stable and possess favorable pharmacokinetic properties. They should be non-toxic. Furthermore, the ideal drug candidate will exist in a physical form that is stable, non-hygroscopic and easily formulated. In particular, it has been desired that compounds must bind potently to the TRPM8 receptor and show functional activity as antagonists. The present invention provides novel compounds which have excellent TRPM8 antagonistic activities.

### Solution to Problem

The compounds of the present invention differ structurally from the cited arts above by the presence of 5 to 7 heterocyclic group at Ar¹ in the formula I.

Then, WO 2009/025793 discloses sulfamoyl benzoic acid compounds. Some of the compounds are formally fallen into the claims in the patent. However, the compounds relate to human type 2 taste receptors for modulating taste perception, particularly bitter taste, which is quite different from TRPM8 receptor antagonist for the treatment of various disorders mediated via the TRPM8 receptor. Namely the sulfamoyl benzoic acid derivatives in the present invention are neither disclosed as working examples in the patent nor TRPM8 receptor antagonist activity which are useful for the treatment of various disorders mediated via the TRPM8 receptor.

The present invention provides a use of a compound of the following formula (I) for the manufacture of a medicament for the treatment of a condition or disorder mediated by TRPM8 receptor antagonistic activity

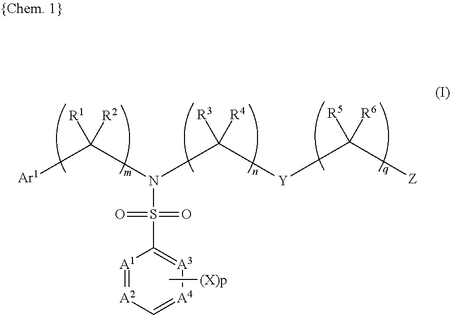

wherein
R¹, R², R³, R⁴, R⁵, and R⁶ are independently selected from the group consisting of hydrogen, C₁-C₄ alkyl, hydroxy C₁-C₄ alkyl, C₁-C₄ alkoxy C₁-C₄ alkyl, and C₃-C₇ cycloalkyl; or alternatively R¹ and R², together with the atom to which they are attached, form a 3 to 6 membered ring which may contain oxygen and/or nitrogen; said ring is optionally substituted with 1 to 4 substituents independently selected from the group consisting of halogen, hydroxy, C₁-C₄ alkyl, and C₁-C₄ alkoxy; R³ and R⁴, together with the atom to which they are attached, form a 3 to 6 membered ring which may contain oxygen and/or nitrogen; said ring is optionally substituted with 1 to 4 substituents independently selected from the group consisting of halogen, hydroxy, C₁-C₄ alkyl, and C₁-C₄ alkoxy; R⁵ and R⁶, together with the atom to which they are attached, form a 3 to 6 membered ring which may contain oxygen and/or nitrogen; said ring is optionally substituted with 1 to 4 substituents independently selected from the group consisting of halogen, hydroxy, C₁-C₄ alkyl, and C₁-C₄ alkoxy;
m is 0 or 1;
n is 0, 1, 2 or 3;
q is 0, 1, 2 or 3;
A¹, A², A³ and A⁴ are independently selected from nitrogen atom and carbon atom; wherein the number of nitrogen is up to two;
Z is H, Ar² or a substituent represented by the formula: R⁷N(R⁸)C(═O)—, in which
R⁷ and R⁸ are independently selected from hydrogen, C₁-C₄ alkyl, hydroxy C₁-C₄ alkyl, C₁-C₄ alkoxy C₁-C₄ alkyl, amino C₁-C₄ alkyl, C₁-C₄ alkylamino C₁-C₄ alkyl, di(C₁-C₄ alkyl)amino C₁-C₄ alkyl, 5 to 10 membered aryl, 5 to 10 membered aryl C₀-C₄ alkyl;

said aryl may be optionally substituted with 1 to 5 substituents independently selected from the group consisting of hydroxy, halogen, C₁-C₄ alkyl, C₁-C₄ alkoxy, hydroxy C₁-C₄ alkyl, amino C₁-C₄ alkyl, C₁-C₄ haloalkyl, C₁-C₄ haloalkoxy, C₃-C₈ cycloalkyl, amino, C₁-C₄ alkylamino, di(C₁-C₄ alkyl)amino, C₁-C₄ alkylthio, and nitro;
C₃-C₈ cycloalkyl, and 3 to 8 membered heterocyclyl C₁-C₄ alkyl;
   said heterocyclyl and alkyl may have independently 1 to 4 substituents independently selected from C₁-C₄ alkyl and halogen;
   or alternatively R⁷ and R⁸ together with nitrogen atom to which they are attached form a 4 to 8 membered ring which may contain nitrogen, oxygen or sulfur, wherein the 4 to 8 membered ring is optionally substituted with 1 to 6 substituents independently selected from the group consisting of hydroxy, C₁-C₄ alkyl, C₁-C₄ alkoxy, C₃-C₇ cycloalkyl, amino, oxo, C₁-C₄ alkylamino, and di(C₁-C₄ alkyl)amino;
Ar¹ is aryl, which may optionally be substituted with halogen, C₁-C₄ alkyl, C₁-C₄ haloalkyl, hydroxy, C₁-C₄ alkoxy, C₁-C₄ haloalkoxy, C₁-C₄ alkylthio, nitro, amino, C₁-C₄ alkylamino, di(C₁-C₄ alkyl)amino, cyano, hydroxy C₁-C₄ alkyl, C₁-C₄ alkoxy C₁-C₄ alkyl, C₁-C₄ alkylsulfonyl, aminosulfonyl, C₁-C₄ alkyl C(═O)—, HO(O═)C—, C₁-C₄ alkyl-O(O═)C—, R⁹N(R¹⁰)C(═O)—, C₁-C₄ alkylsulfonylamino, C₃-C₇ cycloalkyl, R⁹C(═O)N(R¹⁰)—, NH₂(HN═)C—, or 5 to 10 membered aryl C₀-C₄ alkyl; said aryl may be optionally substituted with 1 to 5 substituents independently selected from the group consisting of hydroxy, halogen, C₁-C₄ alkyl, C₁-C₄ alkoxy, hydroxy C₁-C₄ alkyl, amino C₁-C₄ alkyl, C₁-C₄ haloalkyl, C₁-C₄ haloalkoxy, C₃-C₇ cycloalkyl, amino, C₁-C₄ alkylamino, di(C₁-C₄ alkyl)amino, C₁-C₄ alkylthio, and nitro;
Ar² is aryl, which may optionally be substituted with halogen, C₁-C₄ alkyl, C₁-C₄ haloalkyl, hydroxy, C₁-C₄ alkoxy, C₁-C₄ haloalkoxy, C₁-C₄ alkylthio, nitro, C₁-C₄ alkylsilyl, di(C₁-C₄ alkyl)silyl, tri(C₁-C₄ alkyl)silyl, amino, C₁-C₄ alkylamino, di(C₁-C₄ alkyl)amino, cyano, hydroxy C₁-C₄ alkyl, C₁-C₄ alkoxy C₁-C₄ alkyl, C₁-C₄ alkylsulfonyl, aminosulfonyl, C₁-C₄ alkyl C(═O)—, HO(O═)C—, C₁-C₄ alkyl-O(O═)C—, R⁹N(R¹⁰)C(═O)—, C₁-C₄ alkylsulfonylamino, C₃-C₇ cycloalkyl, R⁹C(═O)N(R¹⁰)—, NH₂(HN═)C—, 5 to 10 membered aryloxy or 5 to 10 membered aryl C₀-C₄ alkyl; said aryloxy, aryl and C₃-C₇ cycloalkyl may be optionally substituted with 1 to 5 substituents independently selected from the group consisting of hydroxy, halogen, C₁-C₄ alkyl, C₁-C₄ alkoxy, hydroxy C₁-C₄ alkyl, amino C₁-C₄ alkyl, C₁-C₄ haloalkyl, C₁-C₄ haloalkoxy, C₃-C₇ cycloalkyl, cyano, amino, C₁-C₄ alkylamino, di(C₁-C₄ alkyl)amino, C₁-C₄ alkylthio, R⁹N(R¹⁰)C(═O)— and nitro;
R⁹ and R¹⁰ are independently selected from the definitions of R⁷ and R⁸;
X is independently selected from HO(O═)C—C₀-C₄alkyl, hydroxy, halogen, C₁-C₄ alkyl, C₁-C₄ alkoxy, hydroxy C₁-C₄ alkyl, amino C₁-C₄ alkyl, C₁-C₄ haloalkyl, C₁-C₄ haloalkoxy, C₃-C₇ cycloalkyl, cyano, amino, C₁-C₄ alkylamino, di(C₁-C₄ alkyl)amino, C₁-C₄ alkylthio, nitro, alkylsulfonyl, aminosulfonyl, C₁-C₄ alkyl C(═O)—, C₁-C₄ alkyl-O(O═)C—, C₁-C₄ alkylsulfonylamino, C₁-C₄ alkylsulfonylaminoalkyl, C₃-C₇ cycloalkyl, R¹¹C(═O)N(R¹²)—, R¹¹C(═O)N(R¹²)C₁-C₄alkyl, R¹¹N(R¹²)SO₂N(R¹³)C₀-C₄alkyl, R¹¹N(R¹²)C(═O)N(R¹³)C₀-C₄alkyl, NH₂(HN═)C—, C₃-C₇ cycloalkyl, 3 to 7 membered heterocyclyl, and 5 to 10 membered aryl C₀-C₄ alkyl; said heterocyclyl and alkyl may have independently 1 to 4 substituents independently selected from C₁-C₄ alkyl and halogen;
R¹¹, R¹² and R¹³ are independently selected from the definitions of R⁷ and R⁸;
p is 1, 2, 3, 4 or 5; when p is two or more than two, X may be same or different;
Y is a chemical bond, oxygen atom, sulfur atom, or nitrogen atom; when Y is oxygen atom, sulfur atom, or nitrogen atom, said substituent Y may have a substituent independently selected from the definitions of R⁷ and R⁸;
   or a pharmaceutically acceptable salt thereof, each as described herein, for the manufacture of a medicament for the treatment of a condition or disorder mediated by TRPM8 receptor activity; in particular, TRPM8 antagonistic activity. In order to use the compounds of formula (I) and pharmaceutically acceptable salts thereof in therapy, they will normally be formulated into a pharmaceutical composition in accordance with standard pharmaceutical practice.

The present invention also provides a pharmaceutical composition, which comprises a compound of formula (I) or a pharmaceutically acceptable salt thereof, and a pharmaceutically acceptable carrier or excipient.

The present invention provides a compound as described in formula (I) wherein the definition described above: m is 0, or a pharmaceutically acceptable salt thereof.

Preferable compounds of the invention are represented by formula (I) wherein the definition described above: m is 0; and Ar¹ is a 5 to 7 heterocyclic group.

More preferable compounds of the invention are represented by formula (I) wherein the definition described above: m is 0; and Ar¹ is a 5 to 7 heterocyclic group selected from pyridinyl, pyrimidinyl, pyridazinyl, and triazinyl.

The most preferable compounds of the invention are represented by formula (I), wherein m is 0; Ar¹ is 2-pyridinyl or 3-pyridinyl; and A¹, A², A³ and A⁴ are carbon atom.

Suitable individual compounds of the invention are:
N-Benzyl-N-(3-chloro-5-(trifluoromethyl)pyridin-2-yl)benzenesulfonamide;
4-(N-Benzyl-N-(3-chloro-5-(trifluoromethyl)pyridin-2-yl)sulfamoyl)benzamide;
4-(N-Benzyl-N-(3-chloro-5-(trifluoromethyl)pyridin-2-yl)sulfamoyl)-N-methylbenzamide;
4-(N-Benzyl-N-(3-chloro-5-(trifluoromethyl)pyridin-2-yl)sulfamoyl)-N,N-dimethylbenzamide;
4-(N-Benzyl-N-(3-chloro-5-(trifluoromethyl)pyridin-2-yl)sulfamoyl)-N-(2-hydroxyethyl)benzamide;
4-(N-(3-Chloro-5-(trifluoromethyl)pyridin-2-yl)-N-(4-(trifluoromethoxy)benzyl)sulfamoyl)benzoic acid;
4-(N-(3-Chloro-5-(trifluoromethyl)pyridin-2-yl)-N-(4-methoxybenzyl)sulfamoyl)benzoic acid;
4-(N-(3-Chloro-5-(trifluoromethyl)pyridin-2-yl)-N-(4-fluorobenzyl)sulfamoyl)benzoic acid;
4-(N-(4-tert-Butylbenzyl)-N-(3-chloro-5-(trifluoromethyl)pyridin-2-yl)sulfamoyl)benzoic acid;
4-(N-(3-Chloro-5-(trifluoromethyl)pyridin-2-yl)-N-(2-cyclohexylethyl)sulfamoyl)benzoic acid;
4-(N-(3-Chloro-5-(trifluoromethyl)pyridin-2-yl)-N-(2-fluorobenzyl)sulfamoyl)benzoic acid;
4-(N-(3-Chloro-5-(trifluoromethyl)pyridin-2-yl)-N-(3-(trifluoromethyl)benzyl)sulfamoyl)benzoic acid;
4-(N-(3-Chloro-5-(trifluoromethyl)pyridin-2-yl)-N-(3-methoxybenzyl)sulfamoyl)benzoic acid;
4-(N-(3-Chloro-5-(trifluoromethyl)pyridin-2-yl)-N-(3-(trifluoromethoxy)benzyl)sulfamoyl)benzoic acid;
4-(N-(3-Chloro-5-(trifluoromethyl)pyridin-2-yl)-N-(2,4-difluorobenzyl)sulfamoyl)benzoic acid;
4-(N-(3-Chloro-5-(trifluoromethyl)pyridin-2-yl)-N-(4-isopropylbenzyl)sulfamoyl)benzoic acid;
4-(N-(3-Chloro-5-(trifluoromethyl)pyridin-2-yl)-N-(2-(4-fluorophenoxy)ethyl)sulfamoyl)benzoic acid;
4-(N-(3-Chloro-5-(trifluoromethyl)pyridin-2-yl)-N-(2-(trifluoromethyl)benzyl)sulfamoyl)benzoic acid;
4-(N-(3-Chloro-5-(trifluoromethyl)pyridin-2-yl)-N-(4-chlorobenzyl)sulfamoyl)benzoic acid;
4-(N-(3-Chloro-5-(trifluoromethyl)pyridin-2-yl)-N-(4-cyanobenzyl)sulfamoyl)benzoic acid;
4-(N-Benzyl-N-(3,5-dichloropyridin-2-yl)sulfamoyl)benzoic acid;
4-(N-Benzyl-N-(5-chloro-3-methylpyridin-2-yl)sulfamoyl)benzoic acid;
4-(N-(3-Chloro-5-(trifluoromethyl)pyridin-2-yl)-N-(4-(trifluoromethyl)benzyl)sulfamoyl)benzoic acid;
4-(N-(2-Chloro-4-fluorobenzyl)-N-(3-chloro-5-(trifluoromethyl)pyridin-2-yl)sulfamoyl)benzoic acid;
4-(N-(3-Chloro-5-(trifluoromethyl)pyridin-2-yl)-N-(4-(2,2,2-trifluoroethoxy)benzyl)sulfamoyl)benzoic acid;
4-(N-(3-Chloro-5-(trifluoromethyl)pyridin-2-yl)-N-(3,5-difluorobenzyl)sulfamoyl)benzoic acid;
4-(N-(3-Chloro-5-(trifluoromethyl)pyridin-2-yl)-N-(4-fluoro-3-(trifluoromethyl)benzyl)sulfamoyl)benzoic acid;
4-(N-(3-Chloro-5-(trifluoromethyl)pyridin-2-yl)-N-(3,4-difluorobenzyl)sulfamoyl)benzoic acid;
4-(N-(3-Chloro-5-(trifluoromethyl)pyridin-2-yl)-N-(2,5-difluorobenzyl)sulfamoyl)benzoic acid;
4-(N-(3-Chloro-5-(trifluoromethyl)pyridin-2-yl)-N-(3,4-dichlorobenzyl)sulfamoyl)benzoic acid;
4-(N-(3-Chloro-5-(trifluoromethyl)pyridin-2-yl)-N-(3-chlorobenzyl)sulfamoyl)benzoic acid;
4-(N-(3-Chloro-5-(trifluoromethyl)pyridin-2-yl)-N-(4-(1-methylcyclopropyl)benzyl)sulfamoyl)benzoic acid;
4-(N-(3-Chloro-5-(trifluoromethyl)pyridin-2-yl)-N-(4-(1,1,1-trifluoro-2-methylpropan-2-yl)benz yl)sulfamoyl)benzoic acid;
4-(N-(3-Chloro-5-(trifluoromethyl)pyridin-2-yl)-N-(2-(trifluoromethoxy)benzyl)sulfamoyl)benzoic acid;
4-(N-(3,5-Dichloropyridin-2-yl)-N-(4-(trifluoromethyl)benzyl)sulfamoyl)benzoic acid;
4-(N-(5-Chloro-3-methylpyridin-2-yl)-N-(4-(trifluoromethyl)benzyl)sulfamoyl)benzoic acid;
4-(N-Benzyl-N-(2-chloro-4-(trifluoromethyl)phenyl)sulfamoyl)benzoic acid;
4-(N-(3-Chloro-5-(trifluoromethyl)pyridin-2-yl)-N-(3-phenylpropyl)sulfamoyl)benzoic acid;
4-(N-(3,5-Dichloropyridin-2-yl)-N-(4-fluoro-3-(trifluoromethyl)benzyl)sulfamoyl)benzoic acid;
4-(N-(3,5-Dichloropyridin-2-yl)-N-(4-fluorobenzyl)sulfamoyl)benzoic acid;
4-(N-(4-Chloro-3-fluorobenzyl)-N-(3,5-dichloropyridin-2-yl)sulfamoyl)benzoic acid;
4-(N-(3,5-Dichloropyridin-2-yl)-N-(4-(trifluoromethoxy)benzyl)sulfamoyl)benzoic acid;
4-(N-(3,5-Dichloropyridin-2-yl)-N-(2-(trifluoromethyl)benzyl)sulfamoyl)benzoic acid;
4-(N-(4-Chloro-3-fluorobenzyl)-N-(3-chloro-5-(trifluoromethyl)pyridin-2-yl)sulfamoyl)benzoic acid;
4-(N-(4-Chloro-3-(trifluoromethyl)benzyl)-N-(3,5-dichloropyridin-2-yl)sulfamoyl)benzoic acid;
4-(N-(3,5-Dichloropyridin-2-yl)-N-(3-fluoro-4-methylbenzyl)sulfamoyl)benzoic acid;
4-(N-(3,5-Dichloropyridin-2-yl)-N-(4-methyl-3-(trifluoromethyl)benzyl)sulfamoyl)benzoic acid;
4-(N-(3-Chloro-4-fluorobenzyl)-N-(3,5-dichloropyridin-2-yl)sulfamoyl)benzoic acid;
4-(N-(3,5-Dichloropyridin-2-yl)-N-(4-(1-methylcyclopropyl)benzyl)sulfamoyl)benzoic acid;
4-(N-(3-Chloro-4-fluorobenzyl)-N-(3-chloro-5-(trifluoromethyl)pyridin-2-yl)sulfamoyl)benzoic acid;
4-(N-(4-Chloro-2-(trifluoromethyl)benzyl)-N-(3,5-dichloropyridin-2-yl)sulfamoyl)benzoic acid;
4-(N-(4-Chloro-2-(trifluoromethyl)benzyl)-N-(5-chloro-3-methylpyridin-2-yl)sulfamoyl)benzoic acid;
4-(N-(5-Chloro-3-methylpyridin-2-yl)-N-(4-fluoro-3-(trifluoromethyl)benzyl)sulfamoyl)benzoic acid;
4-(N-(5-Chloro-3-methylpyridin-2-yl)-N-(3-chloro-4-fluorobenzyl)sulfamoyl)benzoic acid;
4-(N-(5-Chloro-3-methylpyridin-2-yl)-N-(4-(trifluoromethoxy)benzyl)sulfamoyl)benzoic acid;
4-(N-(4-Chloro-3-(trifluoromethyl)benzyl)-N-(5-chloro-3-methylpyridin-2-yl)sulfamoyl)benzoic acid;
4-(N-(3-Chloro-5-(trifluoromethyl)pyridin-2-yl)-N-(4-(trimethylsilyl)benzyl)sulfamoyl)benzoic acid;
4-(N-(3-Chloro-5-(trifluoromethyl)pyridin-2-yl)-N-(4-(1-cyanocyclopropyl)benzyl)sulfamoyl)benzoic acid;
4-(N-(3-Chloro-5-(trifluoromethyl)pyridin-2-yl)-N-((1-(pyridin-2-yl)piperidin-4-yl)methyl)sulfamoyl)benzoic acid;
4-(N-Benzyl-N-(3-chloro-5-(trifluoromethyl)pyridin-2-yl)sulfamoyl)-2-methylbenzoic acid;
N-Benzyl-N-(3-chloro-5-(trifluoromethyl)pyridin-2-yl)-6-hydroxypyridine-3-sulfonamide;
4-(N-Benzyl-N-(3-chloro-5-(trifluoromethyl)pyridin-2-yl)sulfamoyl)-3-methylbenzoic acid;
4-(N-Benzyl-N-(3-chloro-5-(trifluoromethyl)pyridin-2-yl)sulfamoyl)-3-methoxybenzoic acid;
2-(4-(N-Benzyl-N-(3-chloro-5-(trifluoromethyl)pyridin-2-yl)sulfamoyl)phenyl)acetic acid;
4-(N-(3-Chloro-5-(trifluoromethyl)pyridin-2-yl)-N-phenethylsulfamoyl)benzoic acid;
4-(N-Benzyl-N-(3-cyclopropyl-5-(trifluoromethyl)pyridin-2-yl)sulfamoyl)benzoic acid;
4-(N-(3-Chloro-5-(trifluoromethyl)pyridin-2-yl)-N-(4-cyclopropylbenzyl)sulfamoyl)benzoic acid;
4-(N-Benzyl-N-(3-bromo-5-(trifluoromethyl)pyridin-2-yl)sulfamoyl)benzoic acid;
4-(N-(3-Chloro-5-(trifluoromethyl)pyridin-2-yl)-N-(4-(1-methyl-1H-pyrazol-4-yl)benzyl)sulfamoyl)benzoic acid;
4-(N-Benzyl-N-(3-methyl-5-(trifluoromethyl)pyridin-2-yl)sulfamoyl)benzoic acid;
4-(N-(3-Chloro-5-(trifluoromethyl)pyridin-2-yl)-N-(4-(pyridin-3-yl)benzyl)sulfamoyl)benzoic acid;
4-(N-(3-Chloro-5-(trifluoromethyl)pyridin-2-yl)-N-(4-(thiophen-2-yl)benzyl)sulfamoyl)benzoic acid;
4-(N-(3-Chloro-5-(trifluoromethyl)pyridin-2-yl)-N-(4-(pyridin-4-yl)benzyl)sulfamoyl)benzoic acid;
4-(N-(3-Chloro-5-(trifluoromethyl)pyridin-2-yl)-N-(4-(furan-2-yl)benzyl)sulfamoyl)benzoic acid;
4-(N-([1,1′-Biphenyl]-4-ylmethyl)-N-(3-chloro-5-(trifluoromethyl)pyridin-2-yl)sulfamoyl)benzoic acid;
4-(N-(3-Chloro-5-(trifluoromethyl)pyridin-2-yl)-N-(4-(oxazol-5-yl)benzyl)sulfamoyl)benzoic acid;
4-(N-(3-Chloro-5-(trifluoromethyl)pyridin-2-yl)-N-((6-(trifluoromethyl)pyridin-3-yl)methyl)sulfamoyl)benzoic acid;
4-(N-(3-Chloro-5-(trifluoromethyl)pyridin-2-yl)-N-(4-(picolinamido)benzyl)sulfamoyl)benzoic acid;
4-(N-(3-Chloro-5-(trifluoromethyl)pyridin-2-yl)-N-(4-(6-methoxypyridin-3-yl)benzyl)sulfamoyl)benzoic acid;
4-(N-(3-Chloro-5-(trifluoromethyl)pyridin-2-yl)-N-(4-(6-methylpyridin-3-yl)benzyl)sulfamoyl)benzoic acid;
4-(N-([1,1′-Biphenyl]-3-ylmethyl)-N-(3-chloro-5-(trifluoromethyl)pyridin-2-yl)sulfamoyl)benzoic acid;
4-(N-([1,1′-Biphenyl]-2-ylmethyl)-N-(3-chloro-5-(trifluoromethyl)pyridin-2-yl)sulfamoyl)benzoic acid;
(R)-4-(N-(3-Chloro-5-(trifluoromethyl)pyridin-2-yl)-N-(1-phenylethyl)sulfamoyl)benzoic acid;
4-(N-(3-Chloro-5-(trifluoromethyl)pyridin-2-yl)-N-(thiophen-2-ylmethyl)sulfamoyl)benzoic acid;
4-(N-(3-Chloro-5-(trifluoromethyl)pyridin-2-yl)-N-(cyclohexylmethyl)sulfamoyl)benzoic acid;
4-(N-(3-Chloro-5-(trifluoromethyl)pyridin-2-yl)-N-(4-phenoxybenzyl)sulfamoyl)benzoic acid;
4-(N-(4-(1H-Pyrazol-1-yl)benzyl)-N-(3-chloro-5-(trifluoromethyl)pyridin-2-yl)sulfamoyl)benzoic acid;
4-(N-(3-Chloro-5-(trifluoromethyl)pyridin-2-yl)-N-(cyclobutylmethyl)sulfamoyl)benzoic acid;
4-(N-(3-Chloro-5-(trifluoromethyl)pyridin-2-yl)-N-(thiophen-3-ylmethyl)sulfamoyl)benzoic acid;
N-Benzyl-N-(3-chloro-5-(trifluoromethyl)pyridin-2-yl)-4-cyanobenzenesulfonamide;
N-Benzyl-N-(3-chloro-5-(trifluoromethyl)pyridin-2-yl)-4-(2H-tetrazol-5-yl)benzenesulfonamide;
N-Benzyl-N-(3-chloro-5-(trifluoromethyl)pyridin-2-yl)-4-methoxybenzenesulfonamide;
N-Benzyl-N-(3-chloro-5-(trifluoromethyl)pyridin-2-yl)-4-(methylsulfonamidomethyl)benzenesulfonamide;
N-(4-(N-Benzyl-N-(3-chloro-5-(trifluoromethyl)pyridin-2-yl)sulfamoyl)benzyl)acetamide;
N-Benzyl-N-(3-chloro-5-(trifluoromethyl)pyridin-2-yl)-4-(((N,N-dimethylsulfamoyl)amino)methyl)benzenesulfonamide;
N-Benzyl-N-(3-chloro-5-(trifluoromethyl)pyridin-2-yl)-4-((3,3-dimethylureido)methyl)benzenesulfonamide;
4-(N-Benzyl-N-(5-bromo-3-chloropyridin-2-yl)sulfamoyl)benzoic acid;
4-(N-Benzyl-N-(3-chloro-5-(trifluoromethyl)pyridin-2-yl)sulfamoyl)-3-chlorobenzoic acid;
4-(N-Benzyl-N-(3-chloro-5-phenylpyridin-2-yl)sulfamoyl)benzoic acid;
4-(N-Benzyl-N-(3-chloro-5-(furan-2-yl)pyridin-2-yl)sulfamoyl)benzoic acid;
4-(N-Benzyl-N-(3-chloro-5-(thiophen-3-yl)pyridin-2-yl)sulfamoyl)benzoic acid;
4-(N-Benzyl-N-(3-chloro-5-(2-methoxyphenyl)pyridin-2-yl)sulfamoyl)benzoic acid;
4-(N-Benzyl-N-(3-chloro-5-(4-methoxyphenyl)pyridin-2-yl)sulfamoyl)benzoic acid;
4-(N-Benzyl-N-(3-chloro-5-(3-methoxyphenyl)pyridin-2-yl)sulfamoyl)benzoic acid;
4-(N-Benzyl-N-(3-chloro-5-(trifluoromethyl)pyridin-2-yl)sulfamoyl)-2-chlorobenzoic acid;
N-Benzyl-N-(3-chloro-5-(trifluoromethyl)pyridin-2-yl)-6-methoxypyridine-3-sulfonamide;
4-(N-Benzyl-N-(3-chloro-5-(trifluoromethyl)pyridin-2-yl)sulfamoyl)-3-fluorobenzoic acid;
4-(N-Benzyl-N-(3-chloro-5-(trifluoromethyl)pyridin-2-yl)sulfamoyl)-2-fluorobenzoic acid;
4-(N-(3-Chloro-5-(trifluoromethyl)pyridin-2-yl)-N-(cyclopentylmethyl)sulfamoyl)benzoic acid;
4-(N-(3-Chloro-5-(trifluoromethyl)pyridin-2-yl)-N-(1-phenylcyclopropyl)sulfamoyl)benzoic acid;
N-Benzyl-N-(3-chloro-5-(trifluoromethyl)pyridin-2-yl)-4-((N,N-dimethylsulfamoyl)amino)benzenesulfonamide;
N-Benzyl-N-(3-chloro-5-(trifluoromethyl)pyridin-2-yl)-4-ureidobenzenesulfonamide;
N-Benzyl-N-(3-chloro-5-(trifluoromethyl)pyridin-2-yl)-4-(sulfamoylamino)benzenesulfonamide;
(S)-4-(N-(3-Chloro-5-(trifluoromethyl)pyridin-2-yl)-N-(1-phenylethyl)sulfamoyl)benzoic acid;
4-(N-(3-Chloro-5-(trifluoromethyl)pyridin-2-yl)-N-((2-phenylthiazol-4-yl)methyl)sulfamoyl)benzoic acid;
4-(N-(3-Chloro-5-(trifluoromethyl)pyridin-2-yl)-N-((5-phenyl-1,2,4-oxadiazol-3-yl)methyl)sulfamoyl)benzoic acid;
4-(N-Benzyl-N-(3-chloro-5-(trifluoromethyl)pyridin-2-yl)sulfamoyl)benzoic acid; and
N-Benzyl-N-(3-chloro-5-(trifluoromethyl)pyridin-2-yl)-4-(hydroxymethyl)benzene-1-sulfonamide;
   or a pharmaceutically acceptable salt thereof.

More suitable individual compounds of the invention are:
4-(N-(3-Chloro-5-(trifluoromethyl)pyridin-2-yl)-N-(4-fluorobenzyl)sulfamoyl)benzoic acid;
4-(N-(3-Chloro-5-(trifluoromethyl)pyridin-2-yl)-N-(2-fluorobenzyl)sulfamoyl)benzoic acid;
4-(N-(3-Chloro-5-(trifluoromethyl)pyridin-2-yl)-N-(3-(trifluoromethyl)benzyl)sulfamoyl)benzoic acid;
4-(N-(3-Chloro-5-(trifluoromethyl)pyridin-2-yl)-N-(3-(trifluoromethoxy)benzyl)sulfamoyl)benzoic acid;
4-(N-(3-Chloro-5-(trifluoromethyl)pyridin-2-yl)-N-(2,4-difluorobenzyl)sulfamoyl)benzoic acid;
4-(N-(3-Chloro-5-(trifluoromethyl)pyridin-2-yl)-N-(2-(4-fluorophenoxy)ethyl)sulfamoyl)benzoic acid;
4-(N-(3-Chloro-5-(trifluoromethyl)pyridin-2-yl)-N-(2-(trifluoromethyl)benzyl)sulfamoyl)benzoic acid;
4-(N-(3-Chloro-5-(trifluoromethyl)pyridin-2-yl)-N-(4-chlorobenzyl)sulfamoyl)benzoic acid;
4-(N-(3-Chloro-5-(trifluoromethyl)pyridin-2-yl)-N-(4-cyanobenzyl)sulfamoyl)benzoic acid;
4-(N-Benzyl-N-(3,5-dichloropyridin-2-yl)sulfamoyl)benzoic acid;
4-(N-Benzyl-N-(5-chloro-3-methylpyridin-2-yl)sulfamoyl)benzoic acid;
4-(N-(2-Chloro-4-fluorobenzyl)-N-(3-chloro-5-(trifluoromethyl)pyridin-2-yl)sulfamoyl)benzoic acid;
4-(N-(3-Chloro-5-(trifluoromethyl)pyridin-2-yl)-N-(3,5-difluorobenzyl)sulfamoyl)benzoic acid;
4-(N-(3-Chloro-5-(trifluoromethyl)pyridin-2-yl)-N-(4-fluoro-3-(trifluoromethyl)benzyl)sulfamoyl)benzoic acid;
4-(N-(3-Chloro-5-(trifluoromethyl)pyridin-2-yl)-N-(3,4-difluorobenzyl)sulfamoyl)benzoic acid;
4-(N-(3-Chloro-5-(trifluoromethyl)pyridin-2-yl)-N-(2,5-difluorobenzyl)sulfamoyl)benzoic acid;
4-(N-(3-Chloro-5-(trifluoromethyl)pyridin-2-yl)-N-(3,4-dichlorobenzyl)sulfamoyl)benzoic acid;
4-(N-(3-Chloro-5-(trifluoromethyl)pyridin-2-yl)-N-(3-chlorobenzyl)sulfamoyl)benzoic acid;
4-(N-(3-Chloro-5-(trifluoromethyl)pyridin-2-yl)-N-(4-(1,1,1-trifluoro-2-methylpropan-2-yl)benzyl)sulfamoyl)benzoic acid;
4-(N-(3-Chloro-5-(trifluoromethyl)pyridin-2-yl)-N-(2-(trifluoromethoxy)benzyl)sulfamoyl)benzoic acid;
4-(N-(3,5-Dichloropyridin-2-yl)-N-(4-(trifluoromethyl)benzyl)sulfamoyl)benzoic acid;
4-(N-(5-Chloro-3-methylpyridin-2-yl)-N-(4-(trifluoromethyl)benzyl)sulfamoyl)benzoic acid;
4-(N-Benzyl-N-(2-chloro-4-(trifluoromethyl)phenyl)sulfamoyl)benzoic acid;
4-(N-(3,5-Dichloropyridin-2-yl)-N-(4-fluoro-3-(trifluoromethyl)benzyl)sulfamoyl)benzoic acid;
4-(N-(3,5-Dichloropyridin-2-yl)-N-(4-fluorobenzyl)sulfamoyl)benzoic acid;
4-(N-(4-Chloro-3-fluorobenzyl)-N-(3,5-dichloropyridin-2-yl)sulfamoyl)benzoic acid;
4-(N-(3,5-Dichloropyridin-2-yl)-N-(4-(trifluoromethoxy)benzyl)sulfamoyl)benzoic acid;
4-(N-(3,5-Dichloropyridin-2-yl)-N-(2-(trifluoromethyl)benzyl)sulfamoyl)benzoic acid;
4-(N-(4-Chloro-3-fluorobenzyl)-N-(3-chloro-5-(trifluoromethyl)pyridin-2-yl)sulfamoyl)benzoic acid;
4-(N-(4-Chloro-3-(trifluoromethyl)benzyl)-N-(3,5-dichloropyridin-2-yl)sulfamoyl)benzoic acid;
4-(N-(3-Chloro-4-fluorobenzyl)-N-(3,5-dichloropyridin-2-yl)sulfamoyl)benzoic acid;
4-(N-(3-Chloro-4-fluorobenzyl)-N-(3-chloro-5-(trifluoromethyl)pyridin-2-yl)sulfamoyl)benzoic acid;
4-(N-(4-Chloro-2-(trifluoromethyl)benzyl)-N-(3,5-dichloropyridin-2-yl)sulfamoyl)benzoic acid;
4-(N-(4-Chloro-2-(trifluoromethyl)benzyl)-N-(5-chloro-3-methylpyridin-2-yl)sulfamoyl)benzoic acid;
4-(N-(5-Chloro-3-methylpyridin-2-yl)-N-(4-fluoro-3-(trifluoromethyl)benzyl)sulfamoyl)benzoic acid;
4-(N-(5-Chloro-3-methylpyridin-2-yl)-N-(3-chloro-4-fluorobenzyl)sulfamoyl)benzoic acid;
4-(N-(5-Chloro-3-methylpyridin-2-yl)-N-(4-(trifluoromethoxy)benzyl)sulfamoyl)benzoic acid;
4-(N-(4-Chloro-3-(trifluoromethyl)benzyl)-N-(5-chloro-3-methylpyridin-2-yl)sulfamoyl)benzoic acid;
4-(N-(3-Chloro-5-(trifluoromethyl)pyridin-2-yl)-N-(4-(1-cyanocyclopropyl)benzyl)sulfamoyl)benzoic acid;
4-(N-Benzyl-N-(3-chloro-5-(trifluoromethyl)pyridin-2-yl)sulfamoyl)-3-methylbenzoic acid;
4-(N-Benzyl-N-(3-bromo-5-(trifluoromethyl)pyridin-2-yl)sulfamoyl)benzoic acid;
4-(N-(3-Chloro-5-(trifluoromethyl)pyridin-2-yl)-N-(4-(pyridin-3-yl)benzyl)sulfamoyl)benzoic acid;
4-(N-(3-Chloro-5-(trifluoromethyl)pyridin-2-yl)-N-(4-(6-methoxypyridin-3-yl)benzyl)sulfamoyl)benzoic acid;
4-(N-(3-Chloro-5-(trifluoromethyl)pyridin-2-yl)-N-(4-(6-methylpyridin-3-yl)benzyl)sulfamoyl)benzoic acid;
4-(N-([1,1′-Biphenyl]-2-ylmethyl)-N-(3-chloro-5-(trifluoromethyl)pyridin-2-yl)sulfamoyl)benzoic acid;
N-Benzyl-N-(3-chloro-5-(trifluoromethyl)pyridin-2-yl)-4-(2H-tetrazol-5-yl)benzenesulfonamide;
4-(N-Benzyl-N-(3-chloro-5-(trifluoromethyl)pyridin-2-yl)sulfamoyl)-3-chlorobenzoic acid;
4-(N-Benzyl-N-(3-chloro-5-(trifluoromethyl)pyridin-2-yl)sulfamoyl)benzoic acid; and
4-(N-(3-Chloro-5-(trifluoromethyl)pyridin-2-yl)-N-(4-(trifluoromethoxy)benzyl)sulfamoyl)benzoic acid;
   or a pharmaceutically acceptable salt thereof.

Also, the present invention provides a pharmaceutical composition comprising a compound of formula (I) or a pharmaceutically acceptable salt thereof, each as described herein, together with a pharmaceutically acceptable carrier for said compound.

Also, the present invention provides a pharmaceutical composition comprising a compound of formula (I) or a pharmaceutically acceptable salt thereof, each as described herein, together with a pharmaceutically acceptable carrier for said compound and another pharmacologically active agent.

Also, the present invention provides a process for preparing a pharmaceutical composition, the process comprising mixing a compound of formula (I) or a pharmaceutically acceptable salt thereof and a pharmaceutically acceptable carrier or excipient.

Also, the present invention provides an intermediate in a process for preparing a compound of formula (I) or a pharmaceutically acceptable salt thereof.

Further, the present invention provides a method for the treatment of a condition or disorder mediated by TRPM8 receptor activity, in a mammalian subject, which comprises administering to a mammal in need of such treatment a therapeutically effective amount of a compound of formula (I) or a pharmaceutically acceptable salt thereof, each as described herein.

In a further aspect, the present invention provides a process for preparing a pharmaceutical composition, the process comprising mixing a compound of formula (I) or a pharmaceutically acceptable salt thereof and a pharmaceutically acceptable carrier or excipient.

Examples of conditions or disorders mediated by TRPM8 receptor activity include, but are not limited to, TRPM8 related diseases.

### Advantageous Effects of Invention

The compounds of the present invention show the TRPM8 receptor antagonistic activity. The compounds of the present invention may show less toxicity, good absorption, distribution, good solubility, less protein binding affinity other than TRPM8 receptor, less drug-drug interaction, and good metabolic stability.

### DESCRIPTION OF EMBODIMENTS

As used herein, the term “alkyl” as a group or part of a group e.g. alkoxy or hydroxyalkyl refers to a straight or branched alkyl group in all isomeric forms. The term “C₁-C₄ alkyl” refers to an alkyl group, as defined above, containing at least 1, and at most 4 carbon atoms. Examples of such alkyl groups include methyl, ethyl, propyl, iso-propyl, n-butyl, iso-butyl, sec-butyl, or tert-butyl. Examples of such alkoxy groups include methoxy, ethoxy, propoxy, iso-propoxy, butoxy, iso-butoxy, sec-butoxy and tert-butoxy.

The term “cycloalkyl”, as used herein, means a mono- or bicyclic ring, but not limited to, cyclopropyl, cyclobutyl, cyclopentyl, cyclohexyl, cycloheptyl, norboranyl, and adamantyl groups and the like.

The term “halogen” refers to fluorine (F), chlorine (Cl), bromine (Br), or iodine (I) and the term “halo” refers to the halogen: fluoro (—F), chloro (—Cl), bromo (—Br) and iodo (—I).

The term “haloalkyl”, as used herein, means an alkyl radical which is substituted by halogen atom(s) as defined above including, but not limited to, fluoromethyl, difluoromethyl, trifluoromethyl, 2-fluoroethyl, 2,2-difluoroethyl, 2,2,2-trifluoroethyl, 2,2,2-trichloroethyl, 3-fluoropropyl, 4-fluorobutyl, chloromethyl, trichloromethyl, iodomethyl and bromomethyl groups and the like.

The term “haloalkoxy”, as used herein, means haloalkyl-O—, including, but not limited to, fluoromethoxy, difluoromethoxy, trifluoromethoxy, 2-fluoroethoxy, 2,2-difluoroethoxy, 2,2,2-trifluoroethoxy, 2,2,2-trichloroethoxy, 3-fluoropropoxy, 4-fluorobutoxy, chforomethoxy, trichloromethoxy, iodomethoxy and bromomethoxy groups and the like.

The term “alkenyl”, as used herein, means a hydrocarbon radical having at least one double bond including, but not limited to, ethenyl, propenyl, 1-butenyl, 2-butenyl and the like.

The term “alkynyl”, as used herein, means a hydrocarbon radical having at least one triple bond including, but not limited to, ethynyl, propynyl, 1-butynyl, 2-butynyl and the like.

The term “alkoxy”, as used herein, means an O-alkyl group wherein “alkyl” is defined above.

The term “aryl”, as used herein, means mono-carbocyclic or mono-heterocyclic group which may contain 0-4 heteroatoms selected from O, N and S; the said heterocyclic group includes both unsaturated and saturated heterocyclic moieties;

wherein the unsaturated heterocyclic moieties include furyl, furazanyl, imidazolyl, isooxazolyl, isothiazolyl, oxazolyl, pyrazolyl, pyridinyl, pyrimidinyl, pyrrolyl, pyrazinyl, pyridazinyl, thienyl, tetrazolyl, thiazolyl, triazinyl, thiophenyl, triazolyl, and N-oxides thereof and S-oxides thereof;
and wherein the saturated heterocyclic moieties include azetidinyl, 1,4-dioxanyl, hexahydroazepinyl, piperazinyl, piperidinyl, pyrrolidinyl, piperidin-2-one-yl, pyrrolidin-2-one-yl, morpholinyl, tetrahydrofuranyl, thiomorpholinyl, and tetrahydrothienyl, and N-oxides thereof and S-oxides thereof.

The term “heterocyclyl”, as used herein, means a saturated ring which comprises one or more heteroatoms selected from nitrogen, oxygen and sulphur. Examples of such heterocyclyl groups include azetidinyl, 1,4-dioxanyl, hexahydroazepinyl, pyrrolidinyl, piperidinyl, piperazinyl, morpholinyl, tetrahydrofuranyl, thiomorpholinyl, and tetrahydrothienyl, and N-oxides thereof and S-oxides thereof.

The term “C₀”, as used herein, means direct bond.

The term “protecting group”, as used herein, means a hydroxy or amino protecting group which is selected from typical hydroxy or amino protecting groups described in Protective Groups in Organic Synthesis edited by T. W. Greene et al. (John Wiley & Sons, 1991);

The term “treating”, as used herein, refers to reversing, alleviating, inhibiting the progress of, or preventing the disorder or condition to which such term applies, or one or more symptoms of such disorder or condition. The term “treatment” as used herein refers to the act of treating, as “treating” is defined immediately above.

The term “treating” and “treatment”, as used herein, refers to curative, palliative and prophylactic treatment, including reversing, alleviating, inhibiting the progress of, or preventing the disorder or condition to which such term applies, or one or more symptoms of such disorder or condition.

As used herein, the article “a” or “an” refers to both the singular and plural form of the object to which it refers unless indicated otherwise.

Included within the scope of the “compounds of the invention” are all salts, solvates, hydrates, complexes, polymorphs, prodrugs, radiolabeled derivatives, stereoisomers and optical isomers of the compounds of formula (I).

The compounds of formula (I) can form acid addition salts thereof. It will be appreciated that for use in medicine the salts of the compounds of formula (I) should be pharmaceutically acceptable. Suitable pharmaceutically acceptable salts will be apparent to those skilled in the art and include those described in J. Pharm. Sci, 1977, 66, 1-19, such as acid addition salts formed with inorganic acids e.g. hydrochloric, hydrobromic, sulfuric, nitric or phosphoric acid; and organic acids e.g. succinic, maleic, formic, acetic, trifluoroacetic, propionic, fumaric, citric, tartaric, benzoic, p-toluenesulfonic, methanesulfonic or naphthalenesulfonic acid. Certain of the compounds of formula (I) may form acid addition salts with one or more equivalents of the acid. The present invention includes within its scope all possible stoichiometric and non-stoichiometric forms. In addition, certain compounds containing an acidic function such as a carboxy can be isolated in the form of their inorganic salt in which the counter ion can be selected from sodium, potassium, lithium, calcium, magnesium and the like, as well as from organic bases.

The compounds of formula (I) and salts thereof may be prepared in crystalline or non-crystalline form, and, if crystalline, may optionally be hydrated or solvated. This invention includes within its scope stoichiometric hydrates or solvates as well as compounds containing variable amounts of water and/or solvent.

Salts and solvates having non-pharmaceutically acceptable counter-ions or associated solvents are within the scope of the present invention, for example, for use as intermediates in the preparation of other compounds of formula (I) and their pharmaceutically acceptable salts.

Additionally, the compounds of formula (I) may be administered as prodrugs. As used herein, a “prodrug” of a compound of formula (I) is a functional derivative of the compound which, upon administration to a patient, eventually liberates the compound of formula (I) in vivo. Administration of a compound of formula (I) as a prodrug may enable the skilled artisan to do one or more of the following: (a) modify the onset of action of the compound in vivo; (b) modify the duration of action of the compound in vivo; (c) modify the transportation or distribution of the compound in vivo; (d) modify the solubility of the compound in vivo; and (e) overcome a side effect or other difficulty encountered with the compound. Typical functional derivatives used to prepare prodrugs include modifications of the compound that are chemically or enzymatically cleaved in vivo. Such modifications, which include the preparation of phosphates, amides, esters, thioesters, carbonates, and carbamates, are well known to those skilled in the art.

In certain of the compounds of formula (I), there may be some chiral carbon atoms. In such cases, compounds of formula (I) exist as stereoisomers. The invention extends to all optical isomers such as stereoisomeric forms of the compounds of formula (I) including enantiomers, diastereoisomers and mixtures thereof, such as racemates. The different stereoisomeric forms may be separated or resolved one from the other by conventional methods or any given isomer may be obtained by conventional stereoselective or asymmetric syntheses.

Certain of the compounds herein can exist in various tautomeric forms and it is to be understood that the invention encompasses all such tautomeric forms.

The invention also includes isotopically-labeled compounds, which are identical to those described herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine, iodine, and chlorine, such as ³H, ¹¹C, ¹⁴C, ¹⁸F, ¹²³I and ¹²⁵I. Compounds of the invention that contain the invention that contain the aforementioned isotopes and/or other isotopes of other atoms are within the scope of the present invention. Isotopically-labeled compounds of the present invention, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. ¹¹C and ¹⁸F isotopes are particularly useful in PET (positron emission tomography), and ¹²⁵I isotopes are particularly useful in SPECT (single photon emission computerized tomography), all useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances, Isotopically labeled compounds of the invention can generally be prepared by carrying out the procedures disclosed in the Schemes and/or in the Examples below, then substituting a readily available isotopically labeled reagent for a non-isotopically labeled reagent.

The potencies and efficacies of the compounds of this invention for TRPM8 can be determined by reporter assay performed on the human cloned receptor as described herein. Compounds of formula (I) have demonstrated antagonistic activity at the TRPM8 receptor, using the functional assay described herein.

Compounds of formula (I) and pharmaceutically acceptable salts thereof are therefore of use in the treatment of conditions or disorders which are mediated via the TRPM8 receptor. In particular the compounds of formula (I) and pharmaceutically acceptable salts thereof are of use in the treatment of a wide range of diseases, syndromes, and disorders, in particular for the treatment of inflammatory, pain and urological diseases or disorders, such as chronic pain, neuropathic pain including cold allodynia and diabetic neuropathy, postoperative pain, osteoarthritis, rheumatoid arthritic pain, cancer pain, neuralgia, neuropathies, algesia, nerve injury, migraine, cluster and tension headaches, ischaemia, irritable bowel syndrome, neurodegeneration, fibromyalgia, stroke, itch, psychiatric disorders including anxiety and depression and inflammatory disorders such as asthma and chronic obstructive pulmonary, or airways, disease i.e., COPD, pulmonary hypertension, anxiety, including other stress-related disorders, urological diseases or disorders such as detrusor overactivity or overactive bladder, urinary incontinence, neurogenic detrusor overactivity or detrusor hyperflexia, idiopathic detrusor overactivity or detrusor instability, benign prostatic hyperplasia, and lower urinary tract symptoms, and combinations thereof.

Activities of the compound (I) for each diseases, syndromes, and disorders described above can be confirmed in the suitable model known to skilled in the arts. For example, activities of compounds of formula (I) for neuropathic pain have been confirmed in chronic constriction injury (CCI)-induced model, such as cold allodynia and static allodynia model.

It is to be understood that “treatment” as used herein includes prophylaxis as well as alleviation of established symptoms.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusible solutions or suspensions or suppositories. Orally administrate compositions are generally preferred. Tablets and capsules for oral administration may be in unit dose form, and may contain conventional excipients, such as binding agents (e.g. pregelatinised maize starch, polyvinylpyrrolidone or hydroxypropyl methylcellulose); fillers (e.g. lactose, microcrystalline cellulose or calcium hydrogen phosphate); tabletting lubricants (e.g. magnesium stearate, talc or silica); disintegrants (e.g. potato starch or sodium starch glycollate); and acceptable wetting agents (e.g. sodium lauryl sulphate). The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents (e.g. sorbitol syrup, cellulose derivatives or hydrogenated edible fats), emulsifying agents (e.g. lecithin or acacia), non-aqueous vehicles (which may include edible oils e.g. almond oil, oily esters, ethyl alcohol or fractionated vegetable oils), preservatives (e.g. methyl or propyl-p-hydroxybenzoates or sorbic acid), and, if desired, conventional flavourings or colorants, buffer salts and sweetening agents as appropriate. Preparations for oral administration may be suitably formulated to give controlled release of the active compound or pharmaceutically acceptable salt thereof.

For parenteral administration, fluid unit dosage forms are prepared utilising a compound of formula (I) or pharmaceutically acceptable salt thereof and a sterile vehicle. Formulations for injection may be presented in unit dosage form e.g. in ampoules or in multi-dose, utilising a compound of formula (I) or pharmaceutically acceptable salt thereof and a sterile vehicle, optionally with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilisation cannot be accomplished by filtration. The compound can be sterilised by exposure to ethylene oxide before suspension in a sterile vehicle. Advantageously, a surfactant or wetting agent is included in the composition to facilitate uniform distribution of the compound.

Lotions may be formulated with an aqueous or oily base and will in general also contain one or more emulsifying agents, stabilising agents, dispersing agents, suspending agents, thickening agents, or colouring agents. Drops may be formulated with an aqueous or non-aqueous base also comprising one or more dispersing agents, stabilising agents, solubilising agents or suspending agents. They may also contain a preservative.

The compounds of formula (I) or pharmaceutically acceptable salts thereof may also be formulated in rectal compositions such as suppositories or retention enemas, e.g. containing conventional suppository bases such as cocoa butter or other glycerides.

The compounds of formula (I) or pharmaceutically acceptable salts may also be formulated as depot preparations. Such long acting formulations may be administered by implantation (for example subcutaneously or intramuscularly) or by intramuscular injection. Thus, for example, the compounds of formula (I) or pharmaceutically acceptable salts may be formulated with suitable polymeric or hydrophobic materials (for example as an emulsion in an acceptable oil) or ion exchange resins, or as sparingly soluble derivatives, for example, as a sparingly soluble salt.

For intranasal administration, the compounds formula (I) or pharmaceutically acceptable salts thereof may be formulated as solutions for administration via a suitable metered or unitary dose device or alternatively as a powder mix with a suitable carrier for administration using a suitable delivery device. Thus compounds of formula (I) or pharmaceutically acceptable salts thereof may be formulated for oral, buccal, parenteral, topical (including ophthalmic and nasal), depot or rectal administration or in a form suitable for administration by inhalation or insufflation (either through the mouth or nose). The compounds of formula (I) and pharmaceutically acceptable salts thereof may be formulated for topical administration in the form of ointments, creams, gels, lotions, pessaries, aerosols or drops (e.g. eye, ear or nose drops). Ointments and creams may, for example, be formulated with an aqueous or oily base with the addition of suitable thickening and/or gelling agents. Ointments for administration to the eye may be manufactured in a sterile manner using sterilized components.

A TRPM8 antagonist may be usefully combined with another pharmacologically active compound, or with two or more other pharmacologically active compounds, particularly in the treatment of inflammatory, pain and urological diseases or disorders. For example, a TRPM8 antagonist, particularly a compound of formula (I), or a pharmaceutically acceptable salt or solvate thereof, as defined above, may be administered simultaneously, sequentially or separately in combination with one or more agents selected from:
an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine or pentazocine;
a nonsteroidal antiinflammatory drug (NSAID), e.g. aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin or zomepirac;
a barbiturate sedative, e.g. amobarbital, aprobarbital, butabarbital, butabital, mephobarbital, metharbital, methohexital, pentobarbital, phenobartital, secobarbital, talbutal, theamylal or thiopental;
a benzodiazepine having a sedative action, e.g. chlordiazepoxide, clorazepate, diazepam, flurazepam, lorazepam, oxazepam, temazepam or triazolam;
an H₁ antagonist having a sedative action, e.g. diphenhydramine, pyrilamine, promethazine, chlorpheniramine or chlorcyclizine;
a sedative such as glutethimide, meprobamate, methaqualone or dichloralphenazone;
a skeletal muscle relaxant, e.g. baclofen, carisoprodol, chlorzoxazone, cyclobenzaprine, methocarbamol or orphrenadine;
an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinine, cis-4-(phosphonomethyl)-2-piperidinecarboxylic acid, budipine, EN-3231 (MorphiDex (registered trademark), a combination formulation of morphine and dextromethorphan), topiramate, neramexane or perzinfotel including an NR2B antagonist, e.g. ifenprodil, traxoprodil or (−)-(R)-6-{2-[4-(3-fluorophenyl)-4-hydroxy-1-piperidinyl]-1-hydroxyethyl-3,4-dihydro-2(1H)-quinolinone;
an alpha-adrenergic, e.g. doxazosin, tamsulosin, clonidine, guanfacine, dexmetatomidine, modafinil, or 4-amino-6,7-dimethoxy-2-(5-methane-sulfonamido-1,2,3,4-tetrahydroisoquinol-2-yl)-5-(2-pyridyl)quinazoline;
a tricyclic antidepressant, e.g. desipramine, imipramine, amitriptyline or nortriptyline;
an anticonvulsant, e.g. carbamazepine, lamotrigine, topiratmate or valproate;
a tachykinin (NK) antagonist, particularly an NK-3, NK-2 or NK-1 antagonist, e.g. (alphaR,9R)-7-[3,5-bis(trifluoromethyl)benzyl]-8,9,10,11-tetrahydro-9-methyl-5-(4-methylphenyl)-7H[1,4]diazocino[2,1-g][1,7]-naphthyridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy-5-(trifluoromethoxy)phenyl]-methylamino]-2-phenylpiperidine (2S,3S);
a muscarinic antagonist, e.g oxybutynin, tolterodine, propiverine, tropsium chloride, darifenacin, solifenacin, temiverine and ipratropium;
a COX-2 selective inhibitor, e.g. celecoxib, rofecoxib, parecoxib, valdecoxib, deracoxib, etoricoxib, or lumiracoxib;
a coal-tar analgesic, in particular paracetamol;
a neuroleptic such as droperidol, chlorpromazine, haloperidol, perphenazine, thioridazine, mesoridazine, trifluoperazine, fluphenazine, clozapine, olanzapine, risperidone, ziprasidone, quetiapine, sertindole, aripiprazole, sonepiprazole, blonanserin, iloperidone, perospirone, raclopride, zotepine, bifeprunox, asenapine, lurasidone, amisulpride, balaperidone, palindore, eplivanserin, osanetant, rimonabant, meclinertant, Miraxion (registered trademark) or sarizotan;
a vanilloid receptor agonist (e.g. resinferatoxin) or antagonist (e.g. capsazepine);
a beta-adrenergic such as propranolol;
a local anaesthetic such as mexiletine;
a corticosteroid such as dexamethasone;
a 5-HT receptor agonist or antagonist, particularly a 5-HT_{1B/1D} agonist such as eletriptan, sumatriptan, naratriptan, zolmitriptan or rizatriptan;
a 5-HT_{2A} receptor antagonist such as R(+)-alpha-(2,3-dimethoxy-phenyl)-1-[2-(4-fluorophenylethyl)]-4-piperidinemethanol (MDL-100907);
a cholinergic (nicotinic) analgesic, such as ispronicline (TC-1734), (E)-N-methyl-4-(3-pyridinyl)-3-buten-1-amine (RJR-2403), (R)-5-(2-azetidinylmethoxy)-2-chloropyridine (ABT-594) or nicotine;
Tramadol (registered trademark);
a PDEV inhibitor, such as 5-[2-ethoxy-5-(4-methyl-1-piperazinyl-sulphonyl)phenyl]-1-methyl-3-n-propyl-1,6-dihydro-7H-pyrazolo[4,3-d]pyrimidin-7-one (sildenafil), (6R,12aR)-2,3,6,7,12,12a-hexahydro-2-methyl-6-(3,4-methylenedioxyphenyl)-pyrazino[2′,1′:6,1]-pyrido[3,4-b]indole-1,4-dione (IC-351 or tadalafil), 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil), 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one, 5-(5-acetyl-2-propoxy-3-pyridinyl)-3-ethyl-2-(1-isopropyl-3-azetidinyl)-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, 4-[(3-chloro-4-methoxybenzyl)amino]-2-[(2S)-2-(hydroxymethyl)pyrrolidin-1-yl]-N-(pyrimidin-2-ylmethyl)pyrimidine-5-carboxamide, 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide;
an alpha-2-delta ligand such as gabapentin, pregabalin, 3-methylgabapentin, (1alpha,3alpha,5alpha)(3-amino-methyl-bicyclo[3.2.0]hept-3-yl)-acetic acid, (3S,5R)-3-aminomethyl-5-methyl-heptanoic acid, (3S,5R)-3-amino-5-methyl-heptanoic acid, (3S,5R)-3-amino-5-methyl-octanoic acid, (2S,4S)-4-(3-chlorophenoxy)proline, (2S,4S)-4-(3-fluorobenzyl)-proline, [(1R,5R,6S)-6-(aminomethyl)bicyclo[3.2.0]hept-6-yl]acetic acid, 3-(1-aminomethyl-cyclohexylmethyl)-4H-[1,2,4]oxadiazol-5-one, C-[1-(1H-tetrazol-5-ylmethyl)-cycloheptyl]-methylamine, (3S,4S)-(1-aminomethyl-3,4-dimethyl-cyclopentyl)-acetic acid, (3S,5R)-3-aminomethyl-5-methyl-octanoic acid, (3S,5R)-3-amino-5-methyl-nonanoic acid, (3S,5R)-3-amino-5-methyl-octanoic acid, (3R,4R,5R)-3-amino-4,5-dimethyl-heptanoic acid and (3R,4R,5R)-3-amino-4,5-dimethyl-octanoic acid;
a cannabinoid;
metabotropic glutamate subtype 1 receptor (mGluR1) antagonist;
a serotonin reuptake inhibitor such as sertraline, sertraline metabolite demethylsertraline, fluoxetine, norfluoxetine (fluoxetine desmethyl metabolite), fluvoxamine, paroxetine, citalopram, citalopram metabolite desmethylcitalopram, escitalopram, d,l-fenfluramine, femoxetine, ifoxetine, cyanodothiepin, litoxetine, dapoxetine, nefazodone, cericlamine and trazodone;
a noradrenaline (norepinephrine) reuptake inhibitor, such as maprotiline, lofepramine, mirtazepine, oxaprotiline, fezolamine, tomoxetine, mianserin, buproprion, buproprion metabolite hydroxybuproprion, nomifensine and viloxazine (Vivalan (registered trademark)), especially a selective noradrenaline reuptake inhibitor such as reboxetine, in particular (S,S)-reboxetine;
a dual serotonin-noradrenaline reuptake inhibitor, such as venlafaxine, venlafaxine metabolite O-desmethylvenlafaxine, clomipramine, clomipramine metabolite desmethylclomipramine, duloxetine, milnacipran and imipramine;
an inducible nitric oxide synthase (iNOS) inhibitor such as S-[2-[(1-iminoethyl)amino]ethyl]-L-homocysteine, S-[2-[(1-iminoethyl)-amino]ethyl]-4,4-dioxo-L-cysteine, S-[2-[(1-iminoethyl)amino]ethyl]-2-methyl-L-cysteine, (2S,5Z)-2-amino-2-methyl-7-[(1-iminoethyl)amino]-5-heptenoic acid, 2-[[(1R,3S)-3-amino-4-hydroxy-1-(5-thiazolyl)-butyl]thio]-5-chloro-3-pyridinecarbonitrile, 2-[[(1R,3S)-3-amino-4-hydroxy-1-(5-thiazolyl)butyl]thio]-4-chlorobenzonitrile, (2S,4R)-2-amino-4-[[2-chloro-5-(trifluoromethyl)phenyl]thio]-5-thiazolebutanol, 2-[[(1R,3S)-3-amino-4-hydroxy-1-(5-thiazolyl)butyl]thio]-6-(trifluoromethyl)-3-pyridinecarbonitrile, 2-[[(1R,3S)-3-amino-4-hydroxy-1-(5-thiazolyl)butyl]thio]-5-chlorobenzonitrile, N-[4-[2-(3-chlorobenzylamino)ethyl]phenyl]thiophene-2-carboxamidine, or guanidinoethyldisulfide;
an acetylcholinesterase inhibitor such as donepezil;
a prostaglandin E₂ subtype 4 (EP4) antagonist such as N-[({2-[4-(2-ethyl-4,6-dimethyl-1H-imidazo[4,5-c]pyridin-1-yl)phenyl]ethyl}amino)-carbonyl]-4-methylbenzenesulfonamide or 4-[(1S)-1-({[5-chloro-2-(3-fluorophenoxy)pyridin-3-yl]carbonyl}amino)ethyl]benzoic acid;
a leukotriene B4 antagonist; such as 1-(3-biphenyl-4-ylmethyl-4-hydroxy-chroman-7-yl)-cyclopentanecarboxylic acid (CP-105696), 5-[2-(2-Carboxyethyl)-3-[6-(4-methoxyphenyl)-5E-hexenyl]oxyphenoxy]-valeric acid (ONO-4057) or DPC-11870;
a 5-lipoxygenase inhibitor, such as zileuton, 6-[(3-fluoro-5-[4-methoxy-3,4,5,6-tetrahydro-2H-pyran-4-yl])phenoxy-methyl]-1-methyl-2-quinolone (ZD-2138), or 2,3,5-trimethyl-6-(3-pyridylmethyl),1,4-benzoquinone (CV-6504);
a sodium channel blocker, such as lidocaine;
a 5-HT3 antagonist, such as ondansetron;
a chemotherapy drug such as oxaliplatin, 5-fluorouracil, leukovolin, paclitaxel
   and the pharmaceutically acceptable salts and solvates thereof.

Such combinations offer significant advantages, including synergistic activity, in therapy.

The composition may contain from 0.1% to 99% by weight, preferably from 10 to 60% by weight, of the active material, depending on the method of administration. The dose of the compound used in the treatment of the aforementioned disorders will vary in the usual way with the seriousness of the disorders, the weight of the sufferer, and other similar factors.

A therapeutically effective amount of a compound of formula (I) or a pharmaceutical composition thereof includes a dose range from about 0.05 mg to about 3000 mg, in particular from about 1 mg to about 1000 mg or, more particularly, from about 10 mg to about 500 mg of active ingredient in a regimen of about once a day or more than once a day, for example two, three or four times a day for an average (70 kg) human; although, it is apparent to one skilled in the art that the therapeutically effective amount for active compounds of the invention will vary as will the diseases, syndromes, conditions, and disorders being treated.

For oral administration, a pharmaceutical composition is preferably provided in the form of tablets containing about 0.01, about 10, about 50, about 100, about 150, about 200, about 250, and about 500 milligrams of the inventive compound as the active ingredient.

Advantageously, a compound of formula (I) may be administered in a single daily dose, or the total daily dosage may be administered in divided doses of two, three and four times daily.

Optimal dosages of a compound of formula (I) to be administered may be readily determined and will vary with the particular compound used, the mode of administration, the strength of the preparation, and the advancement of the disease, syndrome, condition, or disorder. In addition, factors associated with the particular subject being treated, including subject age, weight, diet and time of administration, will result in the need to adjust the dose to achieve an appropriate therapeutic level.

The above dosages are thus exemplary of the average case. There can be, of course, individual instances wherein higher or lower dosage ranges are merited, and such are within the scope of this invention.

Compounds of formula (I) may be administered in any of the foregoing compositions and dosage regimens or by means of those compositions and dosage regimens established in the art whenever use of a compound of formula (I) is required for a subject in need thereof.

As antagonists of the TRPM8 ion channel, the compounds of formula (I) are useful in methods for treating and preventing a disease, a syndrome, a condition, or a disorder in a subject, including an animal, a mammal and a human in which the disease, the syndrome, the condition, or the disorder is affected by the modulation of TRPM8 receptors. Such methods comprise, consist of, and consist essentially of administering to a subject, including an animal, a mammal, and a human in need of such treatment or prevention a therapeutically effective amount of a compound, salt, or solvate of formula (I). In particular, the compounds of formula (I) are useful for preventing or treating pain, or diseases, syndromes, conditions, or disorders causing such pain, or pulmonary or vascular dysfunction. More particularly, the compounds of formula (I) are useful for preventing or treating inflammatory pain, inflammatory hypersensitivity conditions, neuropathic pain, anxiety, depression, and cardiovascular disease aggravated by cold, including peripheral vascular disease, vascular hypertension, pulmonary hypertension, Raynaud's disease, and coronary artery disease, by administering to a subject in need thereof a therapeutically effective amount of a compound of formula (I).

Examples of inflammatory pain include pain due to a disease, condition, syndrome, disorder, or a pain state including inflammatory bowel disease, visceral pain, migraine, post operative pain, osteoarthritis, rheumatoid arthritis, back pain, lower back pain, joint pain, abdominal pain, chest pain, labor, musculoskeletal diseases, skin diseases, toothache, pyresis, burn, sunburn, snake bite, venomous snake bite, spider bite, insect sting, neurogenic bladder, interstitial cystitis, urinary tract infection, rhinitis, contact dermatitis/hypersensitivity, itch, eczema, pharyngitis, mucositis, enteritis, irritable bowel syndrome, cholecystitis, pancreatitis, postmastectomy pain syndrome, menstrual pain, endometriosis, sinus headache, tension headache, or arachnoiditis.

One type of inflammatory pain is inflammatory hyperalgesia, which can be further distinguished as inflammatory somatic hyperalgesia or inflammatory visceral hyperalgesia. Inflammatory somatic hyperalgesia can be characterized by the presence of an inflammatory hyperalgesic state in which a hypersensitivity to thermal, mechanical and/or chemical stimuli exists. Inflammatory visceral hyperalgesia can also be characterized by the presence of an inflammatory hyperalgesic state, in which an enhanced visceral irritability exists.

Examples of inflammatory hyperalgesia include a disease, syndrome, condition, disorder, or pain state including inflammation, osteoarthritis, rheumatoid arthritis, back pain, joint pain, abdominal pain, musculoskeletal diseases, skin diseases, post operative pain, headaches, toothache, burn, sunburn, insect sting, neurogenic bladder, urinary incontinence, interstitial cystitis, urinary tract infection, cough, asthma, chronic obstructive pulmonary disease, rhinitis, contact dermatitis/hypersensitivity, itch, eczema, pharyngitis, enteritis, irritable bowel syndrome, inflammatory bowel diseases including Crohn's Disease or ulcerative colitis.

One embodiment of the present invention is directed to a method for treating inflammatory somatic hyperalgesia in which a hypersensitivity to thermal, mechanical and/or chemical stimuli exists, comprising the step of administering to a mammal in need of such treatment a therapeutically effective amount of a compound, salt or solvate of formula (I).

A further embodiment of the present invention is directed to a method for treating inflammatory visceral hyperalgesia in which a enhanced visceral irritability exists, comprising, consisting of, and/or consisting essentially of the step of administering to a subject in need of such treatment a therapeutically effective amount of a compound, salt or solvate of formula (I).

A further embodiment of the present invention is directed to a method for treating neuropathic cold allodynia in which a hypersensitivity to a cooling stimuli exists, comprising, consisting of, and/or consisting essentially of the step of administering to a subject in need of such treatment a therapeutically effective amount of a compound, salt or solvate of formula (I).

Examples of an inflammatory hypersensitivity condition include urinary incontinence, benign prostatic hypertrophy, cough, asthma, rhinitis and nasal hypersensitivity, itch, contact dermatitis and/or dermal allergy, and chronic obstructive pulmonary disease.

Examples of a neuropathic pain include pain due to a disease, syndrome, condition, disorder, or pain state including cancer, neurological disorders, spine and peripheral nerve surgery, brain tumor, traumatic brain injury (TBI), spinal cord trauma, chronic pain syndrome, fibromyalgia, chronic fatigue syndrome, neuralgias (trigeminal neuralgia, glossopharyngeal neuralgia, postherpetic neuralgia and causalgia), lupus, sarcoidosis, peripheral neuropathy, bilateral peripheral neuropathy, diabetic neuropathy, central pain, neuropathies associated with spinal cord injury, stroke, amyotrophic lateral sclerosis (ALS), Parkinson's disease, multiple sclerosis, sciatic neuritis, mandibular joint neuralgia, peripheral neuritis, polyneuritis, stump pain, phantom limb pain, bony fractures, oral neuropathic pain, Charcot's pain, complex regional pain syndrome I and II (CRPS I/II), radiculopathy, Guillain-Barre syndrome, meralgia paresthetica, burning-mouth syndrome, optic neuritis, postfebrile neuritis, migrating neuritis, segmental neuritis, Gombault's neuritis, neuronitis, cervicobrachial neuralgia, cranial neuralgia, geniculate neuralgia, glossopharyngial neuralgia, migrainous neuralgia, idiopathic neuralgia, intercostals neuralgia, mammary neuralgia, Morton's neuralgia, nasociliary neuralgia, occipital neuralgia, red neuralgia, Sluder's neuralgia, splenopalatine neuralgia, supraorbital neuralgia, vulvodynia, or vidian neuralgia.

One type of neuropathic pain is neuropathic cold allodynia, which can be characterized by the presence of a neuropathy-associated allodynic state in which a hypersensitivity to cooling stimuli exists. Examples of neuropathic cold allodynia include allodynia due to a disease, condition, syndrome, disorder or pain state including neuropathic pain or neuralgia, pain arising from spine and peripheral nerve surgery or trauma, traumatic brain injury (TBI), trigeminal neuralgia, postherpetic neuralgia, causalgia, peripheral neuropathy, diabetic neuropathy, central pain, stroke, peripheral neuritis, polyneuritis, complex regional pain syndrome I and II (CRPS I/II) and radiculopathy.

Examples of anxiety include social anxiety, post traumatic stress disorder, phobias, social phobia, special phobias, panic disorder, obsessive compulsive disorder, acute stress, disorder, separation anxiety disorder, and generalized anxiety disorder.

Examples of depression include major depression, bipolar disorder, seasonal affective disorder, post natal depression, manic depression, and bipolar depression.

### General Synthesis

Throughout the instant application, the following abbreviations are used with the following meanings:

### DMF: N,N-Dimethylformamide

### THF: Tetrahydrofuran

### DMSO: Dimethylsulfoxide

EtOAc: Ethyl acetate

### MeOH: Methanol

### EtOH: Ethanol

### DCM: Dichloromethane

DME: 1,2-dimethoxyethane
TFA: Trifluoroacetic acid

### MeCN: Acetonitrile

### Et3N: Triethylamine

### DMAP: 4-Dimethylaminopyridine

EDC: 1-Ethyl-3-(3-dimethylaminopropyl)carbodiimide Hydrochloride

### FMOC: 9-Fluorenylmethoxycarbonyl

### HOST: 1-Hydroxybenztriazole

HBTU: O-(Benzotriazol-1-yl)-N,N,N′,N′-tetramethyluronium Hexafluorophosphate
BOP: (Benzotriazol-1-yloxy)tris(dimethylamino)phosphonium Hexafluorophosphate
HPLC: High pressure liquid chromatography
tR: Retention time

### MHz: Megahertz

### NMR: Nuclear Magnetic Resonance

TLC: Thin layer chromatography

The term of “base” is likewise no particular restriction on the nature of the bases used, and any base commonly used in reactions of this type may equally be used here. Examples of such bases include: alkali metal hydroxides, such as lithium hydroxide, sodium hydroxide, potassium hydroxide, and barium hydroxide; alkali metal hydrides, such as lithium hydride, sodium hydride, and potassium hydride; alkali metal alkoxides, such as sodium methoxide, sodium ethoxide, and potassium t-butoxide; alkali metal carbonates, such as lithium carbonate, sodium carbonate, potassium carbonate, and cesium carbonate; alkali metal hydrogencarbonates, such as lithium hydrogencarbonate, sodium hydrogencarbonate, and potassium hydrogencarbonate; amines, such as N-methylmorpholine, triethylamine, tripropylamine, tributylamine, diisopropylethylamine, N-methylpiperidine, pyridine, 4-pyrrolidinopyridine, picoline, 2,6-di(t-butyl)-4-methylpyridine, quinoline, N,N-dimethylaniline, N,N-diethylaniline, 1,5-diazabicyclo[4.3.0]non-5-ene (DBN), 1,4-diazabicyclo[2.2.2]octane (DABCO), 1,8-diazabicyclo[5.4.0]undec-7-ene (DBU), lutidine, and colidine; alkali metal amides, such as lithium amide, sodium amide, potassium amide, lithium diisopropyl amide, potassium diisopropyl amide, sodium diisopropyl amide, lithium bis(trimethylsilyl)amide and potassium bis(trimethylsilyl)amide. Of these, triethylamine, diisopropylethylamine, DBU, DBN, DABCO, pyridine, lutidine, colidine, sodium carbonate, sodium hydrogencarbonate, sodium hydroxide, potassium carbonate, potassium hydrogencarbonate, potassium hydroxide, barium hydroxide, and cesium carbonate are preferred.

The reactions are normally and preferably effected in the presence of inert solvent. There is no particular restriction on the nature of the solvent to be employed, provided that it has no adverse effect on the reaction or the reagents involved and that it can dissolve reagents, at least to some extent. Examples of suitable solvents include, but not limited to: halogenated hydrocarbons, such as dichloromethane, chloroform, carbon tetrachloride, and dichloroethane; ethers, such as diethyl ether, diisopropyl ether, THF, and dioxane; aromatic hydrocarbons, such as benzene, toluene and nitrobenzene; amides, such as, DMF, N,N-dimethylacetamide, and hexamethylphosphoric triamide; amines, such as N-methylmorpholine, triethylamine, tripropylamine, tributylamine, diisopropylethylamine, N-methylpiperidine, pyridine, 4-pyrrolidinopyridine, N,N-dimethylaniline, and N,N-diethylaniline; alcohols, such as methanol, ethanol, propanol, isopropanol, and butanol; nitriles, such as acetonitrile and benzonitrile; sulfoxides, such as dimethyl sulfoxide (DMSO) and sulfolane; ketones, such as acetone and diethylketone. Of these solvents, including but not limited to DMF, DMSO, THF, diethylether, diisopropylether, dimethoxyethane, acetonitrile, dichloromethane, dichloroethane and chloroform are preferred.

### EXAMPLES

The invention is illustrated in the following non-limiting examples in which, unless stated otherwise: all reagents are commercially available, all operations were carried out at room or ambient temperature, that is, in the range of about 18-25° C.; evaporation of solvent was carried out using a rotary evaporator under reduced pressure with a bath temperature of up to about 60° C.; reactions were monitored by thin layer chromatography (tlc) and reaction times are given for illustration only; the structure and purity of all isolated compounds were assured by at least one of the following techniques: tlc (Merck silica gel 60 F₂₅₄ precoated TLC plates or Merck NH₂ F₂₅₄ precoated HPTLC plates), mass spectrometry or nuclear magnetic resonance (NMR). Yields are given for illustrative purposes only. Flash column chromatography was carried out using Merck silica gel 60 (230-400 mesh ASTM) or Fuji Silysia Chromatorex (registered trademark) DU3050 (Amino Type, 30-50 micrometer) or Biotage silica (32-63 mm, KP-Sil) or Biotage amino bounded silica (35-75 mm, KP-NH). The purification of compounds using preparative LC-MS system was performed by the following apparatus and conditions (“Process A”); Apparatus; Waters MS-trigger AutoPurification™ system Column; Waters XTerra C18, 19×50 mm, 5 mm particle, Method A; Methanol or acetonitrile/0.05% (v/v) formic acid aqueous solution, Method B; Methanol or acetonitrile/0.01% (v/v) ammonia aqueous solution. The purification using HPLC (“Process B”) was performed by the following apparatus and conditions: Apparatus; UV-trigger preparative HPLC system, Waters (Column; XTerra MS C18, 5 micrometer, 19×50 mm or 30×50 mm), Detector; UV 254 nm, Conditions; acetonitrile: 0.05% formic acid aqueous solution or acetonitrile: 0.01% aqueous ammonia solution; 20 mL/min (19×50 mm) or 40 mL/min (30×50 mm) at room temperature. Low-resolution mass spectral data (ESI) were obtained by the following apparatus and conditions: Apparatus; Waters Alliance HPLC system on ZQ or ZMD mass spectrometer and UV detector. Microwave reaction was conducted by Intiator (registered trademark) Sixty (Biotage). NMR data was determined at 270 MHz (JEOL JNM-LA 270 spectrometer) or 300 MHz (JEOL JNM-LA300) using deuterated chloroform (99.8% D) or dimethylsulfoxide (99.9% D) as solvent unless indicated otherwise, relative to tetramethylsilane (TMS) as internal standard in parts per million (ppm); conventional abbreviations used are: s=singlet, d=doublet, t=triplet, q=quartet, m=multiplet, br=broad, etc. Chemical symbols have their usual meanings; microL (microliter(s)), microg (microgram(s)), M (mol(s) per liter), L (liter(s)), mL (milliliter(s)), g (gram(s)), mg (milligram(s)), mol (moles), mmol (millimoles).

### Conditions for Determining LC-MS Retention Time:

### Method A:

Apparatus: Waters Acquity Ultra Performance LC on TUV Detector and ZQ mass spectrometer
Column: XTerra MS C18 3.5 micrometer, 2.1×30 mm

### Column Temperature: 45° C.

### Solvents:

A1: acetonitrile
B1: 5 mM ammonium acetate aqueous solution

TABLE 1

| Time(min) | A1(%) | B1(%) |
|---|---|---|
| 0 | 4 | 96 |
| 2 | 96 | 4 |
| 4 | 96 | 4 |

run time 4.0 min
flow 0.5 mL/min

### Method B:

Apparatus: Waters Acquity Ultra Performance LC on TUV Detector and ZQ mass spectrometer
Column: XTerra MS C18 3.5 micrometer, 2.1×30 mm

### Column Temperature: 45° C.

### Solvents:

A1: acetonitrile
B1: 5 mM ammonium acetate aqueous solution

TABLE 2

| Time(min) | A1(%) | B1(%) |
|---|---|---|
| 0 | 32 | 68 |
| 2 | 96 | 4 |
| 4 | 96 | 4 |

run time 4.0 min
flow 0.5 mL/min

### Conditions for Determining HPLC Retention Time:

### Method C:

Apparatus: Waters Acquity Ultra Performance LC on TUV Detector and ZQ mass spectrometer
Column: Waters ACQUITY C18, 2.1×100 mm, 1.7 micrometer particle

### Column Temperature: 60° C.

### Solvents:

A1: 10 mM ammonium acetate aqueous solution
B1: Acetonitrile

TABLE 3

| Time(min) | A1(%) | B1(%) |
|---|---|---|
| 0 | 95 | 5 |
| 0.1 | 95 | 5 |
| 1.8 | 5 | 95 |
| 2.3 | 95 | 5 |

run time 3.0 min
flow 0.7 mL/min

### Method D:

Apparatus: Waters Alliance HPLC system on ZQ mass spectrometer and UV detector
Column: Waters SunFire C18 2.1×50 mm, 3.5 micrometer particle

### Column Temperature: 40° C.

### Solvents:

A: Water (Mili-Q)
B: Acetonitrile
C: 1% formic acid aqueous solution
D: 1% ammonia aqueous solution

TABLE 4

| Time(min) | A(%) | B(%) | C(%) | D(%) |
|---|---|---|---|---|
| 0 | 90 | 5 | 5 | 0 |
| 0.5 | 90 | 5 | 5 | 0 |
| 3.5 | 0 | 95 | 5 | 0 |
| 4.5 | 90 | 5 | 5 | 0 |

run time 5 min
flow 1 mL/min

### Method E:

Apparatus: Waters Alliance HPLC system on ZQ mass spectrometer and UV detector
Column: Waters XBridge C18 2.1×50 mm, 3.5 micrometer particle

### Column Temperature: 40° C.

### Solvents:

A: Water (Mili-Q)
B: acetonitrile
C: 1% formic acid aqueous solution
D: 1% ammonia aqueous solution

TABLE 5

| Time(min) | A(%) | B(%) | C(%) | D(%) |
|---|---|---|---|---|
| 0 | 90 | 5 | 0 | 5 |
| 0.5 | 90 | 5 | 0 | 5 |
| 3.5 | 0 | 95 | 0 | 5 |
| 4.5 | 90 | 5 | 0 | 5 |

run time 5 min
flow 1 mL/min

All of the compounds of the formula (I) can be prepared by the procedures described in the general methods presented below or by the specific methods described in the Examples section and the Preparations section, or by routine modifications thereof. The present invention also encompasses any one or more of these processes for preparing the sulfamoyl benzoic acid derivatives of formula (I), in addition to any novel intermediates used therein.

In the following general methods, Ar¹, Z, R¹, R², R³, R⁴, R⁵, R⁶, X, Y, m, n, p, and q are as previously defined for sulfamoyl benzoic acid derivatives of the formula (I) unless otherwise stated.

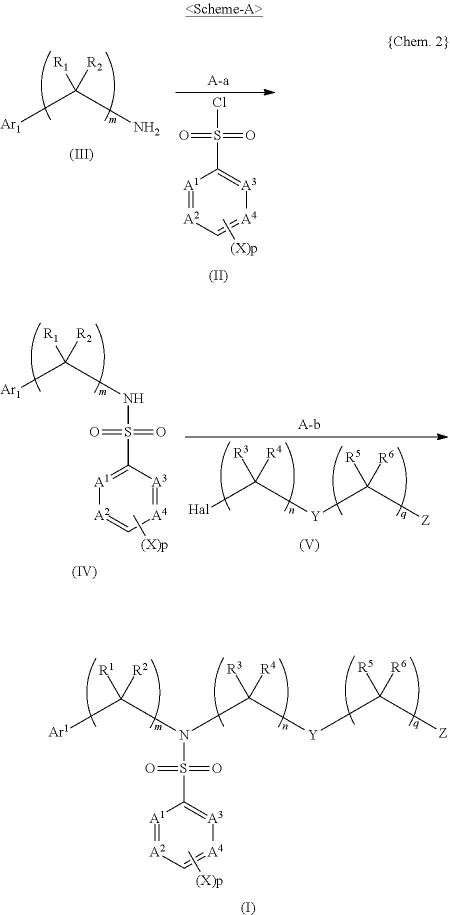

In Step A-a, the compounds of formula (IV) can be prepared by sulfonylation of the compound of formula (III) with the compound of formula (II) under, for example, known sulfonylation conditions in the presence of a base in an inert solvent. A preferred base is selected from, for example, but not limited to: an alkali or alkaline earth metal hydroxide, alkoxide, carbonate, halide or hydride, such as sodium hydroxide, potassium hydroxide, sodium methoxide, sodium ethoxide, potassium tert-butoxide, sodium carbonate, potassium carbonate, potassium fluoride, sodium hydride or potassium hydride; or an amine such as triethylamine, tributylamine, diisopropylethylamine, 2,6-lutidine, pyridine or dimethylaminopyridine. Examples of suitable inert aqueous or non-aqueous organic solvents include: alcohols, such as methanol or ethanol; ethers, such as THF or 1,4-dioxane; acetone; dimethylformamide; halogenated hydrocarbons, such as DCM, 1,2-dichloroethane or chloroform; and pyridine; or mixtures thereof. The reaction can be carried out at a temperature in the range of from −10° C. to 150° C., preferably in the range of from 20° C. to 60° C. Reaction times are, in general, from 10 minutes to 4 days, preferably from 30 minutes to 24 hrs.

When Hal is a halogen group such as iodide, bromide, or chloride, in Step A-b, a compound of formula (I) can be prepared by N-substitution reaction of a compound of formula (IV) with alkyl halide (V) in the presence of a base in an inert solvent.

Examples of suitable bases include: potassium carbonate, sodium carbonate and cesium carbonate. Examples of suitable solvents include: 1,4-dioxane, acetonitrile, DMSO and DMF. This reaction can be carried out in the presence of a suitable additive agent. Examples of such additive agents include: sodium iodide and potassium iodide. The reaction can be carried out at a temperature of from 20° C. to 150° C., more preferably from 20° C. to 100° C. Reaction times are, in general, from 5 minutes to 96 hours, more preferably from 30 minutes to 24 hrs.

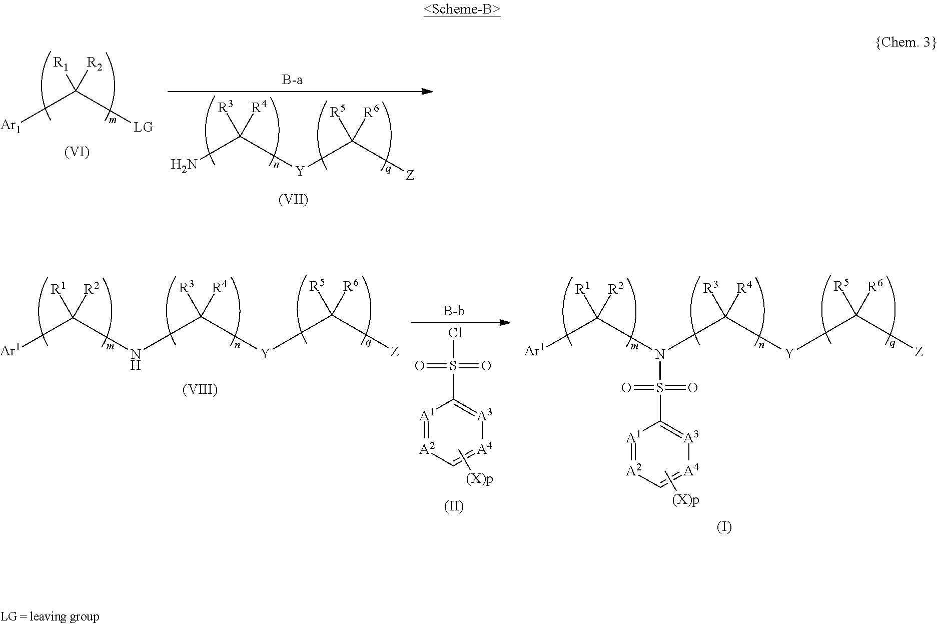

When LG is a suitable leaving group such as O-trifluoromethanesulfonate, O-tosylate, O-mesylate, iodide, bromide, or chloride, in Step B-a, a compound of formula (VIII) can be prepared by N-alkylation and/or N-arylation of a compound of formula (VII) with a compound of formula (VI) in the presence of a base in an inert solvent. Examples of suitable bases include: potassium carbonate, sodium carbonate and cesium carbonate. Examples of suitable solvents include: 1,4-dioxane, acetonitrile, DMSO and DMF. The reaction can be carried out at a temperature of from 0° C. to 200° C., more preferably from 20° C. to 160° C. Reaction times are, in general, from 5 minutes to 96 hrs, more preferably from 30 minutes to 24 hrs. In an alternative case, the reaction can be carried out with microwave system in the presence of a same base in a same inert solvent. The reaction can be carried out at a temperature in the range of from 100° C. to 200° C., preferably in the range of from 120° C. to 150° C. Reaction times are, in general, from 10 minutes to 5 hrs, preferably from 15 minutes to 3 hr.

In Step B-b, a compound of formula (I) can be prepared from a compound of formula (VIII) by the method described in Step A-a above. A preferred inert solvent is pyridine. The reaction can be carried out at a temperature in the range of from 100° C. to 150° C., preferably in the range of from 110° C. to 140° C. Reaction times are, in general, from 10 minutes to 4 days, preferably from 24 hrs to 48 hrs.

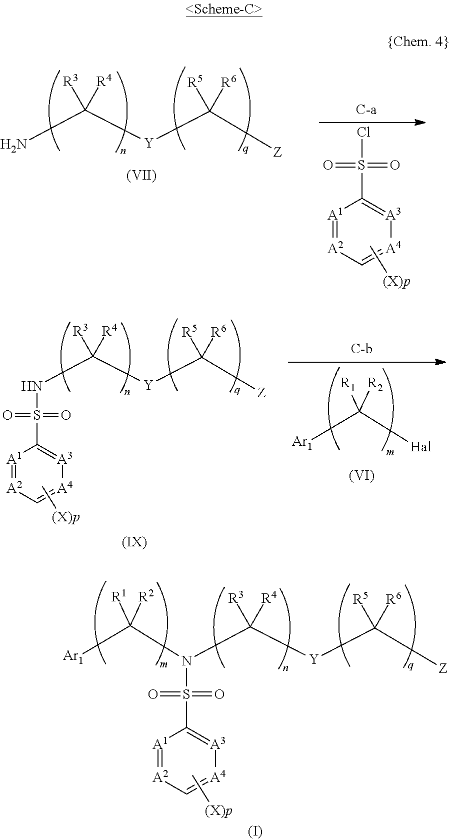

In Step C-a, a compound of formula (IX) can be prepared from a compound of formula (VII) by the method described in Step A-a above.

When Hal is a halogen group such as iodide, bromide, or chloride, in Step C-b, a compound of formula (I) can be prepared from a compound of formula (IX) by the method described in Step B-a above.

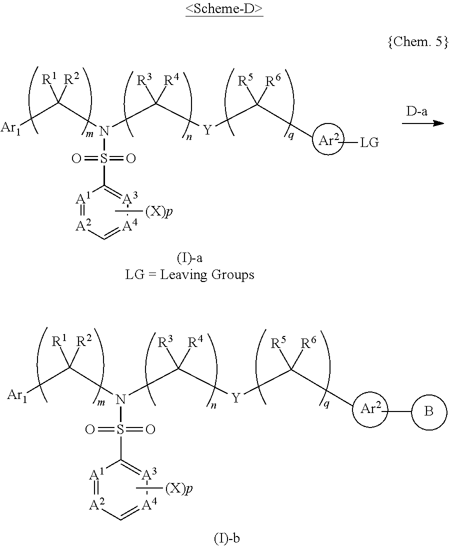

When LG is a suitable leaving group such as O-trifluoromethanesulfonate, O-tosylate, O-mesylate, iodide, bromide, or chloride and B ring is (C₃-C₆)cycloalkyl and aryl, in Step D-a, the compound of formula ((I)-b) can be prepared by coupling of a compound of formula ((I)-a) with (C₃-C₆)cycloalkyl or aryl metal reagents in water-organic co-solvent mixture under coupling conditions in the presence of a suitable transition metal catalyst and in the presence or absence of a base. Examples of suitable transition metal catalysts include: tetrakis(triphenylphosphine)palladium(0), bis(triphenylphosphine)palladium(II) chloride, copper(0), copper(I) acetate, copper(I) bromide, copper(I) chloride, copper(I) iodide, copper(I) oxide, copper(II) trifluoromethanesulfonate, copper(II) acetate, copper(II) bromide, copper(II) chloride, copper(II) iodide, copper(II) oxide, copper(II) trifluoromethanesulfonate, palladium(II) acetate, palladium(II) chloride, bis(acetonitrile)dichloropalladium(II), bis(dibenzylideneacetone)palladium(0), tris(dibenzylideneacetone)dipalladium(0) and [1,1′-bis(diphenylphosphino)ferrocene]palladium(II) dichloride. Preferred catalysts are tetrakis(triphenylphosphine)palladium(0), bis(triphenylphosphine)palladium(II) chloride, palladium(II) acetate, palladium(II) chloride, bis(acetonitrile)dichloropalladium(0), bis(dibenzylideneacetone)palladium(0), tris(dibenzylideneacetone)dipalladium(0) and [1,1-bis(diphenylphosphino)ferrocene]palladium(II) dichloride.

Examples of suitable (C₃-C₆) cycloalkyl and aryl metal reagents include, but not limited to, boronic acids such as cyclopropylboronic acid, benzene boronic acid and 4-pyridinyl boronic acid; boronic esters such as cyclopropylboronic acid pinacol ester and benzeneboronic acid pinacol ester, benzeneboronic acid neopentyl glycol ester; benzeneboronic acid N-methyldiethanolamine ester; and trifluoroborate salts such as potassium phenyltrifluoroborate.

Examples of suitable organic solvent for the water-organic co-solvent mixture include: THF; 1,4-dioxane; DMF; acetonitrile; alcohols, such as methanol or ethanol; halogenated hydrocarbons, such as DCM, 1,2-dichloroethane, chloroform or carbon tetrachloride; and diethylether; in the presence or absence of an aqueous base such as aqueous potassium hydroxide, sodium hydroxide, lithium hydroxide, sodium bicarbonate, sodium cabonate or potassium carbonate. This reaction can be carried out in the presence of a suitable additive agent. Examples of such additive agents include: triphenylphosphine, tri-tert-butylphosphine, 1,1′-bis(diphenylphosphino)ferrocene, tri-2-furylphosphine, tri-o-tolylphosphine, 2-(dichlorohexylphosphino)biphenyl, triphenylarsine, tetrabutylammonium chloride, tetrabutylammonium fluoride, lithium acetate, lithium chloride, triethylamine, potassium or sodium methoxide, sodium hydroxide, cesium carbonate, tripotassium phosphate, sodium carbonate, sodium bicarbonate, and/or sodium iodide. The reaction can be carried out at a temperature of from 0° C. to 200° C., more preferably from 20° C. to 150° C. Reaction times are, in general, from 5 minutes to 96 hrs, more preferably from 30 minutes to 24 hrs. In an alternative case, the reaction can be carried out with microwave system in the presence of a base in an inert solvent. The reaction can be carried out at a temperature in the range of from 100° C. to 200° C., preferably in the range of from 120° C. to 150° C. Reaction times are, in general, from 10 minutes to 3 hrs, preferably from 15 minutes to 1 hr.

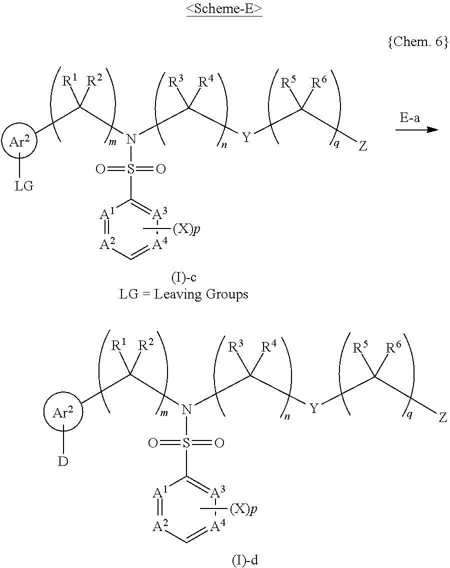

When LG is a suitable leaving group such as O-trifluoromethanesulfonate, O-tosylate, O-mesylate, iodide, bromide, or chloride and D is C₁-C₄ alkyl, C₃-C₆ cycloalkyl and aryl, in Step E-a, an amide compound of formula ((I)-d) can be prepared by coupling reaction of a compound of formula ((I)-c) with alkyl, cycloalkyl or aryl metal reagents (D-metal reagents) according to the method described in Step D-a above. Examples of suitable alkyl metal regents include boronic acids such as methyl boronic acid (or trimethylboroxine), boronic esters such as tert-butyllboronic acid pinacol ester; and trifluoroborate salts such as potassium methyltrifluoroborate

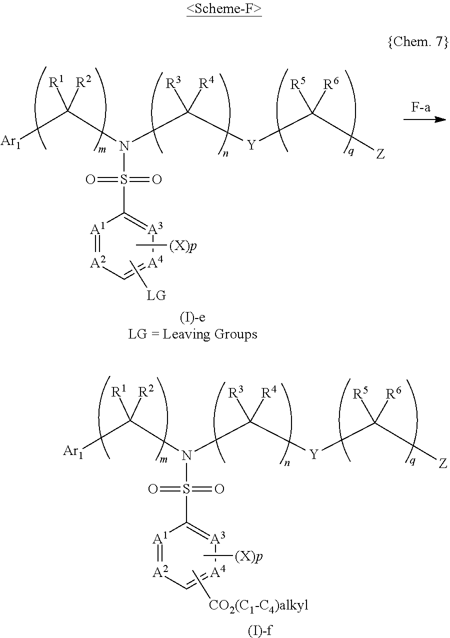

When LG is a suitable leaving group such as O-trifluoromethanesulfonate, O-tosylate, O-mesylate, iodide, bromide, or chloride, in Step F-a, the compound of formula ((I)-f) can be prepared by reacting the compound of formula ((I)-e) with carbon monoxide and alcohol (e.g. MeOH or EtOH) in the presence of a catalyst and/or base in an inert solvent. Examples of suitable catalysts include: palladium reagents, such as palladium(II) acetate or bis(dibenzylideneacetone)palladium(II). Examples of suitable bases include: N,N-diisopropylethylamine, N-methylmorpholine or triethylamine. If desired, this reaction may be carried out in the presence or absence of an additive such as 1,1′-bis(diphenylphosphino)ferrocene, triphenylphosphine or 1,3-bis(diphenylphosphino)propane (DPPP). Examples of suitable solvents include: acetone; nitromethane; DMF; sulfolane; DMSO; NMP; 2-butanone; acetonitrile; halogenated hydrocarbons such as DCM, 1,2-dichloroethane or chloroform; or ethers, such as THF or 1,4-dioxane. The reaction can be carried out at a temperature of from 0° C. to 200° C., more preferably from 20° C. to 120° C. Reaction times are, in general, from 5 minutes to 96 hrs, more preferably from 30 minutes to 24 hrs.

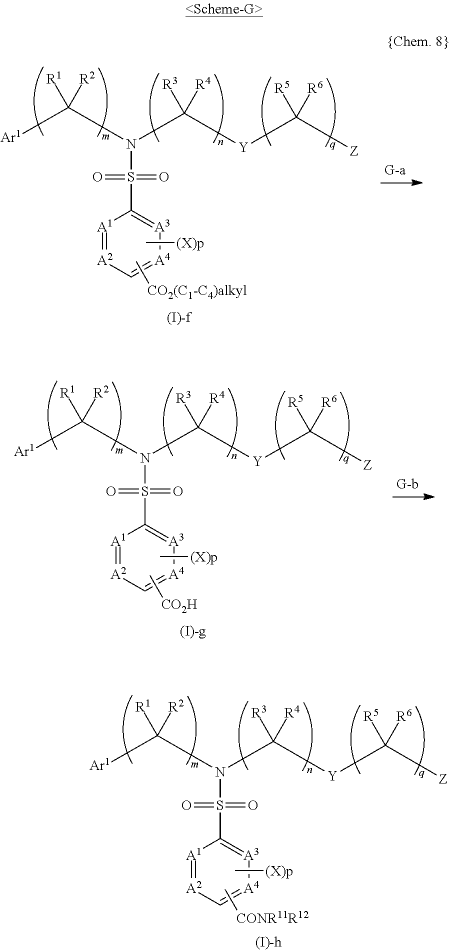

In Step G-a, an acid compound of formula ((I)-g) can be prepared by hydrolysis of the ester compound of formula ((I)-f) in an inert solvent. The hydrolysis can be carried out by conventional procedures. In a typical procedure, the hydrolysis is carried out under basic conditions, e.g. in the presence of sodium hydroxide, potassium hydroxide or lithium hydroxide. Suitable solvents include, for example: alcohols such as methanol, ethanol, propanol, butanol, 2-methoxyethanol, and ethylene gylcol; ethers such as THF, DME, and 1,4-dioxane; amides such as DMF and hexamethylphospholictriamide; and sulfoxides such as DMSO. Preferred solvents are methanol, ethanol, propanol, THF, DME, 1,4-dioxane, DMF, and DMSO. This reaction can be carried out at a temperature in the range of from −20° C. to 100° C., usually from 20° C. to 65° C. for from 30 minutes to 24 hrs, usually from 60 minutes to 10 hrs.

In Step G-b, an amide compound of formula ((I)-h) can be prepared by the coupling reaction of an amine compound (R¹¹R¹²NH) with the acid compound of formula ((I)-g) in the presence or absence of a coupling reagent in an inert solvent. This reaction can be also carried out via activated carboxylic derivatives. Suitable coupling reagents are those typically used in peptide synthesis including, for example, not limited to, phosgene, triphosgene, ethyl chloroformate, isobutyl chloroformate, diphenyl phosphoryl azide (DPPA), diethyl phosphoryl cyanide (DEPC), carbodiimides [e.g., N,N′-dicyclohexylcarbodiimide (DCC), 1-ethyl-3-(3-dimethylaminopropyl)carbodiimide hydrochloride (EDCI)], imidazolium derived reagents [e.g., 1,1′-carbonyldiimidazole (CDI), 2-chloro-1,3-dimethylimidazolidinium hexafluorophosphate (CIP)], phosphonium salts [e.g., Benzotriazol-1-yl-oxy-tris(dimethylamino)phosphonium hexafluorophosphate (BOP), Benzotriazol-1-yl-oxy-tripyrrolidinophosphonium hexafluorophosphate (PyBOP (registered trademark)), Bromo-tripyrrolidinophosphonium hexafluorophosphate (PyBrop (registered trademark)), uronium and guanidinium salts [e.g., O-(benzotriazol-1-yl)-N,N,N′,N′-tetramethyluronium hexafluorophosphate (HBTU), O-(7-azabenzotriazol-1-yl)-N,N,N′,N′-tetramethyluronium hexafluorophosphate (HATU), O-(6-chlorobenzotriazol-1-yl)-N,N,N′,N′-tetramethyluronium hexafluorophosphate (HCTU)], miscellaneous coupling reagents [e.g., 2-chloro-4,6-dimethoxy-1,3,5-triazine (CDMT), (2-bromo-1-ethylpyridinium tetrafluoroborate) (BEP), 2-bromo-1-methylpyridinium iodide (BMPI)]. The reaction can be carried out in the presence of a base such as, 1-hydroxybenzotriazole (HOBt), 1-hydroxy-7-azabenzotriazole (HOAt), N,N-diisopropylethylamine, N-methylmorpholine or triethylamine. The reaction is normally and preferably effected in the presence of a solvent. There is no particular restriction on the nature of the solvent to be employed, provided that it has no adverse effect on the reaction or on the reagents involved and that it can dissolve the reagents, at least to some extent. Examples of suitable solvents include: acetone; nitromethane; DMF; N-Methyl-2-piperidone (NMP); sulfolane; DMSO; 2-butanone; acetonitrile; halogenated hydrocarbons, such as DCM, 1,2-dichloroethane, chloroform; and ethers, such as THF and 1,4-dioxane. The reaction can take place over a wide range of temperatures, and the precise reaction temperature is not critical to the invention. The preferred reaction temperature will depend upon such factors as the nature of the solvent, and the starting material or reagent used. However, in general, we find it convenient to carry out the reaction at a temperature of from −20° C. to 100° C., more preferably from about 0° C. to 60° C. The time required for the reaction can also vary widely, depending on many factors, notably the reaction temperature and the nature of the reagents and solvent employed. However, provided that the reaction is effected under the preferred conditions outlined above, a period of from 5 minutes to 1 week, more preferably from 30 minutes to 24 hrs, will usually suffice.

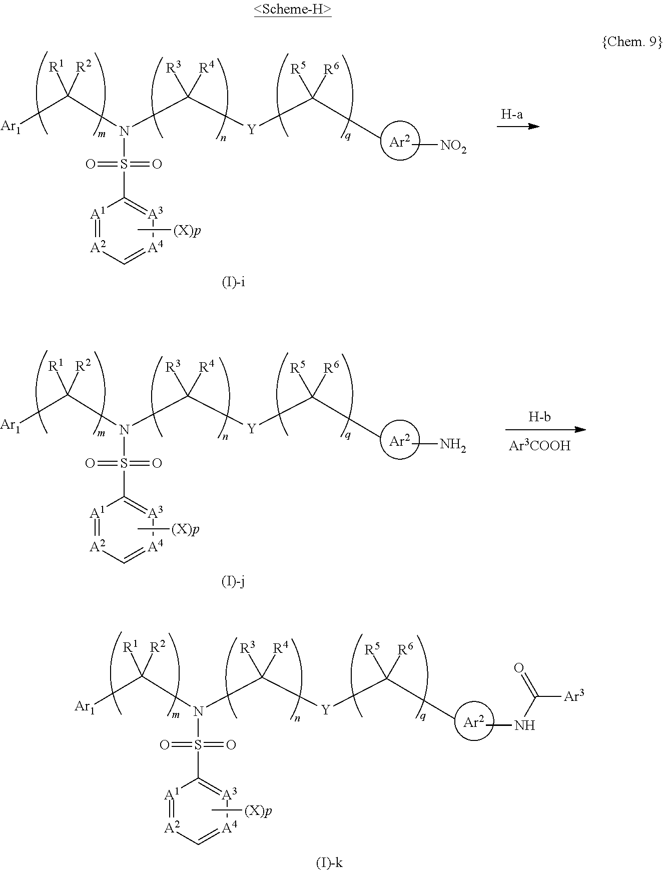

In Step H-a, a compound of formula ((I)-j) can be prepared by hydrogenation of a compound of formula ((I)-i) under, for example, known hydrogenolysis conditions in the presence of a suitable metal catalyst under a hydrogen atmosphere, or in the presence of hydrogen sources such as formic acid or ammonium formate, in an inert solvent. If desired, the reaction is carried out under acidic conditions, for example, in the presence of hydrochloric acid or acetic acid. A preferred metal catalyst is selected from, for example, nickel catalysts such as Raney nickel; Pd—C; palladiumhydroxide-carbon; platinumoxide; platinum-carbon; ruthenium-carbon; Fe; Zn; Sn; and SnCl₂. Examples of suitable inert aqueous or non-aqueous organic solvents include: alcohols, such as methanol, ethanol or ammonic methanol; ethers, such as THF or 1,4-dioxane; acetone; dimethylformamide; halogenated hydrocarbons, such as DCM, 1,2-dichloroethane or chloroform; and acetic acid; or mixtures thereof. The reaction can be carried out at a temperature in the range of from 20° C. to 150° C., preferably in the range of from 20° C. to 80° C. Reaction times are, in general, from 10 minutes to 4 days, preferably from 30 minutes to 24 hrs. This reaction can be carried out under a hydrogen atmosphere at a pressure ranging from 1 to 100 atoms, preferably from 1 to 10 atoms.

When Ar³ is 5 to 10 membered aryl, in Step H-b, the compounds of formula ((I)-k) can be prepared by amidation reaction via the compound of formula ((I)-j) according to the method described in Step G-b above.

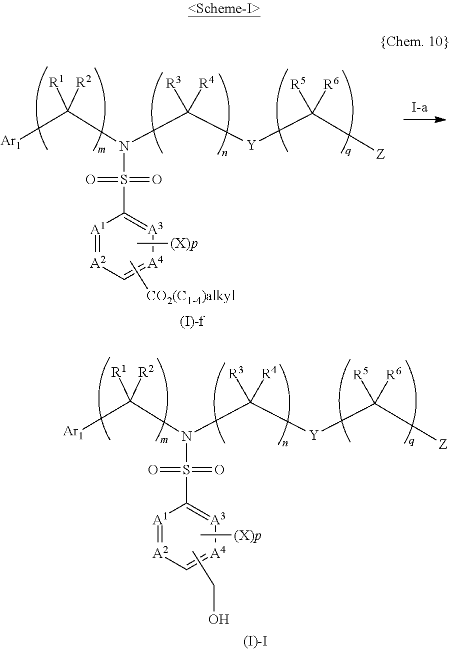

In Step I-a, the compounds of formula ((I)-l) can be prepared by reduction of the compound of formula ((I)-f). The reduction may be carried out in the presence of a suitable reducing agent in an inert solvent or without solvent. A preferred reducing agent is selected from, for example, but not limited to, NaBH₄, LiAlH₄, LiBH₄, BH₃-complex and (iso-butyl)₂AlH. Reaction temperatures are generally in the range of from −78° C. to 100° C., preferably in the range of from −70° C. to 60° C. Reaction times are, in general, from 1 minute to a day, preferably from 3 hrs to 12 hrs. Examples of suitable solvents include: THF; 1,4-dioxane; DMF; acetonitrile; alcohols, such as methanol or ethanol, and halogenated hydrocarbons, such as DCM, 1,2-dichloroethane, chloroform or carbon tetrachloride.

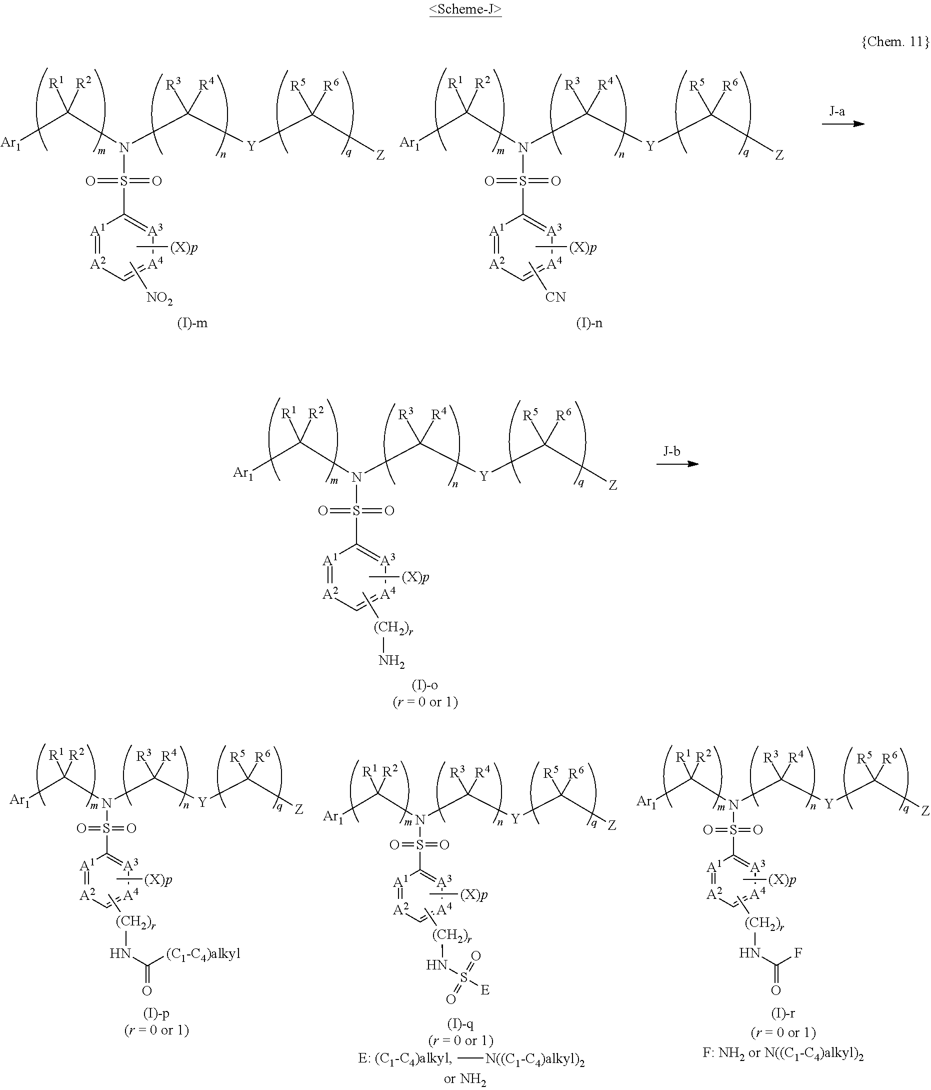

In Step J-a, a compound of formula ((I)-o) can be prepared from a compound of formula ((I)-m) and/or ((I)-n) by the method described in Step H-a above.

In Step J-b, a compound of formula ((I)-p) can be prepared from a compound of formula ((I)-o) by the method described in Step A-a above. Instead of sulfonyl chloride compound (II), a preferred reagent is selected from, for example, but not limited to, acid chloride such as acetyl chloride and acid anhydride such as acetic anhydride.

In Step J-b, a compound of formula ((I)-q) can be prepared from a compound of formula ((I)-o) by the method described in Step A-a above. When E presents NH₂, the corresponding compound is prepared from a compound of formula ((I)-o) and sulfamine.

In Step J-b, a compound of formula ((1)-r) can be prepared from a compound of formula ((I)-o) by the method described in Step A-a above. Instead of sulfonyl chloride compound (II), a preferred reagent is selected from, for example, but not limited to, isocyanate reagent such as trimethylsilyl isocyanate and N-carbonyl chloride such as N,N-dimethylcarbamoyl chloride.

All starting materials in the following general syntheses may be commercially available or obtained by conventional methods known to those skilled in the art. The key intermediate methyl 4-(chlorosulfonyl)benzoate can be prepared by the method described in Chemistry & Biology (2002) 9, 113.

Each of the final compounds was purified by preparative LC-MS system (“Process A”). After purification, mass spectrum (MS) and chemical purity were measured by HPLC-QC method using condition C, D or E. In general, “the preparative LC-MS system in usual manner” means the purification using “Process A”.

### Example 1

### 4-(N-Benzyl-N-(3-chloro-5-(trifluoromethyl)pyridin-2-yl)sulfamoyl)benzoic acid

### Step-1: Methyl 4-(N-(3-chloro-5-(trifluoromethyl)pyridin-2-yl)sulfamoyl)benzoate

To a pyridine (5 mL) solution of 3-chloro-5-(trifluoromethyl)pyridin-2-amine (1.0 g, 5.1 mmol) was added methyl 4-(chlorosulfonyl)benzoate (1.3 g, 5.6 mmol) and the mixture was refluxed for 14 hrs. The mixture was concentrated under reduced pressure. The residue was dissolved in DCM. The organic layer was then washed with 2 M aqueous HCl solution and sat. NaHCO₃, dried over MgSO₄. After the filtration to separate solvent and MgSO₄, the solvent was removed under reduced pressure to give 550 mg (27% yield) of methyl 4-(N-(3-chloro-5-(trifluoromethyl)pyridin-2-yl)sulfamoyl)benzoate as a dark solid that was used in the next step without further purification;

¹H-NMR (300 MHz, DMSO-d₆) δ 8.15-8.05 (2H, m), 8.05-7.95 (2H, m), 7.60-7.50 (2H, m), 3.92 (3H, s);

LC-MS (Method A) m/z: M+1 obs 394.8; tR=2.92 min.

### Step-2: Methyl 4-(N-benzyl-N-(3-chloro-5-(trifluoromethyl)pyridin-2-yl)sulfamoyl)benzoate

Methyl 4-(N-(3-chloro-5-(trifluoromethyl)pyridin-2-yl)sulfamoyl)benzoate (step-1 of Example 1, 550 mg, 1.4 mmol) was dissolved into DMF (5 mL). To the mixture were added K₂CO₃ (2.1 g, 15.0 mmol) and benzyl bromide (1.7 g, 10.0 mmol) at room temperature. The mixture was refluxed for 18 hrs. After being filtered off, the filtrate was concentrated under reduced pressure, the residue was applied to a silica gel chromatography column and eluted with a hexane/EtOAc=4/1 to furnish 310 mg (46% yield) of the titled compound as a white solid;

¹H-NMR (300 MHz, CDCl₃) δ 8.52 (1H, s), 8.21 (2H, d, J=8.8 Hz), 7.95 (1H, d, J=1.5 Hz), 7.88 (2H, d, J=8.8 Hz), 7.19 (5H, s), 4.69 (2H, s), 3.99 (3H, s);

LC-MS (Method A) m/z: M+1 484.8; tR=3.54 min.

### Step-3: 4-(N-Benzyl-N-(3-chloro-5-(trifluoromethyl)pyridin-2-yl)sulfamoyl)benzoic acid

To the solution of methyl 4-(N-benzyl-N-(3-chloro-5-(trifluoromethyl)pyridin-2-yl)sulfamoyl)benzoate (step-2 of Example 1, 1.1 g, 2.2 mmol) in THF (20 mL) was added 2 M aqueous NaOH solution (10 mL, 20.0 mmol) at room temperature and the mixture was refluxed at 90° C. with stirring for 3 hrs. The reaction was quenched with water and the product was extracted with EtOAc. The organic layer was then washed with brine, dried over Na₂SO₄. After the filtration to separate solvent and Na₂SO₄, the solvent was removed under reduced pressure to give the residue, which was recrystallized from diisopropyl ether to furnish 807 mg (99% yield) of the titled compound as a white solid;

¹H-NMR (300 MHz, CDCl₃) δ 8.57 (1H, s), 8.26 (2H, d, J=8.4 Hz), 7.97 (1H, s) 7.92 (2H, d J=8.4 Hz), 7.30-7.10 (5H, m), 4.74 (2H, s);

LC-MS (Method A) m/z: M+1 obs 470.8; tR=3.12 min.

### Example 2

### 4-(N-Benzyl-N-(3-(trifluoromethyl)pyridin-2-yl)sulfamoyl)benzoic acid

### Step-1: N-Benzyl-3-(trifluoromethyl)pyridin-2-amine

To a suspension of 2-chloro-3-(trifluoromethyl)pyridine (500 mg, 2.8 mmol) and K₂CO₃ (1.9 g, 13.8 mmol) in DMF (5 mL) was added benyzyl amine (0.9 g, 8.3 mmol) at room temperature and the mixture was stirred for 16 hrs at 110° C. The reaction was quenched with water and the product was extracted with (EtOAc/toluene=4/1). The organic layer was then washed with brine, dried over Na₂SO₄. After the filtration to separate solvent and Na₂SO₄, the solvent was removed under reduced pressure to give the residue, which was applied to an amino-silica gel chromatography column and eluted with a hexane/EtOAc=19/1 to furnish 363 mg (52% yield) of the titled compound as a white solid;

¹H-NMR (300 MHz, CDCl₃) δ 8.28 (1H, d, J=4.8 Hz), 7.67 (1H, d, J=7.3 Hz), 7.40-7.30 (5H, m), 6.65 (1H, dd, J=7.3, 4.8 Hz), 5.19 (1H, br s), 4.73 (2H, d, J=5.1 Hz).

LC-MS (Method A) m/z: M+1 obs 253.0, tR=3.28 min.

### Step-2: 4-(N-Benzyl-N-(3-(trifluoromethyl)pyridin-2-yl)sulfamoyl)benzoic acid

To a solution of N-benzyl-3-(trifluoromethyl)pyridin-2-amine (step-1 of Example 2, 40 mg; 0.16 mmol) in pyridine (1 mL) was added 4-(chlorosulfonyl)benzoic acid (350 mg, 1.6 mmol) at room temperature. The reaction mixture was stirred at 80° C. for 2 hrs. After the reaction mixture was cooled to room temperature, DMAP (3.9 mg, 0.03 mmol) was added to the mixture. The reaction mixture was refluxed with stirring for 2 days. After the reaction mixture was cooled to room temperature, the reaction was quenched with sat. NaHCO₃ aqueous solutions and the product was extracted with DCM. The organic layer was then washed with brine, dried over Na₂SO₄. After the filtration to separate solvent and Na₂SO₄, the solvent was removed under reduced pressure to give the residue. The residue was diluted with methanol and applied onto a strong cation exchange cartridge (BondElute (registered trademark) SCX, 1 g/6 mL, Varian Inc.), and the solid phase matrix was rinsed with MeOH (6 mL). The crude mixture was eluted in a collection tube with 1 M ammonia in MeOH (6 mL) and concentrated in vacuo. The residue was purified by preparative LC-MS (Process A) to give 2.0 mg (3% yield) of the titled compound.

### Example 3

### N-Benzyl-N-(3-chloro-5-(trifluoromethyl)pyridin-2-yl)benzenesulfonamide

### Step-1: N-(3-Chloro-5-(trifluoromethyl)pyridin-2-yl]benzenesulfonamide

Prepared as in step-1 of Example 1 from benzenesulfonyl chloride;

¹H-NMR (CDCl₃, 300 MHz) δ 8.41 (1H, s), 8.18 (2H, d, J=7.3 Hz), 8.00-7.80 (2H, m), 7.70-7.50 (3H, m);

LC-MS (Method A) m/z: M+1 obs 336.9, tR=2.97 min.

### Step-2: N-Benzyl-N-(3-chloro-5-(trifluoromethyl)pyridin-2-yl)benzenesulfonamide

Prepared as in step-2 of Example 1 from N-(3-chloro-5-(trifluoromethyl)pyridin-2-yl)benzenesulfonamide (step-1 of Example 3);

¹H-NMR (300 MHz, CDCl₃) δ 8.52 (1H, d, J=1.5 Hz), 7.93 (1H, d, J=2.2 Hz), 7.90-7.80 (2H, m), 7.68 (1H, m), 7.60-7.52 (2H, m), 7.37 (1H, m), 7.25-7.10 (4H, m), 4.69 (2H, s).

### Example 4

### N-(3-(Trifluoromethyl)phenyl)benzenesulfonamide

Prepared as in step-1 of Example 1 from 3-(trifluoromethyl)aniline and benzenesulfonyl chloride;

¹H-NMR (300 MHz, CDCl₃) δ 7.80 (2H, d, J=7.4 Hz), 7.58 (1H, m), 7.53-7.42 (2H, m), 7.42-7.25 (4H, m), 6.99 (1H, br s),

LC-MS (Method A) m/z: M+1 obs 301.9, tR=3.09 min.

### Example 5

### N-(2-(4-Methylpiperazin-1-yl)-2-oxoethyl)-N-(3-(trifluoromethyl)phenyl)benzenesulfonamide

### Step-1: tert-Butyl 2-(N-(3-(trifluoromethyl)phenyl)phenylsulfonamido)acetate

To a suspension of sodium hydride (93 mg, 2.3 mmol) in THF (5 mL) was added a solution of methyl N-(3-(trifluoromethyl)phenyl)benzenesulfonamide (Example 4, 500 mg, 1.7 mmol) in THF (1 mL) at 0° C. After being stirred at 0° C. for 20 min, tert-butyl 2-bromoacetate (260 mg, 1.3 mmol) was added to the mixture. The reaction mixture was refluxed at 90° C. with stirring for 4 hrs. The reaction was quenched with sat. ammonium chloride and the product was extracted with EtOAc. The organic layer was then washed with brine, dried over Na₂SO₄. After the filtration to separate solvent and Na₂SO₄, the solvent was removed under reduced pressure to give the residue, which was applied to a silica gel chromatography column and eluted with a hexane/EtOAc=4/1 to furnish 624 mg (91% yield) of the titled compound as a colorless oil;

¹H-NMR (300 MHz, CDCl₃) δ 7.70-7.40 (8H, m), 7.38 (1H, s), 4.31 (2H, s), 1.38 (9H, s).

### Step-2: 2-(N-(3-(Trifluoromethyl)phenyl)phenylsulfonamido)acetic acid

To a solution of tert-butyl 2-(N-(3-(trifluoromethyl)phenyl)phenylsulfonamido)acetate (step-1 of Example 5, 620 mg, 1.5 mmol) in DCM (1 mL) was added trifluoroacetic acid (1 mL) at room temperature. The reaction mixture was stirred at room temperature for 1 hr and concentrated in vacuo to give 453 mg (84% yield) of the titled compound as a white solid;

¹H-NMR (300 MHz, CDCl₃) δ 7.68-7.53 (4H, m), 7.52-7.43 (4H, m), 7.35 (1H, s), 4.46 (2H, s);

LC-MS (Method A) m/z: M+1 obs 359.9, tR=2.90 min.

### Step-3: N-(2-(4-Methylpiperazin-1-yl)-2-oxoethyl)-N-(3-(trifluoromethyl)phenyl)benzenesulfonamide

To a suspension of 2-(N-(3-(trifluoromethyl)phenyl)phenylsulfonamido)acetic acid (step-2 of Example 5, 30 mg, 0.08 mmol) and 1-methylpiperazine (13 mg, 0.13 mmol) in DCM (2 mL) were added Et₃N (25 mg, 0.25 mmol), EDC (21 mg, 0.1 mmol) and HOBt (6.4 mg, 0.04 mmol) respectively. The reaction mixture was stirred at room temperature for 18 hrs. The solvent was evaporated by N₂-flow. The resulting residue was dissolved into EtOAc and water was added to the mixture. The organic layer was then washed with brine, dried over Na₂SO₄. After the filtration to separate solvent and Na₂SO₄, the solvent was removed under reduced pressure to give the residue. The residue was diluted with MeOH and applied onto a strong cation exchange cartridge (BondElute (registered trademark) SCX, 1 g/6 mL, Varian Inc.), and the solid phase matrix was rinsed with MeOH (6 mL). The crude mixture was eluted in a collection tube with 1 M ammonia in MeOH (6 mL) and concentrated in vacuo. The residue was purified by preparative LC-MS to give 15 mg, (41% yield) of the titled compound.

### Example 6

### N-(2-oxo-2-(piperidin-1-yl)ethyl)-N-(3-(trifluoromethyl)phenyl)benzenesulfonamide

Prepared as in Example 5—step-3 from piperidine.

### Example 7

### N-Benzyl-2-(N-(3-(trifluoromethyl)phenyl)benzenesulfonamido)acetamide

Prepared as in Example 5—step-3 from benzyl amine.

### Example 8

### N-Phenyl-2-(N-(3-(trifluoromethyl)phenyl)benzenesulfonamido)acetamide

Prepared as in Example 5—step-3 from aniline.

### Example 9

### N-Benzyl-N-(3-chloro-5-(trifluoromethyl)pyridin-2-yl)-4-(hydroxymethyl)benzene-1-sulfonamide

To a solution of methyl 4-(N-benzyl-N-(3-chloro-5-(trifluoromethyl)pyridin-2-yl)sulfamoyl)benzoate (step-2 of Example 1, 40 mg, 0.08 mmol) in THF (1 mL) was added lithium alminium hydride (3.1 mg, 0.08 mmol) at room temperature. After being stirred at room temperature for 30 min, EtOAc was added to the reaction mixture. H₂O and 2 M NaOH aqueous solution were added carefully until forming white precipitate. MgSO₄ was added to the suspension. After being filtered off, the filtrate was concentrated in vacuo. The residue was diluted with MeOH and applied onto a strong cation exchange cartridge (BondElute (registered trademark) SCX, 1 g/6 mL, Varian Inc.), and the solid phase matrix was rinsed with MeOH (6 mL). The crude mixture was eluted in a collection tube with 1 M ammonia in MeOH (6 mL) and concentrated in vacuo. The residue was purified by preparative LC-MS to give 9.9 mg (26% yield) of the titled compound.

### Example 10

### 4-(Benzyl(3-chloro-5-(trifluoromethyl)pyridin-2-yl)sulfamoyl)benzamide

Prepared as in Example 5—step-3 from 4-(N-benzyl-N-(3-chloro-5-(trifluoromethyl)pyridin-2-yl)sulfamoyl)benzoic acid (Example 1) and ammonium chloride.

### Example 11

### 4-(Benzyl(3-chloro-5-(trifluoromethyl)pyridin-2-yl)sulfamoyl)-N-methylbenzamide

Prepared as in Example 5—step-3 from 4-(N-benzyl-N-(3-chloro-5-(trifluoromethyl)pyridin-2-yl)sulfamoyl)benzoic acid (Example 1) and methyl amine.

### Example 12

### 4-(Benzyl(3-chloro-5-(trifluoromethyl)pyridin-2-yl)sulfamoyl)-N,N-dimethylbenzamide

Prepared as in Example 5—step-3 from 4-(N-benzyl-N-(3-chloro-5-(trifluoromethyl)pyridin-2-yl)sulfamoyl)benzoic acid (Example 1) and dimethyl amine.

### Example 13

### 4-(Benzyl(3-chloro-5-(trifluoromethyl)pyridin-2-yl)sulfamoyl]-N-(2-hydroxyethyl)benzamide

Prepared as in Example 5—step-3 from 4-(N-benzyl-N-(3-chloro-5-(trifluoromethyl)pyridin-2-yl)sulfamoyl)benzoic acid (Example 1) and ethanolamine.

### Example 14

### 4-(N-Benzyl-N-(5-(trifluoromethyl)pyridin-2-yl)sulfamoyl)benzoic acid

### Step-1: Methyl 4-(N-benzylsulfamoyl)benzoate

To a solution of benzyl amine (200 mg, 1.9 mmol) and Et₃N (570 mg, 5.6 mmol) in DCM (3 mL) was added methyl 4-(chlorosulfonyl)benzoate (440 mg, 1.9 mmol) at room temperature. After being stirred at room temperature for 2 hrs, sat. ammonium chloride was added to the mixture. The product was extracted with DCM. The organic layer was then washed with brine, dried over Na₂SO₄. After the filtration to separate solvent and Na₂SO₄, the solvent was removed under reduced pressure to give the residue, which was applied to a silica gel chromatography column and eluted with a hexane/EtOAc=2/1 to furnish 274 mg (48% yield) of the titled compound as a white solid;

¹H-NMR (300 MHz, CDCl₃) δ 8.16 (2H, d, J=8.1 Hz), 7.92 (2H, d, J=8.1 Hz), 7.33-7.25 (4H, m), 7.17 (1H, m), 4.80 (1H, t, J=5.9 Hz), 4.18 (2H, d, J=5.9 Hz), 3.97 (3H, s),

LC-MS (Method A) m/z: M+1 obs 306.0, tR=2.92 min.

### Step-2: Methyl 4-(N-benzyl-N-(5-(trifluoromethyl)pyridin-2-yl)sulfamoyl)benzoate

To a solution of methyl 4-(N-benzylsulfamoyl)benzoate (step-1 of Example 14, 60 mg, 0.20 mmol) and 2-chloro-5-(trifluoromethyl)pyridine (43 mg, 0.24 mmol) in 1,4-dioxane (2 mL) were added Cs₂CO₃ (96 mg, 0.30 mmol) and 4,5-bis(diphenylphosphino)-9,9-dimethylxanthene (17 mg, 0.03 mmol) at room temperature. The mixture was stirred at room temperature for 5 min, palladium acetate (4 mg, 0.02 mmol) was added to the mixture. The mixture was stirred at 115° C. for 3 hrs using microwave oven. The resulting precipitate was removed by filtration and washed with dioxane. The filtrate was concentrated under reduced pressure to give the residue, which was applied to a silica gel chromatography column and eluted with a hexane/EtOAc=4/1 to furnish 42 mg (47% yield) of the titled compound as a brown oil;

¹H-NMR (300 MHz, CDCl₃) δ 8.52 (1H, s), 8.13 (2H, d, J=8.8 Hz), 7.83 (1H, dd, J=8.8, 2.2 Hz), 7.74 (2H, d, J=8.8 Hz), 7.62 (1H, d, J=8.8 Hz), 7.30-7.20 (5H, m), 5.11 (2H, s), 3.95 (3H, s),

LC-MS (Method A) m/z: M+1 obs 450.9, tR=3.54 min.

### Step-3: 4-(N-Benzyl-N-(5-(trifluoromethyl)pyridin-2-yl)sulfamoyl)benzoic acid

Prepared as in Example 1—step-3 from methyl 4-(N-benzyl-N-(5-(trifluoromethyl)pyridin-2-yl)sulfamoyl)benzoate (step-2 of Example 14).

### Example 15

### 4-(N-(3-Chloro-5-(trifluoromethyl)pyridin-2-yl)-N-(4-(trifluoromethoxy)benzyl)sulfamoyl)benzoic acid

### Step-1: Methyl 4-(N-(3-chloro-5-(trifluoromethyl)pyridin-2-yl)-N-(4-(trifluoromethoxy)benzyl)sulfamoyl)benzoate

To a mixture of methyl 4-(N-(3-chloro-5-(trifluoromethyl)pyridin-2-yl)sulfamoyl)benzoate (prepared in step 1 of Example 1, 30 mg, 0.076 mmol) and 1-(bromomethyl)-4-(trifluoromethoxy)benzene (39 mg, 0.152 mmol) in DMF (0.5 mL) were added Cs₂CO₃ (99 mg, 0.304 mmol) and NaI (11 mg, 0.076 mmol) at room temperature. The mixture was stirred at 90° C. for 1 day. The mixture was quenched with H₂O, extracted with EtOAc, dried over Na₂SO₄, filtered and concentrated. The residual oil was used next step without purification.

LC-MS (Method A) m/z: M+1 obs 568.8, tR=3.69 min.

### Step-2: 4-(N-(3-Chloro-5-(trifluoromethyl)pyridin-2-yl)-N-(4-(trifluoromethoxy)benzyl)sulfamoyl)benzoic acid

To a solution of methyl 4-(N-(3-chloro-5-(trifluoromethyl)pyridin-2-yl)-N-(4-(trifluoromethoxy)benzyl)sulfamoyl)benzoate (prepared in step-1 of Example 15, crude) in THF (0.5 mL) was added 2 M aqueous NaOH solution at room temperature. The mixture was stirred at 50° C. for 3 hrs. The mixture was acidified by 2 M aqueous HCl (0.5 mL) solution, extracted with DCM, dried over Na₂SO₄, filtered and concentrated. The further purification was carried out by preparative LC-MS system in usual manner. HPLC-QC method, retention time and observed MS were summarized in Table 6.

### Example 16

### 4-(N-(3-Chloro-5-(trifluoromethyl)pyridin-2-yl)-N-(4-methoxybenzyl)sulfamoyl)benzoic acid

### Step-1: Methyl 4-(N-(3-chloro-5-(trifluoromethyl)pyridin-2-yl)-N-(4-methoxybenzyl)sulfamoyl)benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and 1-(bromomethyl)-4-methoxybenzene;

LC-MS (Method A) m/z: M+1 obs 514.9, tR=3.49 min.

### Step-2: 4-(N-(3-Chloro-5-(trifluoromethyl)pyridin-2-yl)-N-(4-methoxybenzyl)sulfamoyl)benzoic acid

The titled compound was prepared according to the procedure described in step-2 of Example 15 from methyl 4-(N-(3-chloro-5-(trifluoromethyl)pyridin-2-yl)-N-(4-methoxybenzyl)sulfamoyl)benzoate (step-1 of Example 16). The further purification was carried out by preparative LC-MS system in usual manner. HPLC-QC method, retention time and observed MS were summarized in Table 6.

### Example 17

### 4-(N-(3-Chloro-5-(trifluoromethyl)pyridin-2-yl)-N-(4-fluorobenzyl)sulfamoyl)benzoic acid

### Step-1: Methyl 4-(N-(3-chloro-5-(trifluoromethyl)pyridin-2-yl)-N-(4-fluorobenzyl)sulfamoyl)benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and 1-(bromomethyl)-4-fluorobenzene;

LC-MS (Method A) m/z: M+1 obs 502.9, tR=3.54 min.

### Step-2: 4-(N-(3-Chloro-5-(trifluoromethyl)pyridin-2-yl)-N-(4-fluorobenzyl)sulfamoyl)benzoic acid

The titled compound was prepared according to the procedure described in step-2 of Example 15 from methyl 4-(N-(3-chloro-5-(trifluoromethyl)pyridin-2-yl)-N-(4-fluorobenzyl)sulfamoyl)benzoate (step-1 of Example 17). The further purification was carried out by preparative LC-MS system in usual manner. HPLC-QC method, retention time and observed MS were summarized in Table 6.

### Example 18

### 4-(N-(4-tert-Butylbenzyl)-N-(3-chloro-5-(trifluoromethyl)pyridin-2-yl)sulfamoyl)benzoic acid

### Step-1: Methyl 4-(N-(4-(tert-butyl)benzyl)-N-(3-chloro-5-(trifluoromethyl)pyridin-2-yl)sulfamoyl)benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and 1-(bromomethyl)-4-(tert-butyl)benzene;

LC-MS (Method A) m/z: M+1 obs 540.9, tR=3.84 min.

### Step-2: 4-(N-(4-tert-Butylbenzyl)-N-(3-chloro-5-(trifluoromethyl)pyridin-2-yl)sulfamoyl)benzoic acid

The titled compound was prepared according to the procedure described in step-2 of Example 15 from methyl 4-(N-(4-(tert-butyl)benzyl)-N-(3-chloro-5-(trifluoromethyl)pyridin-2-yl)sulfamoyl)benzoate (step-1 of Example 18). The further purification was carried out by preparative LC-MS system in usual manner. HPLC-QC method, retention time and observed MS were summarized in Table 6.

### Example 19

### 4-(N-(3-Chloro-5-(trifluoromethyl)pyridin-2-yl)-N-(2-cyclohexylethyl)sulfamoyl)benzoic acid

### Step-1: Methyl 4-(N-(3-chloro-5-(trifluoromethyl)pyridin-2-yl)-N-(2-cyclohexylethyl)sulfamoyl)benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and (2-bromoethyl)cyclohexane;

LC-MS (Method A) m/z: M+1 obs 505.0, tR=3.87 min.

### Step-2: 4-(N-(3-Chloro-5-(trifluoromethyl)pyridin-2-yl)-N-(2-cyclohexylethyl)sulfamoyl)benzoic acid

The titled compound was prepared according to the procedure described in step-2 of Example 15 from methyl 4-(N-(3-chloro-5-(trifluoromethyl)pyridin-2-yl)-N-(2-cyclohexylethyl)sulfamoyl)benzoate (step-1 of Example 19). The further purification was carried out by preparative LC-MS system in usual manner. HPLC-QC method, retention time and observed MS were summarized in Table 6.

### Example 20

### 4-(N-(3-Chloro-5-(trifluoromethyl)pyridin-2-yl)-N-(2-fluorobenzyl)sulfamoyl)benzoic acid

### Step-1: Methyl 4-(N-(3-chloro-5-(trifluoromethyl)pyridin-2-yl)-N-(2-fluorobenzyl)sulfamoyl)benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and 1-(bromomethyl)-2-fluorobenzene;

LC-MS (Method A) m/z: obs 503.12, tR=3.48 min.

### Step-2: 4-(N-(3-chloro-5-(trifluoromethyl)pyridin-2-yl)-N-(2-fluorobenzyl)sulfamoyl)benzoic acid

The titled compound was prepared according to the procedure described in step-2 of Example 15 from methyl 4-(N-(3-chloro-5-(trifluoromethyl)pyridin-2-yl)-N-(2-fluorobenzyl)sulfamoyl)benzoate (step-1 of Example 20). The further purification was carried out by preparative LC-MS system in usual manner. HPLC-QC method, retention time and observed MS were summarized in Table 6.

### Example 21

### 4-(N-Benzyl-N-(4-(trifluoromethyl)pyridin-2-yl)sulfamoyl)benzoic acid

### Step-1: Methyl 4-(N-benzyl-N-(4-(trifluoromethyl)pyridin-2-yl)sulfamoyl)benzoate

The titled compound was prepared according to the procedure described in step-2 of Example 14 from methyl 4-(N-benzylsulfamoyl)benzoate (step-1 of Example 14) and 2-chloro-4-(trifluoromethyl)pyridine;

LC-MS (Method A) m/z: M+1 obs 451.14, tR=3.48 min.

### Step-2: 4-(N-Benzyl-N-(4-(trifluoromethyl)pyridin-2-yl)sulfamoyl)benzoic acid

The titled compound was prepared according to the procedure described in step-2 of Example 15 from methyl 4-(N-benzyl-N-(4-(trifluoromethyl)pyridin-2-yl)sulfamoyl)benzoate (step-1 of Example 21). The further purification was carried out by preparative LC-MS system in usual manner. HPLC-QC method, retention time and observed MS were summarized in Table 6.

### Example 22

### 4-(N-Benzyl-N-(6-(trifluoromethyl)pyridin-2-yl)sulfamoyl)benzoic acid

### Step-1: Methyl 4-(N-benzyl-N-(6-(trifluoromethyl)pyridin-2-yl)sulfamoyl)benzoate

The titled compound was prepared according to the procedure described in step-2 of Example 14 from methyl 4-(N-benzylsulfamoyl)benzoate (step-1 of Example 14) and 2-chloro-6-(trifluoromethyl)pyridine;

LC-MS (Method A) m/z: M+1 obs 451.14, tR=3.47 min.

### Step-2: 4-(N-Benzyl-N-(6-(trifluoromethyl)pyridin-2-yl)sulfamoyl)benzoic acid

The titled compound was prepared according to the procedure described in step-2 of Example 15 from methyl 4-(N-benzyl-N-(6-(trifluoromethyl)pyridin-2-yl)sulfamoyl)benzoate (step-1 of Example 22). The further purification was carried out by preparative LC-MS system in usual manner. HPLC-QC method, retention time and observed MS were summarized in Table 6.

### Example 23

### 4-(N-(3-Chloro-5-(trifluoromethyl)pyridin-2-yl)-N-(3-(trifluoromethyl)benzyl)sulfamoyl)benzoic acid

### Step-1: Methyl 4-(N-(3-chloro-5-(trifluoromethyl)pyridin-2-yl)-N-(3-(trifluoromethyl)benzyl)sulfamoyl)benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and 1-(chloromethyl)-3-(trifluoromethyl)benzene;

LC-MS (Method A) m/z: M+1 obs 553.08, tR=3.63 min.

### Step-2: 4-(N-(3-Chloro-5-(trifluoromethyl)pyridin-2-yl)-N-(3-(trifluoromethyl)benzyl)sulfamoyl)benzoic acid

The titled compound was prepared according to the procedure described in step-2 of Example 15 from methyl 4-(N-(3-chloro-5-(trifluoromethyl)pyridin-2-yl)-N-(3-(trifluoromethyl)benzyl)sulfamoyl)benzoate (step-1 of Example 23). The further purification was carried out by preparative LC-MS system in usual manner. HPLC-QC method, retention time and observed MS were summarized in Table 6.

### Example 24

### 4-(N-(3-Chloro-5-(trifluoromethyl)pyridin-2-yl)-N-(3-methoxybenzyl)sulfamoyl)benzoic acid

### Step-1: Methyl 4-(N-(3-chloro-5-(trifluoromethyl)pyridin-2-yl)-N-(3-methoxybenzyl)sulfamoyl)benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and 1-(bromomethyl)-3-methoxybenzene;

LC-MS (Method A) m/z: M+1 obs 515.08, tR=3.50 min.

### Step-2: 4-(N-(3-Chloro-5-(trifluoromethyl)pyridin-2-yl)-N-(3-methoxybenzyl)sulfamoyl)benzoic acid

The titled compound was prepared according to the procedure described in step-2 of Example 15 from methyl 4-(N-(3-chloro-5-(trifluoromethyl)pyridin-2-yl)-N-(3-methoxybenzyl)sulfamoyl)benzoate (step-1 of Example 24). The further purification was carried out by preparative LC-MS system in usual manner. HPLC-QC method, retention time and observed MS were summarized in Table 6.

### Example 25

### 4-(N-(3-chloro-5-(trifluoromethyl)pyridin-2-yl)-N-(3-(trifluoromethoxy)benzyl)sulfamoyl)benzoic acid

### Step-1: Methyl 4-(N-(3-chloro-5-(trifluoromethyl)pyridin-2-yl)-N-(3-(trifluoromethoxy)benzyl)sulfamoyl)benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and 1-(bromomethyl)-3-(trifluoromethoxy)benzene;

LC-MS (Method A) m/z: M+1 obs 569.08, tR=3.67 min.

### Step-2: 4-(N-(3-Chloro-5-(trifluoromethyl)pyridin-2-yl)-N-(3-(trifluoromethoxy)benzyl)sulfamoyl)benzoic acid

The titled compound was prepared according to the procedure described in step-2 of Example 15 from methyl 4-(N-(3-chloro-5-(trifluoromethyl)pyridin-2-yl)-N-(3-(trifluoromethoxy)benzyl)sulfamoyl)benzoate (step-1 of Example 25). The further purification was carried out by preparative LC-MS system in usual manner. HPLC-QC method, retention time and observed MS were summarized in Table 6.

### Example 26

### 4-(N-(3-Chloro-5-(trifluoromethyl)pyridin-2-yl)-N-(2,4-difluorobenzyl)sulfamoyl)benzoic acid

### Step-1: Methyl 4-(N-(3-chloro-5-(trifluoromethyl)pyridin-2-yl)-N-(2,4-difluorobenzyl)sulfamoyl)benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and 1-(bromomethyl)-2,4-difluorobenzene;

LC-MS (Method A) m/z: M+1 obs 521.07, tR=3.50 min.

### Step-2: 4-(N-(3-Chloro-5-trifluoromethyl)pyridin-2-yl)-N-(2,4-difluorobenzyl)sulfamoyl)benzoic acid

The titled compound was prepared according to the procedure described in step-2 of Example 15 from methyl 4-(N-(3-chloro-5-(trifluoromethyl)pyridin-2-yl)-N-(2,4-difluorobenzyl)sulfamoyl)benzoate (step-1 of Example 26). The further purification was carried out by preparative LC-MS system in usual manner. HPLC-QC method, retention time and observed MS were summarized in Table 6.

### Example 27

### 4-(N-(3-Chloro-5-(trifluoromethyl)pyridin-2-yl)-N-(4-methylbenzyl)sulfamoyl)benzoic acid

### Step-1: Methyl 4-(N-(3-chloro-5-(trifluoromethyl)pyridin-2-yl)-N-(4-methylbenzyl)sulfamoyl)benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and 1-(bromomethyl)-4-methylbenzene;

LC-MS (Method A) m/z: M+1 obs 499.07, tR=3.59 min.

### Step-2: 4-(N-(3-Chloro-5-(trifluoromethyl)pyridin-2-yl)-N-(4-methylbenzyl)sulfamoyl)benzoic acid

The titled compound was prepared according to the procedure described in step-2 of Example 15 from methyl 4-(N-(3-chloro-5-(trifluoromethyl)pyridin-2-yl)-N-(4-methylbenzyl)sulfamoyl)benzoate (step-1 of Example 27). The further purification was carried out by preparative LC-MS system in usual manner. HPLC-QC method, retention time and observed MS were summarized in Table 6.

### Example 28

### 4-(N-(3-Chloro-5-(trifluoromethyl)pyridin-2-yl)-N-(4-isopropylbenzyl)sulfamoyl)benzoic acid

### Step-1: Methyl 4-(N-(3-chloro-5-(trifluoromethyl)pyridin-2-yl)-N-(4-isopropylbenzyl)sulfamoyl)benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and 1-(chloromethyl)-4-isopropylbenzene;

LC-MS (Method A) m/z: M+1 obs 527.13, tR=3.73 min.

### Step-2: 4-(N-(3-Chloro-5-(trifluoromethyl)pyridin-2-yl)-N-(4-isopropylbenzyl)sulfamoyl)benzoic acid

The titled compound was prepared according to the procedure described in step-2 of Example 15 from methyl 4-(N-(3-chloro-5-(trifluoromethyl)pyridin-2-yl)-N-(4-isopropylbenzyl)sulfamoyl)benzoate (step-1 of Example 28). The further purification was carried out by preparative LC-MS system in usual manner. HPLC-QC method, retention time and observed MS were summarized in Table 6.

### Example 29

### 4-(N-(3-Chloro-5-(trifluoromethyl)pyridin-2-yl)-N-(cyclopropylmethyl)sulfamoyl)benzoic acid

### Step-1: Methyl 4-(N-(3-chloro-5-(trifluoromethyl)pyridin-2-yl)-N-(cyclopropylmethyl)sulfamoyl)benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and (bromomethyl)cyclopropane;

LC-MS (Method A) m/z: obs 449.10, tR=3.43 min.

### Step-2: 4-(N-(3-Chloro-5-(trifluoromethyl)pyridin-2-yl)-N-(cyclopropylmethyl)sulfamoyl)benzoic acid

The titled compound was prepared according to the procedure described in step-2 of Example 15 from methyl 4-(N-(3-chloro-5-(trifluoromethyl)pyridin-2-yl)-N-(cyclopropylmethyl)sulfamoyl)benzoate (step-1 of Example 29). The further purification was carried out by preparative LC-MS system in usual manner. HPLC-QC method, retention time and observed MS were summarized in Table 6.

### Example 30

### 4-(N-(3-Chloro-5-(trifluoromethyl)pyridin-2-yl)-N-(2-(4-fluorophenoxy)ethyl)sulfamoyl)benzoic acid

### Step-1: Methyl 4-(N-(3-chloro-5-(trifluoromethyl)pyridin-2-yl)-N-(2-(4-fluorophenoxy)ethyl)sulfamoyl)benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and 1-(2-bromoethoxy)-4-fluorobenzene;

LC-MS (Method A) m/z: M+1 obs 533.06, tR=3.48 min.

### Step-2: 4-(N-(3-Chloro-5-(trifluoromethyl)pyridin-2-yl)-N-(2-(4-fluorophenoxy)ethyl)sulfamoyl)benzoic acid

The titled compound was prepared according to the procedure described in step-2 of Example 15 from methyl 4-(N-(3-chloro-5-(trifluoromethyl)pyridin-2-yl)-N-(2-(4-fluorophenoxy)ethyl)sulfamoyl)benzoate (step-1 of Example 30). The further purification was carried out by preparative LC-MS system in usual manner. HPLC-QC method, retention time and observed MS were summarized in Table 6.

### Example 31

### 4-(N-(3-Chloro-5-(trifluoromethyl)pyridin-2-yl)-N-(2-(trifluoromethyl)benzyl)sulfamoyl)benzoic acid

### Step-1: Methyl 4-(N-(3-chloro-5-trifluoromethyl)pyridin-2-yl)-N-(2-(trifluoromethyl)benzyl)sulfamoyl)benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and 1-(chloromethyl)-2-(trifluoromethyl)benzene;

LC-MS (Method A) m/z: M+1 obs 533.01, tR=3.60 min.

### Step-2: 4-(N-(3-Chloro-5-(trifluoromethyl)pyridin-2-yl)-N-(2-(trifluoromethyl)benzyl)sulfamoyl)benzoic acid

The titled compound was prepared according to the procedure described in step-2 of Example 15 from methyl 4-(N-(3-chloro-5-(trifluoromethyl)pyridin-2-yl)-N-(2-(trifluoromethyl)benzyl)sulfamoyl)benzoate (step-1 of Example 31). The further purification was carried out by preparative LC-MS system in usual manner. HPLC-QC method, retention time and observed MS were summarized in Table 6.

### Example 32

### 4-(N-(3-Chloro-5-(trifluoromethyl)pyridin-2-yl)-N-(4-chlorobenzyl)sulfamoyl)benzoic acid

### Step-1: Methyl 4-(N-(3-chloro-5-(trifluoromethyl)pyridin-2-yl)-N-(4-chlorobenzyl)sulfamoyl)benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and 1-chloro-4-(chloromethyl)benzene;

LC-MS (Method A) m/z: M+1 obs 519.03, tR=3.60 min.

### Step-2: 4-(N-(3-Chloro-5-(trifluoromethyl)pyridin-2-yl)-N-(4-chlorobenzyl)sulfamoyl)benzoic acid

The titled compound was prepared according to the procedure described in step-2 of Example 15 from methyl 4-(N-(3-chloro-5-(trifluoromethyl)pyridin-2-yl)-N-(4-chlorobenzyl)sulfamoyl)benzoate (step-1 of Example 32). The further purification was carried out by preparative LC-MS system in usual manner. HPLC-QC method, retention time and observed MS were summarized in Table 6.

### Example 33

### 4-(N-(3-Chloro-5-(trifluoromethyl)pyridin-2-yl)-N-(4-cyanobenzyl)sulfamoyl)benzoic acid

### Step-1: Methyl 4-(N-(3-chloro-5-(trifluoromethyl)pyridin-2-yl)-N-(4-cyanobenzyl)sulfamoyl)benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and 4-(bromomethyl)benzonitrile;

LC-MS (Method A) m/z: M+1 obs 510.04, tR=3.37 min.

### Step-2: 4-(N-(3-Chloro-5-(trifluoromethyl)pyridin-2-yl)-N-(4-cyanobenzyl)sulfamoyl)benzoic acid

The titled compound was prepared according to the procedure described in step-2 of Example 15 from methyl 4-(N-(3-chloro-5-(trifluoromethyl)pyridin-2-yl)-N-(4-cyanobenzyl)sulfamoyl)benzoate (step-1 of Example 33). The further purification was carried out by preparative LC-MS system in usual manner. HPLC-QC method, retention time and observed MS were summarized in Table 6.

### Example 34

### 4-(N-Benzyl-N-(3-methylpyridin-2-yl)sulfamoyl)benzoic acid

### Step-1: Methyl 4-(N-(3-methylpyridin-2-yl)sulfamoyl)benzoate

The titled compound was prepared according to the procedure described in step-1 of Example 1 from methyl 4-(chlorosulfonyl)benzoate and 3-methylpyridin-2-amine in the presence of DMAP (0.2 eq.);

¹H-NMR (300 MHz, CDCl₃) δ 8.11 (2H, d, J=8.8 Hz), 8.03 (2H, d, J=8.8 Hz), 7.49 (1H, d, J=6.6 Hz), 7.45 (1H, d, J=6.6 Hz), 6.60 (1H, d, J=6.6 Hz), 3.94 (3H, s), 2.19 (3H, s);

LC-MS (Method A) m/z: M+1 obs 307.27, tR=2.57 min.

### Step-2: Methyl 4-(N-benzyl-N-(3-methylpyridin-2-yl)sulfamoyl)benzoate

The titled compound was prepared according to the procedure described in step-1 of Example 15 from methyl 4-(N-(3-methylpyridin-2-yl)sulfamoyl)benzoate (step-1 of Example 34) and (bromomethyl)benzene;

LC-MS (Method A) m/z: M+1 obs 397.16, tR=3.29 min.

### Step-3: 4-(N-Benzyl-N-(3-methylpyridin-2-yl)sulfamoyl)benzoic acid

The titled compound was prepared according to the procedure described in step-2 of Example 15 from methyl 4-(N-benzyl-N-(3-methylpyridin-2-yl)sulfamoyl)benzoate (step-2 of Example 34). The further purification was carried out by preparative LC-MS system in usual manner. HPLC-QC method, retention time and observed MS were summarized in Table 6.

### Example 35

### 4-(N-Benzyl-N-(3,5-dichloropyridin-2-yl)sulfamoyl)benzoic acid

### Step-1: Methyl 4-(N-(3,5-dichloropyridin-2-yl)sulfamoyl)benzoate

The titled compound was prepared according to the procedure described in step-1 of Example 1 from methyl 4-(chlorosulfonyl)benzoate and 3,5-dichloropyridin-2-amine in the presence of DMAP (0.2 eq.);

¹H-NMR (300 MHz, CDCl₃) δ 8.22 (2H, d, J=8.8 Hz), 8.17 (2H, d, J=8.8 Hz), 8.09 (1H, d, J=2.2 Hz), 7.64 (1H, d, J=2.2 Hz), 3.95 (3H, s);

LC-MS (Method A) m/z: M+1 obs 361.07, tR=2.93 min.

### Step-2: Methyl 4-(N-benzyl-N-(3,5-dichloropyridin-2-yl)sulfamoyl)benzoate

The titled compound was prepared according to the procedure described in step-1 of Example 15 from methyl 4-(N-(3,5-dichloropyridin-2-yl)sulfamoyl)benzoate (step-1 of Example 35) and (bromomethyl)benzene;

LC-MS (Method A) m/z: M+1 obs 451.04, tR=3.44 min.

### Step-3: 4-(N-Benzyl-N-(3,5-dichloropyridin-2-yl)sulfamoyl)benzoic acid

The titled compound was prepared according to the procedure described in step-2 of Example 15 from methyl 4-(N-benzyl-N-(3,5-dichloropyridin-2-yl)sulfamoyl)benzoate (step-2 of Example 35). The further purification was carried out by preparative LC-MS system in usual manner. HPLC-QC method, retention time and observed MS were summarized in Table 6.

### Example 36

### 4-(N-Benzyl-N-(3-chloro-5-methylpyridin-2-yl)sulfamoyl)benzoic acid

### Step-1: Methyl 4-(N-(3-chloro-5-methylpyridin-2-yl)sulfamoyl)benzoate

The titled compound was prepared according to the procedure described in step-1 of Example 1 from methyl 4-(chlorosulfonyl)benzoate and 3-chloro-5-methylpyridin-2-amine in the presence of DMAP (0.2 eq.);

¹H-NMR (300 MHz, CDCl₃) δ 8.24-8.10 (4H, m), 7.91 (1H, br s), 7.45 (1H, s), 3.95 (3H, s), 2.22 (3H, s);

LC-MS (Method A) m/z: M+1 obs 341.11, tR=2.82 min.

### Step-2: Methyl 4-(N-benzyl-N-(3-chloro-5-methylpyridin-2-yl)sulfamoyl)benzoate

The titled compound was prepared according to the procedure described in step-1 of Example 15 from methyl 4-(N-(3-chloro-5-methylpyridin-2-yl)sulfamoyl)benzoate (step-1 of Example 36) and (bromomethyl)benzene;

LC-MS (Method B) m/z: M+1 obs 431.16, tR=2.61 min.

### Step-3: 4-(N-Benzyl-N-(3-chloro-5-methylpyridin-2-yl)sulfamoyl)benzoic acid

The titled compound was prepared according to the procedure described in step-2 of Example 15 from methyl 4-(N-benzyl-N-(3-chloro-5-methylpyridin-2-yl)sulfamoyl)benzoate (step-2 of Example 36). The further purification was carried out by preparative LC-MS system in usual manner. HPLC-QC method, retention time and observed MS were summarized in Table 6.

### Example 37

### 4-(N-Benzyl-N-(3-chloropyridin-2-yl)sulfamoyl)benzoic acid

### Step-1: Methyl 4-(N-(3-chloropyridin-2-yl)sulfamoyl)benzoate

The titled compound was prepared according to the procedure described in step-1 of Example 1 from methyl 4-(chlorosulfonyl)benzoate and 3-chloropyridin-2-amine in the presence of DMAP (0.2 eq.);

¹H-NMR (300 MHz, CDCl₃) δ 8.35-8.11 (5H, m), 7.64-7.60 (1H, m), 6.95-6.87 (1H, m), 3.95 (3H, s);

LC-MS (Method A) m/z: M+1 obs 327.10, tR=2.62 min.

### Step-2: Methyl 4-(N-benzyl-N-(3-chloropyridin-2-yl)sulfamoyl)benzoate

The titled compound was prepared according to the procedure described in step-1 of Example 15 from methyl 4-(N-(3-chloropyridin-2-yl)sulfamoyl)benzoate (step-1 of Example 37) and (bromomethyl)benzene;

LC-MS (Method B) m/z: M+1 obs 417.14, tR=2.43 min.

### Step-3: 4-(N-Benzyl-N-(3-chloropyridin-2-yl)sulfamoyl)benzoic acid

The titled compound was prepared according to the procedure described in step-2 of Example 15 from methyl 4-(N-benzyl-N-(3-chloropyridin-2-yl)sulfamoyl)benzoate (step-2 of Example 37). The further purification was carried out by preparative LC-MS system in usual manner. HPLC-QC method, retention time and observed MS were summarized in Table 6.

### Example 38

### 4-(N-Benzyl-N-(5-chloro-3-methylpyridin-2-yl)sulfamoyl)benzoic acid

### Step-1: Methyl 4-(N-(5-chloro-3-methylpyridin-2-yl)sulfamoyl)benzoate

The titled compound was prepared according to the procedure described in step-1 of Example 1 from methyl 4-(chlorosulfonyl)benzoate and 5-chloro-3-methylpyridin-2-amine in the presence of DMAP (0.2 eq.);

LC-MS (Method A) m/z: M+1 obs 341.15, tR=2.93 min.

### Step-2: Methyl 4-(N-benzyl-N-(5-chloro-3-methylpyridin-2-yl)sulfamoyl)benzoate

The titled compound was prepared according to the procedure described in step-1 of Example 15 from methyl 4-(N-(5-chloro-3-methylpyridin-2-yl)sulfamoyl)benzoate (step-1 of Example 38) and (bromomethyl)benzene;

LC-MS (Method B) m/z: M+1 obs 431.16, tR=2.76 min.

### Step-3: 4-(N-Benzyl-N-(5-chloro-3-methylpyridin-2-yl)sulfamoyl)benzoic acid

The titled compound was prepared according to the procedure described in step-2 of Example 15 from methyl 4-(N-benzyl-N-(5-chloro-3-methylpyridin-2-yl)sulfamoyl)benzoate (step-2 of Example 38). The further purification was carried out by preparative LC-MS system in usual manner. HPLC-QC method, retention time and observed MS were summarized in Table 6.

### Example 39

### 4-(N-(3-Chloro-5-(trifluoromethyl)pyridin-2-yl)-N-(4-(trifluoromethyl)benzyl)sulfamoyl)benzoic acid

### Step-1: Methyl 4-(N-(3-chloro-5-(trifluoromethyl)pyridin-2-yl)-N-(4-(trifluoromethyl)benzyl)sulfamoyl)benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and 1-(bromomethyl)-4-(trifluoromethyl)benzene;

LC-MS (Method A) m/z: M+1 obs 553.06, tR=3.62 min.

### Step-2: 4-(N-(3-Chloro-5-(trifluoromethyl)pyridin-2-yl)-N-(4-(trifluoromethyl)benzyl)sulfamoyl)benzoic acid

The titled compound was prepared according to the procedure described in step-2 of Example 15 from methyl 4-(N-(3-chloro-5-(trifluoromethyl)pyridin-2-yl)-N-(4-(trifluoromethyl)benzyl)sulfamoyl)benzoate (step-1 of Example 39). The further purification was carried out by preparative LC-MS system in usual manner. HPLC-QC method, retention time and observed MS were summarized in Table 6.

### Example 40

### 4-(N-(2-Chloro-4-fluorobenzyl)-N-(3-chloro-5-(trifluoromethyl)pyridin-2-yl)sulfamoyl)benzoic acid

### Step-1: Methyl 4-(N-(2-chloro-4-fluorobenzyl)-N-(3-chloro-5-(trifluoromethyl)pyridin-2-yl)sulfamoyl)benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and 1-(bromomethyl)-2-chloro-4-fluorobenzene;

LC-MS (Method B) m/z: M+1 obs 537.07, tR=2.97 min.

### Step-2: 4-(N-(2-Chloro-4-fluorobenzyl)-N-(3-chloro-5-(trifluoromethyl)pyridin-2-yl)sulfamoyl)benzoic acid

The titled compound was prepared according to the procedure described in step-2 of Example 15 from methyl 4-(N-(2-chloro-4-fluorobenzyl)-N-(3-chloro-5-(trifluoromethyl)pyridin-2-yl)sulfamoyl)benzoate (step-1 of Example 40). The further purification was carried out by preparative LC-MS system in usual manner. HPLC-QC method, retention time and observed MS were summarized in Table 6.

### Example 41

### 4-(N-(3-Chloro-5-(trifluoromethyl)pyridin-2-yl-N-(4-(2,2,2-trifluoroethoxy)benzyl)sulfamoyl)benzoic acid

### Step-1: Methyl 4-(N-(3-chloro-5-(trifluoromethyl)pyridin-2-yl)-N-(4-(2,2,2-trifluoroethoxy)benzyl)sulfamoyl)benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and 1-(bromomethyl)-4-(2,2,2-trifluoroethoxy)benzene;

LC-MS (Method B) m/z: M+1 obs 583.13, tR=2.93 min.

### Step-2: 4-(N-(3-Chloro-5-(trifluoromethyl)pyridin-2-yl)-N-(4-(2,2,2-trifluoroethoxy)benzyl)sulfamoyl)benzoic acid

The titled compound was prepared according to the procedure described in step-2 of Example 15 from methyl 4-(N-(3-chloro-5-(trifluoromethyl)pyridin-2-yl)-N-(4-(2,2,2-trifluoroethoxy)benzyl)sulfamoyl)benzoate (step-1 of Example 41). The further purification was carried out by preparative LC-MS system in usual manner. HPLC-QC method, retention time and observed MS were summarized in Table 6.

### Example 42

### 4-(N-(3-Chloro-5-(trifluoromethyl)pyridin-2-yl)-N-(3,5-difluorobenzyl)sulfamoyl)benzoic acid

### Step-1: Methyl 4-(N-(3-chloro-5-(trifluoromethyl)pyridin-2-yl)-N-(3,5-difluorobenzyl)sulfamoyl)benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and 1-(bromomethyl)-3,5-difluorobenzene;

LC-MS (Method B) M+1 obs 521.13, tR=2.88 min.

### Step-2: 4-(N-(3-Chloro-5-(trifluoromethyl)pyridin-2-yl)-N-(3,5-difluorobenzyl)sulfamoyl)benzoic acid

The titled compound was prepared according to the procedure described in step-2 of Example 15 from methyl 4-(N-(3-chloro-5-(trifluoromethyl)pyridin-2-yl)-N-(3,5-difluorobenzyl)sulfamoyl)benzoate (step-1 of Example 42). The further purification was carried out by preparative LC-MS system in usual manner. HPLC-QC method, retention time and observed MS were summarized in Table 6.

### Example 43

### 4-(N-(3-Chloro-5-(trifluoromethyl)pyridin-2-yl)-N-(4-fluoro-3-(trifluoromethyl)benzyl)sulfamoyl)benzoic acid

### Step-1: Methyl 4-(N-(3-chloro-5-(trifluoromethyl)pyridin-2-yl)-N-(4-fluoro-3-(trifluoromethyl)benzyl)sulfamoyl)benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and 4-(bromomethyl)-1-fluoro-2-(trifluoromethyl)benzene;

LC-MS (Method B) m/z: M+1 obs 571.12, tR=2.97 min.

### Step-2: 4-(N-(3-Chloro-5-(trifluoromethyl)pyridin-2-yl)-N-(4-fluoro-3-(trifluoromethyl)benzyl)sulfamoyl)benzoic acid

The titled compound was prepared according to the procedure described in step-2 of Example 15 from methyl 4-(N-(3-chloro-5-(trifluoromethyl)pyridin-2-yl)-N-(4-fluoro-3-(trifluoromethyl)benzyl)sulfamoyl)benzoate (step-1 of Example 43). The further purification was carried out by preparative LC-MS system in usual manner. HPLC-QC method, retention time and observed MS were summarized in Table 6.

### Example 44

### 4-(N-(3-Chloro-5-(trifluoromethyl)pyridin-2-yl)-N-(2,6-difluorobenzyl)sulfamoyl)benzoic acid

### Step-1: Methyl 4-(N-(3-chloro-5-(trifluoromethyl)pyridin-2-yl)-N-(2,6-difluorobenzyl)sulfamoyl)benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and 2-(bromomethyl)-1,3-difluorobenzene;

LC-MS (Method B) m/z: M+1 obs 521.13, tR=2.79 min.

### Step-2: 4-(N-(3-Chloro-5-(trifluoromethyl)pyridin-2-yl)-N-(2,6-difluorobenzyl)sulfamoyl)benzoic acid

The titled compound was prepared according to the procedure described in step-2 of Example 15 from methyl 4-(N-(3-chloro-5-(trifluoromethyl)pyridin-2-yl)-N-(2,6-difluorobenzyl)sulfamoyl)benzoate (step-1 of Example 44). The further purification was carried out by preparative LC-MS system in usual manner. HPLC-QC method, retention time and observed MS were summarized in Table 6.

### Example 45

### 4-(N-(3-Chloro-5-(trifluoromethyl)pyridin-2-yl)-N-(3,4-difluorobenzyl)sulfamoyl)benzoic acid

### Step-1: Methyl 4-(N-(3-chloro-5-(trifluoromethyl)pyridin-2-yl)-N-(3,4-difluorobenzyl)sulfamoyl)benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and 4-(bromomethyl)-1,2-difluorobenzene;

LC-MS (Method B) m/z: M+1 obs 521.13, tR=2.88 min.

### Step-2: 4-(N-(3-Chloro-5-(trifluoromethyl)pyridin-2-yl)-N-(3,4-difluorobenzyl)sulfamoyl)benzoic acid

The titled compound was prepared according to the procedure described in step-2 of Example 15 from methyl 4-(N-(3-chloro-5-(trifluoromethyl)pyridin-2-yl)-N-(3,4-difluorobenzyl)sulfamoyl)benzoate (step-1 of Example 45). The further purification was carried out by preparative LC-MS system in usual manner. HPLC-QC method, retention time and observed MS were summarized in Table 6.

### Example 46

### 4-(N-(3-Chloro-5-(trifluoromethyl)pyridin-2-yl)-N-(2,5-difluorobenzyl)sulfamoyl)benzoic acid

### Step-1: Methyl 4-(N-(3-chloro-5-(trifluoromethyl)pyridin-2-yl)-N-(2,5-difluorobenzyl)sulfamoyl)benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and 2-(bromomethyl)-1,4-difluorobenzene;

LC-MS (Method B) m/z: M+1 obs 521.13, tR=2.84 min.

### Step-2: 4-(N-(3-Chloro-5-(trifluoromethyl)pyridin-2-yl)-N-(2,5-difluorobenzyl)sulfamoyl)benzoic acid

The titled compound was prepared according to the procedure described in step-2 of Example 15 from methyl 4-(N-(3-chloro-5-(trifluoromethyl)pyridin-2-yl)-N-(2,5-difluorobenzyl)sulfamoyl)benzoate (step-1 of Example 46). The further purification was carried out by preparative LC-MS system in usual manner. HPLC-QC method, retention time and observed MS were summarized in Table 6.

### Example 47

### 4-(N-(3-Chloro-5-(trifluoromethyl)pyridin-2-yl)-N-(3,4-dichlorobenzyl)sulfamoyl)benzoic acid

### Step-1: Methyl 4-(N-(3-chloro-5-(trifluoromethyl)pyridin-2-yl)-N-(3,4-dichlorobenzyl)sulfamoyl)benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and 4-(bromomethyl)-1,2-dichlorobenzene;

LC-MS (Method B) m/z: M+1 obs 555.05, tR=3.07 min.

### Step-2: 4-(N-(3-Chloro-5-(trifluoromethyl)pyridin-2-yl)-N-(3,4-dichlorobenzyl)sulfamoyl)benzoic acid

The titled compound was prepared according to the procedure described in step-2 of Example 15 from methyl 4-(N-(3-chloro-5-(trifluoromethyl)pyridin-2-yl)-N-(3,4-dichlorobenzyl)sulfamoyl)benzoate (step-1 of Example 47). The further purification was carried out by preparative LC-MS system in usual manner. HPLC-QC method, retention time and observed MS were summarized in Table 6.

### Example 48

### 4-(N-(3-Chloro-5-(trifluoromethyl)pyridin-2-yl)-N-(3-chlorobenzyl)sulfamoyl)benzoic acid

### Step-1: Methyl

### 4-(N-(3-chloro-5-(trifluoromethyl)pyridin-2-yl)-N-(3-chlorobenzyl)sulfamoyl)benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and 1-(bromomethyl)-3-chlorobenzene;

LC-MS (Method B) m/z: M+1 obs 519.09, tR=2.95 min.

### Step-2: 4-(N-(3-Chloro-5-(trifluoromethyl)pyridin-2-yl)-N-(3-chlorobenzyl)sulfamoyl)benzoic acid

The titled compound was prepared according to the procedure described in step-2 of Example 15 from methyl 4-(N-(3-chloro-5-(trifluoromethyl)pyridin-2-yl)-N-(3-chlorobenzyl)sulfamoyl)benzoate (step-1 of Example 48). The further purification was carried out by preparative LC-MS system in usual manner. HPLC-QC method, retention time and observed MS were summarized in Table 6.

### Example 49

### 4-(N-(3-Chloro-5-(trifluoromethyl)pyridin-2-yl)-N-(4-(1-methylcyclopropyl)benzyl)sulfamoyl)benzoic acid

### Step-1: Methyl 4-(N-(3-chloro-5-(trifluoromethyl)pyridin-2-yl)-N-(4-(1-methylcyclopropyl)benzyl)sulfamoyl)benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and 1-(chloromethyl)-4-(1-methylcyclopropyl)benzene;

LC-MS (Method B) m/z: M+1 obs 539.2, tR=3.15 min.

### Step-2: 4-(N-(3-Chloro-5-(trifluoromethyl)pyridin-2-yl)-N-(4-(1-methyl)cyclopropyl)benzyl)sulfamoyl)benzoic acid

The titled compound was prepared according to the procedure described in step-2 of Example 15 from methyl 4-(N-(3-chloro-5-(trifluoromethyl)pyridin-2-yl)-N-(4-(1-methylcyclopropyl)benzyl)sulfamoyl)benzoate (step-1 of Example 49). The further purification was carried out by preparative LC-MS system in usual manner. HPLC-QC method, retention time and observed MS were summarized in Table 6.

### Example 50

### 4-(N-(3-chloro-5-(trifluoromethyl)pyridin-2-yl)-N-(4-(1,1,1-trifluoro-2-methylpropan-2-yl)benzyl)sulfamoyl)benzoic acid

### Step-1: Methyl 4-(N-(3-chloro-5-(trifluoromethyl)pyridin-2-yl)-N-(4-(1,1,1-trifluoro-2-methylpropan-2-yl)benzyl)sulfamoyl)benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and 1-(chloromethyl)-4-(1,1,1-trifluoro-2-methylpropan-2-yl)benzene;

LC-MS (Method A) m/z: M+1 obs 595.16, tR=3.70 min.

### Step-2: 4-(N-(3-Chloro-5-(trifluoromethyl)pyridin-2-yl)-N-(4-(1,1,1-trifluoro-2-methylpropan-2-yl)benzyl)sulfamoyl)benzoic acid

The titled compound was prepared according to the procedure described in step-2 of Example 15 from methyl 4-(N-(3-chloro-5-(trifluoromethyl)pyridin-2-yl)-N-(4-(1,1,1-trifluoro-2-methylpropan-2-yl)benz yl)sulfamoyl)benzoate (step-1 of Example 50). The further purification was carried out by preparative LC-MS system in usual manner. HPLC-QC method, retention time and observed MS were summarized in Table 6.

### Example 51

### 4-(N-(3-Chloro-5-(trifluoromethyl)pyridin-2-yl)-N-(2-(trifluoromethoxy)benzyl)sulfamoyl)benzoic acid

### Step-1: Methyl 4-(N-(3-chloro-5-(trifluoromethyl)pyridin-2-yl)-N-(2-(trifluoromethoxy)benzyl)sulfamoyl)benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and 1-(bromomethyl)-2-(trifluoromethoxy)benzene;

LC-MS (Method A) m/z: M+1 obs 569.14, tR=3.60 min.

### Step-2: 4-(N-(3-Chloro-5-(trifluoromethyl)pyridin-2-yl)-N-(2-(trifluoromethoxy)benzyl)sulfamoyl)benzoic acid

The titled compound was prepared according to the procedure described in step-2 of Example 15 from methyl 4-(N-(3-chloro-5-(trifluoromethyl)pyridin-2-yl)-N-(2-(trifluoromethoxy)benzyl)sulfamoyl)benzoate (step-1 of Example 51). The further purification was carried out by preparative LC-MS system in usual manner. HPLC-QC method, retention time and observed MS were summarized in Table 6.

### Example 52

### 3-(N-Benzyl-N-(3-chloro-5-(trifluoromethyl)pyridin-2-yl)sulfamoyl)benzoic acid

### Step-1: Methyl 3-(N-(3-chloro-5-(trifluoromethyl)pyridin-2-yl)sulfamoyl)benzoate

To a suspension of NaH (60% in oil, 146 mg, 3.82 mmol) in THF (30 mL) was added 3-chloro-5-(trifluoromethyl)pyridin-2-amine (500 mg, 2.54 mmol) at 0° C. and stirred at room temperature for 1 h. Then, to the mixture was added methyl 3-(chlorosulfonyl)benzoate (597 mg, 2.54 mmol) at 0° C. and stirred at room temperature for 2 hrs. The mixture was acidified by 2 M aqueous HCl solution, extracted with EtOAc (2 times), dried over Na₂SO₄, filtered and concentrated. The residue was applied to a silica gel column chromatography and eluted with hexane/EtOAc=1/1 to furnish 455 mg (45% yield) of the titled compound as a pale yellow solid;

¹H-NMR (300 MHz, CDCl₃) δ 8.82 (1H, s), 8.45-8.36 (2H, m), 8.29 (1H, d, J=8.1 Hz), 7.84 (1H, s), 7.65 (1H, t, J=8.1 Hz), 3.97 (3H, s), a signal due to SO₂NH was not observed;

LC-MS (Method A) m/z: M+1 obs 395.15, tR=3.00 min.

### Step-2: Methyl 3-(N-benzyl-N-(3-chloro-5-(trifluoromethyl)pyridin-2-yl)sulfamoyl)benzoate

The titled compound was prepared according to the procedure described in step-1 of Example 15 from methyl 3-(N-(3-chloro-5-(trifluoromethyl)pyridin-2-yl)sulfamoyl)benzoate (step-1 of Example 52) and (bromomethyl)benzene;

LC-MS (Method B) m/z: M+1 obs 454.18, tR=2.84 min.

### Step-3: 3-(N-Benzyl-N-(3-chloro-5-(trifluoromethyl)pyridin-2-yl)sulfamoyl)benzoic acid

The titled compound was prepared according to the procedure described in step-2 of Example 15 from methyl 3-(N-benzyl-N-(3-chloro-5-(trifluoromethyl)pyridin-2-yl)sulfamoyl)benzoate (step-2 of Example 52). The further purification was carried out by preparative LC-MS system in usual manner. HPLC-QC method, retention time and observed MS were summarized in Table 6.

### Example 53

### 4-(N-Benzyl-N-(3-fluoro-5-(trifluoromethyl)pyridin-2-yl)sulfamoyl)benzoic acid

### Step-1: Methyl 4-(N-(3-fluoro-5-(trifluoromethyl)pyridin-2-yl)sulfamoyl)benzoate

The titled compound was prepared according to the procedure described in step-1 of Example 1 from methyl 4-(chlorosulfonyl)benzoate and 3-fluoro-5-(trifluoromethyl)pyridin-2-amine;

¹H-NMR (300 MHz, CDCl₃) δ 8.40-8.10 (5H, m), 7.57 (1H, dd, J=9.9 and 2.2 Hz), 3.96 (3H, s), a signal due to SO₂NH was not observed;

LC-MS (Method A) m/z: M+1 obs 379.16, tR=2.85 min.

### Step-2: Methyl 4-(N-benzyl-N-(3-fluoro-5-(trifluoromethyl)pyridin-2-yl)sulfamoyl)benzoate

The titled compound was prepared according to the procedure described in step-1 of Example 15 from methyl 4-(N-(3-fluoro-5-(trifluoromethyl)pyridin-2-yl)sulfamoyl)benzoate (step-1 of Example 53) and (bromomethyl)benzene;

LC-MS (Method B) m/z: M+1 obs 469.17, tR=2.79 min.

### Step-3: 4-(N-Benzyl-N-(3-fluoro-5-(trifluoromethyl)pyridin-2-yl)sulfamoyl)benzoic acid

The titled compound was prepared according to the procedure described in step-2 of Example 15 from methyl 4-(N-Benzyl-N-(3-fluoro-5-(trifluoromethyl)pyridin-2-yl)sulfamoyl)benzoate (step-2 of Example 53). The further purification was carried out by preparative LC-MS system in usual manner. HPLC-QC method, retention time and observed MS were summarized in Table 6.

### Example 54

### 4-(N-(3,5-Dichloropyridin-2-yl)-N-(4-(trifluoromethyl)benzyl)sulfamoyl)benzoic acid

### Step-1: Methyl 4-(N-(3,5-dichloropyridin-2-yl)-N-(4-(trifluoromethyl)benzyl)sulfamoyl)benzoate

The titled compound was prepared according to the procedure described in step-1 of Example 15 from methyl 4-(N-(3,5-dichloropyridin-2-yl)sulfamoyl)benzoate (step-1 of Example 35) and 1-(bromomethyl)-4-(trifluoromethyl)benzene;

LC-MS (Method B) m/z: M+1 obs 519.09, tR=2.97 min.

### Step-2: 4-(N-(3,5-Dichloropyridin-2-yl)-N-(4-(trifluoromethyl)benzyl)sulfamoyl)benzoic acid

The titled compound was prepared according to the procedure described in step-2 of Example 15 from methyl 4-(N-(3,5-dichloropyridin-2-yl)-N-(4-(trifluoromethyl)benzyl)sulfamoyl)benzoate (step-1 of Example 54). The further purification was carried out by preparative LC-MS system in usual manner. HPLC-QC method, retention time and observed MS were summarized in Table 6.

### Example 55

### 4-(N-(5-Chloro-3-methylpyridin-2-yl)-N-(4-(trifluoromethyl)benzyl)sulfamoyl)benzoic acid

### Step-1: Methyl 4-(N-(5-chloro-3-methylpyridin-2-yl)-N-(4-(trifluoromethyl)benzyl)sulfamoyl)benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and 1-(bromomethyl)-4-(trifluoromethyl)benzene;

LC-MS (Method B) m/z: M+1 obs 499.20, tR=3.00 min.

### Step-2: 4-(N-(5-Chloro-3-methylpyridin-2-yl)-N-(4-(trifluoromethyl)benzyl)sulfamoyl)benzoic acid

The titled compound was prepared according to the procedure described in step-2 of Example 15 from methyl 4-(N-(5-chloro-3-methylpyridin-2-yl)-N-(4-(trifluoromethyl)benzyl)sulfamoyl)benzoate (step-1 of Example 55). The further purification was carried out by preparative LC-MS system in usual manner. HPLC-QC method, retention time and observed MS were summarized in Table 6.

### Example 56

### 4-(N-Benzyl-N-(2-chloro-4-(trifluoromethyl)phenyl)sulfamoyl)benzoic acid

### Step-1: Methyl 4-(N-(2-chloro-4-(trifluoromethyl)phenyl)sulfamoyl)benzoate

The titled compound was prepared according to the procedure described in step-1 of Example 1 from methyl 4-(chlorosulfonyl)benzoate and 2-chloro-4-(trifluoromethyl)aniline in the presence of DMAP (0.2 eq.);

¹H-NMR (300 MHz, CDCl₃) δ 8.13 (2H, d, J=8.8 Hz), 7.89 (2H, d, J=8.8 Hz), 7.78 (1H, d, J=8.8 Hz), 7.55 (1H, s), 7.51 (1H, d, J=8.8 Hz), 3.94 (3H, s), a signal due to SO₂NH was not observed;

LC-MS (Method B) m/z: M−1 obs 392.10, tR=2.42 min.

### Step-2: Methyl 4-(N-benzyl-N-(2-chloro-4-(trifluoromethyl)phenyl)sulfamoyl)benzoate

The titled compound was prepared according to the procedure described in step-1 of Example 15 from methyl 4-(N-(2-chloro-4-(trifluoromethyl)phenyl)sulfamoyl)benzoate (step-1 of Example 56) and (bromomethyl)benzene;

LC-MS (Method A) m/z: M+1 obs 484.09, tR=3.55 min.

### Step-3: 4-(N-Benzyl-N-(2-chloro-4-(trifluoromethyl)phenyl)sulfamoyl)benzoic acid

The titled compound was prepared according to the procedure described in step-2 of Example 15 from methyl 4-(N-benzyl-N-(2-chloro-4-(trifluoromethyl)phenyl)sulfamoyl)benzoate (step-2 of Example 56). The further purification was carried out by preparative LC-MS system in usual manner. HPLC-QC method, retention time and observed MS were summarized in Table 6.

### Example 57

### 4-(N-(3-Chloro-5-(trifluoromethyl)pyridin-2-yl)-N-(2-morpholinoethyl)sulfamoyl)benzoic acid

### Step-1: 4-(N-(3-Chloro-5-(trifluoromethyl)pyridin-2-yl)-N-(2-morpholinoethyl)sulfamoyl)benzoic acid

To a mixture of methyl 4-(N-(3-chloro-5-(trifluoromethyl)pyridin-2-yl)sulfamoyl)benzoate (step-1 of Example 1, 30 mg, 0.076 mmol) and 4-(2-chloroethyl)morpholine hydrochloride (28 mg, 0.152 mmol) in DMF (0.3 mL) were added Cs₂CO₃ (99 mg, 0.304 mmol) and NaI (11 mg, 0.076 mmol) at room temperature. The mixture was irradiated with micro-wave (150° C., 1 h). The mixture was acidified by 2 M aqueous HCl solution, extracted with DCM, dried over Na₂SO₄, filtered and concentrated. The further purification was carried out by preparative LC-MS system in usual manner. HPLC-QC method, retention time and observed MS were summarized in Table 6.

### Example 58

### 4-(N-(3-Chloro-5-(trifluoromethyl)pyridin-2-yl)-N-(pyridin-2-ylmethyl)sulfamoyl)benzoic acid

### Step-1: Methyl 4-(N-(3-chloro-5-(trifluoromethyl)pyridin-2-yl)-N-(pyridin-2-ylmethyl)sulfamoyl)benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and 2-(bromomethyl)pyridine hydrobromide;

LC-MS (Method B) m/z: M+1 obs 486.2, tR=2.48 min.

### Step-2: 4-(N-(3-Chloro-5-(trifluoromethyl)pyridin-2-yl)-N-(pyridin-2-ylmethyl)sulfamoyl)benzoic acid

The titled compound was prepared according to the procedure described in step-2 of Example 15 from methyl 4-(N-(3-chloro-5-(trifluoromethyl)pyridin-2-yl)-N-(pyridin-2-ylmethyl)sulfamoyl)benzoate (step-1 of Example 58). The further purification was carried out by preparative LC-MS system in usual manner. HPLC-QC method, retention time and observed MS were summarized in Table 6.

### Example 59

### 4-(N-(3-Chloro-5-(trifluoromethyl)pyridin-2-yl)-N-(3-phenylpropyl)sulfamoyl)benzoic acid

### Step-1: Methyl 4-(N-(3-chloro-5-(trifluoromethyl)pyridin-2-yl)-N-(3-phenylpropyl)sulfamoyl)benzoate

To a mixture of methyl 4-(N-(3-chloro-5-(trifluoromethyl)pyridin-2-yl)sulfamoyl)benzoate (step-1 of Example 1, 30 mg, 0.076 mmol) and (3-bromopropyl)benzene (30 mg, 0.152 mmol) in DMF (0.6 mL) were added Cs₂CO₃ (99 mg, 0.304 mmol) and NaI (11 mg, 0.076 mmol) at room temperature. The mixture was irradiated with micro-wave (150° C., 30 min). The mixture was acidified by 2 M aqueous HCl solution, extracted with DCM, dried over Na₂SO₄, filtered and concentrated. The residue was used next step without purification.

LC-MS (Method B) m/z: M+1 obs 513.2, tR=3.00 min

### Step-2: 4-(N-(3-Chloro-5-(trifluoromethyl)pyridin-2-yl)-N-(3-phenylpropyl)sulfamoyl)benzoic acid

The titled compound was prepared according to the procedure described in step-2 of Example 15 from methyl 4-(N-(3-chloro-5-(trifluoromethyl)pyridin-2-yl)-N-(3-phenylpropyl)sulfamoyl)benzoate (step-1 of Example 59). The further purification was carried out by preparative LC-MS system in usual manner. HPLC-QC method, retention time and observed MS were summarized in Table 6.

### Example 60

### 4-(N-(3,5-Dichloropyridin-2-yl)-N-(4-fluoro-3-(trifluoromethyl)benzyl)sulfamoyl)benzoic acid

### Step-1: Methyl 4-(N-(3,5-dichloropyridin-2-yl)-N-(4-fluoro-3-(trifluoromethyl)benzyl)sulfamoyl)benzoate

The titled compound was prepared according to the procedure described in step-1 of Example 15 from methyl 4-(N-(3,5-dichloropyridin-2-yl)sulfamoyl)benzoate (step-1 of Example 35) and 4-(bromomethyl)-1-fluoro-2-(trifluoromethyl)benzene.

### Step-2: 4-(N-(3,5-Dichloropyridin-2-yl)-N-(4-fluoro-3-(trifluoromethyl)benzyl)sulfamoyl)benzoic acid

The titled compound was prepared according to the procedure described in step-2 of Example 15 from methyl 4-(N-(3,5-dichloropyridin-2-yl)-N-(4-fluoro-3-(trifluoromethyl)benzyl)sulfamoyl)benzoate (step-1 of Example 60). The further purification was carried out by preparative LC-MS system in usual manner. HPLC-QC method, retention time and observed MS were summarized in Table 6.

### Example 61

### 4-(N-(3,5-Dichloropyridin-2-yl)-N-(4-fluorobenzyl)sulfamoyl)benzoic acid

### Step-1: Methyl 4-(N-(3,5-dichloropyridin-2-yl)-N-(4-fluorobenzyl)sulfamoyl)benzoate

The titled compound was prepared according to the procedure described in step-1 of Example 15 from methyl 4-(N-(3,5-dichloropyridin-2-yl)sulfamoyl)benzoate (step-1 of Example 35) and 1-(bromomethyl)-4-fluorobenzene.

### Step-2: 4-(N-(3,5-Dichloropyridin-2-yl)-N-(4-fluorobenzyl)sulfamoyl)benzoic acid

The titled compound was prepared according to the procedure described in step-2 of Example 15 from methyl 4-(N-(3,5-dichloropyridin-2-yl)-N-(4-fluorobenzyl)sulfamoyl)benzoate (step-1 of Example 61). The further purification was carried out by preparative LC-MS system in usual manner. HPLC-QC method, retention time and observed MS were summarized in Table 6.

### Example 62

### 4-(N-(4-Chloro-3-fluorobenzyl)-N-(3,5-dichloropyridin-2-yl)sulfamoyl)benzoic acid

### Step-1: Methyl 4-(N-(4-chloro-3-fluorobenzyl)-N-(3,5-dichloropyridin-2-yl)sulfamoyl)benzoate

The titled compound was prepared according to the procedure described in step-1 of Example 15 from methyl 4-(N-(3,5-dichloropyridin-2-yl)sulfamoyl)benzoate (step-1 of Example 35) and 4-(bromomethyl)-1-chloro-2-fluorobenzene.

### Step-2: 4-(N-(4-Chloro-3-fluorobenzyl)-N-(3,5-dichloropyridin-2-yl)sulfamoyl)benzoic acid

The titled compound was prepared according to the procedure described in step-2 of Example 15 from methyl 4-(N-(4-chloro-3-fluorobenzyl)-N-(3,5-dichloropyridin-2-yl)sulfamoyl)benzoate (step-1 of Example 62). The further purification was carried out by preparative LC-MS system in usual manner. HPLC-QC method, retention time and observed MS were summarized in Table 6.

### Example 63

### 4-(N-(3,5-Dichloropyridin-2-yl)-N-(4-(trifluoromethoxy)benzyl)sulfamoyl)benzoic acid

### Step-1: Methyl 4-(N-(3,5-dichloropyridin-2-yl)-N-(4-(trifluoromethoxy)benzyl)sulfamoyl)benzoate

The titled compound was prepared according to the procedure described in step-1 of Example 15 from methyl 4-(N-(3,5-dichloropyridin-2-yl)sulfamoyl)benzoate (step-1 of Example 35) and 1-(bromomethyl)-4-(trifluoromethoxy)benzene.

### Step-2: 4-(N-(3,5-Dichloropyridin-2-yl)-N-(4-(trifluoromethoxy)benzyl)sulfamoyl)benzoic acid

The titled compound was prepared according to the procedure described in step-2 of Example 15 from methyl 4-(N-(3,5-dichloropyridin-2-yl)-N-(4-(trifluoromethoxy)benzyl)sulfamoyl)benzoate (step-1 of Example 63). The further purification was carried out by preparative LC-MS system in usual manner. HPLC-QC method, retention time and observed MS were summarized in Table 6.

### Example 64

### 4-(N-(3,5-Dichloropyridin-2-yl)-N-(2-(trifluoromethyl)benzyl)sulfamoyl)benzoic acid

### Step-1: Methyl 4-(N-(3,5-dichloropyridin-2-yl)-N-(2-(trifluoromethyl)benzyl)sulfamoyl)benzoate

The titled compound was prepared according to the procedure described in step-1 of Example 15 from methyl 4-(N-(3,5-dichloropyridin-2-yl)sulfamoyl)benzoate (step-1 of Example 35) and 1-(bromomethyl)-2-(trifluoromethyl)benzene.

### Step-2: 4-(N-(3,5-Dichloropyridin-2-yl)-N-(2-(trifluoromethyl)benzyl)sulfamoyl)benzoic acid

The titled compound was prepared according to the procedure described in step-2 of Example 15 from methyl 4-(N-(3,5-dichloropyridin-2-yl)-N-(2-(trifluoromethyl)benzyl)sulfamoyl)benzoate (step-1 of Example 64). The further purification was carried out by preparative LC-MS system in usual manner. HPLC-QC method, retention time and observed MS were summarized in Table 6.

### Example 65

### 4-(N-(4-Chloro-3-fluorobenzyl)-N-(3-chloro-5-(trifluoromethyl)pyridin-2-yl)sulfamoyl)benzoic acid

### Step-1: Methyl 4-(N-(4-chloro-3-fluorobenzyl)-N-(3-chloro-5-(trifluoromethyl)pyridin-2-yl)sulfamoyl)benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and 4-(bromomethyl)-1-chloro-2-fluorobenzene.

### Step-2: 4-(N-(4-Chloro-3-fluorobenzyl)-N-(3-chloro-5-(trifluoromethyl)pyridin-2-yl)sulfamoyl)benzoic acid

The titled compound was prepared according to the procedure described in step-2 of Example 15 from methyl 4-(N-(4-chloro-3-fluorobenzyl)-N-(3-chloro-5-(trifluoromethyl)pyridin-2-yl)sulfamoyl)benzoate (step-1 of Example 65). The further purification was carried out by preparative LC-MS system in usual manner. HPLC-QC method, retention time and observed MS were summarized in Table 6.

### Example 66

### 4-(N-(4-Chloro-3-(trifluoromethyl)benzyl)-N-(3,5-dichloropyridin-2-yl)sulfamoyl)benzoic acid

### Step-1: Methyl 4-(N-(4-chloro-3-(trifluoromethyl)benzyl)-N-(3,5-dichloropyridin-2-yl)sulfamoyl)benzoate

The titled compound was prepared according to the procedure described in step-1 of Example 15 from methyl 4-(N-(3,5-dichloropyridin-2-yl)sulfamoyl)benzoate (step-1 of Example 35) and 4-(bromomethyl)-1-chloro-2-(trifluoromethyl)benzene.

### Step-2: 4-(N-(4-Chloro-3-(trifluoromethyl)benzyl)-N-(3,5-dichloropyridin-2-yl)sulfamoyl)benzoic acid

The titled compound was prepared according to the procedure described in step-2 of Example 15 from methyl 4-(N-(4-chloro-3-(trifluoromethyl)benzyl)-N-(3,5-dichloropyridin-2-yl)sulfamoyl)benzoate (step-1 of Example 66). The further purification was carried out by preparative LC-MS system in usual manner. HPLC-QC method, retention time and observed MS were summarized in Table 6.

### Example 67

### 4-(N-(3,5-Dichloropyridin-2-yl)-N-(3-fluoro-4-methylbenzyl)sulfamoyl)benzoic acid

### Step-1: Methyl 4-(N-(3,5-dichloropyridin-2-yl)-N-(3-fluoro-4-methylbenzyl)sulfamoyl)benzoate

The titled compound was prepared according to the procedure described in step-1 of Example 15 from methyl 4-(N-(3,5-dichloropyridin-2-yl)sulfamoyl)benzoate (step-1 of Example 35) and 4-(bromomethyl)-2-fluoro-1-methylbenzene.

### Step-2: 4-(N-(3,5-Dichloropyridin-2-yl)-N-(3-fluoro-4-methylbenzyl)sulfamoyl)benzoic acid

The titled compound was prepared according to the procedure described in step-2 of Example 15 from methyl 4-(N-(3,5-dichloropyridin-2-yl)-N-(3-fluoro-4-methylbenzyl)sulfamoyl)benzoate (step-1 of Example 67). The further purification was carried out by preparative LC-MS system in usual manner. HPLC-QC method, retention time and observed MS were summarized in Table 6.

### Example 68

### 4-(N-(3,5-dichloropyridin-2-yl)-N-(4-methyl-3-(trifluoromethyl)benzyl)sulfamoyl)benzoic acid

### Step-1: Methyl 4-(N-(3,5-dichloropyridin-2-yl)-N-(4-methyl-3-(trifluoromethyl)benzyl)sulfamoyl)benzoate

The titled compound was prepared according to the procedure described in step-1 of Example 15 from methyl 4-(N-(3,5-dichloropyridin-2-yl)sulfamoyl)benzoate (step-1 of Example 35) and 4-(bromomethyl)-1-methyl-2-(trifluoromethyl)benzene.

### Step-2: 4-(N-(3,5-Dichloropyridin-2-yl)-N-(4-methyl-3-(trifluoromethyl)benzyl)sulfamoyl)benzoic acid

The titled compound was prepared according to the procedure described in step-2 of Example 15 from methyl 4-(N-(3,5-dichloropyridin-2-yl)-N-(4-methyl-3-(trifluoromethyl)benzyl)sulfamoyl)benzoate (step-1 of Example 68). The further purification was carried out by preparative LC-MS system in usual manner. HPLC-QC method, retention time and observed MS were summarized in Table 6.

### Example 69

### 4-(N-(3-Chloro-4-fluorobenzyl)-N-(3,5-dichloropyridin-2-yl)sulfamoyl)benzoic acid

### Step-1: Methyl 4-(N-(3-chloro-4-fluorobenzyl)-N-(3,5-dichloropyridin-2-yl)sulfamoyl)benzoate

The titled compound was prepared according to the procedure described in step-1 of Example 15 from methyl 4-(N-(3,5-dichloropyridin-2-yl)sulfamoyl)benzoate (step-1 of Example 35) and 4-(bromomethyl)-2-chloro-1-fluorobenzene.

### Step-2: 4-(N-(3-Chloro-4-fluorobenzyl)-N-(3,5-dichloropyridin-2-yl)sulfamoyl)benzoic acid

The titled compound was prepared according to the procedure described in step-2 of Example 15 from methyl 4-(N-(3-chloro-4-fluorobenzyl)-N-(3,5-dichloropyridin-2-yl)sulfamoyl)benzoate (step-1 of Example 69). The further purification was carried out by preparative LC-MS system in usual manner. HPLC-QC method, retention time and observed MS were summarized in Table 6.

### Example 70

### 4-(N-(3,5-Dichloropyridin-2-yl)-N-(4-(1-methylcyclopropyl)benzyl)sulfamoyl)benzoic acid

### Step-1: Methyl 4-(N-(3,5-dichloropyridin-2-yl)-N-(4-(1-methylcyclopropyl)benzyl)sulfamoyl)benzoate

The titled compound was prepared according to the procedure described in step-1 of Example 15 from methyl 4-(N-(3,5-dichloropyridin-2-yl)sulfamoyl)benzoate (step-1 of Example 35) and 1-(chloromethyl)-4-(1-methylcyclopropyl)benzene.

### Step-2: 4-(N-(3,5-Dichloropyridin-2-yl)-N-(4-(1-methylcyclopropyl)benzyl)sulfamoyl)benzoic acid

The titled compound was prepared according to the procedure described in step-2 of Example 15 from methyl 4-(N-(3,5-dichloropyridin-2-yl)-N-(4-(1-methylcyclopropyl)benzyl)sulfamoyl)benzoate (step-1 of Example 70). The further purification was carried out by preparative LC-MS system in usual manner. HPLC-QC method, retention time and observed MS were summarized in Table 6.

### Example 71

### 4-(N-(3-Chloro-4-fluorobenzyl)-N-(3-chloro-5-(trifluoromethyl)pyridin-2-yl)sulfamoyl)benzoic acid

### Step-1: Methyl 4-(N-(3-chloro-4-fluorobenzyl)-N-(3-chloro-5-(trifluoromethyl)pyridin-2-yl)sulfamoyl)benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and 4-(bromomethyl)-2-chloro-1-fluorobenzene.

### Step-2: 4-(N-(3-chloro-4-fluorobenzyl)-N-(3-chloro-5-(trifluoromethyl)pyridin-2-yl)sulfamoyl)benzoic acid

The titled compound was prepared according to the procedure described in step-2 of Example 15 from methyl 4-(N-(3-chloro-4-fluorobenzyl)-N-(3-chloro-5-(trifluoromethyl)pyridin-2-yl)sulfamoyl)benzoate (step-1 of Example 71). The further purification was carried out by preparative LC-MS system in usual manner. HPLC-QC method, retention time and observed MS were summarized in Table 6.

### Example 72

### 4-(N-(4-Chloro-2-(trifluoromethyl)benzyl)-N-(3,5-dichloropyridin-2-yl)sulfamoyl)benzoic acid

### Step-1: Methyl 4-(N-(4-chloro-2-(trifluoromethyl)benzyl)-N-(3,5-dichloropyridin-2-yl)sulfamoyl)benzoate

The titled compound was prepared according to the procedure described in step-1 of Example 15 from methyl 4-(N-(3,5-dichloropyridin-2-yl)sulfamoyl)benzoate (step-1 of Example 35) and 1-(bromomethyl)-4-chloro-2-(trifluoromethyl)benzene.

### Step-2: 4-(N-(4-Chloro-2-(trifluoromethyl)benzyl)-N-(3,5-dichloropyridin-2-yl)sulfamoyl)benzoic acid

The titled compound was prepared according to the procedure described in step-2 of Example 15 from methyl 4-(N-(4-chloro-2-(trifluoromethyl)benzyl)-N-(3,5-dichloropyridin-2-yl)sulfamoyl)benzoate (step-1 of Example 72). The further purification was carried out by preparative LC-MS system in usual manner. HPLC-QC method, retention time and observed MS were summarized in Table 6.

### Example 73

### 4-(N-(3,5-Dichloropyridin-2-yl)-N-(2-(4-fluorophenoxy)ethyl)sulfamoyl)benzoic acid

### Step-1: Methyl 4-(N-(3,5-dichloropyridin-2-yl)-N-(2-(4-fluorophenoxy)ethyl)sulfamoyl)benzoate

The titled compound was prepared according to the procedure described in step-1 of Example 15 from methyl 4-(N-(3,5-dichloropyridin-2-yl)sulfamoyl)benzoate (step-1 of Example 35) and 1-(2-bromoethoxy)-4-fluorobenzene.

### Step-2: 4-(N-(3,5-Dichloropyridin-2-yl)-N-(2-(4-fluorophenoxy)ethyl)sulfamoyl)benzoic acid

The titled compound was prepared according to the procedure described in step-2 of Example 15 from methyl 4-(N-(3,5-dichloropyridin-2-yl)-N-(2-(4-fluorophenoxy)ethyl)sulfamoyl)benzoate. The further purification was carried out by preparative LC-MS system in usual manner. HPLC-QC method, retention time and observed MS were summarized in Table 6.

### Example 74

### 4-(N-(3,5-Dichloropyridin-2-yl)-N-(4-(2-oxopyrrolidin-1-yl)benzyl)sulfamoyl)benzoic acid

### Step-1: Methyl

### 4-(N-(3,5-dichloropyridin-2-yl)-N-(4-(2-oxopyrrolidin-1-yl)benzyl)sulfamoyl)benzoate

The titled compound was prepared according to the procedure described in step-1 of Example 15 from methyl 4-(N-(3,5-dichloropyridin-2-yl)sulfamoyl)benzoate (step-1 of Example 35) and 1-(4-(chloromethyl)phenyl)pyrrolidin-2-one.

### Step-2: 4-(N-(3,5-Dichloropyridin-2-yl)-N-(4-(2-oxopyrrolidin-1-yl)benzyl)sulfamoyl)benzoic acid

The titled compound was prepared according to the procedure described in step-2 of Example 15 from methyl 4-(N-(3,5-dichloropyridin-2-yl)-N-(4-(2-oxopyrrolidin-1-yl)benzyl)sulfamoyl)benzoate (step-1 of Example 74). The further purification was carried out by preparative LC-MS system in usual manner. HPLC-QC method, retention time and observed MS were summarized in Table 6.

### Example 75

### 4-(N-(4-Chloro-2-(trifluoromethyl)benzyl)-N-(5-chloro-3-methylpyridin-2-yl)sulfamoyl)benzoic acid

### Step-1: Methyl 4-(N-(4-chloro-2-(trifluoromethyl)benzyl)-N-(5-chloro-3-methylpyridin-2-yl)sulfamoyl)benzoate

The titled compound was prepared according to the procedure described in step-1 of Example 15 from methyl 4-(N-(5-chloro-3-methylpyridin-2-yl)sulfamoyl)benzoate (step-1 of Example 38) and 1-(bromomethyl)-4-chloro-2-(trifluoromethyl)benzene.

### Step-2: 4-(N-(4-Chloro-2-(trifluoromethyl)benzyl)-N-(5-chloro-3-methylpyridin-2-yl)sulfamoyl)benzoic acid

The titled compound was prepared according to the procedure described in step-2 of Example 15 from methyl 4-(N-(4-chloro-2-(trifluoromethyl)benzyl)-N-(5-chloro-3-methylpyridin-2-yl)sulfamoyl)benzoate (step-1 of Example 75). The further purification was carried out by preparative LC-MS system in usual manner. HPLC-QC method, retention time and observed MS were summarized in Table 6.

### Example 76

### 4-(N-(5-Chloro-3-methylpyridin-2-yl)-N-(4-fluoro-3-(trifluoromethyl)benzyl)sulfamoyl)benzoic acid

### Step-1: Methyl 4-(N-(5-chloro-3-methylpyridin-2-yl)-N-(4-fluoro-3-(trifluoromethyl)benzyl)sulfamoyl)benzoate

The titled compound was prepared according to the procedure described in step-1 of Example 15 from methyl 4-(N-(5-chloro-3-methylpyridin-2-yl)sulfamoyl)benzoate (step-1 of Example 38) and 4-(bromomethyl)-1-fluoro-2-(trifluoromethyl)benzene.

### Step-2: 4-(N-(5-Chloro-3-methylpyridin-2-yl)-N-(4-fluoro-3-(trifluoromethyl)benzyl)sulfamoyl)benzoic acid

The titled compound was prepared according to the procedure described in step-2 of Example 15 from methyl 4-(N-(5-chloro-3-methylpyridin-2-yl)-N-(4-fluoro-3-(trifluoromethyl)benzyl)sulfamoyl)benzoate (step-1 of Example 76). The further purification was carried out by preparative LC-MS system in usual manner. HPLC-QC method, retention time and observed MS were summarized in Table 6.

### Example 77

### 4-(N-(5-Chloro-3-methylpyridin-2-yl)-N-(3-chloro-4-fluorobenzyl)sulfamoyl)benzoic acid

### Step-1: Methyl 4-(N-(5-chloro-3-methylpyridin-2-yl)-N-(3-chloro-4-fluorobenzyl)sulfamoyl)benzoate

The titled compound was prepared according to the procedure described in step-1 of Example 15 from methyl 4-(N-(5-chloro-3-methylpyridin-2-yl)sulfamoyl)benzoate (step-1 of Example 38) and 4-(bromomethyl)-2-chloro-1-fluorobenzene.

### Step-2: 4-(N-(5-Chloro-3-methylpyridin-2-yl)-N-(3-chloro-4-fluorobenzyl)sulfamoyl)benzoic acid

The titled compound was prepared according to the procedure described in step-2 of Example 15 from methyl 4-(N-(5-chloro-3-methylpyridin-2-yl)-N-(3-chloro-4-fluorobenzyl)sulfamoyl)benzoate (step-1 of Example 77). The further purification was carried out by preparative LC-MS system in usual manner. HPLC-QC method, retention time and observed MS were summarized in Table 6.

### Example 78

### 4-(N-(5-Chloro-3-methylpyridin-2-yl)-N-(4-(trifluoromethoxy)benzyl)sulfamoyl)benzoic acid

### Step-1: Methyl 4-(N-(5-chloro-3-methylpyridin-2-yl)-N-(4-(trifluoromethoxy)benzyl)sulfamoyl)benzoate

The titled compound was prepared according to the procedure described in step-1 of Example 15 from methyl 4-(N-(5-chloro-3-methylpyridin-2-yl)sulfamoyl)benzoate (step-1 of Example 38) and 1-(bromomethyl)-4-(trifluoromethoxy)benzene.

### Step-2: 4-(N-(5-Chloro-3-methylpyridin-2-yl)-N-(4-(trifluoromethoxy)benzyl)sulfamoyl)benzoic acid

The titled compound was prepared according to the procedure described in step-2 of Example 15 from methyl 4-(N-(5-chloro-3-methylpyridin-2-yl)-N-(4-(trifluoromethoxy)benzyl)sulfamoyl)benzoate (step-1 of Example 78). The further purification was carried out by preparative LC-MS system in usual manner. HPLC-QC method, retention time and observed MS were summarized in Table 6.

### Example 79

### 4-(N-(4-Chloro-3-(trifluoromethyl)benzyl)-N-(5-chloro-3-methylpyridin-2-yl)sulfamoyl)benzoic acid

### Step-1: Methyl 4-(N-(4-chloro-3-(trifluoromethyl)benzyl)-N-(5-chloro-3-methylpyridin-2-yl)sulfamoyl)benzoate

The titled compound was prepared according to the procedure described in step-1 of Example 15 from methyl 4-(N-(5-chloro-3-methylpyridin-2-yl)sulfamoyl)benzoate (step-1 of Example 38) and 4-(bromomethyl)-1-chloro-2-(trifluoromethyl)benzene.

### Step-2: 4-(N-(4-Chloro-3-(trifluoromethyl)benzyl)-N-(5-chloro-3-methylpyridin-2-yl)sulfamoyl)benzoic acid The titled compound was prepared according to the procedure described in step-2 of Example 15 from methyl 4-(N-(4-chloro-3-(trifluoromethyl)benzyl)-N-(5-chloro-3-methylpyridin-2-yl)sulfamoyl)benzoate (step-1 of Example 79). The further purification was carried out by preparative LC-MS system in usual manner. HPLC-QC method, retention time and observed MS were summarized in Table 6.

### Example 80

### 4-(N-(5-Chloro-3-methylpyridin-2-yl)-N-(4-(2-oxopyrrolidin-1-yl)benzyl)sulfamoyl)benzoic acid

### Step-1: Methyl 4-(N-(5-chloro-3-methylpyridin-2-yl)-N-(4-(2-oxopyrrolidin-1-yl)benzyl)sulfamoyl)benzoate

The titled compound was prepared according to the procedure described in step-1 of Example 15 from methyl 4-(N-(5-chloro-3-methylpyridin-2-yl)sulfamoyl)benzoate (step-1 of Example 38) and 1-(4-(bromomethyl)phenyl)pyrrolidin-2-one.

### Step-2: 4-(N-(5-Chloro-3-methylpyridin-2-yl)-N-(4-(2-oxopyrrolidin-1-yl)benzyl)sulfamoyl)benzoic acid

The titled compound was prepared according to the procedure described in step-2 of Example 15 from methyl 4-(N-(5-chloro-3-methylpyridin-2-yl)-N-(4-(2-oxopyrrolidin-1-yl)benzyl)sulfamoyl)benzoate (step-1 of Example 80). The further purification was carried out by preparative LC-MS system in usual manner. HPLC-QC method, retention time and observed MS were summarized in Table 6.

### Example 81

### 4-(N-(2-Chloro-4-(trifluoromethyl)phenyl)-N-(2-morpholinoethyl)sulfamoyl)benzoic acid

### Step-1: Methyl 4-(N-(2-chloro-4-(trifluoromethyl)phenyl)-N-(2-morpholinoethyl)sulfamoyl)benzoate

The titled compound was prepared according to the procedure described in step-1 of Example 59 from methyl 4-(N-(3-chloro-5-(trifluoromethyl)pyridin-2-yl)sulfamoyl)benzoate (step-1 of Example 1) and 4-(2-chloroethyl)morpholine hydrochloride;

LC-MS (Method B) M+1 obs 507.2, tR=2.50 min.

### Step-2: 4-(N-(2-Chloro-4-(trifluoromethyl)phenyl)-N-(2-morpholinoethyl)sulfamoyl)benzoic acid

The titled compound was prepared according to the procedure described in step-2 of Example 15 from methyl 4-(N-(2-chloro-4-(trifluoromethyl)phenyl)-N-(2-morpholinoethyl)sulfamoyl)benzoate (step-1 of Example 81). The further purification was carried out by preparative LC-MS system in usual manner. HPLC-QC method, retention time and observed MS were summarized in Table 6.

### Example 82

### 4-(N-(2-Chloro-4-(trifluoromethyl)phenyl)-N-(pyridin-2-ylmethyl)sulfamoyl)benzoic acid

### Step-1: Methyl 4-(N-(2-chloro-4-(trifluoromethyl)phenyl)-N-(pyridin-2-ylmethyl)sulfamoyl)benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and 2-(bromomethyl)pyridine hydrobromide;

LC-MS (Method B) m/z: M+1 obs 485.2, tR=2.63 min.

### Step-2: 4-(N-(2-Chloro-4-(trifluoromethyl)phenyl)-N-(pyridin-2-ylmethyl)sulfamoyl)benzoic acid

The titled compound was prepared according to the procedure described in step-2 of Example 15 from methyl 4-(N-(2-chloro-4-(trifluoromethyl)phenyl)-N-(pyridin-2-ylmethyl)sulfamoyl)benzoate. The further purification was carried out by preparative LC-MS system in usual manner. HPLC-QC method, retention time and observed MS were summarized in Table 6.

### Example 83

### 4-(N-(2-Chloro-4-(trifluoromethyl)phenyl)-N-(2-(piperidin-1-yl)ethyl)sulfamoyl)benzoic acid

### Step-1: Methyl 4-(N-(2-chloro-4-(trifluoromethyl)phenyl)-N-(2-(piperidin-1-yl)ethyl)sulfamoyl)benzoate

The titled compound was prepared according to the procedure described in step-1 of Example 59 from methyl 4-(N-(3-chloro-5-(trifluoromethyl)pyridin-2-yl)sulfamoyl)benzoate (step-1 of Example 1) and 1-(2-chloroethyl)piperidine hydrochloride.

### Step-2: 4-(N-(2-Chloro-4-(trifluoromethyl)phenyl)-N-(2-(piperidin-1-yl)ethyl)sulfamoyl)benzoic acid

The titled compound was prepared according to the procedure described in step-2 of Example 15 from methyl 4-(N-(2-chloro-4-(trifluoromethyl)phenyl)-N-(2-(piperidin-1-yl)ethyl)sulfamoyl)benzoate. The further purification was carried out by preparative LC-MS system in usual manner. HPLC-QC method, retention time and observed MS were summarized in Table 6.

Examples 84-139 were selected from commercially available compounds.

### Example 140

### 4-(N-(3-Chloro-5-(trifluoromethyl)pyridin-2-yl)-N-(4-(trimethylsilyl)benzyl)sulfamoyl)benzoic acid

### Step-1: Methyl 4-(N-(3-chloro-5-(trifluoromethyl)pyridin-2-yl)-N-(4-(trimethylsilyl)benzyl)sulfamoyl)benzoate

The titled compound was prepared according to the procedure described in step-2 of Example 1 from methyl 4-(N-(3-chloro-5-(trifluoromethyl)pyridin-2-yl)sulfamoyl)benzoate and (4-(bromomethyl)phenyl)trimethylsilane;

LC-MS (Method A) m/z: M+1 obs 556.97, tR=3.28 min.

### Step-2: 4-(N-(3-Chloro-5-(trifluoromethyl)pyridin-2-yl)-N-(4-(trimethyl)silyl)benzyl)sulfamoyl)benzoic acid

The titled compound was prepared according to the procedure described in step-3 of Example 1 from methyl 4-(N-(3-chloro-5-(trifluoromethyl)pyridin-2-yl)-N-(4-(trimethylsilyl)benzyl)sulfamoyl)benzoate (step-1 of Example 140). The further purification was carried out by preparative LC-MS system using the condition in Table 6.

### Example 141

### 4-(N-(3-Chloro-5-(trifluoromethyl)pyridin-2-yl)-N-(4-(1-cyanocyclopropyl)benzyl)sulfamoyl)benzoic acid

### Step-1: Methyl 4-(N-(3-chloro-5-(trifluoromethyl)pyridin-2-yl)-N-(4-(1-cyanocyclopropyl)benzyl)sulfamoyl)benzoate

The titled compound was prepared according to the procedure described in step-2 of Example 1 from methyl 4-(N-(3-chloro-5-(trifluoromethyl)pyridin-2-yl)sulfamoyl)benzoate and 1-(4-(bromomethyl)phenyl)cyclopropanecarbonitrile;

LC-MS (Method A) m/z: M+1 obs 549.89, tR=3.45 min.

### Step-2: 4-(N-(3-Chloro-5-(trifluoromethyl)pyridin-2-yl)-N-(4-(1-cyanocyclopropyl)benzyl)sulfamoyl)benzoic acid

The titled compound was prepared according to the procedure described in step-3 of Example 1 from methyl 4-(N-(3-chloro-5-(trifluoromethyl)pyridin-2-yl)-N-(4-(1-cyanocyclopropyl)benzyl)sulfamoyl)benzoate (step-1 of Example 141). The further purification was carried out by preparative LC-MS system using the condition in Table 6.

### Example 142

### 4-(N-(3-Chloro-5-(trifluoromethyl)pyridin-2-yl)-N-((1-(pyridin-2-yl)piperidin-4-yl)methyl)sulfamoyl)benzoic acid

### Step-1: Methyl 4-(N-((1-(pyridin-2-yl)piperidin-4-yl)methyl)sulfamoyl)benzoate

The titled compound was prepared according to the procedure described in step-1 of Example 148 from methyl 4-(chlorosulfonyl)benzoate and (1-(pyridin-2-yl)piperidin-4-yl)methanamine hydrochloride;

LC-MS (Method A) m/z: M+1 obs 390.08, M−1 obs 388.11, tR=2.57 min.

### Step-2: Methyl 4-(N-(3-chloro-5-(trifluoromethyl)pyridin-2-yl)-N-((1-(pyridin-2-yl)piperidin-4-yl)methyl)sulfamoyl)benzoate

The titled compound was prepared according to the procedure described in step-2 of Example 148 from 2,3-dichloro-5-(trifluoromethyl)pyridine and methyl 4-(N-((1-(pyridin-2-yl)piperidin-4-yl)methyl)sulfamoyl)benzoate (step-1 of Example 142);

LC-MS (Method A) m/z: M+1 obs 568.91, tR=3.42 min.

### Step-3: 4-(N-(3-Chloro-5-(trifluoromethyl)pyridin-2-yl)-N-((1-(pyridin-2-yl)piperidin-4-yl)methyl)sulfamoyl)benzoic acid

The titled compound was prepared according to the procedure described in step-2 of Example 15 from methyl 4-(N-(3-chloro-5-(trifluoromethyl)pyridin-2-yl)-N-((1-(pyridin-2-yl)piperidin-4-yl)methyl)sulfamoyl)benzoate (step-2 of Example 142). The further purification was carried out by preparative LC-MS system using the condition in Table 6.

### Example 143

### 4-(N-Benzyl-N-(3-chloro-5-(trifluoromethyl)pyridin-2-yl)sulfamoyl)-2-methylbenzoic acid

### Step-1: methyl 4-(N-benzylsulfamoyl)-2-methylbenzoate

The titled compound was prepared according to the procedure described in step-1 of Example 148 from N-benzyl-4-bromo-3-methylbenzenesulfonamide;

LC-MS (Method A) m/z: M−1 obs 318.13, tR=3.00 min.

### Step-2: Methyl 4-(N-benzyl-N-(3-chloro-5-(trifluoromethyl)pyridin-2-yl)sulfamoyl)-2-methylbenzoate

The titled compound was prepared according to the procedure described in step-1 of Example 148 from 2,3-dichloro-5-(trifluoromethyl)pyridine and methyl 4-(N-benzylsulfamoyl)-2-methylbenzoate (step-1 of Example 143);

LC-MS (Method A) m/z: M+1 obs 498.95, tR=3.59 min.

### Step-3: 4-(N-Benzyl-N-(3-chloro-5-(trifluoromethyl)pyridin-2-yl)sulfamoyl)-2-methylbenzoic acid

The titled compound was prepared according to the procedure described in step-2 of Example 15 from methyl 4-(N-benzyl-N-(3-chloro-5-(trifluoromethyl)pyridin-2-yl)sulfamoyl)-2-methylbenzoate (step-2 of Example 143). The further purification was carried out by preparative LC-MS system in usual manner. HPLC-QC method, retention time and observed MS were summarized in Table 6.

### Example 144

### N-Benzyl-N-(3-chloro-5-(trifluoromethyl)pyridin-2-yl)-6-hydroxypyridine-3-sulfonamide

### Step-1: N-Benzyl-N-(3-chloro-5-(trifluoromethyl)pyridin-2-yl)-6-hydroxypyridine-3-sulfonamide

A mixture of N-benzyl-N-(3-chloro-5-(trifluoromethyl)pyridin-2-yl)-6-methoxypyridine-3-sulfonamide (step-2 of Example 189, 166.9 mg, 0.365 mmol), 4 M HCl in 1,4-dioxane solution (0.18 mL, 0.729 mmol) in 1,4-dioxane (2 mL) was heated at 55° C. overnight. After cooling, the reaction mixture was concentrated using by a rotary evaporator to give 115 mg (71% yield) of the titled compound as a brown oil. The further purification was carried out by preparative LC-MS system in usual manner. HPLC-QC method, retention time and observed MS were summarized in Table 6.

### Example 145

### 4-(N-Benzyl-N-(3-chloro-5-(trifluoromethyl)pyridin-2-yl)sulfamoyl)-3-methylbenzoic acid

### Step-1: Methyl 4-(N-benzylsulfamoyl)-3-methylbenzoate

The titled compound was prepared according to the procedure described in step-3 of Example 188 from N-benzyl-4-bromo-2-methylbenzenesulfonamide;

LC-MS (Method A) m/z: M−1 obs 318.10, tR=2.97 min.

### Step-2: Methyl 4-(N-benzyl-N-(3-chloro-5-(trifluoromethyl)pyridin-2-yl)sulfamoyl)-3-methylbenzoate

The titled compound was prepared according to the procedure described in step-2 of Example 148 from 2,3-dichloro-5-(trifluoromethyl)pyridine and methyl 4-(N-benzylsulfamoyl)-3-methylbenzoate (step-1 of Example 145);

LC-MS (Method A) m/z: M+1 obs 498.90, tR=3.55 min.

### Step-3: 4-(N-Benzyl-N-(3-chloro-5-(trifluoromethyl)pyridin-2-yl)sulfamoyl)-3-methylbenzoic acid

The titled compound was prepared according to the procedure described in step-2 of Example 15 from methyl 4-(N-benzyl-N-(3-chloro-5-(trifluoromethyl)pyridin-2-yl)sulfamoyl)-3-methylbenzoate (step-2 of Example 145). The further purification was carried out by preparative LC-MS system in usual manner. HPLC-QC method, retention time and observed MS were summarized in Table 6.

### Example 146

### 4-(N-Benzyl-N-(3-chloro-5-(trifluoromethyl)pyridin-2-yl)sulfamoyl)-3-methoxybenzoic acid

### Step-1: Methyl 4-(N-benzylsulfamoyl)-2-methoxybenzoate

The titled compound was prepared according to the procedure described in step-3 of Example 188 from N-benzyl-4-bromo-2-methoxybenzenesulfonamide;

LC-MS (Method A) m/z: M+1 obs 336.06, M−1 obs 334.08, tR=2.88 min.

### Step-2: Methyl 4-(N-benzyl-N-(3-chloro-5-(trifluoromethyl)pyridin-2-yl)sulfamoyl)-3-methoxybenzoate

The titled compound was prepared according to the procedure described in step-2 of Example 148 from 2,3-dichloro-5-(trifluoromethyl)pyridine and methyl 4-(N-benzylsulfamoyl)-3-methoxybenzoate (step-1 of Example 146);

LC-MS (Method A) m/z: M+1 obs 514.98, tR=3.52 min.

### Step-3: 4-(N-Benzyl-N-(3-chloro-5-(trifluoromethyl)pyridin-2-yl)sulfamoyl)-3-methoxybenzoic acid

The titled compound was prepared according to the procedure described in step-2 of Example 15 from methyl 4-(N-benzyl-N-(3-chloro-5-(trifluoromethyl)pyridin-2-yl)sulfamoyl)-3-methoxybenzoate (step-2 of Example 146). The further purification was carried out by preparative LC-MS system in usual manner. HPLC-QC method, retention time and observed MS were summarized in Table 6.

### Example 147

### 2-(4-(N-Benzyl-N-(3-chloro-5-(trifluoromethyl)pyridin-2-yl)sulfamoyl)phenyl)acetic acid

### Step-1: Ethyl 2-(4-(N-benzylsulfamoyl)phenyl)acetate

The titled compound was prepared according to the procedure described in step-1 of Example 148 from ethyl 2-(4-(chlorosulfonyl)phenyl)acetate and benzylamine;

¹H-NMR (300 MHz, CDCl₃) δ 7.83 (2H, d, J=8.1 Hz), 7.44 (2H, d, J=8.1 Hz), 7.34-7.16 (5H, m), 4.74-4.63 (1H, m), 4.23-4.10 (4H, m), 3.70 (2H, s), 1.28 (3H, t, J=7.0 Hz),

LC-MS (Method A) m/z: M+1 obs 334.08, tR=2.93 min.

### Step-2: Ethyl 2-(4-(N-Benzyl-N-(3-chloro-5-(trifluoromethyl)pyridin-2-yl)sulfamoyl)phenyl)acetate

The titled compound was prepared according to the procedure described in step-2 of Example 148 from ethyl 2-(4-(N-benzylsulfamoyl)phenyl)acetate (step-1 of Example 147) and 2,3-dichloro-5-(trifluoromethyl)pyridine;

LC-MS (Method A) m/z: M+1 obs 513.00, tR=3.55 min.

### Step-3: 2-(4-(N-Benzyl-N-(3-chloro-5-(trifluoromethyl)pyridin-2-yl)sulfamoyl)phenyl)acetic acid

The titled compound was prepared according to the procedure described in step-2 of Example 15 from 2-(4-(N-benzyl-N-(3-chloro-5-(trifluoromethyl)pyridin-2-yl)sulfamoyl)phenyl)acetate (step-2 of Example 147).

¹H-NMR (300 MHz, CDCl₃) δ 8.53 (1H, s), 7.93 (1H, s), 7.80 (2H, d, J=8.1 Hz), 7.50 (2H, d, J=8.1 Hz), 7.24-7.11 (5H, m), 4.68 (2H, s), 3.79 (2H, s), (proton signal of COOH was not observed).

The further purification was carried out by preparative LC-MS system using the condition in Table 6.

### Example 148

### 4-(N-(3-Chloro-5-(trifluoromethyl)pyridin-2-yl)-N-phenethylsulfamoyl)benzoic acid

### Step-1: Methyl 4-(N-phenethylsulfamoyl)benzoate

To a solution of 2-phenylethanamine (200 mg, 1.65 mmol) in DCM (10 mL) was added methyl 4-(chlorosulfonyl)benzoate (prepared in step-1 of Example 1, 387 mg, 1.65 mmol) and Et₃N (501 mg, 4.95 mmol) at 0° C., and stirred at room temperature for 1 hr. The mixture was poured into 1 M aqueous HCl solution, extracted with DCM, dried over Na₂SO₄, filtered and concentrated. The residual solid was collected to give the titled compound (298 mg, 57%) as a white solid.

LC-MS (Method A) m/z: M+1 obs 320.11, tR=2.99 min.

### Step-2: Methyl 4-(N-(3-chloro-5-(trifluoromethyl)pyridin-2-yl)-N-phenethylsulfamoyl)benzoate

A mixture of methyl 4-(N-phenethylsulfamoyl)benzoate (step-1 of Example 148, 74 mg, 0.231 mmol), 2,3-dichloro-5-(trifluoromethyl)pyridine (50 mg, 0.231 mmol) and Cs₂CO₃ (226 mg, 0.694 mmol) in MeCN (1.2 mL) was irradiated with micro-wave (160° C., 15 min). The reaction mixture was poured into H₂O, extracted with EtOAc, dried over Na₂SO₄, filtered and concentrated to give the titled compound as crude.

LC-MS (Method B) m/z: M+1 obs 498.95, tR=2.92 min.

### Step-3: 4-(N-(3-Chloro-5-(trifluoromethyl)pyridin-2-yl)-N-phenethylsulfamoyl)benzoic acid

The titled compound was prepared according to the procedure described in step-2 of Example 15 from methyl 4-(N-(3-chloro-5-(trifluoromethyl)pyridin-2-yl)-N-phenethylsulfamoyl)benzoate (step-2 of Example 148). The further purification was carried out by preparative LC-MS system in usual manner. HPLC-QC method, retention time and observed MS were summarized in Table 6.

### Example 149

### 4-(N-Benzyl-N-(3-cyclopropyl-5-(trifluoromethyl)pyridin-2-yl)sulfamoyl)benzoic acid

### Step-1: Methyl 4-(N-(3-bromo-5-(trifluoromethyl)pyridin-2-yl)sulfamoyl)benzoate

The titled compound was prepared according to the procedure described in step-1 of Example 52 from methyl 4-(chlorosulfonyl)benzoate and 3-bromo-5-(trifluoromethyl)pyridin-2-amine;

LC-MS (Method A) m/z: M+1 obs 438.91, tR=2.95 min.

### Step-2: Methyl 4-(N-benzyl-N-(3-bromo-5-(trifluoromethyl)pyridin-2-yl)sulfamoyl)benzoate

To a mixture of methyl 4-(N-(3-bromo-5-(trifluoromethyl)pyridin-2-yl)sulfamoyl)benzoate (step-1 of Example 149, 1.18 g, 2.69 mmol) and (bromomethyl)benzene (1.38 g, 8.06 mmol) in DMF (0.5 mL) was added Cs₂CO₃ (3.51 g, 10.75 mmol) and NaI (403 mg, 2.69 mmol) at room temperature. The mixture was stirred at 90° C. for 3 hr. The mixture was diluted with EtOAc, quenched with 1 M aqueous HCl solution, extracted with EtOAc, dried over Na₂SO₄, filtered and concentrated. The residue, which was applied to an amino-silica gel chromatography column and eluted with a hexane/EtOAc=4/1 to furnish 645 mg (45% yield) of the title as a white solid;

¹H-NMR (300 MHz, CDCl₃) δ 8.56 (1H, d, J=2.2 Hz), 8.21 (2H, d, J=8.1 Hz), 8.12 (1H, d, J=2.2 Hz), 7.87 (2H, d, J=8.1 Hz), 7.19 (5H, s), 4.68 (2H, s), 3.99 (3H, s).

LC-MS (Method A) m/z: M+1 obs 528.88, tR=3.72 min

### Step-3: 4-(N-Benzyl-N-(3-cyclopropyl-5-(trifluoromethyl)pyridin-2-yl)sulfamoyl)benzoic acid

A mixture of methyl 4-(N-benzyl-N-(3-bromo-5-(trifluoromethyl)pyridin-2-yl)sulfamoyl)benzoate (step-2 of Example 149, 40 mg, 0.071 mmol), cyclopropylboronic acid (12 mg, 0.142 mmol), Pd(PPh₃)₄ and sat. NaHCO₃ (0.4 mL) in THF (0.8 mL) was irradiated with micro-wave (150° C., 15 min). Then, to the reaction mixture was added THF (0.5 mL) and 2 M aqueous NaOH solution (0.5 mL), and stirred at 50° C. for 1 hr. The mixture was acidified by 2 M aqueous HCl solution, extracted with DCM, dried over Na₂SO₄, filtered and concentrated. The further purification was carried out by preparative LC-MS system in usual manner. HPLC-QC method, retention time and observed MS were summarized in Table 6.

### Example 150

### 4-(N-(3-Chloro-5-(trifluoromethyl)pyridin-2-yl)-N-(4-cyclopropylbenzyl)sulfamoyl)benzoic acid

### Step-1: Methyl 4-(N-(4-bromobenzyl)-N-(3-chloro-5-(trifluoromethyl)pyridin-2-yl)sulfamoyl)benzoate

The titled compound was prepared according to the procedure described in step-2 of Example 149 from methyl 4-(N-(3-chloro-5-(trifluoromethyl)pyridin-2-yl)sulfamoyl)benzoate (step-1 of Example 1) and 1-bromo-4-(bromomethyl)benzene;

¹H-NMR (300 MHz, CDCl₃) δ 8.52 (1H, br s), 8.22 (2H, d, J=8.8 Hz), 7.99 (1H, d, J=2.2 Hz), 7.86 (2H, d, J=8.8 Hz), 7.32 (2H, d, J=8.8 Hz), 7.09 (2H, d, J=8.8 Hz), 4.64 (2H, s), 3.99 (3H, s).

LC-MS (Method A) m/z: M+1 obs 564.74 tR=3.60 min

### Step-2: 4-(N-(3-Chloro-5-(trifluoromethyl)pyridin-2-yl)-N-(4-cyclopropylbenzyl)sulfamoyl)benzoic acid

The titled compound was prepared according to the procedure described in step-3 of Example 149 from methyl 4-(N-(4-bromobenzyl)-N-(3-chloro-5-(trifluoromethyl)pyridin-2-yl)sulfamoyl)benzoate (step-1 of Example 150) and cyclopropylboronic acid. The further purification was carried out by preparative LC-MS system in usual manner. HPLC-QC method, retention time and observed MS were summarized in Table 6.

### Example 151

### 4-(N-Benzyl-N-(3-bromo-5-(trifluoromethyl)pyridin-2-yl)sulfamoyl)benzoic acid

The titled compound was prepared according to the procedure described in step-2 of Example 15 from methyl 4-(N-benzyl-N-(3-bromo-5-(trifluoromethyl)pyridin-2-yl)sulfamoyl)benzoate (step-2 of Example 149). The further purification was carried out by preparative LC-MS system in usual manner. HPLC-QC method, retention time and observed MS were summarized in Table 6.

### Example 152

### 4-(N-(3-Chloro-5-(trifluoromethyl)pyridin-2-yl)-N-(4-(1-methyl-1H-pyrazol-4-yl)benzyl)sulfamoyl)benzoic acid

The titled compound was prepared according to the procedure described in step-3 of Example 149 from methyl 4-(N-(4-bromobenzyl)-N-(3-chloro-5-(trifluoromethyl)pyridin-2-yl)sulfamoyl)benzoate (step-1 of Example 150) and 1-methyl-4-(4,4,5,5-tetramethyl-1,3,2-dioxaborolan-2-yl)-1H-pyrazole. The further purification was carried out by preparative LC-MS system in usual manner. HPLC-QC method, retention time and observed MS were summarized in Table 6.

### Example 153

### 4-(N-Benzyl-N-(3-methyl-5-(trifluoromethyl)pyridin-2-yl)sulfamoyl)benzoic acid

The titled compound was prepared according to the procedure described in step-3 of Example 149 from methyl 4-(N-benzyl-N-(3-bromo-5-(trifluoromethyl)pyridin-2-yl)sulfamoyl)benzoate (step-1 of Example 150) and 2,4,6-trimethyl-1,3,5,2,4,6-trioxatriborinane. The further purification was carried out by preparative LC-MS system in usual manner. HPLC-QC method, retention time and observed MS were summarized in Table 6.

### Example 154

### 4-(N-(3-Chloro-5-(trifluoromethyl)pyridin-2-yl)-N-(4-(pyridin-3-yl)benzyl)sulfamoyl)benzoic acid

The titled compound was prepared according to the procedure described in step-3 of Example 149 from methyl 4-(N-(4-Bromobenzyl)-N-(3-chloro-5-(trifluoromethyl)pyridin-2-yl)sulfamoyl)benzoate (step-1 of Example 150) and 3-(1,3,2-dioxaborinan-2-yl)pyridine. The further purification was carried out by preparative LC-MS system in usual manner. HPLC-QC method, retention time and observed MS were summarized in Table 6.

### Example 155

### 4-(N-(3-Chloro-5-(trifluoromethyl)pyridin-2-yl)-N-(4-(thiophen-2-yl)benzyl)sulfamoyl)benzoic acid

The titled compound was prepared according to the procedure described in step-3 of Example 149 from methyl 4-(N-(4-bromobenzyl)-N-(3-chloro-5-(trifluoromethyl)pyridin-2-yl)sulfamoyl)benzoate (step-1 of Example 150) and thiophen-3-ylboronic acid. The further purification was carried out by preparative LC-MS system in usual manner. HPLC-QC method, retention time and observed MS were summarized in Table 6.

### Example 156

### 4-(N-(3-Chloro-5-(trifluoromethyl)pyridin-2-yl)-N-(4-(pyridin-4-yl-benzyl)sulfamoyl)benzoic acid

The titled compound was prepared according to the procedure described in step-3 of Example 149 from methyl 4-(N-(4-bromobenzyl)-N-(3-chloro-5-(trifluoromethyl)pyridin-2-yl)sulfamoyl)benzoate (step-1 of Example 150) and pyridin-4-ylboronic acid. The further purification was carried out by preparative LC-MS system in usual manner. HPLC-QC method, retention time and observed MS were summarized in Table 6.

### Example 157

### 4-(N-(3-Chloro-5-(trifluoromethyl)pyridin-2-yl)-N-(4-(furan-2-yl)-benzyl)sulfamoyl)benzoic acid

The titled compound was prepared according to the procedure described in step-3 of Example 149 from methyl 4-(N-(4-bromobenzyl)-N-(3-chloro-5-(trifluoromethyl)pyridin-2-yl)sulfamoyl)benzoate (step-1 of Example 150) and furan-2-ylboronic acid. The further purification was carried out by preparative LC-MS system in usual manner. HPLC-QC method, retention time and observed MS were summarized in Table 6.

### Example 158

### 4-(N-([1,1′-Biphenyl]-4-ylmethyl)-N-(3-chloro-5-(trifluoromethyl)pyridin-2-yl)sulfamoyl)benzoic acid

The titled compound was prepared according to the procedure described in step-3 of Example 149 from methyl 4-(N-(4-bromobenzyl)-N-(3-chloro-5-(trifluoromethyl)pyridin-2-yl)sulfamoyl)benzoate (step-1 of Example 150) and phenylboronic acid. The further purification was carried out by preparative LC-MS system in usual manner. HPLC-QC method, retention time and observed MS were summarized in Table 6.

### Example 159

### 4-(N-(3-Chloro-5-(trifluoromethyl)pyridin-2-yl)-N-(4-(oxazol-5-yl)benzyl)sulfamoyl)benzoic acid

The titled compound was prepared according to the procedure described in step-3 of Example 149 from methyl 4-(N-(4-bromobenzyl)-N-(3-chloro-5-(trifluoromethyl)pyridin-2-yl)sulfamoyl)benzoate (step-1 of Example 150) and 5-(4,4,5,5-tetramethyl-1,3,2-dioxaborolan-2-yl)oxazole. The further purification was carried out by preparative LC-MS system in usual manner. HPLC-QC method, retention time and observed MS were summarized in Table 6.

### Example 160

### 4-(N-(3-Chloro-5-(trifluoromethyl)pyridin-2-yl)-N-((6-(trifluoromethyl)pyridin-3-yl)methyl)sulfamoyl)benzoic acid

### Step-1: Methyl 4-(N-((6-(trifluoromethyl)pyridin-3-yl)methyl)sulfamoyl)benzoate

The titled compound was prepared according to the procedure described in step-1 of Example 148 from (6-(trifluoromethyl)pyridin-3-yl)methanamine.

LC-MS (Method A) m/z: M+1 obs 375.01, M−1 obs 373.04, tR=2.87 min.

### Step-2: Methyl 4-(N-(3-chloro-5-(trifluoromethyl)pyridin-2-yl)-N-((6-(trifluoromethyl)pyridin-3-yl)methyl)sulfamoyl)benzoate

A mixture of methyl 4-({[6-(trifluoromethyl)pyridin-3-yl]methyl}sulfamoyl)benzoate (step-1 of Example 160, 30 mg, 0.080 mmol), 2,3-dichloro-5-(trifluoromethyl)pyridine (104 mg, 0.401 mmol) and Cs₂CO₃ (78 mg, 0.240 mmol) in DMSO (0.7 mL) was stirred at 90° C. for 3 hr. The reaction mixture was used next step without purification.

### Step-3: 4-(N-(3-Chloro-5-(trifluoromethyl)pyridin-2-yl)-N-((6-(trifluoromethyl)pyridin-3-yl)methyl)sulfamoyl)benzoic acid

The titled compound was prepared according to the procedure described in step-2 of Example 15 from the reaction mixture in step-2 of Example 160. The further purification was carried out by preparative LC-MS system in usual manner. HPLC-QC method, retention time and observed MS were summarized in Table 6.

### Example 161

### 4-(N-(3-Chloro-5-(trifluoromethyl)pyridin-2-yl)-N-(4-(picolinamido)benzyl)sulfamoyl)benzoic acid

### Step-1: Methyl 4-(N-(3-chloro-5-(trifluoromethyl)pyridin-2-yl)-N-(4-nitrobenzyl)sulfamoyl)benzoate

The titled compound was prepared according to the procedure described in step-2 of Example 149 from methyl 4-(N-(3-chloro-5-(trifluoromethyl)pyridin-2-yl)sulfamoyl)benzoate (step-1 of Example 1) and 1-(bromomethyl)-4-nitrobenzene;

¹H-NMR (300 MHz, CDCl₃) δ 8.53 (1H, br s), 8.23 (2H, d, J=8.8 Hz), 8.08 (2H, d, J=8.8 Hz), 8.01 (1H, br s), 7.86 (2H, d, J=8.8 Hz), 7.45 (2H, d, J=8.8 Hz), 4.79 (2H, s), 4.00 (3H, s).

LC-MS (Method A) m/z: M+1 obs 529.90 tR=3.43 min

### Step-2: Methyl 4-(N-(4-aminobenzyl)-N-(3-chloro-5-(trifluoromethyl)pyridin-2-yl)sulfamoyl)benzoate

To a solution of methyl 4-(N-(3-chloro-5-(trifluoromethyl)pyridin-2-yl)-N-(4-nitrobenzyl)sulfamoyl)benzoate (step-1 of Example 161, 336 mg, 0.634 mmol) in MeOH (5 mL) and DCM (2 mL) was added SnCl₂ (1.20 g, 6.34 mmol). The mixture was stirred at 50° C. for 3 hr. The mixture was cooled to 0° C., quenched with sat. NaHCO₃, and stirred at room temperature for 1 hr. The mixture was filtered (using Celite pad). The filtrate was extracted with DCM, dried over Na₂SO₄, filtered and concentrated. The residual oil was used next step without purification.

LC-MS (Method A) m/z: M+1 obs 499.90, tR=3.20 min.

### Step-3: Methyl 4-(N-(3-chloro-5-(trifluoromethyl)pyridin-2-yl)-N-(4-(picolinamido)benzyl)sulfamoyl)benzoate

A mixture of methyl 4-(N-(4-aminobenzyl)-N-(3-chloro-5-(trifluoromethyl)pyridin-2-yl)sulfamoyl)benzoate (step-2 of Example 161, 75 mg, 0.15 mmol), picolinic acid (37 mg, 0.30 mmol), HBTU (114 mg, 0.30 mmol) and Et₃N (61 mg, 0.60 mmol) in DCM was stirred at room temperature for 1 day. The mixture was quenched with sat. NaHCO₃, extracted with DCM, dried over Na₂SO₄, filtered and concentrated to give the titled compound as crude.

LC-MS (Method A) m/z: M+1 obs 604.92, tR=3.42 min.

### Step-4: 4-(N-(3-chloro-5-(trifluoromethyl)pyridin-2-yl)-N-(4-(picolinamido)benzyl)sulfamoyl)benzoic acid

The titled compound was prepared according to the procedure described in step-2 of Example 15 from methyl 4-(N-(3-chloro-5-(trifluoromethyl)pyridin-2-yl)-N-(4-(picolinamido)benzyl)sulfamoyl)benzoate (step-3 of Example 161). The further purification was carried out by preparative LC-MS system in usual manner. HPLC-QC method, retention time and observed MS were summarized in Table 6.

### Example 162

### 4-(N-(3-chloro-5-(trifluoromethyl)pyridin-2-yl)-N-(4-(6-methoxypyridin-3-yl)-benzyl)sulfamoyl)benzoic acid

The titled compound was prepared according to the procedure described in step-3 of Example 149 from methyl 4-(N-(4-bromobenzyl)-N-(3-chloro-5-(trifluoromethyl)pyridin-2-yl)sulfamoyl)benzoate (step-1 of Example 150) and (6-methoxypyridin-3-yl)boronic acid. The further purification was carried out by preparative LC-MS system in usual manner. HPLC-QC method, retention time and observed MS were summarized in Table 6.

### Example 163

### 4-(N-(3-chloro-5-(trifluoromethyl)pyridin-2-yl)-N-(4-(6-methylpyridin-3-yl)benzyl)sulfamoyl)benzoic acid

The titled compound was prepared according to the procedure described in step-3 of Example 149 from methyl 4-(N-(4-bromobenzyl)-N-(3-chloro-5-(trifluoromethyl)pyridin-2-yl)sulfamoyl)benzoate (step-1 of Example 150) and (6-methylpyridin-3-yl)boronic acid. The further purification was carried out by preparative LC-MS system in usual manner. HPLC-QC method, retention time and observed MS were summarized in Table 6.

### Example 164

### 4-(N-[(1,1′-Biphenyl]-3-ylmethyl)-N-(3-chloro-5-(trifluoromethyl)pyridin-2-yl)sulfamoyl)benzoic acid

### Step-1: Methyl 4-(N-([1,1′-biphenyl]-3-ylmethyl)-N-(3-chloro-5-(trifluoromethyl)pyridin-2-yl)sulfamoyl)-benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and 3-(bromomethyl)-1,1′-biphenyl.

### Step-2: 4-(N-([1,1′-Biphenyl]-3-ylmethyl)-N-(3-chloro-5-(trifluoromethyl)pyridin-2-yl)sulfamoyl)benzoic acid

The titled compound was prepared according to the procedure described in step-2 of Example 15 from methyl 4-(N-([1,1′-biphenyl]-3-ylmethyl)-N-(3-chloro-5-(trifluoromethyl)pyridin-2-yl)sulfamoyl)benzoate (step-1 of Example 164). The further purification was carried out by preparative LC-MS system in usual manner. HPLC-QC method, retention time and observed MS were summarized in Table 6.

### Example 165

### 4-(N-([1,1′-Biphenyl]-2-ylmethyl)-N-(3-chloro-5-(trifluoromethyl)pyridin-2-yl)sulfamoyl)benzoic acid

### Step-1: Methyl 4-(N-([1,1′-biphenyl]-2-ylmethyl)-N-(3-chloro-5-(trifluoromethyl)pyridin-2-yl)sulfamoyl)benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and 2-(bromomethyl)-1,1′-biphenyl.

### Step-2: 4-(N-([1,1′-Biphenyl]-2-ylmethyl)-N-(3-chloro-5-(trifluoromethyl)pyridin-2-yl)sulfamoyl)benzoic acid

The titled compound was prepared according to the procedure described in step-2 of Example 15 from methyl 4-(N-([1,1′-biphenyl]-2-ylmethyl)-N-(3-chloro-5-(trifluoromethyl)pyridin-2-yl)sulfamoyl)benzoate (step-1 of Example 165). The further purification was carried out by preparative LC-MS system in usual manner. HPLC-QC method, retention time and observed MS were summarized in Table 6.

### Example 166

### (R)-4-(N-(3-Chloro-5-(trifluoromethyl)pyridin-2-yl)-N-(1-phenylethyl)sulfamoyl)benzoic acid

### Step-1: (R)-Methyl 4-(N-(1-phenylethyl)sulfamoyl)benzoate

The titled compound was prepared according to the procedure described in step-1 of Example 148 from methyl 4-(chlorosulfonyl)benzoate (step-1 of Example 1) and (R)-1-phenylethanamine;

¹H-NMR (300 MHz, CDCl₃) δ 8.00 (2H, dd, J=6.6 Hz), 7.74 (2H, d, J=6.6 Hz), 7.22-7.10 (3H, m), 7.08-7.01 (2H, m), 5.01 (1H, d), 4.60-4.50 (1H, m), 3.95 (3H, s), 1.45 (3H, d, J=6.6 Hz),

LC-MS (Method A) m/z: M−1 obs 318.10, tR=2.93 min.

### Step-2: (R)-Methyl 4-(N-(3-chloro-5-(trifluoromethyl)pyridin-2-yl)-N-(1-phenylethyl)sulfamoyl)benzoate

The titled compound was prepared according to the procedure described in step-2 of Example 148 from 2,3-dichloro-5-(trifluoromethyl)pyridine and (R)-methyl 4-(N-(1-phenylethyl)sulfamoyl)benzoate (step-1 of Example 166);

¹H-NMR (300 MHz, CDCl₃) δ 8.71 (1H, s), 8.04-8.01 (3H, m), 7.75-7.70 (2H, br), 7.20-7.09 (5H, m), 5.33 (1H, q, J=7.3 Hz), 3.97 (3H, s), 1.33 (3H, J=7.3 Hz),

LC-MS (Method A) m/z: M+1 obs 394.99 (fragment signal), tR=3.54 min.

### Step-3: (R)-4-(N-(3-Chloro-5-(trifluoromethyl)pyridin-2-yl)-N-(1-phenylethyl)sulfamoyl)benzoic acid

The titled compound was prepared according to the procedure described in step-2 of Example 15 from (R)-methyl 4-(N-(3-chloro-5-(trifluoromethyl)pyridin-2-yl)-N-(1-phenylethyl)sulfamoyl)benzoate (step-2 of Example 166). The further purification was carried out by preparative LC-MS system in usual manner. HPLC-QC method, retention time and observed MS were summarized in Table 6.

### Example 167

### 4-(N-(3-Chloro-5-(trifluoromethyl)pyridin-2-yl)-N-(thiophen-2-ylmethyl)sulfamoyl)benzoic acid

### Step-1: Methyl 4-(N-(thiophen-2-ylmethyl)sulfamoyl)benzoate

The titled compound was prepared according to the procedure described in step-1 of Example 148 from methyl 4-(chlorosulfonyl)benzoate and thiophen-2-ylmethanamine;

¹H-NMR (300 MHz, CDCl₃) δ 8.16 (2H, J=8.6 Hz), 7.92 (2H, J=8.6 Hz), 7.21-7.18 (1H, m), 6.89-6.85 (2H, m), 4.79 (1H, brs), 4.41 (2H, m), 3.97 (3H, s),

LC-MS (Method A) m/z: M−1 obs 310.07, tR=2.82 min.

### Step-2: Methyl 4-(N-(3-chloro-5-(trifluoromethyl)pyridin-2-yl)-N-(thiophen-2-ylmethyl)sulfamoyl)benzoate

The titled compound was prepared according to the procedure described in step-2 of Example 148 from 2,3-dichloro-5-(trifluoromethyl)pyridine and methyl 4-(N-(thiophen-2-ylmethyl)sulfamoyl)benzoate (step-1 of Example 167);

LC-MS (Method A) m/z: M+1 obs 490.82, tR=3.40 min.

### Step-3: 4-(N-(3-Chloro-5-(trifluoromethyl)pyridin-2-yl)-N-(thiophen-2-ylmethyl)sulfamoyl)benzoic acid

The titled compound was prepared according to the procedure described in step-2 of Example 15 from methyl 4-(N-(3-chloro-5-(trifluoromethyl)pyridin-2-yl)-N-(thiophen-2-ylmethyl)sulfamoyl)benzoate (step-2 of Example 167). The further purification was carried out by preparative LC-MS system in usual manner. HPLC-QC method, retention time and observed MS were summarized in Table 6.

### Example 168

### 4-(N-(3-Chloro-5-(trifluoromethyl)pyridin-2-yl)-N-(cyclohexylmethyl)sulfamoyl)benzoic acid

### Step-1: Methyl 4-(N-(cyclohexylmethyl)sulfamoyl)benzoate

The titled compound was prepared according to the procedure described in step-1 of Example 148 from methyl 4-(chlorosulfonyl)benzoate (step-1 of Example 1) and cyclohexylmethanamine;

LC-MS (Method A) m/z: M−1 obs 310.14, tR=3.12 min.

### Step-2: Methyl 4-(N-(3-chloro-5-(trifluoromethyl)pyridin-2-yl)-N-(cyclohexylmethyl)sulfamoyl)benzoate

The titled compound was prepared according to the procedure described in step-2 of Example 148 from 2,3-dichloro-5-(trifluoromethyl)pyridine and methyl 4-(N-(cyclohexylmethyl)sulfamoyl)benzoate (step-1 of Example 168);

LC-MS (Method A) m/z: obs 490.98, tR=3.70 min.

### Step-3: 4-(N-(3-Chloro-5-(trifluoromethyl)pyridin-2-yl)-N-(cyclohexylmethyl)sulfamoyl)benzoic acid

The titled compound was prepared according to the procedure described in step-2 of Example 15 from methyl 4-(N-(3-chloro-5-(trifluoromethyl)pyridin-2-yl)-N-(cyclohexylmethyl)sulfamoyl)benzoate (step-2 of Example 168). The further purification was carried out by preparative LC-MS system in usual manner. HPLC-QC method, retention time and observed MS were summarized in Table 6.

### Example 169

### 4-(N-(3-Chloro-5-(trifluoromethyl)pyridin-2-yl)-N-(4-phenoxybenzyl)sulfamoyl)benzoic acid

### Step-1: Methyl 4-(N-(4-phenoxybenzyl)sulfamoyl)benzoate

The titled compound was prepared according to the procedure described in step-1 of Example 148 from methyl 4-(chlorosulfonyl)benzoate (step-1 of Example 1) and (4-phenoxyphenyl)methanamine; LC-MS (Method A) m/z: M−1 obs 396.11, tR=3.19 min.

### Step-2: Methyl 4-(N-(3-chloro-5-(trifluoromethyl)pyridin-2-yl)-N-(4-phenoxybenzyl)sulfamoyl)benzoate

The titled compound was prepared according to the procedure described in step-2 of Example 148 from 2,3-dichloro-5-(trifluoromethyl)pyridine and methyl 4-(N-(4-phenoxybenzyl)sulfamoyl)benzoate (step-1 of Example 169);

¹H-NMR (300 MHz, CDCl₃) δ 8.53 (1H, s), 8.22 (2H, d, J=8.8 Hz), 7.98 (1H, s), 7.88 (2H, d, J=8.8 Hz), 7.34-7.28 (2H, m), 7.15-7.07 (3H, m), 6.92 (2H, d, J=8.8 Hz), 6.79 (2H, d, J=8.8 Hz), 4.67 (2H, s), 3.99 (3H, s),

LC-MS (Method A) m/z: M+1 obs 183.22 (fragment signal), tR=3.70 min.

### Step-3: 4-(N-(3-Chloro-5-(trifluoromethyl)pyridin-2-yl)-N-(4-phenoxybenzyl)sulfamoyl)benzoic acid

The titled compound was prepared according to the procedure described in step-2 of Example 15 from methyl 4-(N-(3-chloro-5-(trifluoromethyl)pyridin-2-yl)-N-(4-phenoxybenzyl)sulfamoyl)benzoate (step-2 of Example 169). The further purification was carried out by preparative LC-MS system in usual manner. HPLC-QC method, retention time and observed MS were summarized in Table 6.

### Example 170

### 4-(N-(4-(1H-Pyrazol-1-yl)benzyl)-N-(3-chloro-5-(trifluoromethyl)pyridin-2-yl)sulfamoyl)benzoic acid

### Step-1: Methyl 4-(N-(4-(1H-pyrazol-1-yl)benzyl)sulfamoyl)benzoate

The titled compound was prepared according to the procedure described in step-1 of Example 148 from methyl 4-(chlorosulfonyl)benzoate and (4-(1H-pyrazol-1-yl)phenyl)methanamine;

LC-MS (Method A) m/z: M+1 obs 372.02, M−1 obs 370.09, tR=2.80 min.

### Step-2: Methyl 4-(N-(4-(1H-pyrazol-1-yl)benzyl)-N-(3-chloro-5-(trifluoromethyl)pyridin-2-yl)sulfamoyl)benzoate

The titled compound was prepared according to the procedure described in step-2 of Example 15 from 2,3-dichloro-5-(trifluoromethyl)pyridine and methyl 4-(N-(4-(1H-pyrazol-1-yl)benzyl)sulfamoyl)benzoate (step-1 of Example 170);

¹H-NMR (300 MHz, CDCl₃) δ 8.53 (1H, s), 8.23 (2H, d, J=8.1 Hz), 7.96 (1H, s), 7.90-7.85 (3H, m), 7.68 (1H, s), 7.52 (2H, d, J=8.8 Hz), 7.29 (2H, d, J=8.8 Hz), 6.44 (1H, s), 4.72 (2H, s), 3.99 (3H, s),

LC-MS (Method A) m/z: M+1 obs 550.94, tR=3.39 min.

### Step-3: 4-(N-(4-(1H-Pyrazol-1-yl)benzyl)-N-(3-chloro-5-(trifluoromethyl)pyridin-2-yl)sulfamoyl)benzoic acid

The titled compound was prepared according to the procedure described in step-2 of Example 15 from methyl 4-(N-(4-(1H-pyrazol-1-yl)benzyl)-N-(3-chloro-5-(trifluoromethyl)pyridin-2-yl)sulfamoyl)benzoate (step-2 of Example 170). The further purification was carried out by preparative LC-MS system in usual manner. HPLC-QC method, retention time and observed MS were summarized in Table 6.

### Example 171

### 4-(N-(3-Chloro-5-(trifluoromethyl)pyridin-2-yl)-N-(cyclobutylmethyl)sulfamoyl)benzoic acid

### Step-1: Methyl 4-(N-(cyclobutylmethyl)sulfamoyl)benzoate

The titled compound was prepared according to the procedure described in step-1 of Example 148 from methyl 4-(chlorosulfonyl)benzoate (step-1 of Example 1) and cyclobutylmethanamine;

LC-MS (Method A) m/z: M−1 obs 282.18, tR=2.92 min.

### Step-2: Methyl 4-(N-(3-chloro-5-(trifluoromethyl)pyridin-2-yl)-N-(cyclobutylmethyl)sulfamoyl)benzoate

The titled compound was prepared according to the procedure described in step-2 of Example 148 from 2,3-dichloro-5-(trifluoromethyl)pyridine and methyl 4-(N-(cyclobutylmethyl)sulfamoyl)benzoate (step-1 of Example 171);

LC-MS (Method A) m/z: M+1 obs 462.93, tR=3.55 min.

### Step-3: 4-(N-(3-Chloro-5-(trifluoromethyl)pyridin-2-yl)-N-(cyclobutylmethyl)sulfamoyl)benzoic acid

The titled compound was prepared according to the procedure described in step-2 of Example 15 from methyl 4-(N-(3-chloro-5-(trifluoromethyl)pyridin-2-yl)-N-(cyclobutylmethyl)sulfamoyl)benzoate (step-2 of Example 171). The further purification was carried out by preparative LC-MS system in usual manner. HPLC-QC method, retention time and observed MS were summarized in Table 6.

### Example 172

### 4-(N-(3-Chloro-5-(trifluoromethyl)pyridin-2-yl)-N-(thiophen-3-ylmethyl)sulfamoyl)benzoic acid

### Step-1: Methyl 4-(N-(thiophen-3-ylmethyl)sulfamoyl)benzoate

The titled compound was prepared according to the procedure described in step-1 of Example 148 from methyl 4-(chlorosulfonyl)benzoate and thiophen-3-ylmethanamine;

LC-MS (Method A) m/z: M−1 obs 310.07, tR=2.82 min.

### Step-2: Methyl 4-(N-(3-chloro-5-(trifluoromethyl)pyridin-2-yl)-N-(thiophen-3-ylmethyl)sulfamoyl)benzoate

The titled compound was prepared according to the procedure described in step-1 of Example 148 from 2,3-dichloro-5-(trifluoromethyl)pyridine and methyl 4-(N-(thiophen-3-ylmethyl)sulfamoyl)benzoate (step-1 of Example 172);

LC-MS (Method A) m/z: M+1 obs 490.86, tR=3.42 min.

### Step-3: 4-(N-(3-Chloro-5-(trifluoromethyl)pyridin-2-yl)-N-(thiophen-3-ylmethyl)sulfamoyl)benzoic acid

The titled compound was prepared according to the procedure described in step-2 of Example 15 from methyl 4-(N-(3-chloro-5-(trifluoromethyl)pyridin-2-yl)-N-(thiophen-3-ylmethyl)sulfamoyl)benzoate (step-2 of Example 172). The further purification was carried out by preparative LC-MS system in usual manner. HPLC-QC method, retention time and observed MS were summarized in Table 6.

### Example 173

### N-Benzyl-N-(3-chloro-5-(trifluoromethyl)pyridin-2-yl)-4-cyanobenzenesulfonamide

### Step-1: N-benzyl-4-cyanobenzenesulfonamide

The titled compound was prepared according to the procedure described in step-1 of Example 148 from 4-cyanobenzene-1-sulfonyl chloride and phenylmethanamine;

LC-MS (Method A) m/z: M−1 obs 271.11, tR=2.79 min.

### Step-2: N-Benzyl-N-(3-chloro-5-(trifluoromethyl)pyridin-2-yl)-4-cyanobenzenesulfonamide

The titled compound was prepared according to the procedure described in step-2 of Example 148 from 2,3-dichloro-5-(trifluoromethyl)pyridine and N-benzyl-4-cyanobenzenesulfonamide (step-1 of Example 173). The further purification was carried out by preparative LC-MS system in usual manner. HPLC-QC method, retention time and observed MS were summarized in Table 6.

### Example 174

### N-Benzyl-N-(3-chloro-5-(trifluoromethyl)pyridin-2-yl)-4-(2H-tetrazol-5-yl)benzenesulfonamide

### Step-1: N-Benzyl-N-(3-chloro-5-(trifluoromethyl)pyridin-2-yl)-4-(2H-tetrazol-5-yl)benzenesulfonamide

A mixture of N-benzyl-N-(3-chloro-5-(trifluoromethyl)pyridin-2-yl)-4-cyanobenzenesulfonamide (step-2 of Example 173, 60 mg, 0.13 mmol), NaN₃ (52 mg, 0.80 mmol), and NH₄Cl (43 mg, 0.80 mmol) in DMF (5 mL) was heated at 110° C. overnight. After cooling, quenched by 1 M HCl aqueous solution, and extracted with toluene/EtOAc (2/1), washed with water, dried over MgSO₄, filtered, and the filtrate was concentrated to give 73 mg (quant. yield) of the titled compound as a yellow solid. The further purification was carried out by preparative LC-MS system in usual manner. HPLC-QC method, retention time and observed MS were summarized in Table 6.

### Example 175

### N-Benzyl-N-(3-chloro-5-(trifluoromethyl)pyridin-2-yl)-4-methoxybenzenesulfonamide

### Step-1: N-Benzyl-4-methoxybenzenesulfonamide

The titled compound was prepared according to the procedure described in step-1 of Example 148 from 4-methoxybenzene-1-sulfonyl chloride and phenylmethanamine;

LC-MS (Method A) m/z: M−1 obs 276.13, tR=2.82 min.

### Step-2: N-Benzyl-N-(3-chloro-5-(trifluoromethyl)pyridin-2-yl)-4-methoxybenzenesulfonamide

The titled compound was prepared according to the procedure described in step-2 of Example 148 from 2,3-dichloro-5-(trifluoromethyl)pyridine and N-benzyl-4-methoxybenzenesulfonamide (step-1 of Example 175). The further purification was carried out by preparative LC-MS system in usual manner. HPLC-QC method, retention time and observed MS were summarized in Table 6.

### Example 176

### N-Benzyl-N-(3-chloro-5-(trifluoromethyl)pyridin-2-yl)-4-(methylsulfonamidomethyl)benzenesulfonamide

### Step-1: 4-(Aminomethyl)-N-benzyl-N-(3-chloro-5-(trifluoromethyl)pyridin-2-yl)benzenesulfonamide

A mixture of N-benzyl-N-(3-chloro-5-(trifluoromethyl)pyridin-2-yl)-4-cyanobenzenesulfonamide (step-2 of Example 173, 188 mg, 0.42 mmol), and Raney Nickel (0.05 mL, Raney (registered trademark) 2800) in 1 M NH₃ in MeOH solution (20 mL) was stirred under hydrogen atmosphere for 16 hrs. The mixture was filtered through a pad of celite, washed with MeOH, the filtrate was concentrated to give a brown oil. The residual oil was diluted with MeOH, applied onto a strong cation exchange cartridge (BondElute (registered trademark) SCX, 1 g/6 mL, Varian Inc.), and the solid phase matrix was rinsed with methanol (6 mL). The crude mixture was eluted in a collection tube with 1 M NH₃ in MeOH (6 mL) and concentrated gave 179 mg (94% yield) of the titled compound as a clear colorless oil.

LC-MS (Method A) m/z: M+1 obs 455.95; tR=2.87 min

### Step-2: N-Benzyl-N-(3-chloro-5-(trifluoromethyl)pyridin-2-yl)-4-(methylsulfonamidomethyl)benzenesulfonamide

To a solution of 4-(aminomethyl)-N-benzyl-N-(3-chloro-5-(trifluoromethyl)pyridin-2-yl)benzenesulfonamide (step-1 of Example 176, 40 mg, 0.088 mmol) and Et₃N (18 mg, 0.18 mmol) in DCM (4 mL) was added and methanesulfonyl chloride (10 mg, 0.88 mmol) at room temperature, and stirred for 2 hrs at room temperature. The mixture was washed with 2 M aqueous HCl solution, dried over MgSO₄, filtered, and the filtrate was concentrated gave 30 mg (64% yield) of the titled compound as a pale yellow oil. The further purification was carried out by preparative LC-MS system in usual manner. HPLC-QC method, retention time and observed MS were summarized in Table 6.

### Example 177

### N-(4-(N-Benzyl-N-(3-chloro-5-(trifluoromethyl)pyridin-2-yl)sulfamoyl)benzyl)acetamide

### Step-1: N-(4-(N-Benzyl-N-(3-chloro-5-(trifluoromethyl)pyridin-2-yl)sulfamoyl)benzyl)acetamide

The titled compound was prepared according to the procedure described in step-2 of Example 176 from 4-(aminomethyl)-N-benzyl-N-(3-chloro-5-(trifluoromethyl)pyridin-2-yl)benzenesulfonamide (step-1 of Example 176) and acetyl chloride. The further purification was carried out by preparative LC-MS system in usual manner. HPLC-QC method, retention time and observed MS were summarized in Table 6.

### Example 178

### N-Benzyl-N-(3-chloro-5-(trifluoromethyl)pyridin-2-yl)-4-(((N,N-dimethylsulfamoyl)amino)methyl)benzenesulfonamide

### Step-1: N-Benzyl-N-(3-chloro-5-(trifluoromethyl)pyridin-2-yl)-4-(((N,N-dimethylsulfamoyl)amino)methyl)benzenesulfonamide

The titled compound was prepared according to the procedure described in step-2 of Example 176 from 4-(aminomethyl)-N-benzyl-N-(3-chloro-5-(trifluoromethyl)pyridin-2-yl)benzenesulfonamide (step-1 of Example 176) and dimethylsulfamoyl chloride. The further purification was carried out by preparative LC-MS system in usual manner. HPLC-QC method, retention time and observed MS were summarized in Table 6.

### Example 179

### N-Benzyl-N-(3-chloro-5-(trifluoromethyl)pyridin-2-yl)-4-((3,3-dimethylureido)methyl)benzene sulfonamide

### Step-1: N-Benzyl-N-(3-chloro-5-(trifluoromethyl)pyridin-2-yl)-4-(3,3-dimethylureido)methyl)benzene sulfonamide

The titled compound was prepared according to the procedure described in step-2 of Example 176 from 4-(aminomethyl)-N-benzyl-N-(3-chloro-5-(trifluoromethyl)pyridin-2-yl)benzenesulfonamide (step-1 of Example 176) and dimethylcarbamic chloride. The further purification was carried out by preparative LC-MS system in usual manner. HPLC-QC method, retention time and observed MS were summarized in Table 6.

### Example 180

### 4-(N-Benzyl-N-(5-bromo-3-chloropyridin-2-yl)sulfamoyl)benzoic acid

### Step-1: Methyl 4-(N-(5-bromo-3-chloropyridin-2-yl)sulfamoyl)benzoate

The titled compound was prepared according to the procedure described in step-1 of Example 1 from 5-bromo-3-chloropyridin-2-amine and methyl 4-(chlorosulfonyl)benzoate.

LC-MS (Method A) m/z: obs 406.87, M−1 obs 404.90; tR=2.94 min.

### Step-2: Methyl 4-(N-benzyl-N-(5-bromo-3-chloropyridin-2-yl)sulfamoyl)benzoate

The titled compound was prepared according to the procedure described in step-1 of Example 15 from methyl 4-(N-(5-bromo-3-chloropyridin-2-yl)sulfamoyl)benzoate (step-1 of Example 180) and (bromomethyl)benzene.

LC-MS (Method A) m/z: M+1 obs 496.78; tR=3.43 min.

### Step-3: 4-(N-Benzyl-N-(5-bromo-3-chloropyridin-2-yl)sulfamoyl)benzoic acid

The titled compound was prepared according to the procedure described in step-2 of Example 15 from methyl 4-(N-benzyl-N-(5-bromo-3-chloropyridin-2-yl)sulfamoyl)benzoate (step-2 of Example 180). The further purification was carried out by preparative LC-MS system in usual manner. HPLC-QC method, retention time and observed MS were summarized in Table 6.

### Example 181

### 4-(N-Benzyl-N-(3-chloro-5-(trifluoromethyl)pyridin-2-yl)sulfamoyl)-3-chlorobenzoic acid

### Step-1: Ethyl 4-(N-benzylsulfamoyl)-3-chlorobenzoate

The titled compound was prepared according to the procedure described in step-1 of Example 148 from ethyl 3-chloro-4-(chlorosulfonyl)benzoate and phenylmethanamine;

LC-MS (Method A) m/z: M+1 obs 353.98 M−1 obs 351.98, tR=3.12 min.

### Step-2: Ethyl 4-(N-Benzyl-N-(3-chloro-5-(trifluoromethyl)pyridin-2-yl)sulfamoyl)-3-chlorobenzoate

The titled compound was prepared according to the procedure described in step-2 of Example 148 from 2,3-dichloro-5-(trifluoromethyl)pyridine and ethyl 4-(N-benzylsulfamoyl)-3-chlorobenzoate (step-1 of Example 181);

LC-MS (Method A) m/z: M+1 obs 532.86, tR=3.65 min.

### Step-3: 4-(N-Benzyl-N-(3-chloro-5-(trifluoromethyl)pyridin-2-yl)sulfamoyl)-3-chlorobenzoic acid

The titled compound was prepared according to the procedure described in step-2 of Example 15 from ethyl 4-(N-benzyl-N-(3-chloro-5-(trifluoromethyl)pyridin-2-yl)sulfamoyl)-3-chlorobenzoate (step-2 of Example 181). The further purification was carried out by preparative LC-MS system using the condition in Table 6.

### Example 182

### 4-(N-Benzyl-N-(3-chloro-5-phenylpyridin-2-yl)sulfamoyl)benzoic acid

### Step-1: 4-(N-Benzyl-N-(3-chloro-5-phenylpyridin-2-yl)sulfamoyl)benzoic acid

A mixture of methyl 4-(N-benzyl-N-(5-bromo-3-chloropyridin-2-yl)sulfamoyl)benzoate (step-2 of Example 180, 35 mg, 0.071 mmol), phenylboronic acid (17 mg, 0.14 mmol), Pd(PPh₃)₄ (8 mg, 7 micromol), and sat. NaHCO₃ (0.4 mL) in dioxane (0.8 mL) was irradiated with micro-wave (150° C., 15 min). After cooling to room temperature, the mixture was added 2 M aqueous NaOH solution (0.5 mL), THF (1 mL), and the mixture was heated at 60° C. for 2 hrs. After cooling to room temperature, 2M aqueous HCl solution (0.5 mL) was added and extracted with DCM, dried over Na₂SO₄, filtered and concentrated gave crude product as brown oil. The further purification was carried out by preparative LC-MS system in usual manner. HPLC-QC method, retention time and observed MS were summarized in Table 6.

### Example 183

### 4-(N-Benzyl-N-(3-chloro-5-(furan-2-yl)pyridin-2-yl)sulfamoyl)benzoic acid

### Step-1: 4-(N-Benzyl-N-(3-chloro-5-(furan-2-yl)pyridin-2-yl)sulfamoyl)benzoic acid

The titled compound was prepared according to the procedure described in step-1 of Example 182 from methyl 4-(N-benzyl-N-(5-bromo-3-chloropyridin-2-yl)sulfamoyl)benzoate (step-2 of Example 180) and furan-2-ylboronic acid. The further purification was carried out by preparative LC-MS system in usual manner. HPLC-QC method, retention time and observed MS were summarized in Table 6.

### Example 184

### 4-(N-Benzyl-N-(3-chloro-5-(thiophen-3-yl)pyridin-2-yl)sulfamoyl)benzoic acid

### Step-1: 4-(N-Benzyl-N-(3-chloro-5-(thiophen-3-yl)pyridin-2-yl)sulfamoyl)benzoic acid

The titled compound was prepared according to the procedure described in step-1 of Example 182 from methyl 4-(N-benzyl-N-(5-bromo-3-chloropyridin-2-yl)sulfamoyl)benzoate (step-2 of Example 180) and thiophen-3-ylboronic acid. The further purification was carried out by preparative LC-MS system in usual manner. HPLC-QC method, retention time and observed MS were summarized in Table 6.

### Example 185

### 4-(N-Benzyl-N-(3-chloro-5-(2-methoxyphenyl)pyridin-2-yl)sulfamoyl)benzoic acid

### Step-1: 4-(N-Benzyl-N-(3-chloro-5-(2-methoxyphenyl)pyridin-2-yl)sulfamoyl)benzoic acid

The titled compound was prepared according to the procedure described in step-1 of Example 182 from methyl 4-(N-benzyl-N-(5-bromo-3-chloropyridin-2-yl)sulfamoyl)benzoate (step-2 of Example 180) and (2-methoxyphenyl)boronic acid. The further purification was carried out by preparative LC-MS system in usual manner. HPLC-QC method, retention time and observed MS were summarized in Table 6.

### Example 186

### 4-(N-Benzyl-N-(3-chloro-5-(4-methoxyphenyl)pyridin-2-yl)sulfamoyl)benzoic acid

### Step-1: 4-(N-Benzyl-N-(3-chloro-5-(4-methoxyphenyl)pyridin-2-yl)sulfamoyl)benzoic acid

The titled compound was prepared according to the procedure described in step-1 of Example 182 from methyl 4-(N-benzyl-N-(5-bromo-3-chloropyridin-2-yl)sulfamoyl)benzoate (step-2 of Example 180) and (4-methoxyphenyl)boronic acid. The further purification was carried out by preparative LC-MS system in usual manner. HPLC-QC method, retention time and observed MS were summarized in Table 6.

### Example 187

### 4-(N-Benzyl-N-(3-chloro-5-(3-methoxyphenyl)pyridin-2-yl)sulfamoyl)benzoic acid

### Step-1: 4-(N-Benzyl-N-(3-chloro-5-(3-methoxyphenyl)pyridin-2-yl)sulfamoyl)benzoic acid

The titled compound was prepared according to the procedure described in step-1 of Example 182 from methyl 4-(N-benzyl-N-(5-bromo-3-chloropyridin-2-yl)sulfamoyl)benzoate (step-2 of Example 180) and (3-methoxyphenyl)boronic acid. The further purification was carried out by preparative LC-MS system in usual manner. HPLC-QC method, retention time and observed MS were summarized in Table 6.

### Example 188

### 4-(N-Benzyl-N-(3-chloro-5-(trifluoromethyl)pyridin-2-yl)sulfamoyl)-2-chlorobenzoic acid

### Step-1: N-Benzyl-4-bromo-3-chlorobenzenesulfonamide

The titled compound was prepared according to the procedure described in step-1 of Example 148 from 4-bromo-3-chlorobenzene-1-sulfonyl chloride and phenylmethanamine;

LC-MS (Method A) m/z: M−1 obs 359.88, tR=3.18 min.

### Step-2: N-Benzyl-4-bromo-3-chloro-N-(3-chloro-5-(trifluoromethyl)pyridin-2-yl)benzenesulfonamide

The titled compound was prepared according to the procedure described in step-1 of Example 148 from 2,3-dichloro-5-(trifluoromethyl)pyridine and N-benzyl-4-bromo-3-chlorobenzenesulfonamide (step-1 of Example 188);

LC-MS (Method A) m/z: M−1 obs 540.71, tR=3.73 min.

### Step-3: Methyl 4-(N-Benzyl-N-(3-chloro-5-(trifluoromethyl)pyridin-2-yl)sulfamoyl)-2-chlorobenzoate

A mixture of N-benzyl-4-bromo-3-chloro-N-(3-chloro-5-(trifluoromethyl)pyridin-2-yl)benzenesulfonamide (step-2 of Example 188, 20 mg, 0.037 mmol), Pd(OAc)₂ (0.8 mg, 4 micromol), 1,3-bis(diphenylphosphino)propane (1.5 mg, 4 micromol), and Et₃N (11 mg, 0.11 mmol) in DMF/MeOH (5 mL/2 mL) was heated at 100° C. overnight under carbon monooxide atmosphere. After cooling, the mixture was added water, extracted with diethyl ether (2 times). The combined organic phase were washed with water, brine, dried over Na₂SO₄, filtered, and the filtrae was concentrated to give a yellow brown solid, which was applied to a silica gel column chromatography and eluted with hexane/EtOAc=6/1 to furnish 11 mg (57% yield) of the titled compound as a clear yellow oil;

¹H-NMR (300 MHz, CDCl₃) δ 8.53 (1H, s), 7.97-7.92 (3H, m), 7.87 (1H, d, J=1.4 Hz), 7.71 (1H, dd, J=8.8, 2.2 Hz), 7.20 (5H, s), 4.71 (2H, s), 4.00 (3H, s),

LC-MS (Method A) m/z: M+1 obs 518.83, tR=3.54 min.

### Step-4: 4-(N-Benzyl-N-(3-chloro-5-(trifluoromethyl)pyridin-2-yl)sulfamoyl)-2-chlorobenzoic acid

The titled compound was prepared according to the procedure described in step-2 of Example 15 from methyl 4-(N-benzyl-N-(3-chloro-5-(trifluoromethyl)pyridin-2-yl)sulfamoyl)-2-chlorobenzoate (step-3 of Example 188). The further purification was carried out by preparative LC-MS system in usual manner. HPLC-QC method, retention time and observed MS were summarized in Table 6.

### Example 189

### N-Benzyl-N-(3-chloro-5-(trifluoromethyl)pyridin-2-yl)-6-methoxypyridine-3-sulfonamide

### Step-1: N-Benzyl-6-methoxypyridine-3-sulfonamide

The titled compound was prepared according to the procedure described in step-1 of Example 148 from 6-methoxypyridine-3-sulfonyl chloride and phenylmethanamine;

LC-MS (Method A) m/z: M+1 obs 279.13 M−1 obs 277.15, tR=2.82 min.

### Step-2: N-Benzyl-N-(3-chloro-5-(trifluoromethyl)pyridin-2-yl)-6-methoxypyridine-3-sulfonamide

The titled compound was prepared according to the procedure described in step-2 of Example 148 from 2,3-dichloro-5-(trifluoromethyl)pyridine and N-benzyl-6-methoxypyridine-3-sulfonamide (step-1 of Example 189). The further purification was carried out by preparative LC-MS system in usual manner. HPLC-QC method, retention time and observed MS were summarized in Table 6.

### Example 190

### 4-(N-Benzyl-N-(3-chloro-5-(trifluoromethyl)pyridin-2-yl)sulfamoyl)-3-fluorobenzoic acid

### Step-1: N-Benzyl-4-bromo-2-fluorobenzenesulfonamide

The titled compound was prepared according to the procedure described in step-1 of Example 148 from 4-bromo-2-fluorobenzene-1-sulfonyl chloride and phenylmethanamine;

LC-MS (Method A) m/z: M−1 obs 343.96, tR=3.07 min.

### Step-2: Methyl 4-(N-benzylsulfamoyl)-3-fluorobenzoate

The titled compound was prepared according to the procedure described in step-3 of Example 188 from N-benzyl-4-bromo-2-fluorobenzenesulfonamide (step-1 of Example 190);

LC-MS (Method A) m/z: M−1 obs 322.08, tR=2.92 min.

### Step-3: Methyl 4-(N-benzyl-N-(3-chloro-5-(trifluoromethyl)pyridin-2-yl)sulfamoyl)-3-fluorobenzoate

The titled compound was prepared according to the procedure described in step-2 of Example 148 from 2,3-dichloro-5-(trifluoromethyl)pyridine and methyl 4-(N-benzylsulfamoyl)-3-fluorobenzoate (step-2 of Example 190);

LC-MS (Method A) m/z: M+1 obs 502.90, tR=3.48 min.

### Step-4: 4-(N-Benzyl-N-(3-chloro-5-(trifluoromethyl)pyridin-2-yl)sulfamoyl)-3-fluorobenzoic acid

The titled compound was prepared according to the procedure described in step-2 of Example 15 from methyl 4-(N-benzyl-N-(3-chloro-5-(trifluoromethyl)pyridin-2-yl)sulfamoyl)-3-fluorobenzoate (step-3 of Example 190). The further purification was carried out by preparative LC-MS system in usual manner. HPLC-QC method, retention time and observed MS were summarized in Table 6.

### Example 191

### 4-(N-Benzyl-N-(3-chloro-5-(trifluoromethyl)pyridin-2-yl)sulfamoyl)-2-fluorobenzoic acid

### Step-1: N-Benzyl-4-bromo-3-fluorobenzenesulfonamide

The titled compound was prepared according to the procedure described in step-1 of Example 148 from 4-bromo-3-fluorobenzene-1-sulfonyl chloride and phenylmethanamine;

LC-MS (Method A) m/z: M−1 obs 343.99, tR=3.09 min.

### Step-2: Methyl 4-(N-benzylsulfamoyl)-2-fluorobenzoate

The titled compound was prepared according to the procedure described in step-3 of Example 188 from N-benzyl-4-bromo-3-fluorobenzenesulfonamide (step-1 of Example 191);

LC-MS (Method A) m/z: M−1 obs 322.08, tR=2.97 min.

### Step-3: Methyl 4-(N-benzyl-N-(3-chloro-5-(trifluoromethyl)pyridin-2-yl)sulfamoyl)-2-fluorobenzoate

The titled compound was prepared according to the procedure described in step-2 of Example 148 from 2,3-dichloro-5-(trifluoromethyl)pyridine and methyl 4-(N-benzylsulfamoyl)-2-fluorobenzoate (step-2 of Example 191);

LC-MS (Method A) m/z: M+1 obs 502.90, tR=3.47 min.

### Step-4: 4-(N-Benzyl-N-(3-chloro-5-(trifluoromethyl)pyridin-2-yl)sulfamoyl)-2-fluorobenzoic acid

The titled compound was prepared according to the procedure described in step-2 of Example 15 from methyl 4-(N-benzyl-N-(3-chloro-5-(trifluoromethyl)pyridin-2-yl)sulfamoyl)-2-fluorobenzoate (step-3 of Example 191). The further purification was carried out by preparative LC-MS system in usual manner. HPLC-QC method, retention time and observed MS were summarized in Table 6.

### Example 192

### 4-(N-(3-Chloro-5-(trifluoromethyl)pyridin-2-yl)-N-(cyclopentylmethyl)sulfamoyl)benzoic acid

### Step-1: Methyl 4-(N-(cyclopentylmethyl)sulfamoyl)benzoate

The titled compound was prepared according to the procedure described in step-1 of Example 148 from methyl 4-(chlorosulfonyl)benzoate and cyclopentylmethanamine;

LC-MS (Method A) m/z: M−1 obs 296.14, tR=2.99 min.

### Step-2: Methyl 4-(N-(3-chloro-5-(trifluoromethyl)pyridin-2-yl)-N-(cyclopentylmethyl)sulfamoyl)benzoate

The titled compound was prepared according to the procedure described in step-2 of Example 148 from 2,3-dichloro-5-(trifluoromethyl)pyridine and methyl 4-(N-(cyclopentylmethyl)sulfamoyl)benzoate (step-1 of Example 192);

¹H-NMR (300 MHz, CDCl₃) δ 8.58 (1H, d, J=1.5 Hz), 8.19 (2H, d, J=8.8 Hz), 8.12 (1H, d, J=2.2 Hz), 7.83 (2H, d, J=8.8 Hz), 3.98 (3H, s), 3.44 (2H, d, J=7.3 Hz), 1.76-1.43 (7H, m), 1.19-1.07 (2H, m).

LC-MS (Method A) m/z: M+1 obs 476.99, tR=3.62 min.

### Step-3: 4-(N-(3-Chloro-5-(trifluoromethyl)pyridin-2-yl)-N-(cyclopentylmethyl)sulfamoyl)benzoic acid

The titled compound was prepared according to the procedure described in step-2 of Example 15 from methyl 4-(N-(3-chloro-5-(trifluoromethyl)pyridin-2-yl)-N-(cyclopentylmethyl)sulfamoyl)benzoate (step-2 of Example 192). The further purification was carried out by preparative LC-MS system in usual manner. HPLC-QC method, retention time and observed MS were summarized in Table 6.

### Example 193

### 4-(N-(3-Chloro-5-(trifluoromethyl)pyridin-2-yl)-N-(1-phenylcyclopropyl)sulfamoyl)benzoic acid

### Step-1: Methyl 4-(N-(1-phenylcyclopropyl)sulfamoyl)-benzoate

The titled compound was prepared according to the procedure described in step-1 of Example 148 from methyl 4-(chlorosulfonyl)benzoate and 1-phenylcyclopropanamine, hydrochloride;

LC-MS (Method A) m/z: M−1 obs 330.10, tR=2.93 min.

### Step-2: Methyl 4-(N-(3-chloro-5-trifluoromethyl)pyridin-2-yl-N-(1-phenylcyclopropyl)sulfamoyl)benzoate

The titled compound was prepared according to the procedure described in step-2 of Example 148 from 2,3-dichloro-5-(trifluoromethyl)pyridine and methyl 4-(N-(1-phenylcyclopropyl)sulfamoyl)benzoate (step-1 of Example 193); LC-MS (Method A) m/z: M+1 obs 510.80, tR=3.54 min.

### Step-3: 4-(N-(3-Chloro-5-(trifluoromethyl)pyridin-2-yl)-N-(1-phenylcyclopropyl)-sulfamoyl)benzoic acid

The titled compound was prepared according to the procedure described in step-2 of Example 15 from methyl 4-(N-(3-chloro-5-(trifluoromethyl)pyridin-2-yl)-N-(1-phenylcyclopropyl)sulfamoyl)benzoate (step-2 of Example 193). The further purification was carried out by preparative LC-MS system in usual manner. HPLC-QC method, retention time and observed MS were summarized in Table 6.

### Example 194

### N-Benzyl-N-(3-chloro-5-(trifluoromethyl)pyridin-2-yl)-4-((N,N-dimethylsulfamoyl)amino)benzenesulfonamide

### Step-1: N-Benzyl-4-nitrobenzenesulfonamide

The titled compound was prepared according to the procedure described in step-1 of Example 148 from 4-nitrobenzene-1-sulfonyl chloride and phenylmethanamine;

¹H-NMR (300 MHz, CDCl₃) δ 8.34-8.29 (2H, m), 8.10-7.97 (2H, m), 7.28-7.25 (3H, m), 7.20-7.15 (2H, m), 4.90 (1H, t, J=5.5 Hz), 4.24 (2H, d, J=6.6 Hz).

LC-MS (Method A) m/z: M−1 obs 291.11, tR=2.86 min.

### Step-2: N-Benzyl-N-(3-chloro-5-(trifluoromethyl)pyridin-2-yl)-4-nitrobenzenesulfonamide

The titled compound was prepared according to the procedure described in step-2 of Example 148 from 2,3-dichloro-5-(trifluoromethyl)pyridine and N-benzyl-4-nitrobenzenesulfonamide (step-1 of Example 194);

¹H-NMR (300 MHz, CDCl₃) δ 8.55 (1H, s), 8.38 (2H, d, J=8.8 Hz), 8.00-7.97 (3H, m), 7.19 (5H, s), 4.73 (2H, s).

LC-MS (Method A) m/z: M+1 obs 471.91, tR=3.45 min.

### Step-3: 4-Amino-N-benzyl-N-(3-chloro-5-(trifluoromethyl)pyridin-2-yl)benzenesulfonamide

A MeOH (8 mL) solution of N-benzyl-N-(3-chloro-5-(trifluoromethyl)pyridin-2-yl)-4-nitrobenzenesulfonamide (step-2 of Example 194, 381 mg, 0.807 mmol) and 5% Platinum on charcoal type 128 PASTE (Johnson Matthey, 0.05 g) was stirred at room temperature under hydrogen atmosphere for 3 hrs. The catalyst (0.05 g) was added and stirred for 10 hrs. The catalyst was filtered off through a celite pad and the filtrate was concentrated. The residue was purified by silica gel column chromatography (hexane/EtOAc=95/5 to 4/1) to give 328 mg (92% yield) of the titled compound as yellow grease.

¹H-NMR (300 MHz, CDCl₃) δ 8.52 (1H, s), 7.91 (1H, s), 7.59 (2H, d, J=8.8 Hz), 7.26-7.15 (5H, m), 6.72 (2H, d, J=8.8 Hz), 4.66 (2H, s), 4.22 (2H, br).

LC-MS (Method A) m/z: M+1 obs 441.95, tR=3.20 min.

### Step-4: N-Benzyl-N-(3-chloro-5-(trifluoromethyl)pyridin-2-yl)-4-((N,N-dimethylsulfamoyl)amino)benzenesulfonamide

To a solution of 4-amino-N-benzyl-N-(3-chloro-5-(trifluoromethyl)pyridin-2-yl)benzenesulfonamide (step-4 of Example 194, 25 mg, 0.057 mmol) and Et₃N (7.44 mg, 0.074 mmol) in CH₃CN (2 mL) was added dimethylsulfamoyl chloride (9.09 microL, 0.085 mmol) at room temperature. The resulting solution was stirred for 2 hrs at room temperature, then 70° C. for 3 hrs. The resulting mixture was concentrated. The whole was added pyridine (1 mL) and dimethylsulfamoyl chloride (12 microL, 0.112 mmol), The resulting mixture was stirred at 70° C. for 8 hrs. The reaction mixture was extracted with EtOAc (15 mL) and water (30 mL). The organic layer was dried over MgSO₄ and concentrated to give 37 mg (119% Yield) of the title compound. The further purification was carried out by preparative LC-MS system in usual manner. HPLC-QC method, retention time and observed MS were summarized in Table 6.

### Example 195

### N-Benzyl-N-(3-chloro-5-(trifluoromethyl)pyridin-2-yl)-4-ureidobenzenesulfonamide

To a solution of 4-amino-N-benzyl-N-(3-chloro-5-(trifluoromethyl)pyridin-2-yl)benzenesulfonamide (step-3 of Example 194, 30.6 mg, 0.069 mmol) and 4-dimethylaminopyridine (0.85 mg, 0.0069 mmol) in THF (2 mL) was added trimethylsilyl isocyanate (21.4 microL, 0.173 mmol) at room temperature. The resulting solution was stirred for 3 hrs, and then refluxed for 5 hr and cooled to room temperature. The reaction mixture was added trimethylsilyl isocyanate (15 microL, 0.121 mmol) and refluxed overnight. The resulting mixture was quenched with water and extracted with EtOAc, dried over MgSO₄ and concentrated. The crude product was purified by silylca gel column chromatograph (hexane/EtOAc=1/0 to 0/1) to give 25.8 mg (77% Yield) of the title compound. The further purification was carried out by preparative LC-MS system in usual manner. HPLC-QC method, retention time and observed MS were summarized in Table 6.

### Example 196

### N-Benzyl-N-(3-chloro-5-(trifluoromethyl)pyridin-2-yl)-4-(sulfamoylamino)benzenesulfonamide

To the mixture of 4-amino-N-benzyl-N-(3-chloro-5-(trifluoromethyl)pyridin-2-yl)benzenesulfonamide (step-3 of Example 194, 22.3 mg, 0.050 mmol) and 1,4-dioxane (2 mL) was added sulfamide (19.4 mg, 0.202 mmol) and the whole was refluxed for 18 hrs. The reaction mixture was concentrated and the crude product was purified by silyca gel column chromatography (hexane/EtOAc=1/0 to 2/1) to give 11.3 mg (43% Yield) of the title compound. The further purification was carried out by preparative LC-MS system in usual manner. HPLC-QC method, retention time and observed MS were summarized in Table 6.

### Example 197

### (S)-4-(N-(3-Chloro-5-(trifluoromethyl)pyridin-2-yl)-N-(1-phenylethyl)sulfamoyl)benzoic acid

### Step-1: (S)-Methyl 4-(N-(1-phenylethyl)sulfamoyl)benzoate

The titled compound was prepared according to the procedure described in step-1 of Example 148 from methyl 4-(chlorosulfonyl)benzoate (step-1 of Example 1) and (S)-1-phenylethanamine;

LC-MS (Method A) m/z: M−1 obs 318.10, tR=2.93 min.

### Step-2: (S)-Methyl 4-(N-(3-chloro-5-(trifluoromethyl)pyridin-2-yl)-N-(1-phenylethyl)sulfamoyl)benzoate

The titled compound was prepared according to the procedure described in step-2 of Example 148 from 2,3-dichloro-5-(trifluoromethyl)pyridine and (S)-methyl 4-(N-(1-phenylethyl)sulfamoyl)benzoate (step-1 of Example 197);

LC-MS (Method A) m/z: M+1 obs 394.94 (fragment signal), tR=3.48 min.

### Step-3: (S)-4-(N-(3-Chloro-5-(trifluoromethyl)pyridin-2-yl)-N-(1-phenylethyl)sulfamoyl)benzoic acid

The titled compound was prepared according to the procedure described in step-2 of Example 15 from (S)-methyl 4-(N-(3-chloro-5-(trifluoromethyl)pyridin-2-yl)-N-(1-phenylethyl)sulfamoyl)benzoate (step-2 of Example 194). The further purification was carried out by preparative LC-MS system in usual manner. HPLC-QC method, retention time and observed MS were summarized in Table 6.

### Example 198

### 4-(N-(3-Chloro-5-(trifluoromethyl)pyridin-2-yl)-N-((2-phenylthiazol-4-yl)methyl)sulfamoyl)benzoic acid

### Step-1: Methyl 4-(N-(3-chloro-5-(trifluoromethyl)pyridin-2-yl)-N-((2-phenylthiazol-4-yl)methyl)sulfamoyl)benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and 4-(chloromethyl)-2-phenylthiazole;

LC-MS (Method B) m/z: M+1 obs 566.9, tR=2.70 min.

### Step-2: 4-(N-(3-Chloro-5-(trifluoromethyl)pyridin-2-yl)-N-((2-phenylthiazol-4-yl)methyl)sulfamoyl)benzoic acid

The titled compound was prepared according to the procedure described in step-2 of Example 15 from methyl 4-(N-(3-Chloro-5-(trifluoromethyl)pyridin-2-yl)-N-((2-phenylthiazol-4-yl)methyl)sulfamoyl)benzoate (step-1 of Example 198). The further purification was carried out by preparative LC-MS system in usual manner. HPLC-QC method, retention time and observed MS were summarized in Table 6.

### Example 199

### 4-(N-(3-Chloro-5-(trifluoromethyl)pyridin-2-yl)-N-((5-phenyl-1,2,4-oxadiazol-3-yl)methyl)sulfamoyl)benzoic acid

### Step-1: Methyl 4-(N-(3-chloro-5-(trifluoromethyl)pyridin-2-yl)-N-((5-phenyl-1,2,4-oxadiazol-3-yl)methyl)sulfamoyl)benzoate

The titled compound was prepared according to the procedure described in step-1 of Example 15 from methyl 4-(N-(3-chloro-5-(trifluoromethyl)pyridin-2-yl)sulfamoyl)benzoate (step-1 of Example 1) and 3-(chloromethyl)-5-phenyl-1,2,4-oxadiazole;

LC-MS (Method B) m/z: M+1 obs 552.9, tR=2.84 min.

### Step-2: 4-(N-(3-chloro-5-(trifluoromethyl)pyridin-2-yl)-N-((5-phenyl-1,2,4-oxadiazol-3-yl)methyl)sulfamoyl)benzoic acid

The titled compound was prepared according to the procedure described in step-2 of Example 15 from methyl 4-(N-(3-chloro-5-(trifluoromethyl)pyridin-2-yl)-N-((5-phenyl-1,2,4-oxadiazol-3-yl)methyl)sulfamoyl)benzoate (step-1 of Example 199). The further purification was carried out by preparative LC-MS system in usual manner. HPLC-QC method, retention time and observed MS were summarized in Table 6.

Quality control analytical condition (Method B) used in Table 6 are described below for all examples.

All examples were identified as the described compounds with the chemical purity of greater than 90% by preparative LC-MS and HPLC-QC method. In Table 6, HPLC retention time and observed MS were measured by HPLC-QC method using condition C, D or E.

TABLE 6

| | | | HPLC | |
|---|---|---|---|---|
| | | | Retention | |
| | | | Time | MS |
| | | | (HPLC | (ESI): |
| Example | Name | Structure | method) | m/z |
| Example 1 | 4-(N-Benzyl-N-(3-chloro- 5-(trifluoromethyl)pyridin- 2-yl)sulfamoyl)benzoic acid | 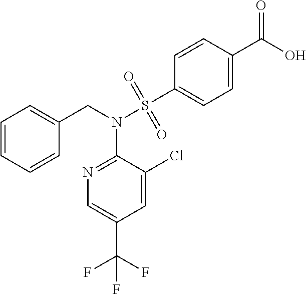 | 1.45 min (C) | 471.0 (M + H)⁺ |
| Example 2 | 4-(N-Benzyl-N-(3- (trifluoromethyl)pyridin- 2-yl)sulfamoyl)benzoic acid | 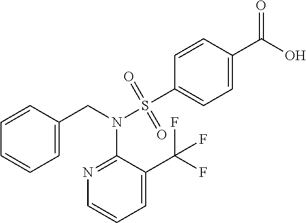 | 1.27 min (C) | 437.2 (M + H)⁺ |
| Example 3 | N-Benzyl-N-(3-chloro-5- (trifluoromethyl)pyridin- 2-yl)benzenesulfonamide | 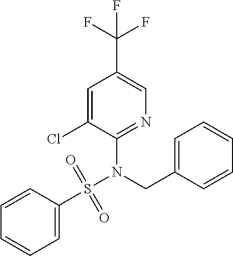 | 2.04 min (C) | 427.0 (M + H)⁺ |
| Example 4 | N-(3-(Trifluoromethyl) phenyl)benzenesulfonamide | 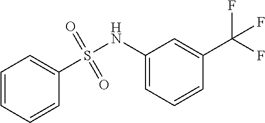 | 1.73 min (C) | 302.1 (M + H)⁺ |
| Example 5 | N-(2-(4-Methylpiperazin- 1-yl)-2-oxoethyl)-N-(3- (trifluoromethyl)phenyl) benzenesulfonamide | 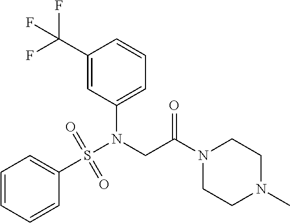 | 1.61 min (C) | 442.2 (M + H)⁺ |
| Example 6 | N-(2-Oxo-2-(piperidin-1- yl)ethyl)-N-(3-(trifluoro methyl)phenyl)benzene sulfonamide | 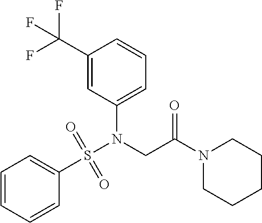 | 1.85 min (C) | 427.2 (M + H)⁺ |
| Example 7 | N-Benzyl-2-(N-(3- (trifluoromethyl)phenyl) benzenesulfonamido) acetamide | 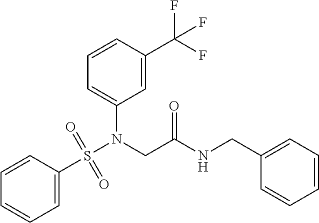 | 1.83 min (C) | 449.2 (M + H)⁺ |
| Example 8 | N-Phenyl-2-(N-(3- (trifluoromethyl)phenyl) benzenesulfonamido) acetamide | 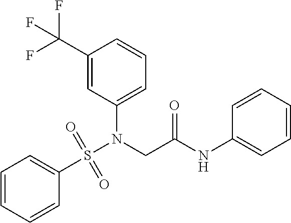 | 1.86 min (C) | 435.1 (M + H)⁺ |
| Example 9 | N-Benzyl-N-(3-chloro-5- (trifluoromethyl)pyridin- 2-yl)-4-(hydroxymethyl) benzene-1-sulfonamide | 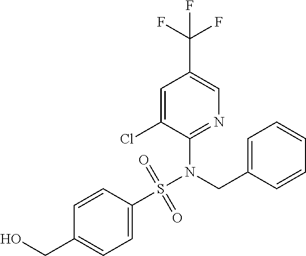 | 1.85 min (C) | 457.1 (M + H)⁺ |
| Example 10 | 4-(Benzyl(3-chloro-5- (trifluoromethyl)pyridin-2- yl)sulfamoyl)benzamide | 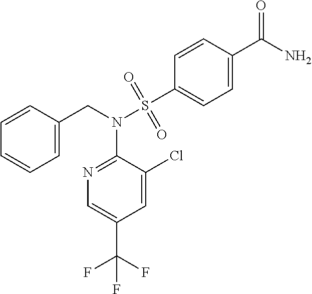 | 1.76 min (C) | 470.1 (M + H)⁺ |
| Example 11 | 4-(Benzyl(3-chloro-5- (trifluoromethyl)pyridin-2- yl)sulfamoyl)-N-methyl- benzamide | 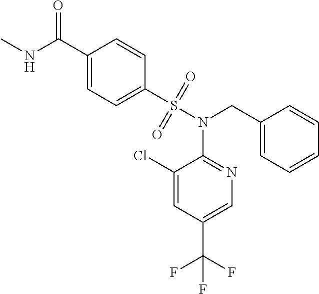 | 1.81 min (C) | 484.1 (M + H)⁺ |
| Example 12 | 4-(Benzyl(3-chloro-5- (trifluoromethyl)pyridin-2- yl)sulfamoyl)-N,N-dimethyl- benzamide | 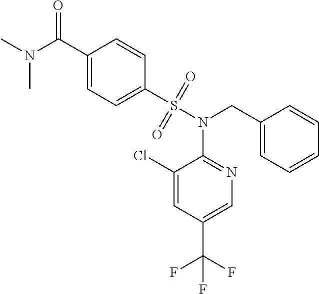 | 1.86 min (C) | 498.2 (M + H)⁺ |
| Example 13 | 4-(Benzyl(3-chloro-5- (trifluoromethyl)pyridin-2- yl)sulfamoyl)-N-(2-hydroxy- ethyl)benzamide | 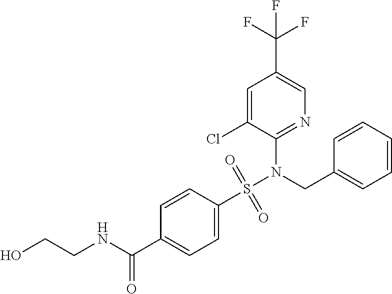 | 1.71 min (C) | 514.2 (M + H)⁺ |
| Example 14 | 4-(N-Benzyl-N-(5- (trifluoromethyl)pyridin- 2-yl)sulfamoyl)benzoic acid | 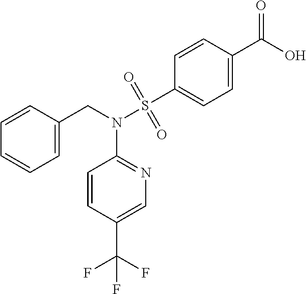 | 1.44 min (C) | 436.9 (M + H)⁺ |
| Example 15 | 4-(N-(3-Chloro-5- (trifluoromethyl)pyridin-2- yl)-N-(4-(trifluoromethoxy) benzyl)sulfamoyl)benzoic acid | 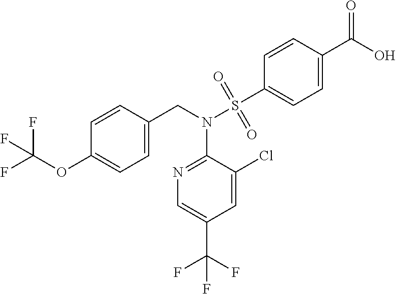 | 1.60 min (C) | 554.8 (M + H)⁺ |
| Example 16 | 4-(N-(3-Chloro-5- (trifluoromethyl)pyridin-2- yl)-N-(4-methoxybenzyl) sulfamoyl)benzoic acid | 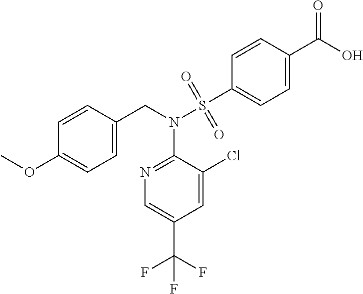 | 1.43 min (C) | 500.8 (M + H)⁺ |
| Example 17 | 4-(N-(3-Chloro-5- (trifluoromethyl)pyridin- 2-yl)-N-(4-fluorobenzyl) sulfamoyl) benzoic acid | 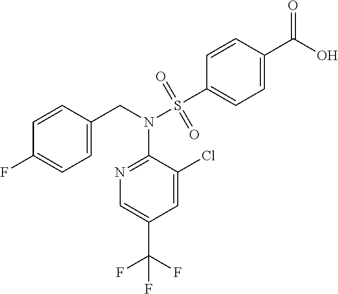 | 1.47 min (C) | 488.8 (M + H)⁺ |
| Example 18 | 4-(N-(4-tert-Butylbenzyl)- N-(3-chloro-5-(trifluoro- methyl)pyridin-2-yl) sulfamoyl)benzoic acid | 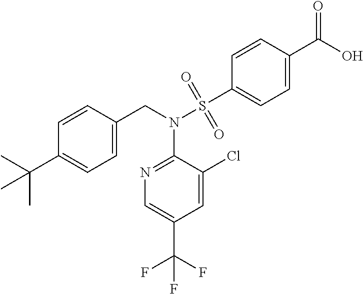 | 1.68 min (C) | 526.9 (M + H)⁺ |
| Example 19 | 4-(N-(3-Chloro-5- (trifluoromethyl)pyridin- 2-yl)-N-(2-cyclohexylethyl) sulfamoyl)benzoic acid | 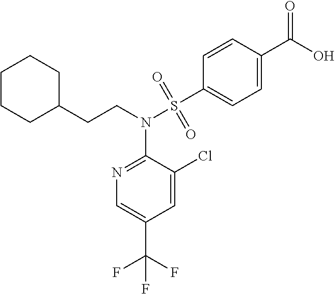 | 1.66 min (C) | 490.9 (M + H)⁺ |
| Example 20 | 4-(N-(3-Chloro-5- (trifluoromethyl)pyridin-2- yl)-N-(2-fluorobenzyl) sulfamoyl)benzoic acid | 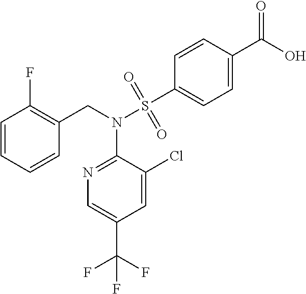 | 1.43 min (C) | 488.8 (M + H)⁺ |
| Example 21 | 4-(N-Benzyl-N-(4- (trifluoromethyl)pyridin- 2-yl)sulfamoyl)benzoic acid | 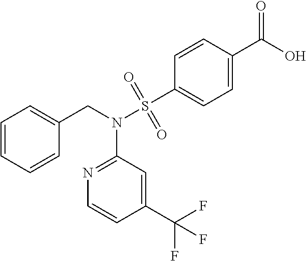 | 1.44 min (C) | 436.8 (M + H)⁺ |
| Example 22 | 4-(N-Benzyl-N-(6- (trifluoromethyl)pyridin-2- yl)sulfamoyl)benzoic acid | 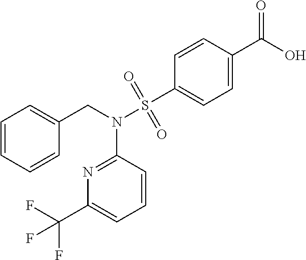 | 1.42 min (C) | 436.8 (M + H)⁺ |
| Example 23 | 4-(N-(3-Chloro-5- (trifluoromethyl)pyridin-2- yl)-N-(3-(trifluoromethyl) benzyl)sulfamoyl)benzoic acid | 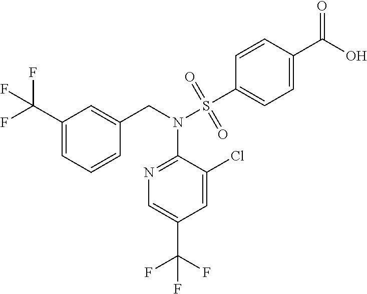 | 1.56 min (C) | 538.8 (M + H)⁺ |
| Example 24 | 4-(N-(3-Chloro-5- (trifluoromethyl)pyridin- 2-yl)-N-(3-methoxybenzyl) sulfamoyl)benzoic acid | 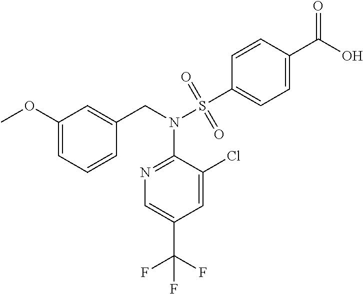 | 1.45 min (C) | 500.8 (M + H)⁺ |
| Example 25 | 4-(N-(3-Chloro-5- (trifluoromethyl)pyridin- 2-yl)-N-(3-(trifluoromethoxy) benzyl)sulfamoyl)benzoic acid | 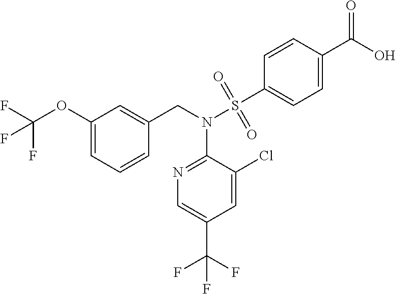 | 1.59 min (C) | 554.8 (M + H)⁺ |
| Example 26 | 4-(N-(3-Chloro-5- (trifluoromethyl)pyridin-2- yl)-N-(2,4-difluorobenzyl) sulfamoyl)benzoic acid | 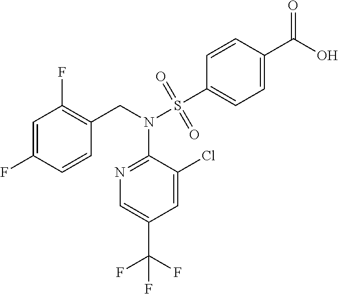 | 1.47 min (C) | 506.8 (M + H)⁺ |
| Example 27 | 4-(N-(3-Chloro-5- (trifluoromethyl)pyridin-2- yl)-N-(4-methylbenzyl) sulfamoyl)benzoic acid | 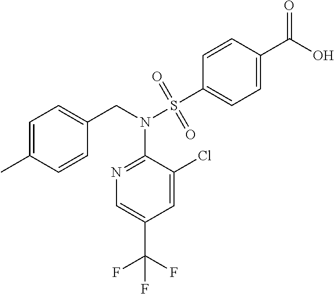 | 1.52 min (C) | 484.8 (M + H)⁺ |
| Example 28 | 4-(N-(3-Chloro-5- (trifluoromethyl)pyridin- 2-yl)-N-(4-isopropylbenzyl) sulfamoyl)benzoic acid | 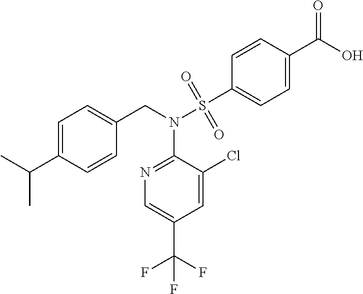 | 1.64 min (C) | 512.9 (M + H)⁺ |
| Example 29 | 4-(N-(3-Chloro-5- (trifluoromethyl)pyridin-2- yl)-N-(cyclopropylmethyl) sulfamoyl)benzoic acid | 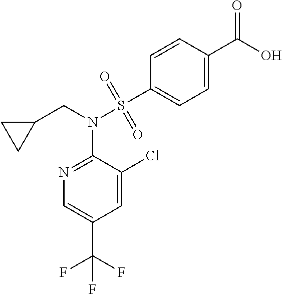 | 1.39 min (C) | 434.8 (M + H)⁺ |
| Example 30 | 4-(N-(3-Chloro-5- (trifluoromethyl)pyridin-2- yl)-N-(2-(4-fluorophenoxy) ethyl)sulfamoyl)benzoic acid | 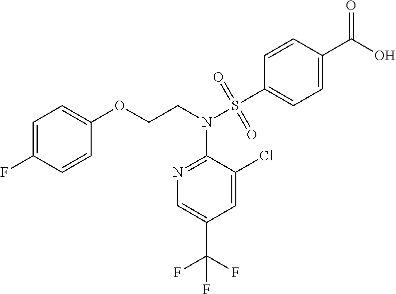 | 3.27 min (D) | 518.9 (M + H)⁺ |
| Example 31 | 4-(N-(3-Chloro-5- (trifluoromethyl)pyridin-2- yl)-N-(2-(trifluoromethyl) benzyl)sulfamoyl)benzoic acid | 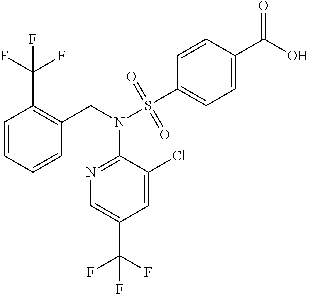 | 3.39 min (D) | 538.9 (M + H)⁺ |
| Example 32 | 4-(N-(3-Chloro-5- (trifluoromethyl)pyridin-2- yl)-N-(4-chlorobenzyl) sulfamoyl)benzoic acid | 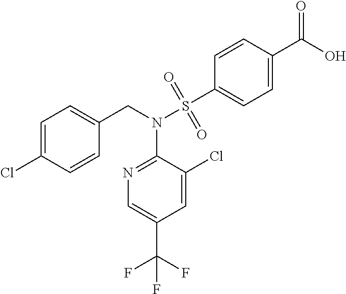 | 2.32 min (E) | 505 (M + H)⁺ |
| Example 33 | 4-(N-(3-Chloro-5- (trifluoromethyl)pyridin-2- yl)-N-(4-cyanobenzyl) sulfamoyl)benzoic acid | 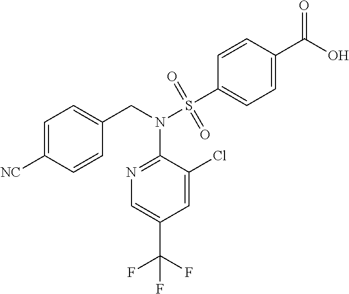 | 3.10 min (D) | 495.9 (M + H)⁺ |
| Example 34 | 4-(N-Benzyl-N-(3- methylpyridin-2-yl)sulfamoyl) benzoic acid | 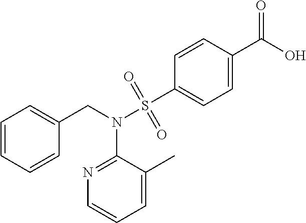 | 2.92 min (D) | 383.1 (M + H)⁺ |
| Example 35 | 4-(N-Benzyl-N-(3,5- dichloropyridin-2-yl) sulfamoyl)benzoic acid | 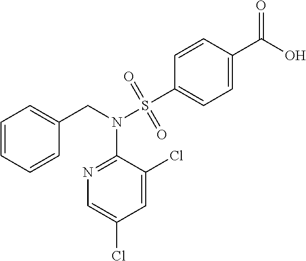 | 1.40 min (C) | 436.8 (M + H)⁺ |
| Example 36 | 4-(N-Benzyl-N-(3-chloro- 5-methylpyridin-2-yl) sulfamoyl)benzoic acid | 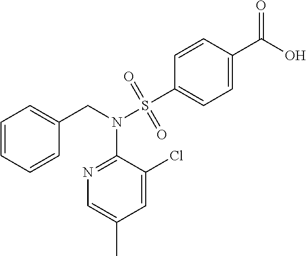 | 1.34 min (C) | 416.9 (M + H)⁺ |
| Example 37 | 4-(N-Benzyl-N-(3- chloropyridin-2-yl) sulfamoyl)benzoic acid | 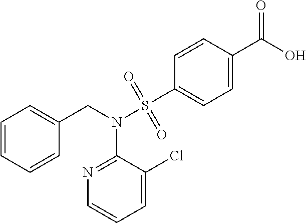 | 1.28 min (C) | 402.8 (M + H)⁺ |
| Example 38 | 4-(N-Benzyl-N-(5- chloro-3-methylpyridin- 2-yl)sulfamoyl)benzoic acid | 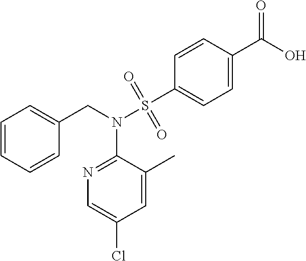 | 1.40 min (C) | 416.9 (M + H)⁺ |
| Example 39 | 4-(N-(3-Chloro-5- (trifluoromethyl)pyridin- 2-yl)-N-(4-(trifluoromethyl) benzyl)sulfamoyl)benzoic acid | 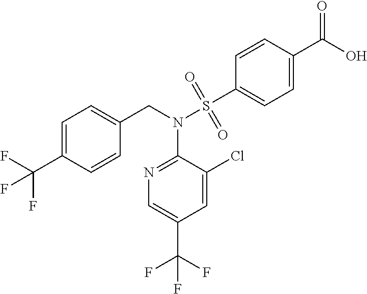 | 1.98 min (C) | 538.7 (M + H)⁺ |
| Example 40 | 4-(N-(2-Chloro-4-fluoro benzyl)-N-(3-chloro-5- (trifluoromethyl)pyridin-2- yl)sulfamoyl)benzoic acid | 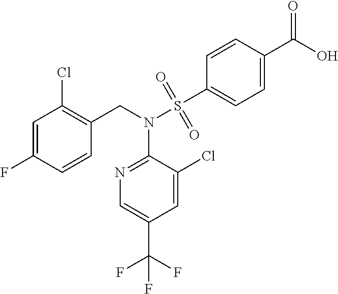 | 1.52 min (C) | 520.9 (M − H)⁻ |
| Example 41 | 4-(N-(3-Chloro-5- (trifluoromethyl)pyridin- 2-yl)-N-(4-(2,2,2-trifluoro- ethoxy)benzyl)sulfamoyl) benzoic acid | 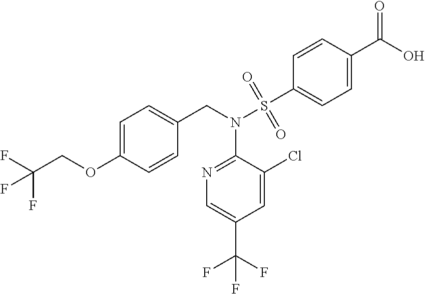 | 1.55 min (C) | 567 (M − H)⁻ |
| Example 42 | 4-(N-(3-Chloro-5- (trifluoromethyl)pyridin- 2-yl)-N-(3,5-difluoro- benzyl)sulfamoyl)benzoic acid | 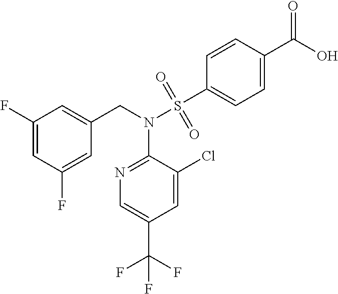 | 1.50 min (C) | 505 (M − H)⁻ |
| Example 43 | 4-(N-(3-Chloro-5- (trifluoromethyl)pyridin- 2-yl)-N-(4-fluoro-3- (trifluoromethyl)benzyl) sulfamoyl)benzoic acid | 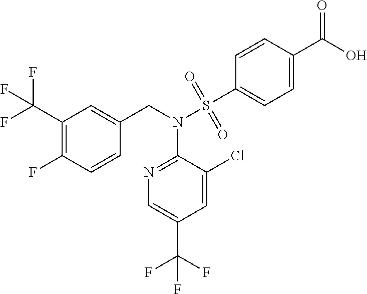 | 1.57 min (C) | 555 (M − H)⁻ |
| Example 44 | 4-(N-(3-Chloro-5- (trifluoromethyl)pyridin-2- yl)-N-(2,6-difluorobenzyl) sulfamoyl)benzoic acid | 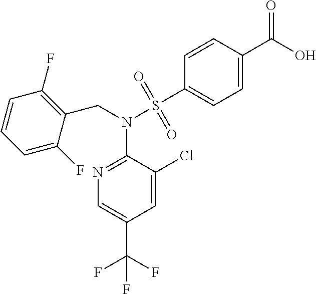 | 1.42 min (C) | 505 (M − H)⁻ |
| Example 45 | 4-(N-(3-Chloro-5- (trifluoromethyl)pyridin-2- yl)-N-(3,4-difluorobenzyl) sulfamoyl)benzoic acid | 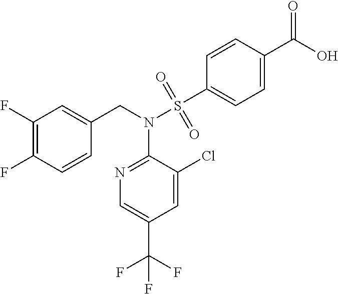 | 1.50 min (C) | 505 (M − H)⁻ |
| Example 46 | 4-(N-(3-Chloro-5- (trifluoromethyl)pyridin- 2-yl)-N-(2,5-difluorobenzyl) sulfamoyl)benzoic acid | 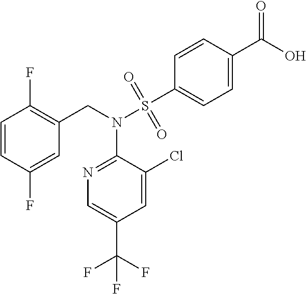 | 1.45 min (C) | 505 (M − H)⁻ |
| Example 47 | 4-(N-(3-Chloro-5- (trifluoromethyl)pyridin-2- yl)-N-(3,4-dichlorobenzyl) sulfamoyl)benzoic acid | 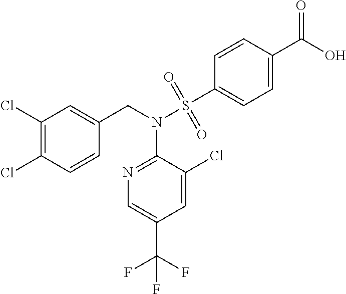 | 1.61 min (C) | 536.9 (M − H)⁻ |

| | | | | |
|---|---|---|---|---|
| Example 48 | 4-(N-(3-Chloro-5- (trifluoromethyl)pyridin- 2-yl)-N-(3-chlorobenzyl) sulfamoyl)benzoic acid | 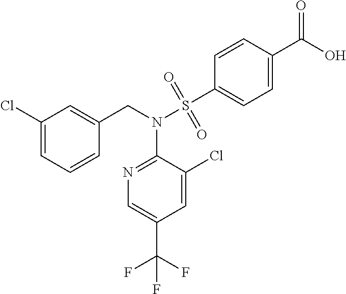 | 1.52 min (C) | 502.9 (M − H)⁻ |
| Example 49 | 4-(N-(3-Chloro-5- (trifluoromethyl)pyridin- 2-yl)-N-(3-chlorobenzyl) sulfamoyl)benzoic acid | 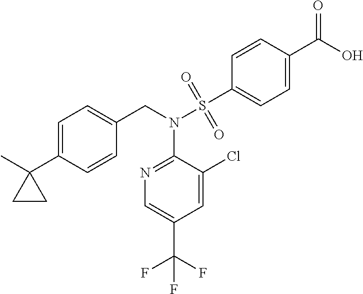 | 3.57 min (D) | 523 (M − H)⁻ |
| Example 50 | 4-(N-(3-Chloro-5- (trifluoromethyl)pyridin- 2-yl)-N-(4-(1,1,1-trifluoro- 2-methylpropan-2-yl)benzyl) sulfamoyl)benzoic acid | 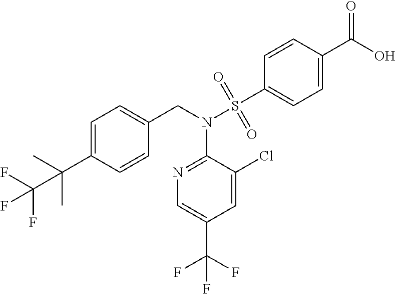 | 3.55 min (D) | 579 (M − H)⁻ |
| Example 51 | 4-(N-(3-Chloro-5- (trifluoromethyl)pyridin- 2-yl)-N-(2-(trifluoro- methoxy)benzyl)sulfamoyl) benzoic acid | 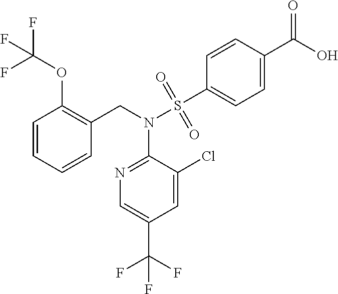 | 3.40 min (D) | 553 (M − H)⁻ |
| Example 52 | 3-(N-Benzyl-N-(3-chloro- 5-(trifluoromethyl)pyridin-2- yl)sulfamoyl)benzoic acid | 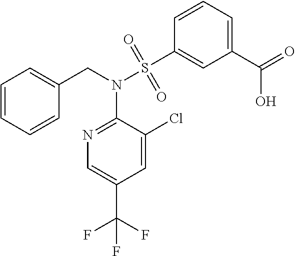 | 1.42 min (C) | 469 (M − H)⁻ |
| Example 53 | 4-(N-Benzyl-N-(3-fluoro- 5-(trifluoromethyl)pyridin- 2-yl)sulfamoyl)benzoic acid | 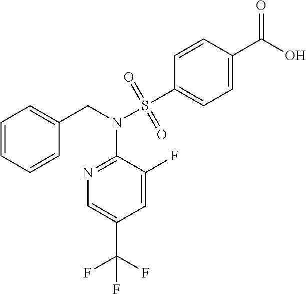 | 1.40 min (C) | 453.1 (M − H)⁻ |
| Example 54 | 4-(N-(3,5-Dichloropyridin- 2-yl)-N-(4-(trifluoromethyl) benzyl)sulfamoyl)benzoic acid | 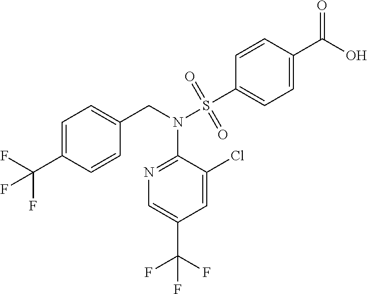 | 1.52 min (C) | 503 (M − H)⁻ |
| Example 55 | 4-(N-(5-Chloro-3- methylpyridin-2-yl)- N-(4-(trifluoromethyl) benzyl)sulfamoyl)benzoic acid | 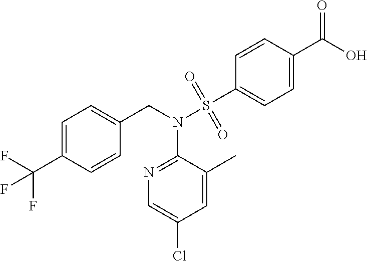 | 1.52 min (C) | 483.1 (M − H)⁻ |
| Example 56 | 4-(N-Benzyl-N-(2-chloro- 4-(trifluoromethyl)phenyl) sulfamoyl)benzoic acid | 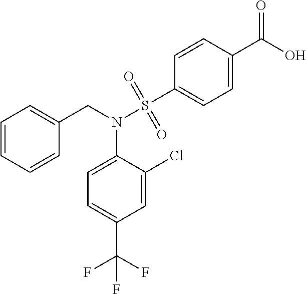 | 2.29 min (E) | 470.1 (M + H)⁺ |
| Example 57 | 4-(N-(3-Chloro-5- (trifluoromethyl)pyridin- 2-yl)-N-(2-morpholino- ethyl)sulfamoyl)benzoic acid | 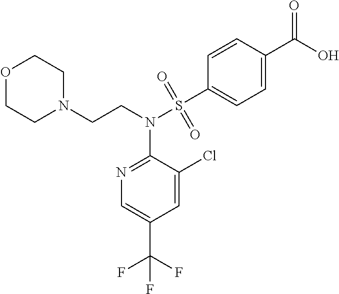 | 1.30 min (C) | 492.1 (M − H)⁻ |
| Example 58 | 4-(N-(3-Chloro-5- (trifluoromethyl)pyridin- 2-yl)-N-(pyridin-2-ylmethyl) sulfamoyl)benzoic acid | 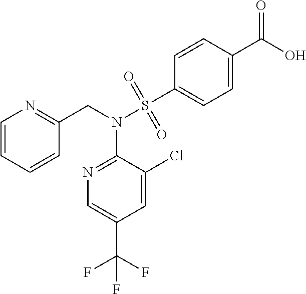 | 1.28 min (C) | 470.1 (M − H)⁻ |
| Example 59 | 4-(N-(3-Chloro-5- (trifluoromethyl)pyridin-2- yl)-N-(3-phenylpropyl) sulfamoyl)benzoic acid | 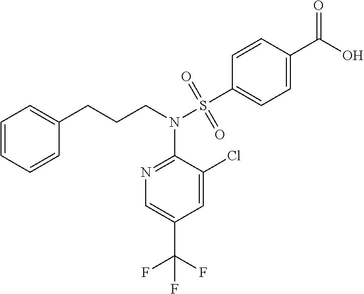 | 1.53 min (C) | 498.8 (M + H)⁺ |
| Example 60 | 4-(N-(3,5-Dichloropyridin- 2-yl)-N-(4-fluoro-3- (trifluoromethyl)benzyl) sulfamoyl)benzoic acid | 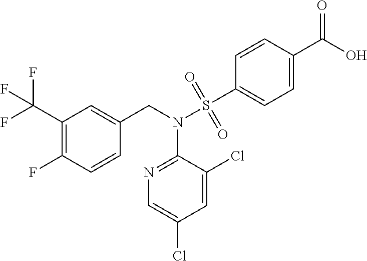 | 1.53 min (C) | 520.9 (M − H)⁻ |
| Example 61 | 4-(N-(3,5-Dichloropyridin- 2-yl)-N-(4-fluorobenzyl) sulfamoyl)benzoic acid | 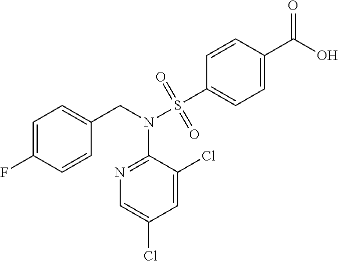 | 1.42 min (C) | 452.9 (M − H)⁻ |
| Example 62 | 4-(N-(4-Chloro-3-fluoro- benzyl)-N-(3,5-dichloro pyridin-2-yl)sulfamoyl) benzoic acid | 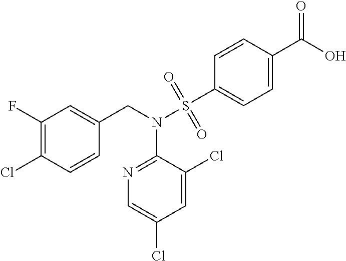 | 1.52 min (C) | 486.9 (M − H)⁻ |
| Example 63 | 4-(N-(3,5-Dichloropyridin- 2-yl)-N-(4-(trifluoromethoxy) benzyl)sulfamoyl)benzoic acid | 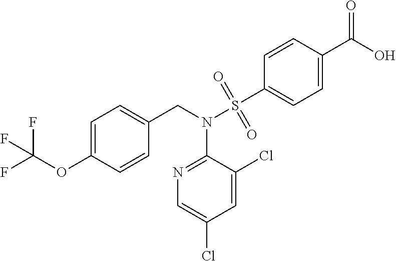 | 1.56 min (C) | 518.9 (M + H)⁺ |
| Example 64 | 4-(N-(3,5-Dichloropyridin- 2-yl)-N-(2-(trifluoromethyl) benzyl)sulfamoyl)benzoic acid | 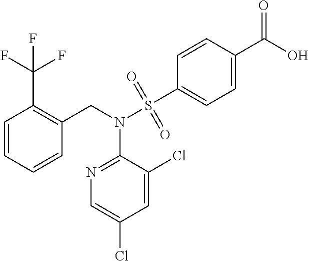 | 1.49 min (C) | 502.9 (M − H)⁻ |
| Example 65 | 4-(N-(4-Chloro-3-fluoro benzyl)-N-(3-chloro-5- (trifluoromethyl)pyridin-2- yl)sulfamoyl)benzoic acid | 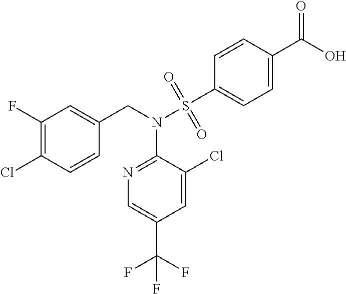 | 1.56 min (C) | 520.9 (M − H)⁻ |
| Example 66 | 4-(N-(4-Chloro-3- (trifluoromethyl)benzyl)- N-(3,5-dichloropyridin-2- yl)sulfamoyl)benzoic acid | 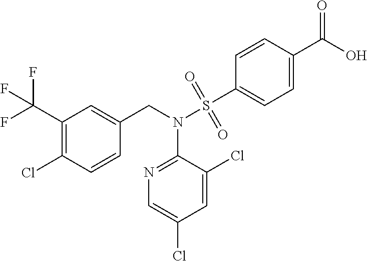 | 1.60 min (C) | 536.9 (M − H)⁻ |
| Example 67 | 4-(N-(3,5-Dichloropyridin- 2-yl)-N-(3-fluoro-4-methyl- benzyl)sulfamoyl)benzoic acid | 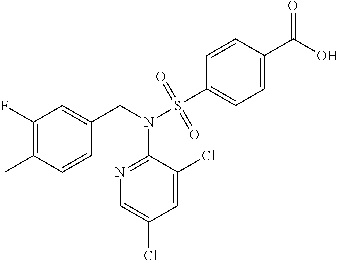 | 1.49 min (C) | 467 (M − H)⁻ |
| Example 68 | 4-(N-(3,5-Dichloropyridin- 2-yl)-N-(4-methyl-3- (trifluoromethyl)benzyl) sulfamoyl)benzoic acid | 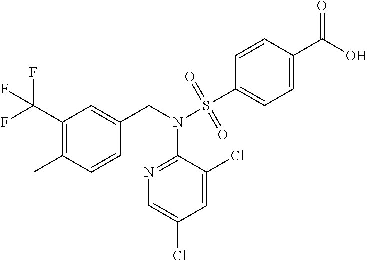 | 1.57 min (C) | 517 (M − H)⁻ |
| Example 69 | 4-(N-(3-Chloro-4-fluoro- benzyl)-N-(3,5-dichloro pyridin-2-yl)sulfamoyl) benzoic acid | 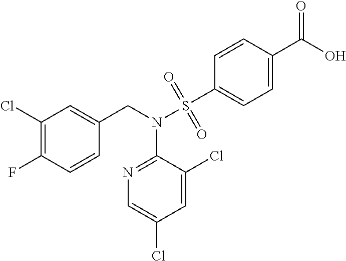 | 1.51 min (C) | 486.9 (M − H)⁻ |
| Example 70 | 4-(N-(3,5-Dichloropyridin- 2-yl)-N-(4-(1-methylcyclo- propyl)benzyl)sulfamoyl) benzoic acid | 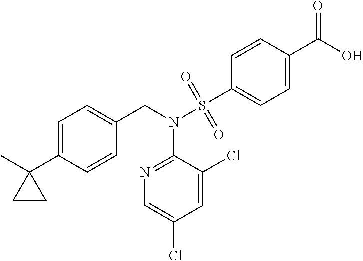 | 1.60 min (C) | 489 (M − H)⁻ |
| Example 71 | 4-(N-(3-Chloro-4-fluoro- benzyl)-N-(3-chloro-5- (trifluoromethyl)pyridin-2- yl)sulfamoyl)benzoic acid | 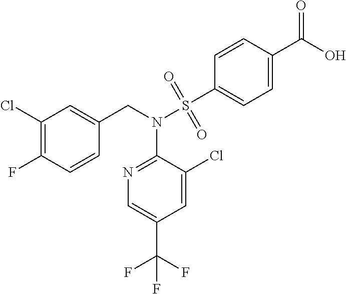 | 1.55 min (C) | 521 (M − H)⁻ |
| Example 72 | 4-(N-(4-Chloro-2- (trifluoromethyl)benzyl)- N-(3,5-dichloropyridin-2- yl)sulfamoyl)benzoic acid | 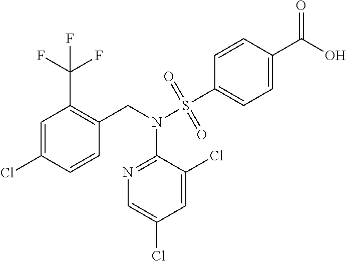 | 1.62 min (C) | 536.9 (M − H)⁻ |
| Example 73 | 4-(N-(3,5-Dichloropyridin- 2-yl)-N-(2-(4-fluorophenoxy) ethyl)sulfamoyl)benzoic acid | 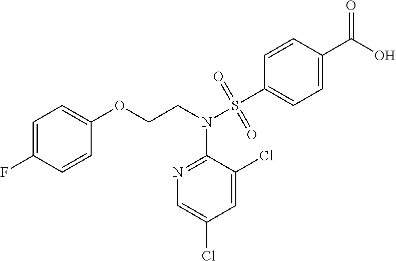 | 1.44 min (C) | 482.9 (M − H)⁻ |
| Example 74 | 4-(N-(3,5-Dichloropyridin- 2-yl)-N-(4-(2-oxopyrrolidin- 1-yl)benzyl)sulfamoyl)benzoic acid | 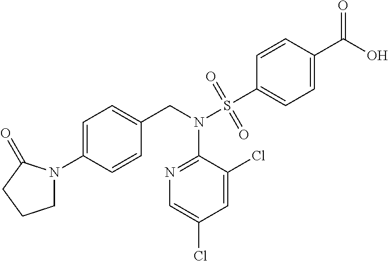 | 1.25 min (C) | 517.83 (M − H)⁻ |
| Example 75 | 4-(N-(4-Chloro-2- (trifluoromethyl)benzyl)- N-(5-chloro-3-methylpyridin- 2-yl)sulfamoyl)benzoic acid | 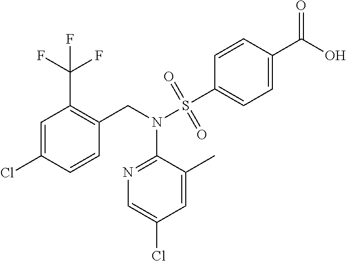 | 1.58 min (C) | 516.81 (M − H)⁻ |
| Example 76 | 4-(N-(5-Chloro-3- methylpyridin-2-yl)-N- (4-fluoro-3-(trifluoromethyl) benzyl)sulfamoyl)benzoic acid | 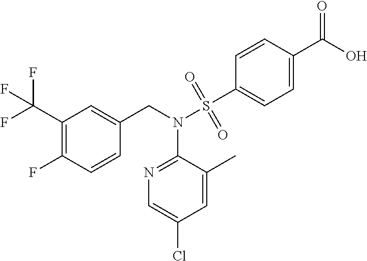 | 1.51 min (C) | 500.82 (M − H)⁻ |
| Example 77 | 4-(N-(5-Chloro-3- methylpyridin-2-yl)-N- (3-chloro-4-fluorobenzyl) sulfamoyl)benzoic acid | 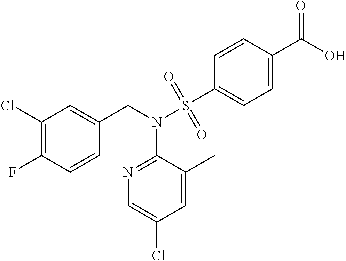 | 1.48 min (C) | 466.8 (M − H)⁻ |
| Example 78 | 4-(N-(5-Chloro-3- methylpyridin-2-yl)-N- (4-(trifluoromethoxy)benzyl) sulfamoyl)benzoic acid | 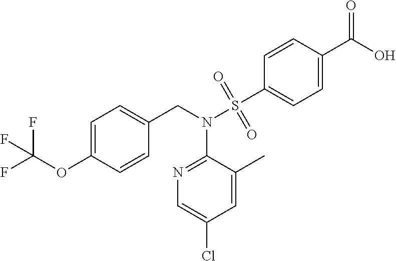 | 1.54 min (C) | 498.85 (M − H)⁻ |
| Example 79 | 4-(N-(4-Chloro-3- (trifluoromethyl)benzyl)- N-(5-chloro-3-methylpyridin- 2-yl)sulfamoyl)benzoic acid | 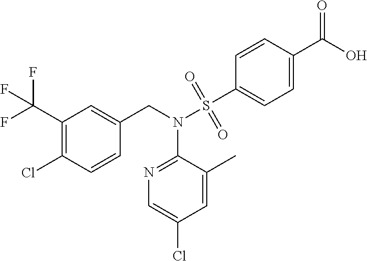 | 1.58 min (C) | 516.82 (M − H)⁻ |
| Example 80 | 4-(N-(5-Chloro-3- methylpyridin-2-yl)-N- (4-(2-oxopyrrolidin-1- yl)benzyl)sulfamoyl)benzoic acid | 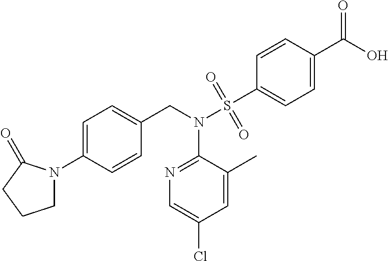 | 1.24 min (C) | 497.91 (M − H)⁻ |
| Example 81 | 4-(N-(2-Chloro-4- (trifluoromethyl)phenyl)- N-(2-morhpholinoethyl) sulfamoyl)benzoic acid | 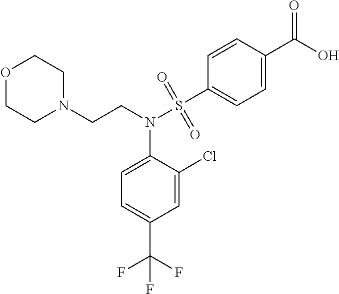 | 1.31 min (C) | 492.9 (M + H)⁺ |
| Example 82 | 4-(N-(2-Chloro-4- (trifluoromethyl)phenyl)- N-(pyridin-2-ylmethyl) sulfamoyl)benzoic acid | 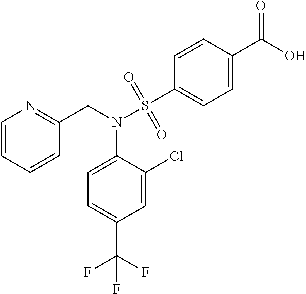 | 1.33 min (C) | 470.8 (M + H)⁺ |
| Example 83 | 4-(N-(2-Chloro-4- (trifluoromethyl)phenyl)- N-(2-(piperidin-1-yl)ethyl) sulfamoyl)benzoic acid | 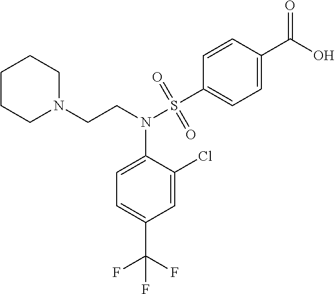 | 1.40 min (C) | 490.9 (M + H)⁺ |
| Example 84 | N-(2-(4-Benzylpiperazin- 1-yl)-2-oxoethyl)-N-(3- methoxyphenyl)benzene- sulfonamide | 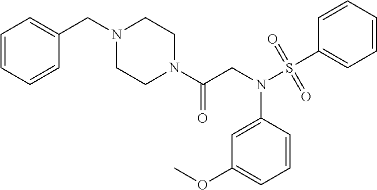 | 1.83 min (C) | 480 (M + H)⁺ |
| Example 85 | N-(1-(2,5-Dimethoxyphenyl) ethyl)-4-methyl-N-p- tolylbenzenesulfonamide | 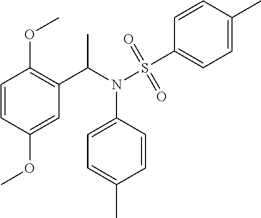 | 2.08 min (C) | 426 (M + H)⁺ |
| Example 86 | N-(3-Chlorophenyl)-N- (1-(2,5-dimethoxyphenyl) ethyl)-4-methylbenzene- sulfonamide | 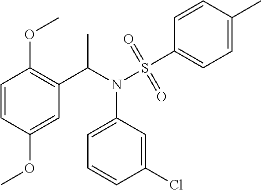 | 2.10 min (C) | 446 (M + H)⁺ |
| Example 87 | N-(1-(4-Chlorophenyl) ethyl)-N-(4-methoxyphenyl) benzenesulfonamide | 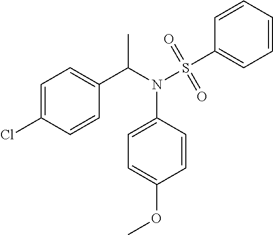 | 2.10 min (C) | 401.9 (M + H)⁺ |
| Example 88 | 4-Fluoro-N-(1-(2-fluoro phenyl)ethyl)-N-(4- methoxyphenyl)benzene- sulfonamide | 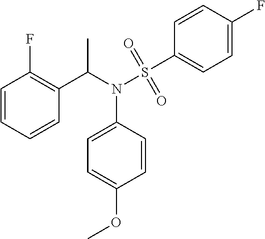 | 2.01 min (C) | 404 (M + H)⁺ |
| Example 89 | N-(4-Methoxyphenyl)-N- (1-(2-methoxyphenyl) ethyl)-4-methylbenzene- sulfonamide | 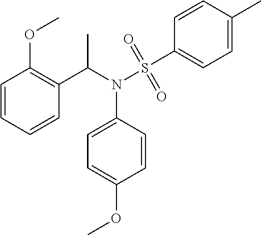 | 2.06 min (C) | 412 (M + H)⁺ |
| Example 90 | N-(1-(2,5-Dimethoxyphenyl) ethyl)-2-fluoro-N-(3- methoxyphenyl)benzene- sulfonamide | 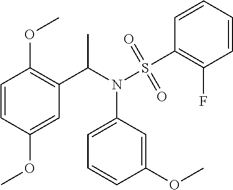 | 1.95 min (C) | 446 (M + H)⁺ |
| Example 91 | 2-Fluoro-N-(1-(2-fluoro- phenyl)ethyl)-N-m-tolyl benzenesulfonamide | 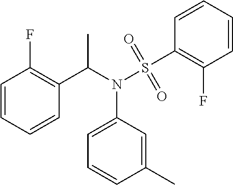 | 2.05 min (C) | 387.9 (M + H)⁺ |
| Example 92 | 2-Fluoro-N-(1-(2-fluoro phenyl)ethyl)-N-(3- methoxyphenyl)benzene- sulfonamide | 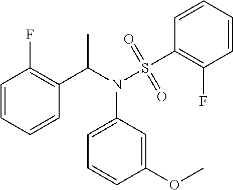 | 1.97 min (C) | 403.9 (M + H)⁺ |
| Example 93 | N-(3,4-Dimethoxyphenyl)- 2-fluoro-N-(1-(2-methoxy- phenyl)ethyl)benzene- sulfonamide | 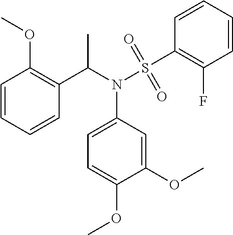 | 1.90 min (C) | 446 (M + H)⁺ |
| Example 94 | 3-Chloro-4-fluoro-N-(3- methoxyphenyl)-N-(1- phenylethyl)benzene- sulfonamide | 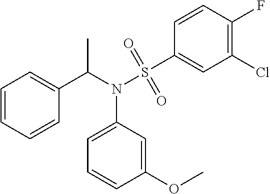 | 2.13 min (C) | 419.9 (M + H)⁺ |
| Example 95 | 3-Chloro-N-(3-methoxy- phenyl)-4-methyl-N- (1-o-tolylethyl)benzene- sulfonamide | 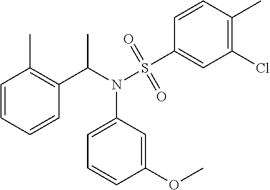 | 2.23 min (C) | 429.9 (M + H)⁺ |
| Example 96 | N-(1-(2,5-Dimethoxyphenyl) ethyl)-4-fluoro-2-methyl-N- p-tolylbenzenesulfonamide | 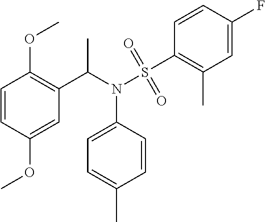 | 2.13 min (C) | 444 (M + H)⁺ |
| Example 97 | 3-Chloro-4-fluoro-N-(1-(2- fluorophenyl)ethyl)-N- (4-methoxyphenyl) benzenesulfonamide | 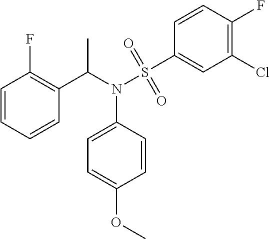 | 2.11 min (C) | 437.9 (M + H)⁺ |
| Example 98 | 4-Fluoro-N-(1-(2-fluoro phenyl)ethyl)-2-methyl- N-m-tolylbenzenesulfonamide | 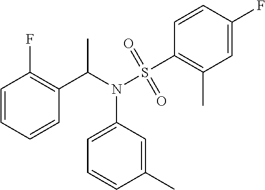 | 2.15 min (C) | 402 (M + H)⁺ |
| Example 99 | 5-Fluoro-N-(1-(2-fluoro- phenyl)ethyl)-2-methyl- N-m-tolylbenzenesulfonamide | 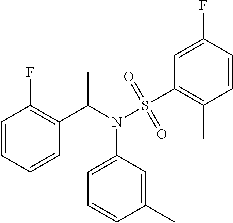 | 2.15 min (C) | 402 (M + H)⁺ |
| Example 100 | N-(1-(2-Fluorophenyl) ethyl)-2-methyl-N-tolyl benzenesulfonamide | 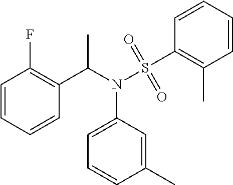 | 2.13 min (C) | 384 (M + H)⁺ |
| Example 101 | 3-Fluoro-N-(1-(3- methoxyphenyl)ethyl)- 4-methyl-N-phenylbenzene- sulfonamide | 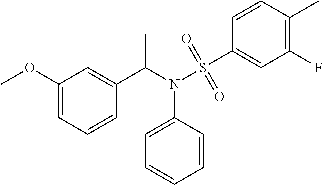 | 2.08 min (C) | 399.9 (M + H)⁺ |
| Example 102 | 3-Chloro-N-(3-methoxy phenyl)-N-(1-(3-methoxy- phenyl)ethyl)-4-methyl benzenesulfonamide | 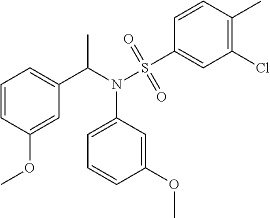 | 2.15 min (C) | 445.9 (M + H)⁺ |
| Example 103 | 3-Fluoro-N-(3-methoxy phenyl)-N-(1-(3-methoxy- phenyl)ethyl)-4-methyl benzenesulfonamide | 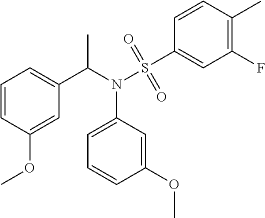 | 2.07 min (C) | 430 (M + H)⁺ |
| Example 104 | 3-Chloro-N-(4-methoxy phenyl)-N-(1-(2-methoxy- phenyl)ethyl)-4-methyl benzenesulfonamide | 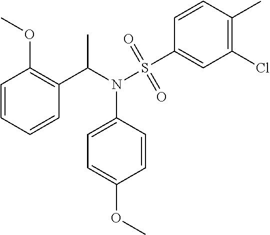 | 2.17 min (C) | 445.9 (M + H)⁺ |
| Example 105 | 3-Chloro-N-(3- fluorophenyl)-N-(1- (2-methoxyphenyl) ethyl)-4-methylbenzene- sulfonamide | 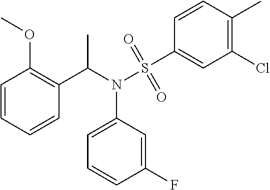 | 2.19 min (C) | 433.9 (M + H)⁺ |
| Example 106 | N-Benzyl-4-methyl- N-(pyridin-2-yl) benzenesulfonamide | 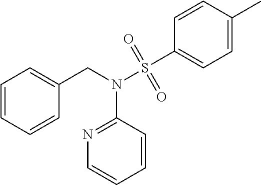 | 1.95 min (C) | 338.9 (M + H)⁺ |
| Example 107 | N-(4-(N-Benzyl-N- (pyridin-2-yl)sulfamoyl) phenyl)acetamide | 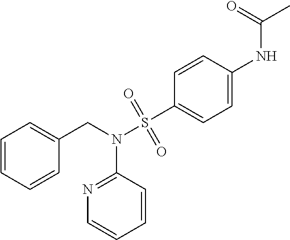 | 1.64 min (C) | 381.9 (M + H)⁺ |
| Example 108 | N-Benzyl-4-fluoro-N- (pyridin-2-yl)benezene- sulfonamide | 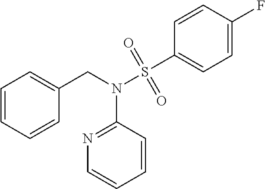 | 1.89 min (C) | 342.9 (M + H)⁺ |
| Example 109 | N-Benzyl-2,5-dichloro- N-(pyridin-2-yl)benzene sulfonamide | 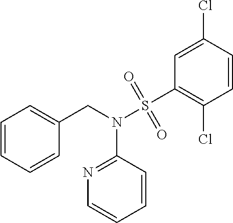 | 2.06 min (C) | 392.8 (M + H)⁺ |
| Example 110 | 3-(N-Benzyl-N-(4- chlorophenyl)sulfamoyl)-4- chlorobenzoic acid | 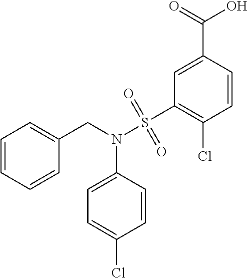 | 1.46 min (C) | 435.8 (M + H)⁺ |
| Example 111 | 3-(N-Benzyl-N-(3- (trifluoromethyl)phenyl) sulfamoyl)benzoic acid | 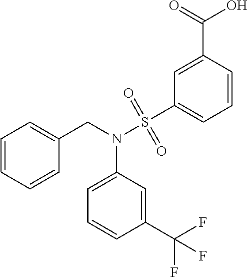 | 1.43 min (C) | 435.9 (M + H)⁺ |
| Example 112 | 3-(N-Benzyl-N-(2- chlorophenyl)sulfamoyl) benzoic acid | 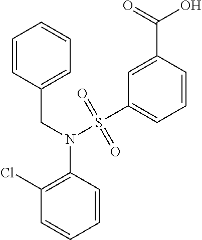 | 1.35 min (C) | 401.9 (M + H)⁺ |
| Example 113 | 3-(N-Benzyl-N-(4- methoxyphenyl)sulfamoyl) benzoic acid | 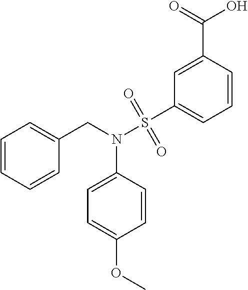 | 1.32 min (C) | 397.9 (M + H)⁺ |
| Example 114 | 3-(N-Benzyl-N-(2- methoxyphenyl)sulfamoyl) benzoic acid | 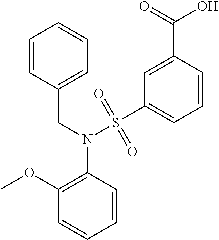 | 1.32 min (C) | 397.9 (M + H)⁺ |
| Example 115 | 3-(N-Benzyl-N-(4- chlorophenyl)sulfamoyl) benzoic acid | 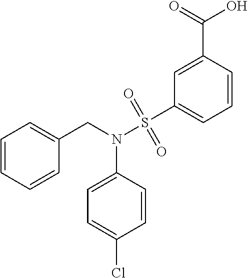 | 1.40 min (C) | 401.8 (M + H)⁺ |
| Example 116 | 3-(N-Benzyl-N- phenylsulfamoyl)benozic acid | 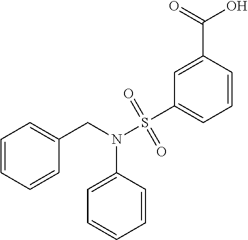 | 1.31 min (C) | 367.9 (M + H)⁺ |
| Example 117 | 5-(N-Benzyl-N- phenylsulfamoyl)-2- chlorobenzoic acid | 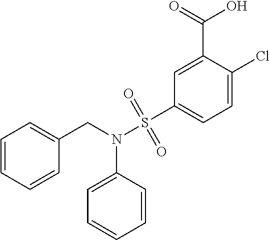 | 1.32 min (C) | 401.9 (M + H)⁺ |
| Example 118 | 3-(N-Benzyl-N-(2- methoxyphenyl)sulfamoyl)- 4-chlorobenzoic acid | 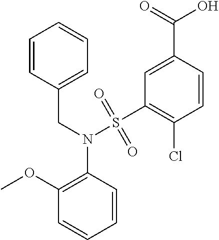 | 1.40 min (C) | 431.8 (M + H)⁺ |
| Example 119 | 5-(N-Benzyl-N-(4- methoxyphenyl)sulfamoyl)- 2-chlorobenzoic acid | 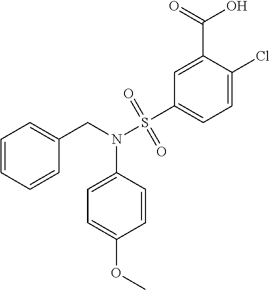 | 1.33 min (C) | 431.9 (M + H)⁺ |
| Example 120 | 3-(N-Benzyl-N-(2- chlorophenyl)sulfamoyl)- 4-chlorobenzoic acid | 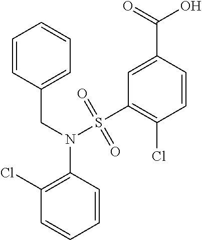 | 1.43 min (C) | 435.8 (M + H)⁺ |
| Example 121 | 5-(N-Benzyl-N-(2- chlorophenyl)sulfamoyl)- 2-chlorobenzoic acid | 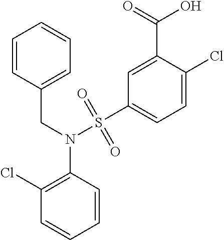 | 1.35 min (C) | 435.8 (M + H)⁺ |
| Example 122 | 5-(N-Benzyl-N-(4- chlorophenyl)sulfamoyl)- 2-chlorobenzoic acid | 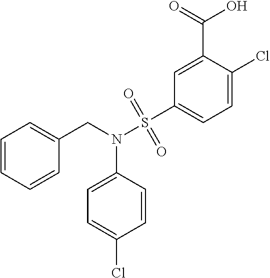 | 1.41 min (C) | 435.8 (M + H)⁺ |
| Example 123 | 3-(N-Benzyl-N-(4-fluoro phenyl)sulfamoyl)-4- chlorobenzoic acid | 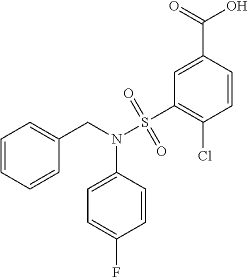 | 1.40 min (C) | 419.9 (M + H)⁺ |
| Example 124 | 3-(N-Benzyl-N-(4- methoxyphenyl)sulfamoyl)- 4-chlorobenzoic acid | 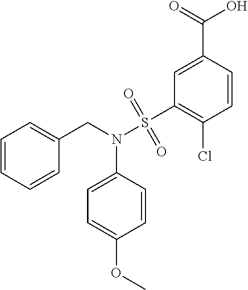 | 1.39 min (C) | 431.8 (M + H)⁺ |
| Example 125 | 3-(N-(4-Chlorobenzyl)- N-(2-ethoxyphenyl)sulfamoyl) benzoic acid | 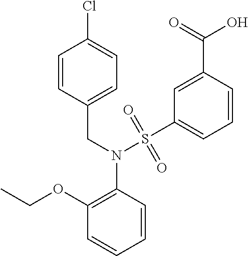 | 1.39 min (C) | 445.9 (M + H)⁺ |
| Example 126 | 3-(N-Benzyl-N- phenylsulfamoyl)-4- chlorobenzoic acid | 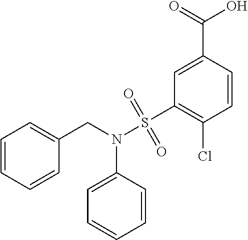 | 1.39 min (C) | 401.8 (M + H)⁺ |
| Example 127 | N-Benzyl-2,4,6-trimethyl- N-(pyridin-2-yl)benzene- sulfonamide | 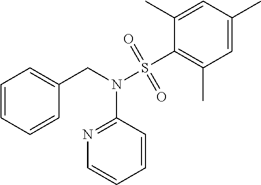 | 2.11 min (C) | 366.9 (M + H)⁺ |
| Example 128 | N-Benzyl-N-(pyridin-2-yl) quinoline-8-sulfonamide | 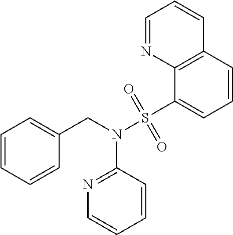 | 1.83 min (C) | 375.9 (M + H)⁺ |
| Example 129 | N-Benzyl-4-chloro-N- (pyridin-2-yl)benzene- sulfonamide | 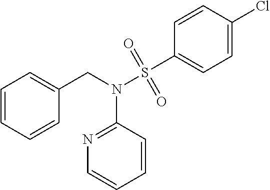 | 2.00 min (C) | 358.9 (M + H)⁺ |
| Example 130 | N-Benzyl-4-nitro-N- (pyridin-2-yl)benzene- sulfonamide | 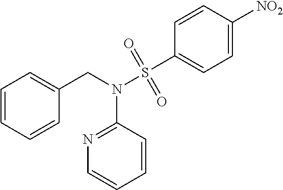 | 1.88 min (C) | 369.9 (M + H)⁺ |
| Example 131 | N-Benzyl-N-(pyridin-2- yl)benzenesulfonamide | 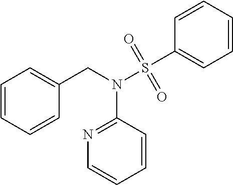 | 1.86 min (C) | 324.9 (M + H)⁺ |
| Example 132 | N-Benzyl-4-methoxy-N- (pyridin-2-yl)benzene- sulfonamide | 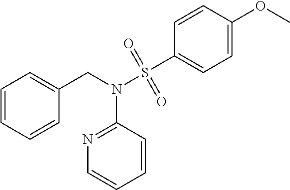 | 1.87 min (C) | 354.9 (M + H)⁺ |
| Example 133 | N-Benzyl-4-methyl-3- nitro-N-(pyridin-2-yl) benzenesulfonamide | 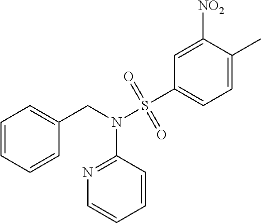 | 1.93 min (C) | 383.9 (M + H)⁺ |
| Example 134 | 5-Chloro-2-fluoro-N-(4- methoxyphenyl)-N-(1- p-tolylethyl)benezene- sulfonamide | 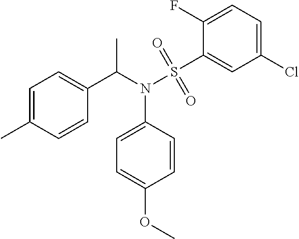 | 2.18 min (C) | 450.9 (M + NH₄)⁺ |
| Example 135 | N-(1-(2,5-Dimethoxyphenyl) ethyl)-4-fluoro-N-(4-fluoro- phenyl)-2-methylbenzene- sulfonamide | 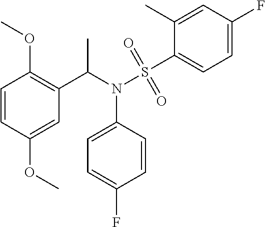 | 2.08 min (C) | 464.9 (M + NH₄)⁺ |
| Example 136 | N-(1-(2,5-Dimethoxyphenyl) ethyl)-5-fluoro-N-(3- fluorophenyl)-2-methyl- benzenesulfonamide | 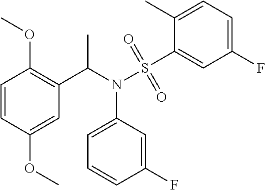 | 2.09 min (C) | 464.9 (M + NH₄)⁺ |
| Example 137 | 3-Chloro-N-(4-methoxy phenyl)-N-(1-(4-methoxy- phenyl)ethyl)-4-methyl benzenesulfonamide | 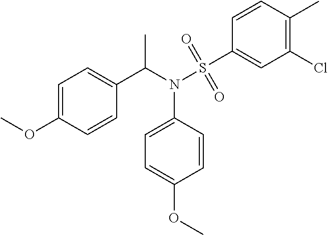 | 2.16 min (C) | 462.9 (M + NH₄)⁺ |
| Example 138 | 3-Fluoro-N-(1-(3-methoxy- phenyl)ethyl)-N-phenyl- benzenesulfonamide | 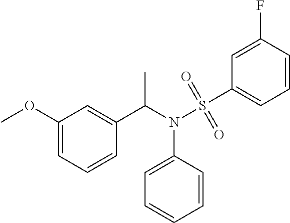 | 2.02 min (C) | 402.9 (M + NH₄)⁺ |
| Example 139 | N-(3-Chlorophenyl)-4- fluoro-N-(1-(3-methoxy- phenyl)ethyl)-2-methyl- benzenesulfonamide | 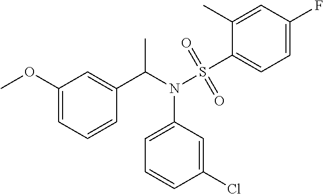 | 2.15 min (C) | 450.9 (M + NH₄)⁺ |
| Example 140 | 4-(N-(3-Chloro-5- (trifluoromethyl)pyridin- 2-yl)-N-(4-(trimethylsilyl) benzyl)sulfamoyl)benzoic acid | 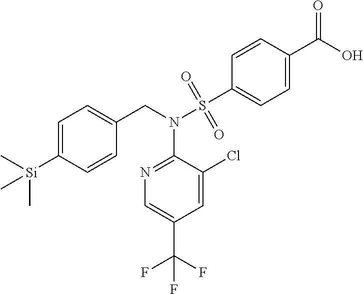 | 1.77 min (C) | 540.7 (M − H)⁻ |
| Example 141 | 4-(N-(3-Chloro-5- (trifluoromethyl)pyridin-2- yl)-N-(4-(1-cyanocyclopropyl) benzyl)sulfamoyl)benzoic acid | 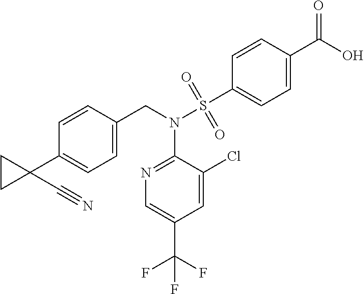 | 1.43 min (C) | 533.8 (M − H)⁻ |
| Example 142 | 4-(N-(3-Chloro-5- (trifluoromethyl)pyridin- 2-yl)-N-((1-(pyridin-2-yl) piperidin-4-yl)methyl) sulfamoyl)benzoic acid | 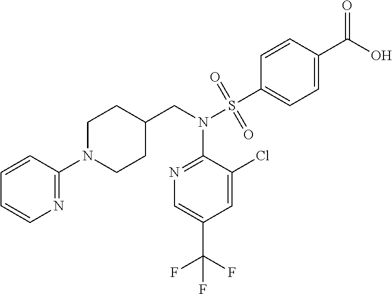 | 2.58 min (C) | 553.1 (M − H)⁻ |
| Example 143 | 4-(N-Benzyl-N-(3-chloro- 5-(trifluoromethyl)pyridin- 2-yl)sulfamoyl)-2-methyl- benzoic acid | 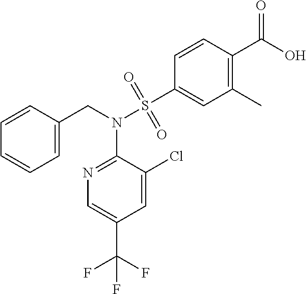 | 1.44 min (C) | 482.9 (M − H)⁻ |
| Example 144 | N-Benzyl-N-(3-chloro-5- (trifluoromethyl)pyridin- 2-yl)-6-hydroxypyridine- 3-sulfonamide | 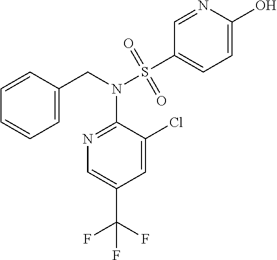 | 1.70 min (C) | 441.9 (M − H)⁻ |
| Example 145 | 4-(N-Benzyl-N-(3-chloro- 5-(trifluoromethyl)pyridin- 2-yl)sulfamoyl)-3-methyl- benzoic acid | 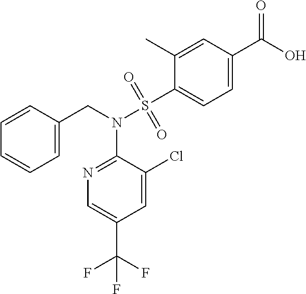 | 1.47 min (C) | 482.9 (M − H)⁻ |
| Example 146 | 4-(N-Benzyl-N-(3-chloro- 5-(trifluoromethyl)pyridin- 2-yl)sulfamoyl)-3-methoxy- benzoic acid | 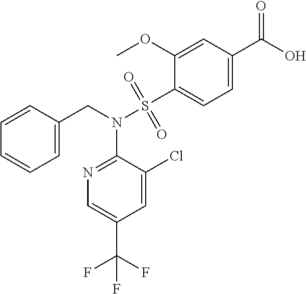 | 1.44 min (C) | 498.9 (M − H)⁻ |
| Example 147 | 2-(4-(N-Benzyl-N-(3-chloro- 5-(trifluoromethyl)pyridin- 2-yl)sulfamoyl)phenyl)acetic acid | 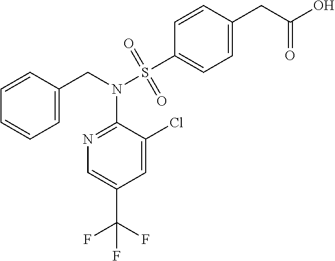 | 1.45 min (C) | 482.9 (M − H)⁻ |
| Example 148 | 4-(N-(3-Chloro-5- (trifluoromethyl)pyridin- 2-yl)-N-phenethylsulfamoyl) benzoic acid | 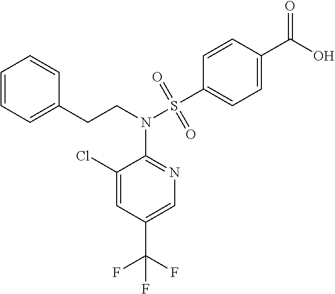 | 1.50 min (C) | 482.8 (M − H)⁻ |
| Example 149 | 4-(N-Benzyl-N-(3-cyclo propyl-5-(trifluoromethyl) pyridin-2-yl)sulfamoyl) benzoic acid | 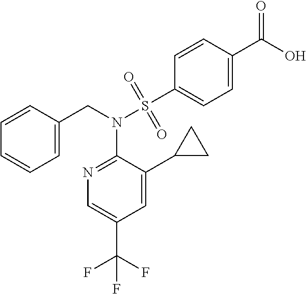 | 1.49 min (C) | 474.9 (M − H)⁻ |
| Example 150 | 4-(N-(3-Chloro-5-(trifluoro- methyl)pyridin-2-yl)-N-(4- cyclopropylbenzyl) sulfamoyl)benzoic acid | 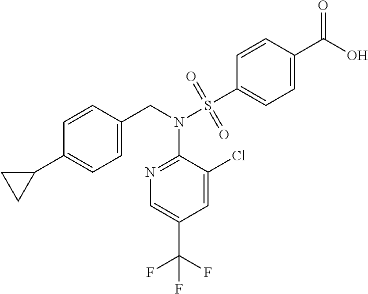 | 1.57 min (C) | 508.9 (M − H)⁻ |
| Example 151 | 4-(N-Benzyl-N-(3-bromo- 5-(trifluoromethyl) pyridin-2-yl)sulfamoyl) benzoic acid | 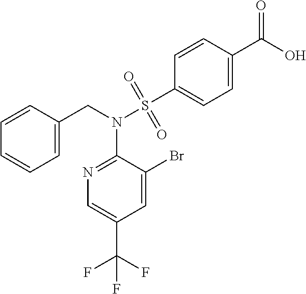 | 1.45 min (C) | 512.8 (M − H)⁻ |
| Example 152 | 4-(N-(3-Chloro-5- (trifluoromethyl)pyridin- 2-yl)-N-(4-(1-methyl-1H- pyrazol-4-yl)benzyl)sulfamoyl) benzoic acid | 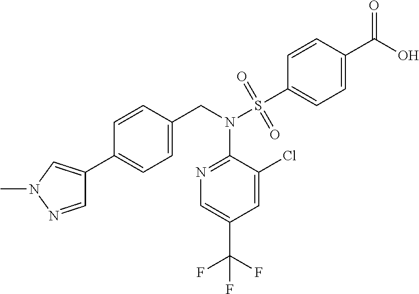 | 1.34 min (C) | 548.9 (M − H)⁻ |
| Example 153 | 4-(N-Benzyl-N-(3-methyl- 5-(trifluoromethyl)pyridin-2- yl)sulfamoyl)benzoic acid | 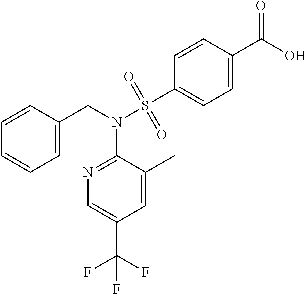 | 1.45 min (C) | 448.9 (M − H)⁻ |
| Example 154 | 4-(N-(3-Chloro-5-(trifluoro- methyl)pyridin-2-yl)-N- (4-(pyridin-3-yl)benzyl) sulfamoyl)benzoic acid | 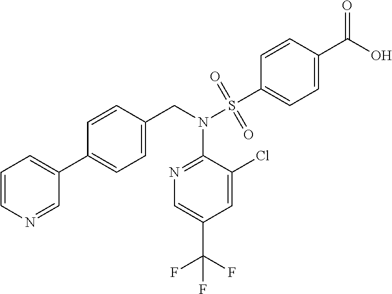 | 2.38 min (C) | 546.1 (M − H)⁻ |
| Example 155 | 4-(N-(3-Chloro-5-(trifluoro- methyl)pyridin-2-yl)-N- (4-(thiophen-2-yl)benzyl) sulfamoyl)benzoic acid | 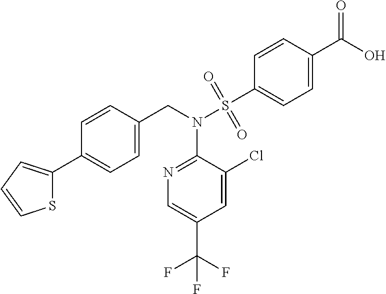 | 2.80 min (C) | 551.0 (M − H)⁻ |
| Example 156 | 4-(N-(3-Chloro-5-(trifluoro- methyl)pyridin-2-yl)-N-(4- (pyridin-4-yl)benzyl) sulfamoyl)benzoic acid | 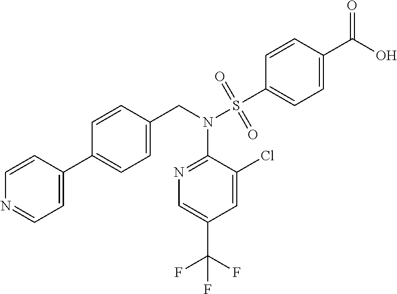 | 2.35 min (C) | 546.1 (M − H)⁻ |
| Example 157 | 4-(N-(3-Chloro-5-(trifluoro- methyl)pyridin-2-yl)-N-(4- (furan-2-yl)benzyl)sulfamoyl) benzoic acid | 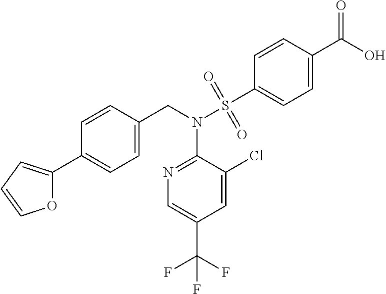 | 2.72 min (C) | 535.1 (M − H)⁻ |
| Example 158 | 4-(N-([1,1′-Biphenyl]-4- ylmethyl)-N-(3-chloro-5- (trifluoromethyl)pyridin- 2-yl)sulfamoyl)benzoic acid | 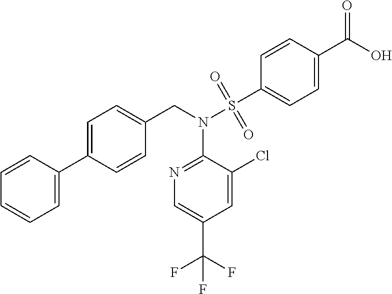 | 2.87 min (C) | 545.1 (M − H)⁻ |
| Example 159 | 4-(N-(3-Chloro-5-(trifluoro- methyl)pyridin-2-yl)-N-(4- (oxazol-5-yl)benzyl) sulfamoyl)benzoic acid | 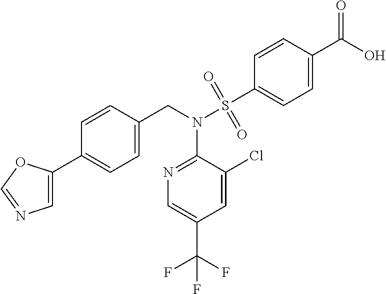 | 2.35 min (C) | 536.0 (M − H)⁻ |
| Example 160 | 4-(N-(3-Chloro-5-(trifluoro- methyl)pyridin-2-yl)-N-((6- (trifluoromethyl)pyridin-3- yl)methyl)sulfamoyl) benzoic acid | 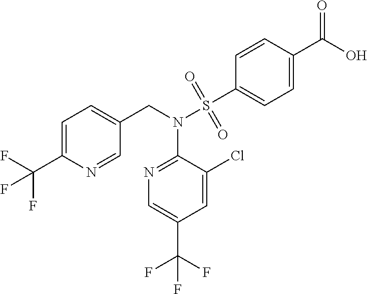 | 2.53 min (C) | 538.0 (M − H)⁻ |
| Example 161 | 4-(N-(3-Chloro-5-(trifluoro- methyl)pyridin-2-yl)-N-(4- (picolinamido)benzyl) sulfamoyl)benzoic acid | 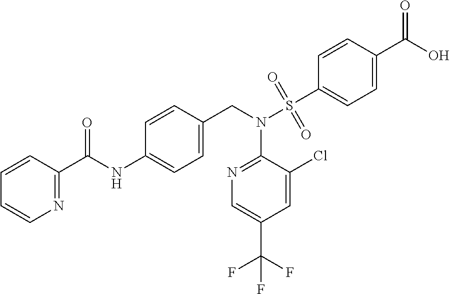 | 1.43 min (C) | 588.9 (M − H)⁻ |
| Example 162 | 4-(N-(3-Chloro-5-(trifluoro- methyl)pyridin-2-yl)-N-(4-(6- methoxypyridin-3-yl)benzyl) sulfamoyl)benzoic acid | 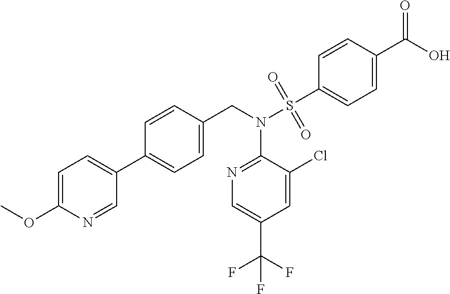 | 1.53 min (C) | 575.9 (M − H)⁻ |
| Example 163 | 4-(N-(3-Chloro-5-(trifluoro- methyl)pyridin-2-yl)-N-(4-(6- methylpyridin-3-yl)benzyl) sulfamoyl)benzoic acid | 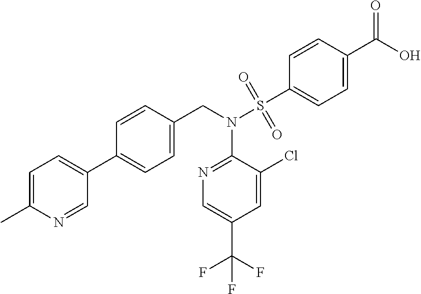 | 1.42 min (C) | 559.9 (M − H)⁻ |
| Example 164 | 4-(N-([1,1′-Biphenyl]-3- ylmethyl)-N-(3-chloro-5- (trifluoromethyl)pyridin- 2-yl)sulfamoyl)benzoic acid | 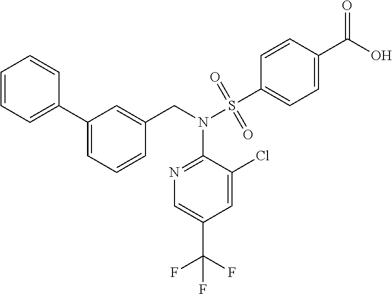 | 1.63 min (C) | 544.9 (M − H)⁻ |
| Example 165 | 4-(N-([1,1′-Biphenyl]-2- ylmethyl)-N-(3-chloro-5- (trifluoromethyl)pryidin- 2-yl)sulfamoyl)benzoic acid | 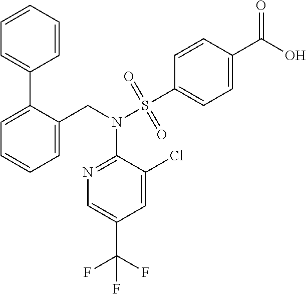 | 1.60 min (C) | 544.9 (M − H)⁻ |
| Example 166 | (R)-4-(N-(3-Chloro-5- (trifluoromethyl)pyridin-2- yl)-N-(1-phenylethyl) sulfamoyl)benozic acid | 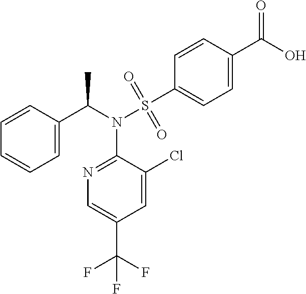 | 1.47 min (C) | 482.9 (M − H)⁻ |
| Example 167 | 4-(N-(3-Chloro-5-(trifluoro- methyl)pyridin-2-yl)-N- (thiophen-2-ylmethyl) sulfamoyl)benzoic acid | 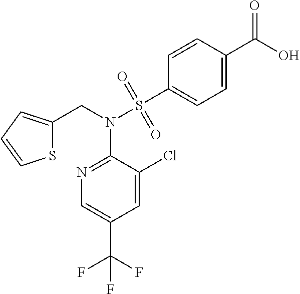 | 1.41 min (C) | 474.7 (M − H)⁻ |
| Example 168 | 4-(N-(3-Chloro-5-(trifluoro- methyl)pyridin-2-yl)-N- (cyclohexylmethyl)sulfamoyl) benzoic acid | 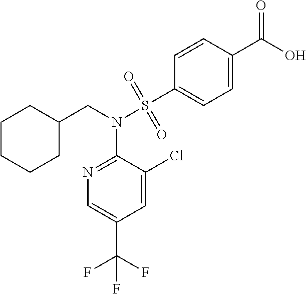 | 1.60 min (C) | 474.9 (M − H)⁻ |
| Example 169 | 4-(N-(3-Chloro-5-(trifluoro- methyl)pyridin-2-yl)-N-(4- phenoxybenzyl)sulfamoyl) benzoic acid | 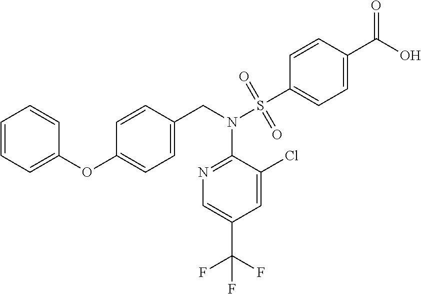 | 1.64 min (C) | 560.8 (M − H)⁻ |
| Example 170 | 4-(N-(4-(1H-Pyrazol-1-yl) benzyl)-N-(3-chloro-5- (trifluoromethyl)pyridin- 2-yl)sulfamoyl)benzoic acid | 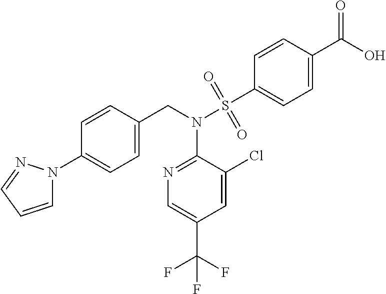 | 1.42 min (C) | 534.8 (M − H)⁻ |
| Example 171 | 4-(N-(3-Chloro-5-(trifluoro- methyl)pyridin-2-yl)-N- (cyclobutylmethyl) sulfamoyl)benzoic acid | 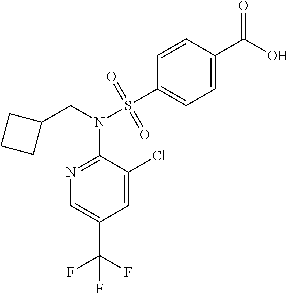 | 1.48 min (C) | 446.9 (M − H)⁻ |
| Example 172 | 4-(N-(3-Chloro-5-(trifluoro- methyl)pyridin-2-yl)-N- (thiophen-3-ylmethyl) sulfamoyl)benzoic acid | 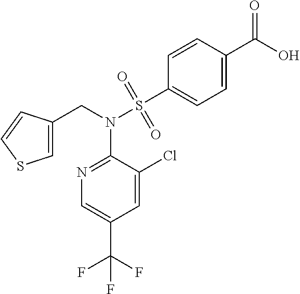 | 1.42 min (C) | 474.8 (M − H)⁻ |
| Example 173 | N-Benzyl-N-(3-chloro-5- (trifluoromethyl)pyridin- 2-yl)-4-cyanobenzene- sulfonamide | 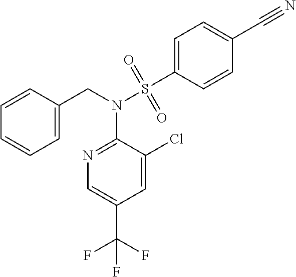 | 2.03 min (C) | 451.7 (M + H)⁺ |
| Example 174 | N-Benzyl-N-(3-chloro-5- (trifluoromethyl)pyridin- 2-yl)-4-(2H-tetrazol-5-yl) benzenesulfonamide | 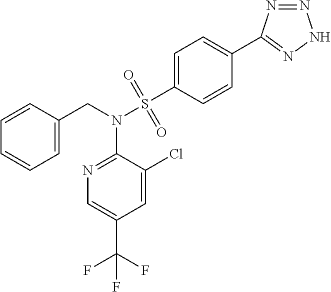 | 1.50 min (C) | 492.9 (M − H)⁻ |
| Example 175 | N-Benzyl-N-(3-chloro-5- (trifluoromethyl)pyridin- 2-yl)-4-methoxybenzene- sulfonamide | 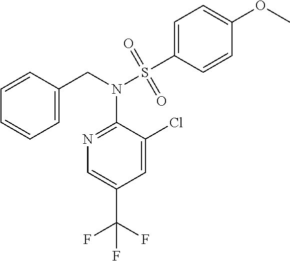 | 2.08 min (C) | 456.7 (M + H)⁺ |
| Example 176 | N-Benzyl-N-(3-chloro-5- (trifluoromethyl)pyridin- 2-yl)-4-(methylsulfonamido- methyl)benzenesulfonamide | 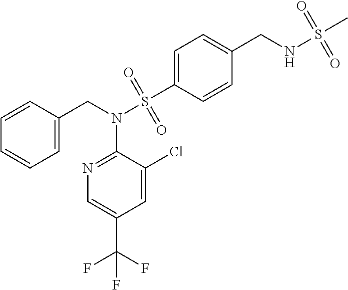 | 1.86 min (C) | 531.9 (M − H)⁻ |
| Example 177 | N-(4-(N-Benzyl-N-(3-chloro- 5-(trifluoromethyl)pyridin-2- yl)sulfamoyl)benzyl)acetamide | 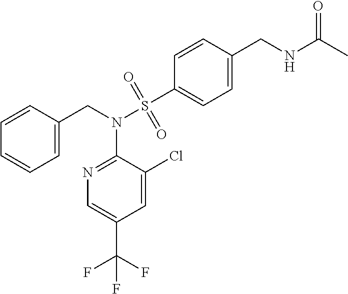 | 1.80 min (C) | 495.9 (M − H)⁻ |
| Example 178 | N-Benzyl-N-(3-chloro-5- (trifluoromethyl)pyridin-2- yl)-4-(((N,N-dimethyl- sulfamoyl)amino)methyl) benzenesulfonamide | 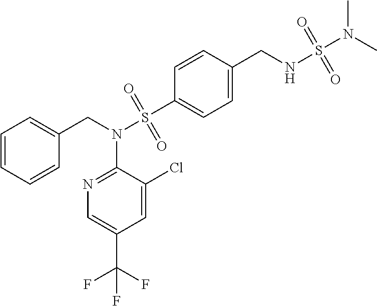 | 1.94 min (C) | 560.9 (M − H)⁻ |
| Example 179 | N-Benzyl-N-(3-chloro-5- (trifluoromethyl)pyridin- 2-yl)-4-((3,3-dimethylureido) methyl)benzenesulfonamide | 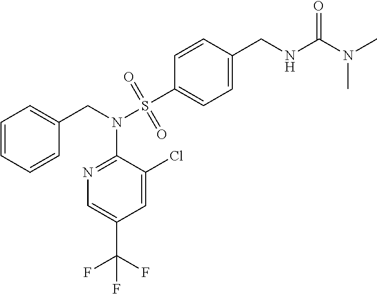 | 1.84 min (C) | 524.9 (M − H)⁻ |
| Example 180 | 4-(N-Benzyl-N-(5-bromo- 3-chloropyridin-2-yl) sulfamoyl)benzoic acid | 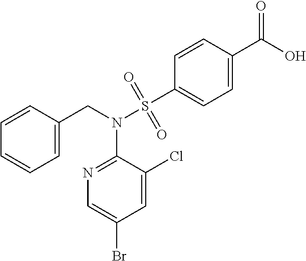 | 1.41 min (C) | 478.8 (M − H)⁻ |
| Example 181 | 4-(N-Benzyl-N-(3-chloro- 5-(trifluoromethyl)pyridin-2- yl)sulfamoyl)-3-chlorobenzoic acid | 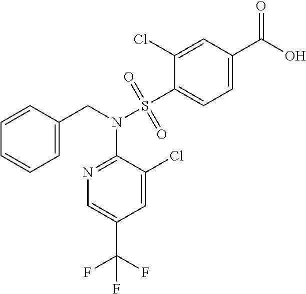 | 1.49 min (C) | 502.8 (M − H)⁻ |
| Example 182 | 4-(N-Benzyl-N-(3-chloro- 5-phenylpyridin-2-yl) sulfamoyl)benzoic acid | 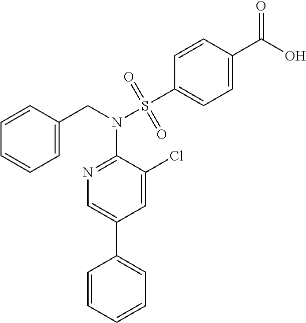 | 1.50 min (C) | 476.9 (M − H)⁻ |
| Example 183 | 4-(N-Benzyl-N-(3-chloro- 5-(furan-2-yl)pyridin-2- yl)sulfamoyl)benzoic acid | 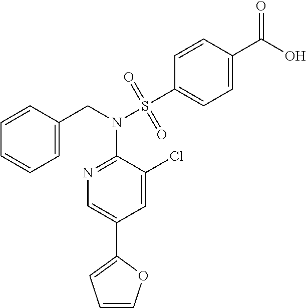 | 1.45 min (C) | 466.9 (M − H)⁻ |
| Example 184 | 4-(N-Benzyl-N-(3-chloro- 5-(thiophen-3-yl)pyridin- 2-yl)sulfamoyl)benzoic acid | 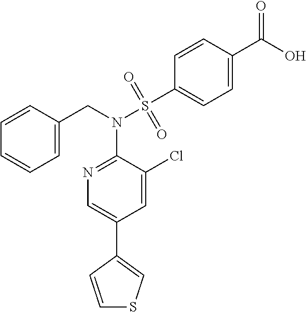 | 1.47 min (C) | 482.9 (M − H)⁻ |
| Example 185 | 4-(N-Benzyl-N-(3-chloro- 5-(2-methoxyphenyl)pyridin- 2-yl)sulfamoyl)benzoic acid | 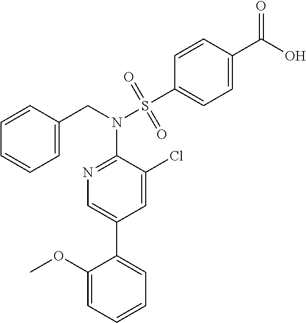 | 1.51 min (C) | 506.9 (M − H)⁻ |
| Example 186 | 4-(N-Benzyl-N-(3-chloro- 5-(4-methoxyphenyl)pyridin- 2-yl)sulfamoyl)benzoic acid | 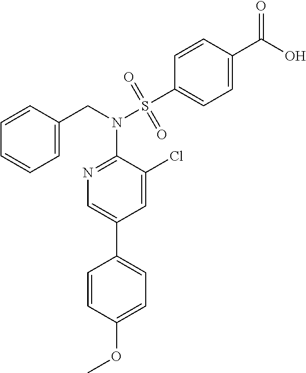 | 1.50 min (C) | 506.9 (M − H)⁻ |
| Example 187 | 4-(N-Benzyl-N-(3-chloro- 5-(3-methoxyphenyl)pyridin- 2-yl)sulfamoyl)benzoic acid | 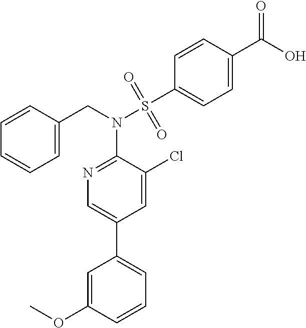 | 1.51 min (C) | 506.9 (M − H)⁻ |
| Example 188 | 4-(N-Benzyl-N-(3-chloro- 5-(trifluoromethyl)pyridin- 2-yl)sulfamoyl)-2-chloro- benzoic acid | 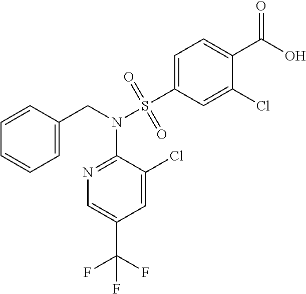 | 2.55 min (C) | 503.0 (M − H)⁻ |
| Example 189 | N-Benzyl-N-(3-chloro-5- (trifluoromethyl)pyridin- 2-yl)-6-methoxypyridine- 3-sulfonamide | 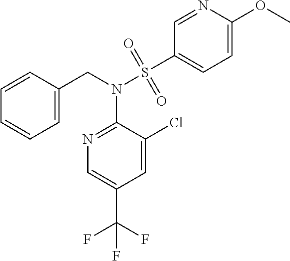 | 2.07 min (C) | 457.8 (M + H)⁺ |
| Example 190 | 4-(N-Benzyl-N-(3-chloro- 5-(trifluoromethyl)pyridin- 2-yl)sulfamoyl)-3-fluoro- benzoic acid | 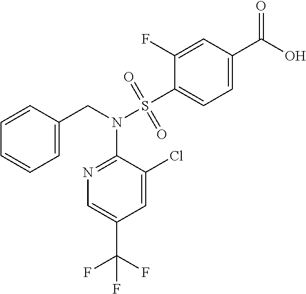 | 1.47 min (C) | 486.9 (M − H)⁻ |
| Example 191 | 4-(N-Benzyl-N-(3-chloro- 5-(trifluoromethyl)pyridin- 2-yl)sulfamoyl)-2-fluoro- benzoic acid | 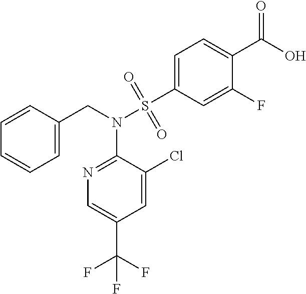 | 1.45 min (C) | 486.9 (M − H)⁻ |
| Example 192 | 4-(N-(3-Chloro-5-(trifluoro- methyl)pyridin-2-yl)-N- (cyclopentylmethyl)sulfamoyl) benzoic acid | 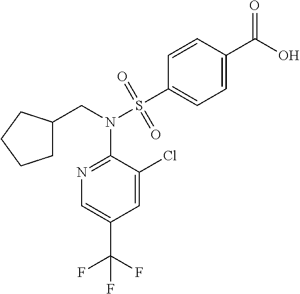 | 1.54 min (C) | 460.9 (M − H)⁻ |
| Example 193 | 4-(N-(3-Chloro-5-(trifluoro- methyl)pyridin-2-yl)-N-(1- phenylcyclopropyl) sulfamoyl)benzoic acid | 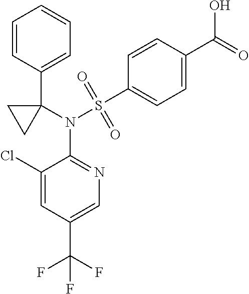 | 1.50 min (C) | 494.9 (M − H)⁻ |
| Example 194 | N-Benzyl-N-(3-chloro-5- (trifluoromethyl)pyridin- 2-yl)-4-((N,N-dimethyl- sulfamoyl)amino)benzene- sulfonamide | 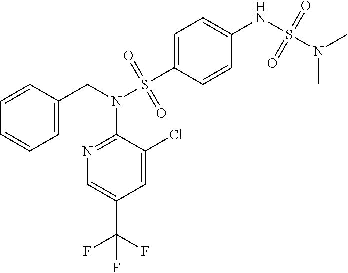 | 3.25 min (C) | 547.0 (M − H)⁻ |
| Example 195 | N-Benzyl-N-(3-chloro-5- (trifluoromethyl)pyridin- 2-yl)-4-ureidobenzene- sulfonamide | 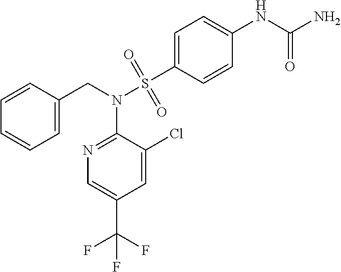 | 1.76 min (C) | 483.0 (M − H)⁻ |
| Example 196 | N-Benzyl-N-(3-chloro-5- (trifluoromethyl)pyridin- 2-yl)-4-(sulfamoylamino) benzenesulfonamide | 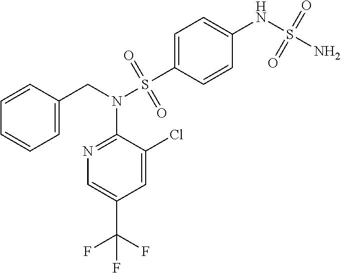 | 1.79 min (C) | 518.9 (M − H)⁻ |
| Example 197 | (S)-4-(N-(3-Chloro-5- (trifluoromethyl)pyridin-2- yl)-N-(1-phenethyl)sulfamoyl) benzoic acid | 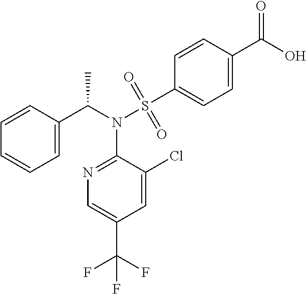 | 1.47 min (C) | 482.9 (M − H)⁻ |
| Example 198 | 4-(N-(3-Chloro-5-(trifluoro- methyl)pyridin-2-yl)-N-((2- phenylthiazol-4-yl)methyl) sulfamoyl)benzoic acid | 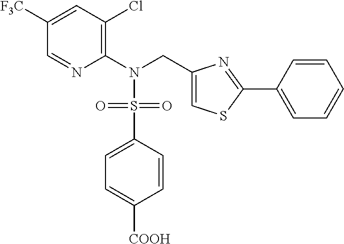 | 1.55 min (C) | 551.8 (M − H)⁻ |
| Example 199 | 4-(N-(3-Chloro-5-(trifluoro- methyl)pyridin-2-yl)-N-((5- phenyl-1,2,4-oxadiazol-3-yl) methyl)sulfamoyl)benzoic acid | 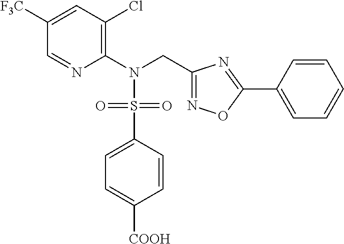 | 1.49 min (C) | 536.8 (M − H)⁻ |

In Vitro human TRPM8 Functional Assay

The functional activity of compounds was determined by measuring changes in intracellular calcium concentration using a Ca²⁺-sensitive fluorescent dye, Fluo4 (Molecular Probes). The changes in fluorescent signal were monitored by the cell imaging technology by Hamamatsu Photonics's Functional Drug Screening System (FDSS). Increases in intracellular Ca²⁺ concentration were readily detected upon activation with menthol.

### Cell Maintenance:

HEK293 cells stably expressing human TRPM8 were grown in T175 flasks, in a 5% CO₂ humidified incubator to about 80% confluence. Media composition consisted of Dulbecco's Modified Eagle Medium (high glucose), 10% fetal calf serum (FCS), 100 units/ml Penicillin, 100 microg/ml Streptomycin and 600 microg/ml Geneticine.

### Assay Protocol:

### Day One:

1. Plate-out HEK293-TRPM8 cells (40 microL medium containing 30,000 cells per well) into poly-D-lysine coated 384-well plates (BD FALCON) at 24 hours prior to assay.

### 2. Incubate at 37° C. in 5% CO₂.

### Day Two:

1. Wash each well with 80 microl of assay buffer (see bellow) twice and leave 20 microl using plate washer, ELx-405 Select CW (BIO-TEK).
2. Add 20 microL of assay buffer containing 0.5 microM Fluo-4-AM (Molecular Probes) and 0.005% Pluronic F-127 to each well.
3. Place the plate at room temperature in dark for 1 hour.
4. Wash each well with 80 microL of assay buffer (see bellow) twice and leave 20 microL using plate washer, ELx-405 Select CW (BIO-TEK).
5. Add 20 microL of compound solutions into each well and leave the plate for 5 minutes under the dark at room temperature.
6. Measure activity by FDSS as follows:
Set the assay plate on the stacker of FDSS
Start the detection of fluorescence intensity
After 30 sec, add 20 microL of 90 microM L-(−)-Menthol

IC₅₀ values for compounds of the present invention were determined from 11-point dose-response studies. Curves were generated using the average of duplicate wells for each data point. Finally, the IC₅₀ values are calculated with the best-fit dose curve determined by XLfit (ID Business Solutions Ltd.). The resultant data are displayed in Table 8.

TABLE 7
Assay buffer

| Regent | Final conc. | Volume (mL) |
|---|---|---|
| HEPES buffer, 1M | 19.4 mM | 20 |
| Probenecid | 2.5 mM | 10 |
| HBSS, 10x | 1 × HBSS | 100 |
| MQ water | — | 900 |

TABLE 8
Summary of the assay

| Example | IC50 |
|---|---|
| Example 1 | *** |
| Example 2 | * |
| Example 3 | * |
| Example 4 | * |
| Example 5 | *** |
| Example 6 | * |
| Example 7 | * |
| Example 8 | ** |
| Example 9 | *** |
| Example 10 | *** |
| Example 11 | *** |
| Example 12 | *** |
| Example 13 | *** |
| Example 14 | ** |
| Example 15 | *** |
| Example 16 | *** |
| Example 17 | *** |
| Example 18 | *** |
| Example 19 | *** |
| Example 20 | *** |
| Example 21 | ** |
| Example 22 | ** |
| Example 23 | *** |
| Example 24 | *** |
| Example 25 | *** |
| Example 26 | *** |
| Example 27 | *** |
| Example 28 | *** |
| Example 29 | ** |
| Example 30 | *** |
| Example 31 | *** |
| Example 32 | *** |
| Example 33 | *** |
| Example 34 | ** |
| Example 35 | *** |
| Example 36 | *** |
| Example 37 | ** |
| Example 38 | *** |
| Example 39 | *** |
| Example 40 | *** |
| Example 41 | *** |
| Example 42 | *** |
| Example 43 | *** |
| Example 44 | *** |
| Example 45 | *** |
| Example 46 | *** |
| Example 47 | *** |
| Example 48 | *** |
| Example 49 | *** |
| Example 50 | *** |
| Example 51 | *** |
| Example 52 | *** |
| Example 53 | *** |
| Example 54 | *** |
| Example 55 | *** |
| Example 56 | *** |
| Example 57 | ** |
| Example 58 | ** |
| Example 59 | *** |
| Example 60 | *** |
| Example 61 | *** |
| Example 62 | *** |
| Example 63 | *** |
| Example 64 | *** |
| Example 65 | *** |
| Example 66 | *** |
| Example 67 | *** |
| Example 68 | *** |
| Example 69 | *** |
| Example 70 | *** |
| Example 71 | *** |
| Example 72 | *** |
| Example 73 | * |
| Example 74 | ** |
| Example 75 | *** |
| Example 76 | *** |
| Example 77 | *** |
| Example 78 | *** |
| Example 79 | *** |
| Example 80 | ** |
| Example 81 | ** |
| Example 82 | ** |
| Example 83 | ** |
| Example 84 | ** |
| Example 85 | ** |
| Example 86 | ** |
| Example 87 | ** |
| Example 88 | *** |
| Example 89 | ** |
| Example 90 | ** |
| Example 91 | ** |
| Example 92 | ** |
| Example 93 | ** |
| Example 94 | ** |
| Example 95 | * |
| Example 96 | ** |
| Example 97 | ** |
| Example 98 | ** |
| Example 99 | ** |
| Example 100 | ** |
| Example 101 | ** |
| Example 102 | ** |
| Example 103 | ** |
| Example 104 | * |
| Example 105 | ** |
| Example 106 | ** |
| Example 107 | ** |
| Example 108 | ** |
| Example 109 | ** |
| Example 110 | ** |
| Example 111 | ** |
| Example 112 | * |
| Example 113 | ** |
| Example 114 | * |
| Example 115 | ** |
| Example 116 | * |
| Example 117 | * |
| Example 118 | ** |
| Example 119 | * |
| Example 120 | ** |
| Example 121 | ** |
| Example 122 | ** |
| Example 123 | * |
| Example 124 | ** |
| Example 125 | ** |
| Example 126 | * |
| Example 127 | ** |
| Example 128 | ** |
| Example 129 | ** |
| Example 130 | ** |
| Example 131 | ** |
| Example 132 | ** |
| Example 133 | ** |
| Example 134 | ** |
| Example 135 | ** |
| Example 136 | ** |
| Example 137 | * |
| Example 138 | ** |
| Example 139 | ** |
| Example 140 | *** |
| Example 141 | *** |
| Example 142 | *** |
| Example 143 | *** |
| Example 144 | *** |
| Example 145 | *** |
| Example 146 | *** |
| Example 147 | *** |
| Example 148 | *** |
| Example 149 | *** |
| Example 150 | *** |
| Example 151 | *** |
| Example 152 | *** |
| Example 153 | *** |
| Example 154 | *** |
| Example 155 | *** |
| Example 156 | *** |
| Example 157 | *** |
| Example 158 | *** |
| Example 159 | *** |
| Example 160 | *** |
| Example 161 | *** |
| Example 162 | *** |
| Example 163 | *** |
| Example 164 | *** |
| Example 165 | *** |
| Example 166 | *** |
| Example 167 | *** |
| Example 168 | *** |
| Example 169 | *** |
| Example 170 | *** |
| Example 171 | *** |
| Example 172 | *** |
| Example 173 | *** |
| Example 174 | *** |
| Example 175 | *** |
| Example 176 | *** |
| Example 177 | *** |
| Example 178 | *** |
| Example 179 | *** |
| Example 180 | *** |
| Example 181 | *** |
| Example 182 | *** |
| Example 183 | *** |
| Example 184 | *** |
| Example 185 | *** |
| Example 186 | *** |
| Example 187 | *** |
| Example 188 | *** |
| Example 189 | *** |
| Example 190 | *** |
| Example 191 | *** |
| Example 192 | *** |
| Example 193 | *** |
| Example 194 | *** |
| Example 195 | *** |
| Example 196 | *** |
| Example 197 | *** |
| Example 198 | *** |
| Example 199 | *** |

IC50:
*** <1 microM,
** 1 to10 microM,
* 10 to 30 microM

### Chronic Constriction Injury (CCI)-Induced Model of Neuropathic Pain; Cold Allodynia

Male Sprague-Dawley rats were housed in groups of two under a 12-h light/dark cycle (lights on at 07:00) with free access to food and water ad libitum. The CCI was made according to the method of Bennett G J and Xie Y K (Pain 1988, 33: 87-107). Rats (253-322 g) were anesthetized with intraperitoneal injection of sodium pentobarbital. The left common sciatic nerve was exposed at the level of the middle of the thigh and four ligatures were loosely tided around it by using 4-0 silk thread with about 1 mm space. Sham operation was performed in the same manner except of sciatic nerve ligation. Seven days following CCI surgery, cold allodynia was assessed using a cold plate (LHP-1700CP, TECA, USA) with a temperature controller (Model 3300-0, CAL Controls Inc, USA) as described by Tanimoto-Mori S et al. (Behav Pharmacol. 2008, 19: 85-90). The animals were habituated to the apparatus which consisted of a transparent acrylic box (10×12×12 cm) on a stainless-steel plate (15×33 cm). The surface of the cold plate held on 10° C. and the temperature of the plate was monitored continuously with a precision of 0.1° C. For testing, the rat was placed on the cold plate and the paw withdrawal latency (PWL) was measured before and after the compound administration, with a cut-off value of 120 sec. The compounds of the invention or their vehicles were administered perorally, subcutaneously or intraperitoneally. The percentages of inhibition were calculated as follows;

$$Inhibition ( \% ) = \frac{{PWL}_{drug} - {PWL}_{vehicle}}{{PWL}_{sham} - {PWL}_{vehicle}} \times 100. \qquad \{ Math . 1 \}$$

Some compounds of the invention showed potent activities in this model (>30% inhibition).

### Chronic Constriction Injury (CCI)-Induced Model of Neuropathic Pain; Static Allodynia

Male Sprague-Dawley rats were housed in groups of two under a 12-h light/dark cycle (lights on at 07:00) with free access to food and water ad libitum. The CCI was made according to the method of Bennett G J and Xie Y K (Pain 1988, 33: 87-107). Rats (253-322 g) were anesthetized with intraperitoneal injection of sodium pentobarbital. The left common sciatic nerve was exposed at the level of the middle of the thigh and four ligatures were loosely tided around it by using 4-0 silk thread with about 1 mm space. Sham operation was performed in the same manner except of sciatic nerve ligation. Static allodynia was assessed using von Frey hairs (VFHs) at 14 days following CCI surgery as described by Field M J et al. (Pain 1999, 83: 303-311). The animals were habituated to grid bottom cages prior to the start of experiment. VFHs in ascending order of force (0.16, 0.4, 0.6, 1, 1.4, 2, 4, 6, 8, 10, 15 and 26 g) were applied to the plantar surface of the hind paw. Each VFH was applied to the ipsilateral paw for 6 seconds or until a withdrawal response occurred. Once a withdrawal response was happened, the paw was re-test, starting with the next descending VFH until no response occurred. The lowest amount of force required to elicit a response was recorded as paw withdrawal threshold (PWT). Static allodynia was defined as present if animals responded to or below the innocuous 1.4 gram VFH. The compounds of the invention or their vehicles were administered perorally, subcutaneously or intraperitonealy. The percentages of inhibition were calculated as follows;

$$Inhibition ( \% ) = \frac{{log}_{10} ( {PWT}_{drug} ) - {log}_{10} ( {PWT}_{vehicle} )}{{log}_{10} ( {PWT}_{sham} ) - {log}_{10} ( {PWT}_{vehicle} )} \times 100. \qquad \{ Math . 2 \}$$

Some compounds of the invention showed potent activities in this model (>30% inhibition).

### Oxaliplatin-Induced Model of Neuropathic Pain; Cold and Mechanical Allodynia

Male C57BL/6 mice or Sprague-Dawley rats 6 or 7 weeks old at the start of the experiment, were used. They were housed in groups under a 12-h light/dark cycle (lights on at 07:00) with free access to food and water ad libitum. The study was conducted according to the method of Gauchan, P et al. (Gauchan, P., et al. NeuroSci Lett, 2009, 458, 93-95). Oxaliplatin (Sigma, St. Louis, Mo.) was dissolved in 5% glucose. Oxaliplatin (3-4 mg/kg) was injected intraperitoneally once or twice a week for one or two-week. For the assessment of cold allodynia, licking time or behavioral score to acetone or PWL and paw withdrawal count on cold plate were measured. For acetone test, the animal was held by the hand and a droplet (50 microL) of acetone, formed on the flat-tip needle of a syringe, was gently touched to the plantar surface of the hind paw. The animal was immediately put in an acrylic cage with a transparent floor and the behaviors were videotaped from beneath. Measurement of time spent in licking of the plantar region over a 60 s period was made by video playback. Acetone was applied three times at a 10-15 min interval and the average of licking time was calculated. For cold plate test, cold allodynia was assessed using a cold plate (LHP-1700CP, TECA, USA) with a temperature controller (Model 3300-0, CAL Controls Inc, USA) as described by Tanimoto-Mori S et al. (Behav Pharmacol. 2008, 19: 85-90). The animals were habituated to the apparatus which consisted of a transparent acrylic box (10×12×12 cm) on a stainless-steel plate (15×33 cm). The surface of the cold plate held on 10° C. and the temperature of the plate was monitored continuously with a precision of 0.1° C. For testing, the animal was placed on the cold plate and PWL was measured before and after the compound administration, with a cut-off value of 180 sec. Mechanical allodynia was assessed using VFHs. The animals were habituated to grid or mesh bottom cages prior to the start of experiment. VFHs in ascending order of force (0.16, 0.4, 0.6, 1, 1.4, 2, 4, 6, 8, 10, 15 and 26 g) were applied to the plantar surface of the hind paw. Once a withdrawal response was happened, the paw was re-test, starting with the next descending VFH until no response occurred. The lowest amount of force required to elicit a response was recorded as paw withdrawal threshold (PWT). For testing, PWT was measured before and after the compound administration. The compounds of the invention or their vehicles were administered perorally, subcutaneously or intraperitonealy.

Compounds of the invention showed potent activities on cold or mechanical allodynia in this model.

All publications, including but not limited to, issued patents, patent applications, and journal articles, cited in this application are each herein incorporated by reference in their entirety. Although the invention has been described above with reference to the disclosed embodiments, those skilled in the art will readily appreciate that the specific experiments detailed are only illustrative of the invention. It should be understood that various modifications can be made without departing from the spirit of the invention. Accordingly, the invention is limited only by the following claims.

1-2. (canceled)

3. A method for the treatment of a condition or disorder mediated by TRPM8 receptor antagonistic activity, in a mammalian subject, including a human, which comprises administering to a mammal in need of such treatment a therapeutically effective amount of the compound of formula (I), 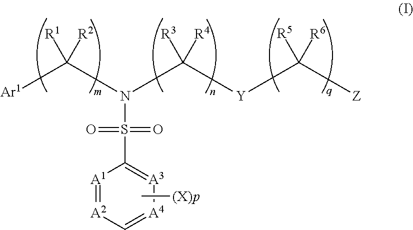
wherein
R¹, R², R³,
R⁴, R⁵, and R⁶ are independently selected from the group consisting of hydrogen, C₁-C₄ alkyl, hydroxy C₁-C₄ alkyl, C₁-C₄ alkoxy C₁-C₄ alkyl, and C₃-C₇ cycloalkyl; or alternatively R¹ and R², together with the atom to which they are attached, form a 3 to 6 membered ring which may contain oxygen and/or nitrogen; said ring is optionally substituted with 1 to 4 substituents independently selected from the group consisting of halogen, hydroxy, C₁-C₄ alkyl, and C₁-C₄ alkoxy; R³ and R⁴, together with the atom to which they are attached, form a 3 to 6 membered ring which may contain oxygen and/or nitrogen; said ring is optionally substituted with 1 to 4 substituents independently selected from the group consisting of halogen, hydroxy, C₁-C₄ alkyl, and C₁-C₄ alkoxy; R⁵ and R⁶, together with the atom to which they are attached, form a 3 to 6 membered ring which may contain oxygen and/or nitrogen; said ring is optionally substituted with 1 to 4 substituents independently selected from the group consisting of halogen, hydroxy, C₁-C₄ alkyl, and C₁-C₄ alkoxy;
m is 0 or 1;
n is 0, 1, 2 or 3;
q is 0, 1, 2 or 3;
A¹, A², A³ and A⁴ are independently selected from nitrogen atom and carbon atom; wherein the number of nitrogen is up to two;
Z is H, Ar² or a substituent represented by the formula: R⁷N(R⁸)C(═O)—, in which
R⁷ and R⁸ are independently selected from hydrogen, C₁-C₄ alkyl, hydroxy C₁-C₄ alkyl, C₁-C₄ alkoxy C₁-C₄ alkyl, amino C₁-C₄ alkyl, C₁-C₄ alkylamino C₁-C₄ alkyl, di(C₁-C₄ alkyl)amino C₁-C₄ alkyl, 5 to 10 membered aryl, 5 to 10 membered aryl C₀-C₄ alkyl;
said aryl may be optionally substituted with 1 to 5 substituents independently selected from the group consisting of hydroxy, halogen, C₁-C₄ alkyl, C₁-C₄ alkoxy, hydroxy C₁-C₄ alkyl, amino C₁-C₄ alkyl, C₁-C₄ haloalkyl, C₁-C₄ haloalkoxy, C₃-C₈ cycloalkyl, amino, C₁-C₄ alkylamino, di(C₁-C₄ alkyl)amino, C₁-C₄ alkylthio, and nitro;
C₃-C₈ cycloalkyl, and 3 to 8 membered heterocyclyl C₁-C₄ alkyl;
said heterocyclyl and alkyl may have independently 1 to 4 substituents independently selected from C₁-C₄ alkyl and halogen;
or alternatively R⁷ and R⁸ together with nitrogen atom to which they are attached form a 4 to 8 membered ring which may contain nitrogen, oxygen or sulfur, wherein the 4 to 8 membered ring is optionally substituted with 1 to 6 substituents independently selected from the group consisting of hydroxy, C₁-C₄ alkyl, C₁-C₄ alkoxy, C₃-C₇ cycloalkyl, amino, oxo, C₁-C₄ alkylamino, and di(C₁-C₄ alkyl)amino;
Ar¹ is aryl, which may optionally be substituted with halogen, C₁-C₄ alkyl, C₁-C₄ haloalkyl, hydroxy, C₁-C₄ alkoxy, C₁-C₄ haloalkoxy, C₁-C₄ alkylthio, nitro, amino, C₁-C₄ alkylamino, di(C₁-C₄ alkyl)amino, cyano, hydroxy C₁-C₄ alkyl, C₁-C₄ alkoxy C₁-C₄ alkyl, C₁-C₄ alkylsulfonyl, aminosulfonyl, C₁-C₄ alkyl C(═O)—, HO(O═)C—, C₁-C₄ alkyl-O(O═)C—, R⁹N(R¹⁰)C(═O)—, C₁-C₄ alkylsulfonylamino, C₃-C₇ cycloalkyl, R⁹C(═O)N(R¹⁰)—, NH₂(HN═)C—, or 5 to 10 membered aryl C₀-C₄ alkyl; said aryl may be optionally substituted with 1 to 5 substituents independently selected from the group consisting of hydroxy, halogen, C₁-C₄ alkyl, C₁-C₄ alkoxy, hydroxy C₁-C₄ alkyl, amino C₁-C₄ alkyl, C₁-C₄ haloalkyl, C₁-C₄ haloalkoxy, C₃-C₇ cycloalkyl, amino, C₁-C₄ alkylamino, di(C₁-C₄ alkyl)amino, C₁-C₄ alkylthio, and nitro;
Ar² is aryl, which may optionally be substituted with halogen, C₁-C₄ alkyl, C₁-C₄ haloalkyl, hydroxy, C₁-C₄ alkoxy, C₁-C₄ haloalkoxy, C₁-C₄ alkylthio, nitro, C₁-C₄ alkylsilyl, di(C₁-C₄ alkyl)silyl, tri(C₁-C₄ alkyl)silyl, amino, C₁-C₄ alkylamino, di(C₁-C₄ alkyl)amino, cyano, hydroxy C₁-C₄ alkyl, C₁-C₄ alkoxy C₁-C₄ alkyl, C₁-C₄ alkylsulfonyl, aminosulfonyl, C₁-C₄ alkyl C(═O)—, HO(O═)C—, C₁-C₄ alkyl-O(O═)C—, R⁹N(R¹⁰)C(═O)—, C₁-C₄ alkylsulfonylamino, C₃-C₇ cycloalkyl, R⁹C(═O)N(R¹⁰)—, NH₂(HN═)C—, 5 to 10 membered aryloxy or 5 to 10 membered aryl C₀-C₄ alkyl; said aryloxy, aryl and C₃-C₇ cycloalkyl may be optionally substituted with 1 to 5 substituents independently selected from the group consisting of hydroxy, halogen, C₁-C₄ alkyl, C₁-C₄ alkoxy, hydroxy C₁-C₄ alkyl, amino C₁-C₄ alkyl, C₁-C₄ haloalkyl, C₁-C₄ haloalkoxy, C₃-C₇ cycloalkyl, cyano, amino, C₁-C₄ alkylamino, di(C₁-C₄ alkyl)amino, C₁-C₄ alkylthio, R⁹N(R¹⁰)C(═O)— and nitro;
R⁹ and R¹⁰ are independently selected from the definitions of R⁷ and R⁸;
X is independently selected from HO(O═)C—C₀-C₄alkyl, hydroxy, halogen, C₁-C₄ alkyl, C₁-C₄ alkoxy, hydroxy C₁-C₄ alkyl, amino C₁-C₄ alkyl, C₁-C₄ haloalkyl, C₁-C₄ haloalkoxy, C₃-C₇ cycloalkyl, cyano, amino, C₁-C₄ alkylamino, di(C₁-C₄ alkyl)amino, C₁-C₄ alkylthio, nitro, alkylsulfonyl, aminosulfonyl, C₁-C₄ alkyl C(═O)—, C₁-C₄ alkyl-O(O═)C—, R¹¹N(R¹²)C(═O)—, C₁-C₄ alkylsulfonylamino, C₁-C₄ alkylsulfonylaminoalkyl, C₃-C₇ cycloalkyl, R¹¹C(═O)N(R¹²)—, R¹¹C(═O)N(R¹²)C₁-C₄alkyl, R¹¹N(R¹²)SO₂N(R¹³)C₀-C₄alkyl, R¹¹N(R¹²)C(═O)N(R¹³)C₀-C₄alkyl, NH₂(HN═)C—, C₃-C₇ cycloalkyl, 3 to 7 membered heterocyclyl, and 5 to 10 membered aryl C₀-C₄ alkyl; said heterocyclyl and alkyl may have independently 1 to 4 substituents independently selected from C₁-C₄ alkyl and halogen;
R¹¹, R¹² and R¹³ are independently selected from the definitions of R⁷ and R⁸;
p is 1, 2, 3, 4 or 5; when p is two or more than two, X may be same or different;
Y is a chemical bond, oxygen atom, sulfur atom, or nitrogen atom; when Y is oxygen atom, sulfur atom, or nitrogen atom, said substituent Y may have a substituent independently selected from the definitions of R⁷ and R⁸;
or a pharmaceutically acceptable salt thereof.

4. The method as claimed in claim 3, wherein said condition or disorder is one or more of inflammatory, pain and urological diseases or disorders, including chronic pain, neuropathic pain including cold allodynia and diabetic neuropathy, postoperative pain, osteoarthritis, rheumatoid arthritic pain, cancer pain, neuralgia, neuropathies, algesia, nerve injury, migraine, cluster and tension headaches, ischaemia, irritable bowel syndrome, neurodegeneration, fibromyalgia, stroke, itch, psychiatric disorders including anxiety and depression and inflammatory disorders such as asthma and chronic obstructive pulmonary or airways, disease i.e., COPD, pulmonary hypertension, anxiety, including other stress-related disorders, urological diseases or disorders such as detrusor overactivity or overactive bladder, urinary incontinence, neurogenic detrusor overactivity or detrusor hyperflexia, idiopathic detrusor overactivity or detrusor instability, benign prostatic hyperplasia, and lower urinary tract symptoms, and combinations thereof.

5. A compound of formula (I) 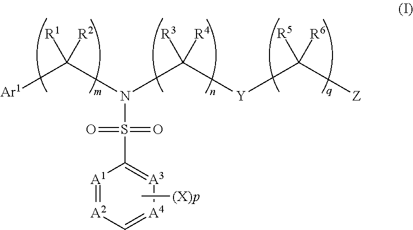
wherein
R¹, R², R³, R⁴, R⁵, and R⁶ are independently selected from the group consisting of hydrogen, C₁-C₄ alkyl, hydroxy C₁-C₄ alkyl, C₁-C₄ alkoxy C₁-C₄ alkyl, and C₃-C₇ cycloalkyl; or alternatively R¹ and R², together with the atom to which they are attached, form a 3 to 6 membered ring which may contain oxygen and/or nitrogen; said ring is optionally substituted with 1 to 4 substituents independently selected from the group consisting of halogen, hydroxy, C₁-C₄ alkyl, and C₁-C₄ alkoxy; R³ and R⁴, together with the atom to which they are attached, form a 3 to 6 membered ring which may contain oxygen and/or nitrogen; said ring is optionally substituted with 1 to 4 substituents independently selected from the group consisting of halogen, hydroxy, C₁-C₄ alkyl, and C₁-C₄ alkoxy; R⁵ and R⁶, together with the atom to which they are attached, form a 3 to 6 membered ring which may contain oxygen and/or nitrogen; said ring is optionally substituted with 1 to 4 substituents independently selected from the group consisting of halogen, hydroxy, C₁-C₄ alkyl, and C₁-C₄ alkoxy;
m is 0;
n is 0, 1, 2 or 3;
q is 0, 1, 2 or 3;
A¹, A², A³ and A⁴ are independently selected from nitrogen atom and carbon atom; wherein the number of nitrogen is up to two;
Z is H, Ar² or a substituent represented by the formula: R⁷N(R⁸)C(═O)—, in which
R⁷ and R⁸ are independently selected from hydrogen C₁-C₄ alkyl, hydroxy C₁-C₄ alkyl, C₁-C₄ alkoxy C₁-C₄ alkyl, amino C₁-C₄ alkyl, C₁-C₄ alkylamino C₁-C₄ alkyl, di(C₁-C₄ alkyl)amino C₁-C₄ alkyl, 5 to 10 membered aryl, 5 to 10 membered aryl C₀-C₄ alkyl;
said aryl may be optionally substituted with 1 to 5 substituents independently selected from the group consisting of hydroxy, halogen, C₁-C₄ alkyl, C₁-C₄ alkoxy, hydroxy C₁-C₄ alkyl, amino C₁-C₄ alkyl, C₁-C₄ haloalkyl, C₁-C₄ haloalkoxy, C₃-C₈ cycloalkyl, amino, C₁-C₄ alkylamino, di(C₁-C₄ alkyl)amino, C₁-C₄ alkylthio, and nitro;
C₁-C₈ cycloalkyl, and 3 to 8 membered heterocyclyl C₁-C₄ alkyl;
said heterocyclyl and alkyl may have independently 1 to 4 substituents independently selected from C₁-C₄ alkyl and halogen;
or alternatively R⁷ and R⁸ together with nitrogen atom to which they are attached form a 4 to 8 membered ring which may contain nitrogen, oxygen or sulfur, wherein the 4 to 8 membered ring is optionally substituted with 1 to 6 substituents independently selected from the group consisting of hydroxy, C₁-C₄ alkyl, C₁-C₄ alkoxy, C₃-C₇ cycloalkyl, amino, oxo, C₁-C₄ alkylamino, and di(C₁-C₄ alkyl)amino;
Ar¹ is 5 to 7 membered heterocyclic group, which may optionally be substituted with halogen, C₁-C₄ alkyl, C₁-C₄ haloalkyl, hydroxy, C₁-C₄ alkoxy, C₁-C₄ haloalkoxy, C₁-C₄ alkylthio, nitro, amino, C₁-C₄ alkylamino, di(C₁-C₄ alkyl)amino, cyano, hydroxy C₁-C₄ alkyl, C₁-C₄ alkoxy C₁-C₄ alkyl, C₁-C₄ alkylsulfonyl, aminosulfonyl, C₁-C₄ alkyl C(═O)C—, HO(O═)C—, C₁-C₄ alkyl-O(O═)C—, R⁹N(R¹⁰)C(═O)—, C₁-C₄ alkylsulfonylamino, C₃-C₇ cycloalkyl, R⁹C(═O)N(R¹⁰)—, NH₂(HN═)C—, or 5 to 10 membered aryl C₀-C₄ alkyl; may be optionally substituted with 1 to 5 substituents independently selected from the group consisting of hydroxy, halogen, C₁-C₄ alkyl, C₁-C₄ alkoxy, hydroxy C₁-C₄ alkyl, amino C₁-C₄ alkyl, C₁-C₄ haloalkyl, C₁-C₄ haloalkoxy, C₃-C₇ cycloalkyl, amino, C₁-C₄ alkylamino, di(C₁-C₄ alkyl)amino, C₁-C₄ alkylthio, and nitro;
Ar² is aryl, which may optionally be substituted with halogen, C₁-C₄ alkyl, C₁-C₄ haloalkyl, hydroxy, C₁-C₄ alkoxy, C₁-C₄ haloalkoxy, C₁-C₄ alkylthio, nitro, C₁-C₄ alkylsilyl, di(C₁-C₄ alkyl)silyl, tri(C₁-C₄ alkyl)silyl, amino, C₁-C₄ alkylamino, di(C₁-C₄ alkyl)amino, cyano, hydroxy C₁-C₄ alkyl, C₁-C₄ alkoxy C₁-C₄ alkyl, C₁-C₄ alkylsulfonyl, aminosulfonyl, C₁-C₄ alkyl C(═O)—, HO(O═)C—, C₁-C₄ alkyl-O(O═)C—R⁹N(R¹⁰)C(═O)—C₁-C₄ alkylsulfonylamino C₃-C₇ cycloalkyl, R⁹C(═O)N(R¹⁰)—, NH₂(HN═)C—, 5 to 10 membered aryloxy or 5 to 10 membered aryl C₀-C₄ alkyl; said aryloxy, aryl and C₃-C₇ cycloalkyl may be optionally substituted with 1 to 5 substituents independently selected from the group consisting of hydroxy, halogen, C₁-C₄ alkyl, C₁-C₄ alkoxy, hydroxy C₁-C₄ alkyl, amino C₁-C₄ alkyl, C₁-C₄ haloalkyl, C₁-C₄ haloalkoxy, C₃-C₇ cycloalkyl, cyano, amino, C₁-C₄ alkylamino, di(C₁-C₄ alkyl)amino, C₁-C₄ alkylthio, R⁹N(R¹⁰)C(═O)— and nitro;
R⁹ and R¹⁰ are independently selected from the definitions of R⁷ and R⁸;
X is independently selected from HO(O═)C—C₀-C₄ alkyl, hydroxy, halogen, C₁-C₄ alkyl, C₁-C₄ alkoxy, hydroxy C₁-C₄ alkyl, amino C₁-C₄ alkyl, C₁-C₄ haloalkyl, C₁-C₄ haloalkoxy, C₃-C₇ cycloalkyl, cyano, amino, C₁-C₄ alkylamino, di(C₁-C₄ alkyl)amino, C₁-C₄ alkylthio, nitro, alkylsulfonyl, aminosulfonyl, C₁-C₄ alkyl C(═O)—, C₁-C₄ alkyl-O(O═)C—, R¹¹N(R¹²)C(═O)—, C₁-C₄ alkylsulfonylamino, C₁-C₄ alkylsulfonylaminoalkyl, C₁-C₇ cycloalkyl, R¹¹C(═O)N(R¹²)—, R¹¹C(═O)N(R¹²)C₁-C₄alkyl, R¹¹N(R¹²)SO₂N(R¹³)C₀-C₄alkyl, R¹¹N(R¹²)C(═O)N(R¹³)C₀-C₄alkyl, NH₂(HN═)C—, C₃-C₇ cycloalkyl, 3 to 7 membered heterocyclyl, and 5 to 10 membered aryl C₀-C₄ alkyl; said heterocyclyl and alkyl may have independently 1 to 4 substituents independently selected from C₁-C₄ alkyl and halogen;
R¹¹, R¹² and R¹³ are independently selected from the definitions of R⁷ and R⁸;
p is 1, 2, 3, 4 or 5; when p is two or more than two, X may be same or different;
Y is a chemical bond, oxygen atom, sulfur atom, or nitrogen atom; when Y is oxygen atom, sulfur atom, or nitrogen atom, said substituent Y may have a substituent independently selected from the definitions of R⁷ and R⁸;
or a pharmaceutically acceptable salt thereof.

6. The compound of formula (I) as claimed in claim 5, wherein m is 0; and Ar¹ is a 5 to 7 heterocyclic group selected from pyridinyl, pyrimidinyl, pyridazinyl, and triazinyl.

7. The compound of formula (I) as claimed in claim 6, wherein m is 0; Ar¹ is 2-pyridinyl or 3-pyridinyl; and A¹, A², A³ and A⁴ are carbon atom.

8. The compound as claimed in claim 5 which is selected from:
N-Benzyl-N-(3-chloro-5-(trifluoromethyl)pyridin-2-yl)benzenesulfonamide;
4-(N-Benzyl-N-(3-chloro-5-(trifluoromethyl)pyridin-2-yl)sulfamoyl)benzamide;
4-(N-Benzyl-N-(3-chloro-5-(trifluoromethyl)pyridin-2-yl)sulfamoyl)-N-methylbenzamide;
4-(N-Benzyl-N-(3-chloro-5-(trifluoromethyl)pyridin-2-yl)sulfamoyl)-N,N-dimethylbenzamide;
4-(N-Benzyl-N-(3-chloro-5-(trifluoromethyl)pyridin-2-yl)sulfamoyl)-N-(2-hydroxyethyl)benzamide;
4-(N-(3-Chloro-5-(trifluoromethyl)pyridin-2-yl)-N-(4-(trifluoromethoxy)benzyl)sulfamoyl)benzoic acid;
4-(N-(3-Chloro-5-(trifluoromethyl)pyridin-2-yl)-N-(4-methoxybenzyl)sulfamoyl)benzoic acid;
4-(N-(3-Chloro-5-(trifluoromethyl)pyridin-2-yl)-N-(4-fluorobenzyl)sulfamoyl)benzoic acid;
4-(N-(4-tert-Butylbenzyl)-N-(3-chloro-5-(trifluoromethyl)pyridin-2-yl)sulfamoyl)benzoic acid;
4-(N-(3-Chloro-5-(trifluoromethyl)pyridin-2-yl)-N-(2-cyclohexylethyl)sulfamoyl)benzoic acid;
4-(N-(3-Chloro-5-(trifluoromethyl)pyridin-2-yl)-N-(2-fluorobenzyl)sulfamoyl)benzoic acid;
4-(N-(3-Chloro-5-(trifluoromethyl)pyridin-2-yl)-N-(3-(trifluoromethyl)benzyl)sulfamoyl)benzoic acid;
4-(N-(3-Chloro-5-(trifluoromethyl)pyridin-2-yl)-N-(3-methoxybenzyl)sulfamoyl)benzoic acid;
4-(N-(3-Chloro-5-(trifluoromethyl)pyridin-2-yl)-N-(3-(trifluoromethoxy)benzyl)sulfamoyl)benzoic acid;
4-(N-(3-Chloro-5-(trifluoromethyl)pyridin-2-yl)-N-(2,4-difluorobenzyl)sulfamoyl)benzoic acid;
4-(N-(3-Chloro-5-(trifluoromethyl)pyridin-2-yl)-N-(4-isopropylbenzyl)sulfamoyl)benzoic acid;
4-(N-(3-Chloro-5-(trifluoromethyl)pyridin-2-yl)-N-(2-(4-fluorophenoxy)ethyl)sulfamoyl)benzoic acid;
4-(N-(3-Chloro-5-(trifluoromethyl)pyridin-2-yl)-N-(2-(trifluoromethyl)benzyl)sulfamoyl)benzoic acid;
4-(N-(3-Chloro-5-(trifluoromethyl)pyridin-2-yl)-N-(4-chlorobenzyl)sulfamoyl)benzoic acid;
4-(N-(3-Chloro-5-(trifluoromethyl)pyridin-2-yl)-N-(4-cyanobenzyl)sulfamoyl)benzoic acid;
4-(N-Benzyl-N-(3,5-dichloropyridin-2-yl)sulfamoyl)benzoic acid;
4-(N-Benzyl-N-(5-chloro-3-methylpyridin-2-yl)sulfamoyl)benzoic acid;
4-(N-(3-Chloro-5-(trifluoromethyl)pyridin-2-yl)-N-(4-(trifluoromethyl)benzyl)sulfamoyl)benzoic acid;
4-(N-(2-Chloro-4-fluorobenzyl)-N-(3-chloro-5-(trifluoromethyl)pyridin-2-yl)sulfamoyl)benzoic acid;
4-(N-(3-Chloro-5-(trifluoromethyl)pyridin-2-yl)-N-(4-(2,2,2-trifluoroethoxy)benzyl)sulfamoyl)benzoic acid;
4-(N-(3-Chloro-5-(trifluoromethyl)pyridin-2-yl)-N-(3,5-difluorobenzyl)sulfamoyl)benzoic acid;
4-(N-(3-Chloro-5-(trifluoromethyl)pyridin-2-yl)-N-(4-fluoro-3-(trifluoromethyl)benzyl)sulfamoyl)benzoic acid;
4-(N-(3-Chloro-5-(trifluoromethyl)pyridin-2-yl)-N-(3,4-difluorobenzyl)sulfamoyl)benzoic acid;
4-(N-(3-Chloro-5-(trifluoromethyl)pyridin-2-yl)-N-(2,5-difluorobenzyl)sulfamoyl)benzoic acid;
4-(N-(3-Chloro-5-(trifluoromethyl)pyridin-2-yl)-N-(3,4-dichlorobenzyl)sulfamoyl)benzoic acid;
4-(N-(3-Chloro-5-(trifluoromethyl)pyridin-2-yl)-N-(3-chlorobenzyl)sulfamoyl)benzoic acid;
4-(N-(3-Chloro-5-(trifluoromethyl)pyridin-2-yl)-N-(4-(1-methylcyclopropyl)benzyl)sulfamoyl)benzoic acid;
4-(N-(3-Chloro-5-(trifluoromethyl)pyridin-2-yl)-N-(4-(1,1,1-trifluoro-2-methylpropan-2-yl)benzyl)sulfamoyl)benzoic acid;
4-(N-(3-Chloro-5-(trifluoromethyl)pyridin-2-yl)-N-(2-(trifluoromethoxy)benzyl)sulfamoyl)benzoic acid;
4-(N-(3,5-Dichloropyridin-2-yl)-N-(4-(trifluoromethyl)benzyl)sulfamoyl)benzoic acid;
4-(N-(5-Chloro-3-methylpyridin-2-yl)-N-(4-(trifluoromethyl)benzyl)sulfamoyl)benzoic acid;
4-(N-Benzyl-N-(2-chloro-4-(trifluoromethyl)phenyl)sulfamoyl)benzoic acid;
4-(N-(3-Chloro-5-(trifluoromethyl)pyridin-2-yl)-N-(3-phenylpropyl)sulfamoyl)benzoic acid;
4-(N-(3,5-Dichloropyridin-2-yl)-N-(4-fluoro-3-(trifluoromethyl)benzyl)sulfamoyl)benzoic acid;
4-(N-(3,5-Dichloropyridin-2-yl)-N-(4-fluorobenzyl)sulfamoyl)benzoic acid;
4-(N-(4-Chloro-3-fluorobenzyl)-N-(3,5-dichloropyridin-2-yl)sulfamoyl)benzoic acid;
4-(N-(3,5-Dichloropyridin-2-yl)-N-(4-(trifluoromethoxy)benzyl)sulfamoyl)benzoic acid;
4-(N-(3,5-Dichloropyridin-2-yl)-N-(2-(trifluoromethyl)benzyl)sulfamoyl)benzoic acid;
4-(N-(4-Chloro-3-fluorobenzyl)-N-(3-chloro-5-(trifluoromethyl)pyridin-2-yl)sulfamoyl)benzoic acid;
4-(N-(4-Chloro-3-(trifluoromethyl)benzyl)-N-(3,5-dichloropyridin-2-yl)sulfamoyl)benzoic acid;
4-(N-(3,5-Dichloropyridin-2-yl)-N-(3-fluoro-4-methylbenzyl)sulfamoyl)benzoic acid;
4-(N-(3,5-Dichloropyridin-2-yl)-N-(4-methyl-3-(trifluoromethyl)benzyl)sulfamoyl)benzoic acid;
4-(N-(3-Chloro-4-fluorobenzyl)-N-(3,5-dichloropyridin-2-yl)sulfamoyl)benzoic acid;
4-(N-(3,5-Dichloropyridin-2-yl)-N-(4-(1-methylcyclopropyl)benzyl)sulfamoyl)benzoic acid;
4-(N-(3-Chloro-4-fluorobenzyl)-N-(3-chloro-5-(trifluoromethyl)pyridin-2-yl)sulfamoyl)benzoic acid;
4-(N-(4-Chloro-2-(trifluoromethyl)benzyl)-N-(3,5-dichloropyridin-2-yl)sulfamoyl)benzoic acid;
4-(N-(4-Chloro-2-(trifluoromethyl)benzyl)-N-(5-chloro-3-methylpyridin-2-yl)sulfamoyl)benzoic acid;
4-(N-(5-Chloro-3-methylpyridin-2-yl)-N-(4-fluoro-3-(trifluoromethyl)benzyl)sulfamoyl)benzoic acid;
4-(N-(5-Chloro-3-methylpyridin-2-yl)-N-(3-chloro-4-fluorobenzyl)sulfamoyl)benzoic acid;
4-(N-(5-Chloro-3-methylpyridin-2-yl)-N-(4-(trifluoromethoxy)benzyl)sulfamoyl)benzoic acid;
4-(N-(4-Chloro-3-(trifluoromethyl)benzyl)-N-(5-chloro-3-methylpyridin-2-yl)sulfamoyl)benzoic acid;
4-(N-(3-Chloro-5-(trifluoromethyl)pyridin-2-yl)-N-(4-(trimethylsilyl)benzyl)sulfamoyl)benzoic acid;
4-(N-(3-Chloro-5-(trifluoromethyl)pyridin-2-yl)-N-(4-(1-cyanocyclopropyl)benzyl)sulfamoyl)benzoic acid;
4-(N-(3-Chloro-5-(trifluoromethyl)pyridin-2-yl)-N-((1-(pyridin-2-yl)piperidin-4-yl)methyl)sulfamoyl)benzoic acid;
4-(N-Benzyl-N-(3-chloro-5-(trifluoromethyl)pyridin-2-yl)sulfamoyl)-2-methylbenzoic acid;
N-Benzyl-N-(3-chloro-5-(trifluoromethyl)pyridin-2-yl)-6-hydroxypyridine-3-sulfonamide;
4-(N-Benzyl-N-(3-chloro-5-(trifluoromethyl)pyridin-2-yl)sulfamoyl)-3-methylbenzoic acid;
4-(N-Benzyl-N-(3-chloro-5-(trifluoromethyl)pyridin-2-yl)sulfamoyl)-3-methoxybenzoic acid;
2-(4-(N-Benzyl-N-(3-chloro-5-(trifluoromethyl)pyridin-2-yl)sulfamoyl)phenyl)acetic acid;
4-(N-(3-Chloro-5-(trifluoromethyl)pyridin-2-yl)-N-phenethylsulfamoyl)benzoic acid;
4-(N-Benzyl-N-(3-cyclopropyl-5-(trifluoromethyl)pyridin-2-yl)sulfamoyl)benzoic acid;
4-(N-(3-Chloro-5-(trifluoromethyl)pyridin-2-yl)-N-(4-cyclopropylbenzyl)sulfamoyl)benzoic acid;
4-(N-Benzyl-N-(3-bromo-5-(trifluoromethyl)pyridin-2-yl)sulfamoyl)benzoic acid;
4-(N-(3-Chloro-5-(trifluoromethyl)pyridin-2-yl)-N-(4-(1-methyl-1H-pyrazol-4-yl)benzyl)sulfamoyl)benzoic acid;
4-(N-Benzyl-N-(3-methyl-5-(trifluoromethyl)pyridin-2-yl)sulfamoyl)benzoic acid;
4-(N-(3-Chloro-5-(trifluoromethyl)pyridin-2-yl)-N-(4-(pyridin-3-yl)benzyl)sulfamoyl)benzoic acid;
4-(N-(3-Chloro-5-(trifluoromethyl)pyridin-2-yl)-N-(4-(thiophen-2-yl)benzyl)sulfamoyl)benzoic acid;
4-(N-(3-Chloro-5-(trifluoromethyl)pyridin-2-yl)-N-(4-(pyridin-4-yl)benzyl)sulfamoyl)benzoic acid;
4-(N-(3-Chloro-5-(trifluoromethyl)pyridin-2-yl)-N-(4-(furan-2-yl)benzyl)sulfamoyl)benzoic acid;
4-(N-([1,1′-Biphenyl]-4-ylmethyl)-N-(3-chloro-5-(trifluoromethyl)pyridin-2-yl)sulfamoyl)benzoic acid;
4-(N-(3-Chloro-5-(trifluoromethyl)pyridin-2-yl)-N-(4-(oxazol-5-yl)benzyl)sulfamoyl)benzoic acid;
4-(N-(3-Chloro-5-(trifluoromethyl)pyridin-2-yl)-N-((6-(trifluoromethyl)pyridin-3-yl)methyl)sulfamoyl)benzoic acid;
4-(N-(3-Chloro-5-(trifluoromethyl)pyridin-2-yl)-N-(4-(picolinamido)benzyl)sulfamoyl)benzoic acid;
4-(N-(3-Chloro-5-(trifluoromethyl)pyridin-2-yl)-N-(4-(6-methoxypyridin-3-yl)benzyl)sulfamoyl)benzoic acid;
4-(N-(3-Chloro-5-(trifluoromethyl)pyridin-2-yl)-N-(4-(6-methylpyridin-3-yl)benzyl)sulfamoyl)benzoic acid;
4-(N-([1,1′-Biphenyl]-3-ylmethyl)-N-(3-chloro-5-(trifluoromethyl)pyridin-2-yl)sulfamoyl)benzoic acid;
4-(N-([1,1′-Biphenyl]-2-ylmethyl)-N-(3-chloro-5-(trifluoromethyl)pyridin-2-yl)sulfamoyl)benzoic acid;
(R)-4-(N-(3-Chloro-5-(trifluoromethyl)pyridin-2-yl)-N-(1-phenylethyl)sulfamoyl)benzoic acid;
4-(N-(3-Chloro-5-(trifluoromethyl)pyridin-2-yl)-N-(thiophen-2-ylmethyl)sulfamoyl)benzoic acid;
4-(N-(3-Chloro-5-(trifluoromethyl)pyridin-2-yl)-N-(cyclohexylmethyl)sulfamoyl)benzoic acid;
4-(N-(3-Chloro-5-(trifluoromethyl)pyridin-2-yl)-N-(4-phenoxybenzyl)sulfamoyl)benzoic acid;
4-(N-(4-(1H-Pyrazol-1-yl)benzyl)-N-(3-chloro-5-(trifluoromethyl)pyridin-2-yl)sulfamoyl)benzoic acid;
4-(N-(3-Chloro-5-(trifluoromethyl)pyridin-2-yl)-N-(cyclobutylmethyl)sulfamoyl)benzoic acid;
4-(N-(3-Chloro-5-(trifluoromethyl)pyridin-2-yl)-N-(thiophen-3-ylmethyl)sulfamoyl)benzoic acid;
N-Benzyl-N-(3-chloro-5-(trifluoromethyl)pyridin-2-yl)-4-cyanobenzenesulfonamide;
N-Benzyl-N-(3-chloro-5-(trifluoromethyl)pyridin-2-yl)-4-(2H-tetrazol-5-yl)benzenesulfonamide;
N-Benzyl-N-(3-chloro-5-(trifluoromethyl)pyridin-2-yl)-4-methoxybenzenesulfonamide;
N-Benzyl-N-(3-chloro-5-(trifluoromethyl)pyridin-2-yl)-4-(methylsulfonamidomethyl)benzenesulfonamide;
N-(4-(N-Benzyl-N-(3-chloro-5-(trifluoromethyl)pyridin-2-yl)sulfamoyl)benzyl)acetamide;
N-Benzyl-N-(3-chloro-5-(trifluoromethyl)pyridin-2-yl)-4-(((N,N-dimethylsulfamoyl)amino)methyl)benzenesulfonamide;
N-Benzyl-N-(3-chloro-5-(trifluoromethyl)pyridin-2-yl)-4-((3,3-dimethylureido)methyl)benzenesulfonamide;
4-(N-Benzyl-N-(5-bromo-3-chloropyridin-2-yl)sulfamoyl)benzoic acid;
4-(N-Benzyl-N-(3-chloro-5-(trifluoromethyl)pyridin-2-yl)sulfamoyl)-3-chlorobenzoic acid;
4-(N-Benzyl-N-(3-chloro-5-phenylpyridin-2-yl)sulfamoyl)benzoic acid;
4-(N-Benzyl-N-(3-chloro-5-(furan-2-yl)pyridin-2-yl)sulfamoyl)benzoic acid;
4-(N-Benzyl-N-(3-chloro-5-(thiophen-3-yl)pyridin-2-yl)sulfamoyl)benzoic acid;
4-(N-Benzyl-N-(3-chloro-5-(2-methoxyphenyl)pyridin-2-yl)sulfamoyl)benzoic acid;
4-(N-Benzyl-N-(3-chloro-5-(4-methoxyphenyl)pyridin-2-yl)sulfamoyl)benzoic acid;
4-(N-Benzyl-N-(3-chloro-5-(3-methoxyphenyl)pyridin-2-yl)sulfamoyl)benzoic acid;
4-(N-Benzyl-N-(3-chloro-5-(trifluoromethyl)pyridin-2-yl)sulfamoyl)-2-chlorobenzoic acid;
N-Benzyl-N-(3-chloro-5-(trifluoromethyl)pyridin-2-yl)-6-methoxypyridine-3-sulfonamide;
4-(N-Benzyl-N-(3-chloro-5-(trifluoromethyl)pyridin-2-yl)sulfamoyl)-3-fluorobenzoic acid;
4-(N-Benzyl-N-(3-chloro-5-(trifluoromethyl)pyridin-2-yl)sulfamoyl)-2-fluorobenzoic acid;
4-(N-(3-Chloro-5-(trifluoromethyl)pyridin-2-yl)-N-(cyclopentylmethyl)sulfamoyl)benzoic acid;
4-(N-(3-Chloro-5-(trifluoromethyl)pyridin-2-yl)-N-(1-phenylcyclopropyl)sulfamoyl)benzoic acid;
N-Benzyl-N-(3-chloro-5-(trifluoromethyl)pyridin-2-yl)-4-((N,N-dimethylsulfamoyl)amino)benzenesulfonamide;
N-Benzyl-N-(3-chloro-5-(trifluoromethyl)pyridin-2-yl)-4-ureidobenzenesulfonamide;
N-Benzyl-N-(3-chloro-5-(trifluoromethyl)pyridin-2-yl)-4-(sulfamoylamino)benzenesulfonamide;
(S)-4-(N-(3-Chloro-5-(trifluoromethyl)pyridin-2-yl)-N-(1-phenylethyl)sulfamoyl)benzoic acid;
4-(N-(3-Chloro-5-(trifluoromethyl)pyridin-2-yl)-N-((2-phenylthiazol-4-yl)methyl)sulfamoyl)benzoic acid;
4-(N-(3-Chloro-5-(trifluoromethyl)pyridin-2-yl)-N-((5-phenyl-1,2,4-oxadiazol-3-yl)methyl)sulfamoyl)benzoic acid;
4-(N-Benzyl-N-(3-chloro-5-(trifluoromethyl)pyridin-2-yl)sulfamoyl)benzoic acid; and
N-Benzyl-N-(3-chloro-5-(trifluoromethyl)pyridin-2-yl)-4-(hydroxymethyl)benzene-1-sulfonamide.

9. A compound of formula (IVa), 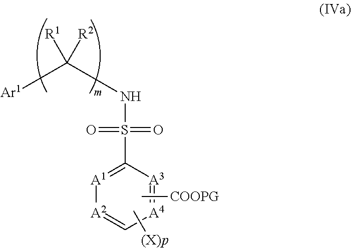
wherein
R¹ and R² are independently selected from the group consisting of hydrogen, C₁-C₄ alkyl, hydroxy C₁-C₄ alkyl, C₁-C₄ alkoxy C₁-C₄ alkyl, and C₃-C₇ cycloalkyl; or alternatively R¹ and R², together with the atom to which they are attached, form a 3 to 6 membered ring which may contain oxygen and/or nitrogen; said ring is optionally substituted with 1 to 4 substituents independently selected from the group consisting of halogen, hydroxy, C₁-C₄ alkyl, and C₁-C₄ alkoxy;
m is 0;
A¹, A², A³ and A⁴ are carbon atom;
Ar¹ is 2-pyridinyl or 3-pyridinyl; which may optionally be substituted with halogen, C₁-C₄ alkyl, C₁-C₄ haloalkyl, hydroxy, C₁-C₄ alkoxy, C₁-C₄ haloalkoxy, C₁-C₄ alkylthio, nitro, amino, C₁-C₄ alkylamino, di(C₁-C₄ alkyl)amino, cyano, hydroxy C₁-C₄ alkyl, C₁-C₄ alkoxy C₁-C₄ alkyl, C₁-C₄ alkylsulfonyl, aminosulfonyl, C₁-C₄ alkyl C(═O)—, HO(O═)C—, C₁-C₄ alkyl-O(O═)C—, R⁹N(R¹⁰⁾C(═O)—, C₁-C₄ alkylsulfonylamino, C₃-C₇ cycloalkyl, R⁹C(═O)N(R¹⁰)—, NH₂(HN═)C—, or 5 to 10 membered aryl C₀-C₄ alkyl; said aryl may be optionally substituted with 1 to 5 substituents independently selected from the group consisting of hydroxy, halogen, C₁-C₄ alkyl, C₁-C₄ alkoxy, hydroxy C₁-C₄ alkyl, amino C₁-C₄ alkyl, C₁-C₄ haloalkyl, C₁-C₄ haloalkoxy, C₃-C₇ cycloalkyl, amino, C₁-C₄ alkylamino, di(C₁-C₄ alkyl)amino, C₁-C₄ alkylthio, and nitro;
R⁹ and R¹⁰ are independently selected from hydrogen, C₁-C₄ alkyl, hydroxy C₁-C₄ alkyl, C₁-C₄ alkoxy C₁-C₄ alkyl, amino C₁-C₄ alkyl, C₁-C₄ alkylamino C₁-C₄ alkyl, di(C₁-C₄ alkyl)amino C₁-C₄ alkyl, 5 to 10 membered aryl, 5 to 10 membered aryl C₀-C₄ alkyl;
said aryl may be optionally substituted with 1 to 5 substituents independently selected from the group consisting of hydroxy, halogen, C₁-C₄ alkyl, C₁-C₄ alkoxy, hydroxy C₁-C₄ alkyl, amino C₁-C₄ alkyl, C₁-C₄ haloalkyl, C₁-C₄ haloalkoxy, C₃-C₈ cycloalkyl, amino, C₁-C₄ alkylamino, di(C₁-C₄ alkyl)amino, C₁-C₄ alkylthio, and nitro;
C₃-C₈ cycloalkyl, and 3 to 8 membered heterocyclyl C₁-C₄ alkyl;
said heterocyclyl and alkyl may have independently 1 to 4 substituents independently selected from C₁-C₄ alkyl and halogen;
or alternatively R⁹ and R¹⁰ together with nitrogen atom to which they are attached form a 4 to 8 membered ring which may contain nitrogen, oxygen or sulfur, wherein the 4 to 8 membered ring is optionally substituted with 1 to 6 substituents independently selected from the group consisting of hydroxy, C₁-C₄ alkyl, C₁-C₄ alkoxy, C₃-C₇ cycloalkyl, amino, oxo, C₁-C₄ alkylamino, and di(C₁-C₄ alkyl)amino;
X is independently selected from halogen and C₁-C₄ alkyl;
p is 1, 2, 3, or 4; when p is two or more than two, X may be same or different;
PG is a protecting group;
or a pharmaceutically acceptable salt thereof.

10. A pharmaceutical composition comprising the compound or the pharmaceutically acceptable salt thereof, as claimed in claim 5, and a pharmaceutically acceptable carrier.

11. A pharmaceutical composition as claimed in claim 10, further comprising another pharmacologically active agent.

12. (canceled)
